(19) United States
(12) Patent Application Publication
(10) Pub. No.: US 2012/0129846 A1
(43) Pub. Date: May 24, 2012

(54) GAMMA SECRETASE MODULATORS

(76) Inventors: Zhaoning Zhu, Plainsboro, NJ (US); William J. Greenlee, Teaneck, NJ (US); Mihir Baran Mandal, Westfield, NJ (US); Duane A. Burnett, Bernardsville, NJ (US); Chad E. Bennett, Metuchen, NJ (US); Troy McCracken, Garwood, NJ (US)

(21) Appl. No.: 13/126,680

(22) PCT Filed: Nov. 5, 2009
(86) PCT No.: PCT/US2009/063385

§ 371 Date: Dec. 8, 2011

Related U.S. Application Data
(60) Provisional application No. 61/111,829, filed on Nov. 6, 2008.

(51) Int. Cl. A61K 31/5386; A61K 31/53; A61K 31/55; C07D 498/04; A61K 31/5383; A61P 25/28; A61K 31/438; C07D 471/10; C07D 498/10; C07D 513/04; C07D 403/10; C07D 471/04; A61K 31/437

(52) U.S. Cl. 514/230.2; 544/183; 514/243; 514/303; 540/554; 544/105; 514/230.5; 546/119; 514/278; 546/15; 544/70; 546/114; 514/301; 540/501; 540/543; 544/182; 514/242

(57) ABSTRACT

In its many embodiments, the present invention provides a novel class of heterocyclic compounds of the formula: as modulators of gamma secretase, methods of preparing such compounds, pharmaceutical compositions containing one or more such compounds, methods of preparing pharmaceutical formulations comprising one or more such compounds, and methods of treatment, prevention, inhibition, or amelioration of one or more diseases associated with the central nervous system using such compounds or pharmaceutical compositions.

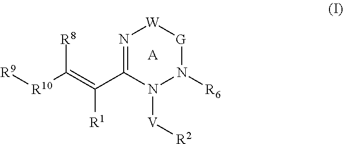

### REFERENCE TO RELATED APPLICATION

This application claims the benefit of U.S. Provisional Application No. 61/111,838 filed Nov. 6, 2008.

### FIELD OF THE INVENTION

The present invention relates to certain heterocyclic compounds useful as gamma secretase modulators (including inhibitors, antagonists and the like), pharmaceutical compositions containing the compounds, and methods of treatment using the compounds and compositions to treat various diseases including central nervous system disorders such as, for example, neurodegenerative diseases such as Alzheimer's disease and other diseases relating to the deposition of amyloid protein. They are especially useful for reducing Amyloid beta (hereinafter referred to as Aβ) production which is effective in the treatment of diseases caused by Aβ such as, for example, Alzheimers and Down Syndrome.

### BACKGROUND OF THE INVENTION

Alzheimer's disease is a disease characterized by degeneration and loss of neurons and also by the formation of senile plaques and neurofibrillary change. Presently, treatment of Alzheimer's disease is limited to symptomatic therapies with a symptom-improving agent represented by an acetylcholinesterase inhibitor, and the basic remedy which prevents progress of the disease has not been developed. A method of controlling the cause of onset of pathologic conditions needs to be developed for creation of the basic remedy of Alzheimer's disease.

Aβ protein, which is a metabolite of amyloid precursor protein (hereinafter referred to as APP), is considered to be greatly involved in degeneration and loss of neurons as well as onset of demential conditions (for example, see Klein W L, et al Proceeding National Academy of Science USA, Sep. 2, 2003, 100(18), p. 10417-22, suggest a molecular basis for reversible memory loss.

Nitsch R M, and 16 others, Antibodies against β-amyloid slow cognitive decline in Alzheimer's disease, Neuron, May 22, 2003, 38(4), p. 547-554) suggest that the main components of Aβ protein are Aβ40 consisting of 40 amino acids and Aβ42 having two additional amino acids at the C-terminal. The Aβ40 and Aβ42 tend to aggregate (for example, see Jarrell J T et al, The carboxy terminus of the β amyloid protein is critical for the seeding of amyloid formation: implications for the pathogenesis of Alzheimer's disease, Biochemistry, May 11, 1993, 32(18), p. 4693-4697) and constitute main components of senile plaques (for example, (Glenner G G, et al, Alzheimer's disease: initial report of the purification and characterization of a novel cerebrovascular amyloid protein, Biochemical and Biophysical Research Communications, May 16, 1984, 120(3), p. 885-90. See also Masters C L, et al, Amyloid plaque core protein in Alzheimer disease and Down syndrome, Proceeding National Academy of Science USA, June 1985, 82(12), p. 4245-4249.).

Furthermore, it is known that mutations of APP and presenelin genes, which is observed in familial Alzheimer's disease, increase production of Aβ40 and Aβ42 (for example, see Gouras G K, et al, Intraneuronal Aβ142 accumulation in human brain, American Journal of Pathology, January 2000, 156(1), p. 15-20. Also, see Scheuner D, et al, Nature Medicine, August 1996, 2(8), p. 864-870; and Forman M S, et al, Differential effects of the Swedish mutant amyloid precursor protein on β-amyloid accumulation and secretion in neurons and normeuronal cells, Journal of Biological Chemistry, Dec. 19, 1997, 272(51), p. 32247-32253.). Therefore, compounds which reduce production of Aβ40 and Aβ42 are expected as an agent for controlling progress of Alzheimer's disease or for preventing the disease.

These Aβs are produced when APP is cleaved by beta secretase and subsequently clipped by gamma secretase. In consideration of this, creation of inhibitors of γ secretase and β secretase has been attempted for the purpose of reducing production of Aβs. Many of these secretase inhibitors already known are peptides or peptidomimetics such as L-685,458. L-685,458, an aspartyl protease transition stale mimic, is a potent inhibitor of amyloid β-protein precursor γ-secretase activity, Biochemistry, Aug. 1, 2000, 39(30), p. 8698-8704).

Also of interest in connection with the present invention are: US 2007/0117798 (Eisai, published May 24, 2007); US 2007/0117839 (Eisai, published May 24, 2007); US 2006/0004013 (Eisai, published Jan. 5, 2006); WO 2005/110422 (Boehringer Ingelheim, published Nov. 24, 2005); WO 2006/045554 (Cellzone A G, published May 4, 2006); WO 2004/110350 (Neurogenetics™, published Dec. 23, 2004); WO 2004/071431 (Myriad Genetics, published Aug. 26, 2004); US 2005/0042284 (Myriad Genetics, published Feb. 23, 2005) and WO 2006/001877 (Myriad Genetics, published Jan. 5, 2006).

There is a need for new compounds, formulations, treatments and therapies to treat diseases and disorders associated with Aβ. It is, therefore, an object of this invention to provide compounds useful in the treatment or prevention or amelioration of such diseases and disorders.

### SUMMARY OF THE INVENTION

In its many embodiments, the present invention provides a novel class of heterocyclic compounds as gamma secretase modulators (including inhibitors, antagonists and the like), methods of preparing such compounds, pharmaceutical compositions comprising one or more such compounds, methods of preparing pharmaceutical formulations comprising one or more such compounds, and methods of treatment, prevention, inhibition or amelioration of one or more diseases associated with the Aβ using such compounds or pharmaceutical compositions.

The compounds of this invention (Formula I) can be useful as gamma secretase modulators and can be useful in the treatment and prevention of diseases such as, for example, Alzheimers disease, mild cognitive impairment (MCI), Downs Syndrome, Glaucoma (Guo et. al., Proc. Natl. Acad. Sci. USA 104, 13444-13449 (2007)), Cerebral amyloid angiopathy, stroke or dementia (Frangione et al., Amyloid: J. Protein folding Disord. 8, suppl. 1, 36-42 (2001), Microgliosis and brain inflammation (M P Lamber, Proc. Natl. Acad. Sci. USA 95, 6448-53 (1998)), and Olfactory function loss (Getchell, et. al. Neurobiology of Aging, 663-673, 24, 2003).

This invention provides compounds of formula I:

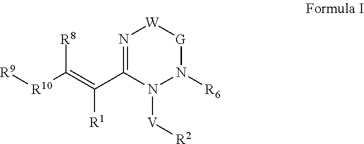

wherein R¹, R², R⁶, R⁸, R⁹, R¹⁰, G, V and W are as defined below.

The compounds of Formula I can be useful as gamma secretase modulators and can be useful in the treatment and prevention of diseases such as, for example, central nervous system disorders such as Alzheimers disease and Downs Syndrome.

This invention also provides compounds of formula I.

This invention also provides pharmaceutically acceptable salts of the compounds of formula I.

This invention also provides pharmaceutically acceptable esters of the compounds of formula I.

This invention also provides pharmaceutically solvates of the compounds of formula I.

The present invention further includes the compounds of formula I in all its isolated forms.

This invention also provides compounds of formula I in pure and isolated form.

This invention also provides compounds of formula I in pure form. This invention also provides compounds of formula I in isolated form.

This invention also provides compounds of formulas 3-50 as defined herein, formulas 51-53 as defined herein, A6-A17, D9-D20, E4-E21, F1-12, G6-G23, H1-H17, I4-I12, J5-J12, M6-M16, O11 and N6.

This invention also provides pharmaceutical compositions comprising an effective amount of one or more (e.g., one) compounds of formula I, or a pharmaceutically acceptable salt, ester or solvate thereof, and a pharmaceutically acceptable carrier.

This invention also provides pharmaceutical compositions comprising an effective amount of one or more (e.g., one) compounds of formula I, or a pharmaceutically acceptable salt, ester or solvate thereof, and an effective amount of one or more (e.g., one) other pharmaceutically active ingredients (e.g., drugs), and a pharmaceutically acceptable carrier.

The compounds of Formula (I) can be useful as gamma secretase modulators and can be useful in the treatment and prevention of diseases such as, for example, central nervous system disorders such as Alzheimers disease and Downs Syndrome.

Thus, this invention also provides methods for: (1) method for modulating (including inhibiting, antagonizing and the like) gamma-secretase; (2) treating one or more neurodegenerative diseases; (3) inhibiting the deposition of amyloid protein (e.g., amyloid beta protein) in, on or around neurological tissue (e.g., the brain); (4) Alzheimer's disease; and (5) treating Downs syndrome; wherein each method comprises administering an effective amount of one or more (e.g., one) compounds of formula (I) to a patient in need of such treatment.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of an effective amount of one or more (e.g. one) compounds of formula (I) and the administration of an effective amount of one or more (e.g., one) other pharmaceutical active ingredients (e.g., drugs).

This invention also provides methods for: (1) treating mild cognitive impairment; (2) treating glaucoma; (3) treating cerebral amyloid angiopathy; (4) treating stroke; (5) treating dementia; (6) treating microgliosis; (7) treating brain inflammation; and (8) treating olfactory function loss; wherein each method comprises administering an effective amount of one or more (e.g., one) compounds of formula (I) to a patient in need of such treatment.

This invention also provides a method of treating one or more neurodegenerative diseases, comprising administering an effective (i.e., therapeutically effective) amount of one or more compounds of formula I to a patient in need of treatment.

This invention also provides a method of treating one or more neurodegenerative diseases, comprising administering an effective (i.e., therapeutically effective) amount of a compound of formula I to a patient in need of treatment.

This invention also provides a method of inhibiting the deposition of amyloid protein (e.g., amyloid beta protein) in, on or around neurological tissue (e.g., the brain), comprising administering an effective (i.e., therapeutically effective) amount of one or more compounds of formula I to a patient in need of treatment.

This invention also provides a method of inhibiting the deposition of amyloid protein (e.g., amyloid beta protein) in, on or around neurological tissue (e.g., the brain), comprising administering an effective (i.e., therapeutically effective) amount of a compound of formula I to a patient in need of treatment.

This invention also provides a method of treating Alzheimer's disease, comprising administering an effective (i.e., therapeutically effective) amount of one or more compounds of formula I to a patient in need of treatment.

This invention also provides a method of treating Alzheimer's disease, comprising administering an effective (i.e., therapeutically effective) amount of a compound of formula I to a patient in need of treatment.

This invention also provides a method of treating Alzheimer's disease, comprising administering an effective (i.e., therapeutically effective) amount of one or more compounds of formula I, in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

This invention also provides a method of treating Alzheimer's disease, comprising administering an effective (i.e., therapeutically effective) amount of one or more compounds of formula I, in combination with an effective (i.e., therapeutically effective) amount of one or more compounds selected from the group consisting of Aβ antibody inhibitors, gamma secretase inhibitors and beta secretase inhibitors.

This invention also provides combinations comprising an effective (i.e., therapeutically effective) amount of one or more compounds of formula I, in combination with an effective (i.e., therapeutically effective) amount of one or more compounds selected from the group consisting of cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), Aβ antibody inhibitors, gamma secretase inhibitors and beta secretase inhibitors.

This invention also provides a method of treating Alzheimer's disease, comprising administering an effective (i.e., therapeutically effective) amount of a compound of formula I, in combination with an effective (i.e., therapeutically effective) amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

This invention also provides a method of treating Downs syndrome, comprising administering an effective (i.e., therapeutically effective) amount of one or more compounds of formula I to a patient in need of treatment.

This invention also provides a method of treating Downs syndrome, comprising administering an effective (i.e., therapeutically effective) amount of a compound of formula I to a patient in need of treatment.

This invention also provides a method of treating Downs syndrome, comprising administering an effective (i.e., therapeutically effective) amount of one or more compounds of formula I, in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

This invention also provides a method of treating Downs syndrome, comprising administering an effective (i.e., therapeutically effective) amount of a compound of formula I, in combination with an effective (i.e., therapeutically effective) amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of an effective amount of one or more (e.g. one) compounds of formula I and the administration of an effective amount of one or more (e.g., one) other pharmaceutical active ingredients (e.g., drugs).

This invention also provides a method of treating mild cognitive impairment, comprising administering an effective amount of one or more (e.g., one) compounds of formula I to a patient in need of treatment.

This invention also provides a method of treating glaucoma, comprising administering an effective amount of one or more (e.g., one) compounds of formula I to a patient in need of treatment.

This invention also provides a method of treating cerebral amyloid angiopathy, comprising administering an effective amount of one or more (e.g., one) compounds of formula I to a patient in need of treatment.

This invention also provides a method of treating stroke, comprising administering an effective amount of one or more (e.g., one) compounds of formula I to a patient in need of treatment.

This invention also provides a method of treating dementia, comprising administering an effective amount of one or more (e.g., one) compounds of formula I to a patient in need of treatment.

This invention also provides a method of treating microgliosis, comprising administering an effective amount of one or more (e.g., one) compounds of formula I to a patient in need of treatment.

This invention also provides a method of treating brain inflammation, comprising administering an effective amount of one or more (e.g., one) compounds of formula I to a patient in need of treatment.

This invention also provides a method of treating olfactory function loss, comprising administering an effective amount of one or more (e.g., one) compounds of formula I to a patient in need of treatment.

This invention also provides pharmaceutical compositions comprising a combination of an effective amount of one or more (e.g., one) compounds of formula I, or a pharmaceutically acceptable salt, solvate, or ester thereof, and at least one pharmaceutically acceptable carrier, and a therapeutically effective amount of one or more compounds selected from the group consisting of cholinesterase inhibitors, Aβ antibody inhibitors, gamma secretase inhibitors and beta secretase inhibitors. The pharmaceutical compositions also comprise a pharmaceutically acceptable carrier.

This invention also provides a kit comprising, in separate containers, in a single package, pharmaceutical compositions for use in combination, wherein one container comprises an effective amount of a compound of formula (I) in a pharmaceutically acceptable carrier, and another container (i.e., a second container) comprises an effective amount of another pharmaceutically active ingredient (as described below), the combined quantities of the compound of formula (I) and the other pharmaceutically active ingredient being effective to treat the diseases or conditions mentioned in any of the above methods.

This invention also provides a kit comprising, in separate containers, in a single package, pharmaceutical compositions for use in combination, wherein one container comprises an effective amount of a compound of formula (I) in a pharmaceutically acceptable carrier, and another container (i.e., a second container) comprises an effective amount of another pharmaceutically active ingredient (as described above), the combined quantities of the compound of formula (I) and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase, or (e) mild cognitive impairment, or (f) glaucoma, or (g) cerebral amyloid angiopathy, or (h) stroke, or (i) dementia, or (j) microgliosis, or (k) brain inflammation, or (I) olfactory function loss.

This invention also provides a kit comprising, in separate containers, in a single package, pharmaceutical compositions for use in combination, wherein one container comprises an effective amount of a compound of formula (I) in a pharmaceutically acceptable carrier, and another container (i.e., a second container) comprises an effective amount of another pharmaceutically active ingredient (as described above), the combined quantities of the compound of formula (I) and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase.

This invention also provides any one of the methods disclosed above and below wherein the compound is selected from the group consisting of the compounds formulas 3-50 as defined herein, formulas 51-53 as defined herein, A6-A17, D9-D20, E4-E21, F1-12, G6-G23, H1-H17, I4-I12, J5-J12, M6-M16, O11 and N6.

This invention also provides any one of the methods disclosed above and below wherein the compound of formula I is selected from the group consisting of the compounds formulas 3-50 as defined herein, formulas 51-53 as defined herein, A6-A17, D9-D20, E4-E21, F1-12, G6-G23, H1-H17, I4-I12, J5-J12, M6-M16, O11 and N6.

This invention also provides any one of the pharmaceutical compositions disclosed above and below wherein the compound is selected from the group consisting of the compounds formulas 3-50 as defined herein, formulas 51-53 as defined herein, A6-A17, D9-D20, E4-E21, F1-12, G6-G23, H1-H17, I4-I12, J5-J12, M6-M16, O11 and N6.

This invention also provides any one of the methods, pharmaceutical compositions or kits disclosed above and below wherein the compound is any one of the compounds formulas 3-50 as defined herein, formulas 51-53 as defined herein, A6-A17, D9-D20, E4-E21, F1-12, G6-G23, H1-H17, I4-I12, J5-J12, M6-M16, O11 and N6.

### DETAILED DESCRIPTION

In one embodiment, the present invention discloses compounds which are represented by structural Formula I, or a pharmaceutically acceptable salt, solvate, ester or prodrug thereof, wherein the various moieties are described below.

In one embodiment, the present application discloses a compound, or pharmaceutically acceptable salts, solvates, esters or prodrugs of said compound, said compound having the general structure shown in Formula I:

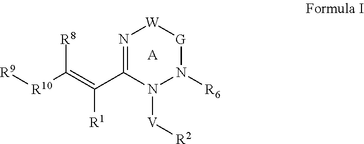

wherein:

either
(i) R¹ and R² are joined together to form 5-8 membered heterocyclyl or 5-8 membered heterocyclenyl moiety, wherein: (a) said heterocyclyl or heterocyclenyl moiety is optionally substituted with 1-5 independently selected R²¹ substituents, and (b) said heterocyclyl or heterocyclenyl moiety is optionally fused with an aryl or heteroaryl ring, and the ring moiety resulting from the fusion is optionally substituted with 1-5 independently selected R²¹ substituents; or
(ii) R² and R⁶ are joined together to form 5-8 membered heterocyclyl or 5-8 membered heterocyclenyl moiety, wherein: (a) said heterocyclyl or heterocyclenyl moiety is optionally substituted with 1-5 independently selected R²¹ substituents, and (b) said heterocyclyl or heterocyclenyl moiety is optionally fused with an aryl or heteroaryl ring, and the ring moiety resulting from the fusion is optionally substituted with 1-5 independently selected R²¹ substituents; or
(iii)
   (a) R¹ and R² are joined together to form 5-8 membered heterocyclyl or 5-8 membered heterocyclenyl moiety, wherein: said heterocyclyl or heterocyclenyl moiety is optionally substituted with 1-5 independently selected R²¹ substituents, and
   (b) R² and R⁶ are joined together to form 5-8 membered heterocyclyl or 5-8 membered heterocyclenyl moiety, wherein: said heterocyclyl or heterocyclenyl moiety is optionally substituted with 1-5 independently selected R²¹ substituents; and
   (c) said R² and R⁶ heterocyclyl or heterocyclenyl moiety is optionally fused with an aryl or heteroaryl ring, and the ring moiety resulting from the fusion is optionally substituted with 1-5 independently selected R²¹ substituents; or
(iv) R⁶ and one R³ of the —(CR³R⁴)_{1 or 2}-G moiety are joined together to form 5-8 membered heterocyclyl or 5-8 membered heterocyclenyl moiety, wherein: (a) said heterocyclyl or heterocyclenyl moiety is optionally substituted with 1-5 independently selected R²¹ substituents, and (b) said heterocyclyl or heterocyclenyl moiety is optionally fused with an aryl or heteroaryl ring, and the ring moiety resulting from the fusion is optionally substituted with 1-5 independently selected R²¹ substituents; or
(v) R¹ and R² are not joined together to form 5-8 membered heterocyclyl or 5-8 membered heterocyclenyl moiety, R² and R⁶ are not joined together to form 5-8 membered heterocyclyl or 5-8 membered heterocyclenyl moiety, and R⁶ and one R³ of the —(CR³R⁴)_{1 or 2}-G moiety are not joined together to form 5-8 membered heterocyclyl or 5-8 membered heterocyclenyl moiety; and

R¹ (when R¹ is not joined to R² and when R¹ does not together with R⁸ form a bond), R² (when R² is not joined to R¹ or R⁶), and R⁶ (when R⁶ is not joined to R² or R³) can be the same or different, each being independently selected from the group consisting of H, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each of said alkyl-, alkenyl- and alkynyl-, arylalkyl-, alkylaryl-, cycloalkyl-, cycloalkylalkyl-, heteroaryl-, heteroarylalkyl-, heterocyclyl- and heterocyclylalkyl- can be unsubstituted or optionally substituted with 1-5 independently selected R²¹ substituents;

or, alternatively, R¹ (when R¹ is not joined to R²) and R⁸ can be taken together to form a bond (i.e., there is a triple bond between the carbon atom to which R¹ was bonded to and the carbon to which R⁸ was bonded to, i.e., the compound of formula I is a compound of formula II:

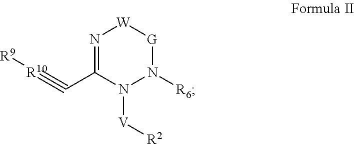

W is selected from the group consisting of a bond, —C(O)—, —S(O)—, —S(O₂)— and —(CR¹¹R¹²)_{1 or 2}—, e.g., —CR¹¹R¹²—, —CR¹¹R¹²—CH₂—, —CR¹¹R¹²—CR¹¹R¹² , and —CH₂—C(R¹¹)(R¹²)—, with the proviso that ring A is a 5-, 6- or 7-membered ring;

G is selected from the group consisting of —C(O)—, —S(O)—, —S(O₂)— and —(CR³R⁴)_{1 or 2}—, e.g., —CR³R⁴—, —CR³R⁴—CH₂—, —CR³R⁴— CR³R⁴—, and —CH₂—CR³R⁴—, with the provisos that ring A is a 5-, 6- or 7-membered ring and that no combination of W and G can be —C(O)—S(O)—, C(O)—S(O)₂—, —S(O)—C(O)—, —S(O)₂—C(O)—, —S(O)—S(O)—, S(O)—S(O)₂—, —S(O)₂—S(O)— or S(O)₂—S(O)₂—;

V is selected from the group consisting of a bond and —C(O)—;

each R³ (when R³ does not form a ring with R⁶ or with R⁴) can be the same or different and is independently selected from the group consisting of H, halo (and in one example, F), —OR¹⁵ (and in one example R¹⁵ is H), —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —C(O)R¹⁵, —C(O)OR¹⁵, —C(═NOR¹⁵)R¹⁶, —C(O)N(R¹⁵)(R¹⁶), —S(O)N(R¹⁵)(R¹⁶), S(O)₂N(R¹⁵)(R¹⁶), —S(O)R¹⁵, —S(O)₂R^{15A}, —P(O)(OR¹⁵)(OR¹⁶), ═NOR¹⁵, —N₃, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each of said alkyl-, alkenyl- and alkynyl-, aryl-, arylalkyl-, alkylaryl, cycloalkyl-, cycloalkylalkyl-, heteroaryl-, heteroarylalkyl-, heterocyclyl- and heterocyclylalkyl- can be unsubstituted or optionally substituted with 1-5 independently selected R²¹ substituents; or

each R⁴ (when R⁴ does not form a ring with R³), R¹¹ (when R¹¹ does not from a ring with R¹²) and R¹² (when R¹² does not for a ring with R¹¹) can be the same or different and is independently selected from the group consisting of H, halo (and in one example, F), —OR¹⁵ (and in one example R¹⁵ is H), —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —C(O)R¹⁵, —C(O)OR¹⁵, —C(═NOR¹⁵)R¹⁶, —C(O)N(R¹⁵)(R¹⁶), —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, —N₃, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- can be unsubstituted or optionally substituted with 1-5 independently selected R²¹ substituents;

alternatively, when W is —CR¹¹R¹²— and G is —CR³R⁴—, R³ (when R³ does not form a ring with R⁴ or R⁶) and R¹¹ (when R¹¹ does not form a ring with R¹²) can be joined together to form a bond;

alternatively, (a) R³ (when R³ does not form a ring with R⁶ or a bond with R¹¹) and R⁴ can be joined together to form a C₃-C₈ spirocycloalkyl, C₄-C₈ spirocycloalkenyl, 5-8 membered spiroheterocyclyl or 5-8 membered spiroheterocyclenyl moiety, with each of said spirocycloalkyl or spiroheterocyclyl or spirocycloalkenyl moiety being unsubstituted or optionally substituted with 1-5 independently selected R²¹ substituents, or (b) R¹¹ and R¹² can be joined together to form a C₃-C₈ spirocycloalkyl, C₄-C₈ spirocycloalkenyl, 5-8 membered spiroheterocyclyl or 5-8 membered spiroheterocyclenyl moiety, with each of said spirocycloalkyl or spiroheterocyclyl or spirocycloalkenyl moiety being unsubstituted or optionally substituted with 1-5 independently selected R²¹ substituents, and (c) with the proviso that ring A can have only one C₃-C₈ spirocycloalkyl, C₄-C₈ spirocycloalkenyl, 5-8 membered spiroheterocyclyl or 5-8 membered spiroheterocyclenyl moiety;

provided that when one of R³ or R⁴ is selected from the group consisting of: —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R¹⁵)(R¹⁶), —S(O)R¹⁵, —S(O)₂R^{15A}—P(O)(OR¹⁵)(OR¹⁶), ═NOR¹⁵, and —N₃, then the other is not selected from the group consisting of: —OR¹⁵, —CN, —SR¹⁵, and —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)²N(R¹⁵)(R¹⁶), —S(O)R¹⁵, —S(O)₂R^{15A}, —P(O)(OR¹⁵)(OR¹⁶), ═NOR¹⁵, and —N₃ (i.e., if one of R³ or R⁴ is —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)²N(R¹⁵)(R¹⁶), —S(O)R¹⁵, —S(O)₂R^{15A}, —P(O)(OR¹⁵)(OR¹⁶), ═NOR¹⁵, or —N₃, then the other one is not —OR¹⁵, —CN, —SR¹⁵, and —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, or —N₃);

provided that when one of R¹¹ or R¹² is selected from the group consisting of: —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, and —N₃, then the other is not selected from the group consisting of: —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, and —N₃ (i.e., if one of R¹¹ or R¹² is —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷) —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)²N(R¹⁵)(R¹⁶), —S(O)R¹⁵, —S(O)₂R^{15A}, —P(O)(OR¹⁵)(OR¹⁶), ═NOR¹⁵, or —N₃, then the other is not —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)²N(R¹⁵)(R¹⁶), —S(O)R¹⁵, —S(O)₂R^{15A}, —P(O)(OR¹⁵)(OR¹⁶), ═NOR¹⁵, or —N₃);

R⁸ (when R¹ is not joined to R⁸) is selected from the group consisting of H, alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl-, with each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- being unsubstituted or optionally substituted with 1-3 independently selected R²¹ substituents;

R⁹ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each R⁹ group is optionally substituted with 1-3 independently selected R²¹ substituents;

R¹⁰ is selected from the group consisting of a bond, alkyl-, alkenyl-, alkynyl-, aryl-, arylalkyl-, alkylaryl-, cycloalkyl-, cycloalkylalkyl-, heteroaryl-, heteroarylalkyl-, heterocyclyl-, heterocyclylalkyl- and the moieties:

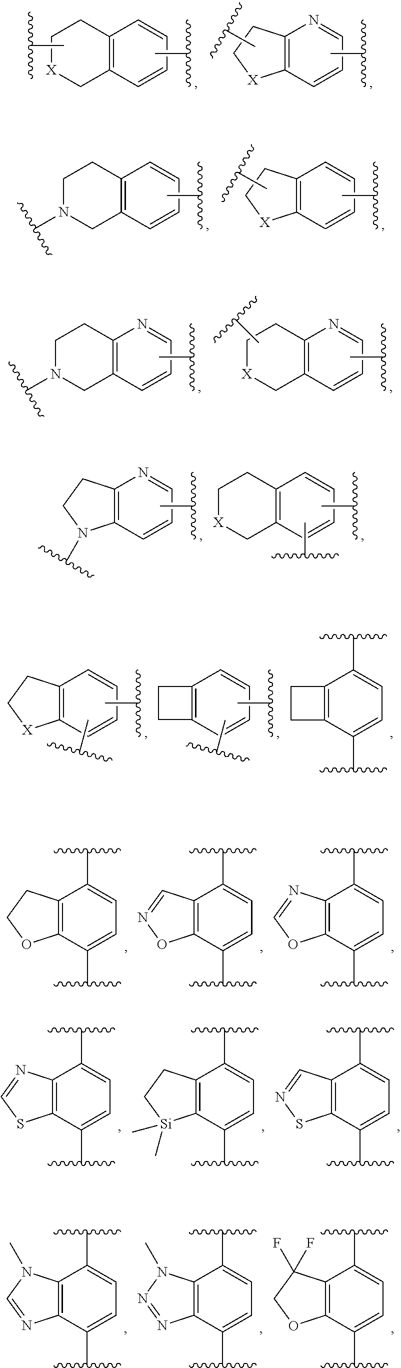

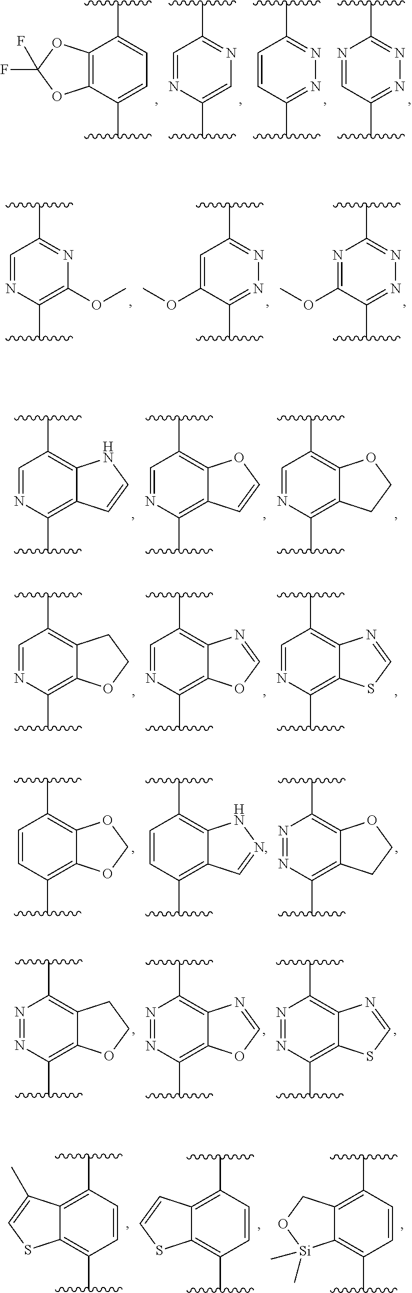

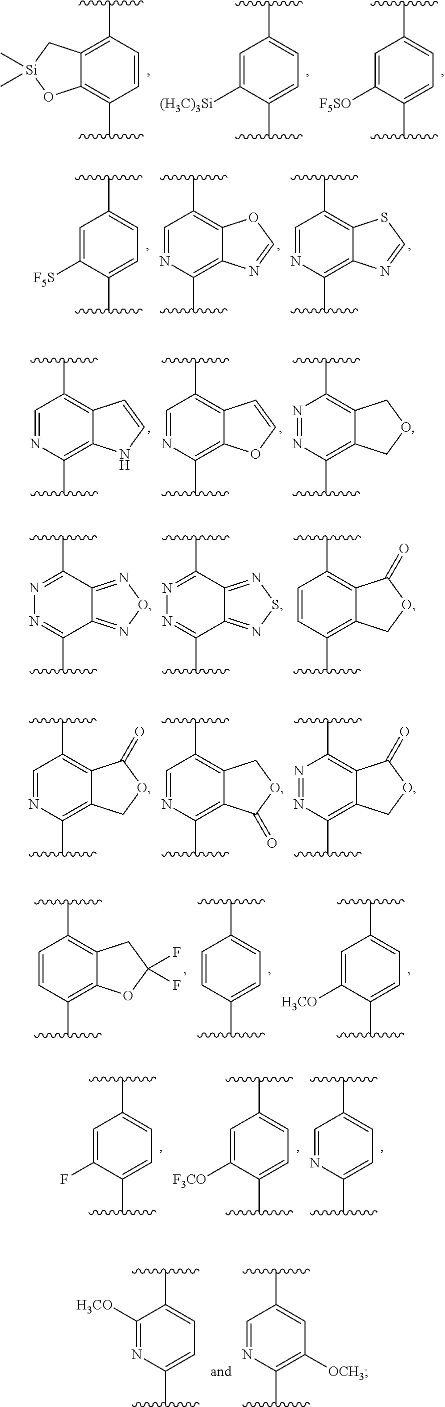

wherein X is O, N(R¹⁴) or S and wherein each R¹⁹ group (except for the bond) is optionally substituted with 1-3 independently selected R²¹ substituents;

R¹⁴ is selected from the group consisting of H, alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), C(═NOR¹⁵)R¹⁶, and —P(O)(OR¹⁵)(OR¹⁶), wherein each of said alkyl, alkenyl, alkynyl, cycloalkyl, cycloalkylalkyl-, cycloalkenyl, heterocyclyl, heterocyclylalkyl-, aryl, arylalkyl-, heteroaryl, and heteroarylalkyl- is optionally substituted with 1-5 independently selected R²¹ substitutents;

R^{15A} is selected from the group consisting of alkyl, alkenyl, alkynyl, cycloalkyl, cycloalkylalkyl-, heterocyclyl, heterocyclylalkyl-, aryl, arylalkyl-, heteroaryl, heteroarylalkyl-, arylcycloalkyl-, arylheterocyclyl-, (R¹⁸)ᵣ-alkyl, (R¹⁸)ᵣ-cycloalkyl, (R¹⁸)ᵣ-cycloalkylalkyl-, (R¹⁸)ᵣ-heterocyclyl, (R¹⁸)ᵣ-heterocyclylalkyl-, (R¹⁸)ᵣ-aryl, (R¹⁸)ᵣ-arylalkyl-, (R¹⁸)ᵣ-heteroaryl and (R¹⁸)ᵣ-heteroarylalkyl-; wherein r is 1-5;

R¹⁵, R¹⁶ and R¹⁷ can be the same or different and are each independently selected from the group consisting of H, alkyl, alkenyl, alkynyl, cycloalkyl, cycloalkylalkyl-, heterocyclyl, heterocyclylalkyl-, aryl, arylalkyl-, heteroaryl, heteroarylalkyl-, arylcycloalkyl-, arylheterocyclyl-, (R¹⁸)ᵣ-alkyl, (R¹⁸)ᵣ-cycloalkyl, (R¹⁸)ᵣ-cycloalkylalkyl-, (R¹⁸)ᵣ-heterocyclyl, (R¹⁸)ᵣ-heterocyclylalkyl-, (R¹⁸)ᵣ-aryl, (R¹⁸)ᵣ-arylalkyl-, (R¹⁸)ᵣ-heteroaryl and (R¹⁸)ᵣ-heteroarylalkyl-; wherein r is 1-5;

each R¹⁸ is independently selected from the group consisting of alkyl, alkenyl, alkynyl, aryl, arylalkyl-, arylalkenyl-, arylalkynyl-, —NO₂, halo, heteroaryl, HO-alkyoxyalkyl-, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

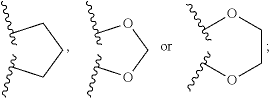

R¹⁹ is selected from the group consisting of: alkyl, cycloalkyl, aryl, arylalkyl- and heteroarylalkyl-;

R²⁰ is selected from the group consisting of: alkyl, cycloalkyl, aryl, halo substituted aryl, arylalkyl-, heteroaryl and heteroarylalkyl-;

each R²¹ group is independently selected from the group consisting of alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, halo, —CN, —OR¹⁵, —C(O)R¹⁵, —C(O)OR¹⁶, —C(O)N(R¹⁵)(R¹⁶), —SF₅, —OSF₅, —Si(R^{15A})₃, —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁶)C(O)R¹⁶, —CH₂—N(R¹⁵)C(O)R¹⁶, —CH₂—N(R¹⁶)C(O)N(R¹⁶)(R¹⁷), —CH₂—R¹⁵; —CH₂N(R¹⁵)(R¹⁶), —N(R¹⁵)S(O)R¹⁶, —N(R¹⁶)S(O)₂R¹⁶, —CH₂—N(R¹⁶)S(O)₂R¹⁶, —N(R¹⁵)S(O)₂N(R¹⁶)(R¹⁷), —N(R¹⁶)S(O)N(R¹⁶)(R¹⁷), —N(R₁₆)C(O)N(R₁₆)(R₁₇), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁶, —N₃, —NO₂ and —S(O)₂R^{15A}; and wherein each of the R²¹ alkyl, cycloalkenyl, cycloalkyl, cycloalkylalkyl-, heterocyclyl, heterocyclylalkyl-, aryl, arylalkyl-, heteroaryl, heteroarylalkyl-, alkenyl and alkynyl groups is optionally substituted with 1 to 5 independently selected R²² groups; and

each R²² is independently selected from the group consisting of: alkyl, cycloalkyl, cycloalkenyl, heterocyclyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SF₅, —OSF₅, —Si(R^{15A})₃, —SR¹⁵, —S(O)N(R¹⁵)(R¹⁶), —S(O)₂N(R¹⁵)(R¹⁶), —C(═NOR¹⁵)R¹⁶, —P(O)(OR¹⁶)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁶)S(O)₂R¹⁶, —CH₂—N(R¹⁶)S(O)₂R¹⁶, —N(R¹⁶)S(O)₂N(R¹⁶)(R¹⁷), —N(R¹⁶)S(O)N(R¹⁶)(R¹⁷), —N(R¹⁶)C(O)N(R¹⁶)(R¹⁷), —CH₂—N(R¹⁶)C(O)N(R¹⁶)(R¹⁷), —N(R¹⁵)C(O)OR¹⁶, —CH₂—N(R¹⁵)C(O)OR¹⁶, —N₃, ═NOR¹⁶, —NO₂, —S(O)R¹⁵ and —S(O)₂R^{15A}.

Preferably, in the embodiment described immediately above, R⁹ is selected from the group consisting of alkyl-, alkenyl-, alkynyl-, aryl-, arylalkyl-, alkylaryl, cycloalkyl-, cycloalkylalkyl-, heteroaryl-, heteroarylalkyl-, heterocyclyl- and heterocyclylalkyl-, wherein each R⁹ group is optionally substituted with 1-3 independently selected R²¹ substituents.

In another embodiment, the present application discloses a compound, or pharmaceutically acceptable salts, solvates, esters or prodrugs of said compound, said compound having the general structure shown in Formula I:

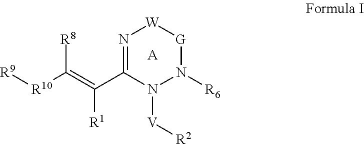

wherein:

R¹ and R² are joined together to form 5-8 membered heterocyclyl or 5-8 membered heterocyclenyl moiety, wherein: (a) said heterocyclyl or heterocyclenyl moiety is optionally substituted with 1-5 independently selected R²¹ substituents, and (b) said heterocyclyl or heterocyclenyl moiety is optionally fused with an aryl or heteroaryl ring, and the ring moiety resulting from the fusion is optionally substituted with 1-5 independently selected R²¹ substituents;

R⁶ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- can be unsubstituted or optionally substituted with 1-5 independently selected R²¹ substituents;

W is selected from the group consisting of a bond, —C(O)—, —S(O)—, —S(O₂)— and —(CR¹¹R¹²)_{1 or 2}—, e.g., —CR¹¹R¹²—, —CR¹¹R¹²—CR¹¹R¹²—, and —CH₂—C(R¹¹)(R¹²)—, with the proviso that ring A is a 5-, 6- or 7-membered ring;

G is selected from the group consisting of —C(O)—, —S(O)—, —S(O₂)— and —(CR³R⁴)_{1 or 2}—) e.g., —CR³R⁴—, —CR³R⁴—CH₂—, —CR³R⁴— CR³R⁴—, and —CH₂—CR³R⁴—, with the provisos that ring A is a 5-, 6- or 7-membered ring and that no combination of W and G can be —C(O)—S(O)—, C(O)—S(O)₂—, —S(O)—C(O)—, —S(O)₂—C(O)—, —S(O)—S(O)—, S(O)—S(O)₂—, —S(O)₂—S(O)— or S(O)₂—S(O)₂—;

V is selected from the group consisting of a bond and —C(O)—;

each R³ (when R³ does not form a ring with R⁴) can be the same or different and is independently selected from the group consisting of H, halo (and in one example, F), —OR¹⁵ (and in one example R¹⁵ is H), —CN, —SR¹⁵, —NR¹⁵R¹⁶, —N(R¹⁵)C(O)R¹⁶, —N(R¹⁵)S(O)R¹⁶, —N(R¹⁶)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —C(O)R¹⁵, —C(O)OR¹⁵, —C(═NOR¹⁵)R¹⁶, —C(O)N(R¹⁵)(R¹⁶), —S(O)N(R¹⁵)(R¹⁶), S(O)₂N(R¹⁵)(R¹⁶), —S(O)R¹⁵, —S(O)₂R^{15A}, —P(O)(OR¹⁵)(OR¹⁶), ═NOR¹⁵, —N₃, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- can be unsubstituted or optionally substituted with 1-5 independently selected R²¹ substituents; or

each R⁴ (when R⁴ does not form a ring with R³), R¹¹ (when R¹¹ does not from a ring with R¹²) and R¹² (when R¹² does not for a ring with R¹¹) can be the same or different and is independently selected from the group consisting of H, halo (and in one example, F), —OR¹⁵ (and in one example R¹⁵ is H), —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —C(O)R¹⁵, —C(O)OR¹⁵, —C(═NOR¹⁵)R¹⁶, —C(O)N(R¹⁵)(R¹⁶), —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, —N₃, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- can be unsubstituted or optionally substituted with 1-5 independently selected R²¹ substituents;

alternatively, when W is —CR¹¹R¹²— and G is —CR³R⁴—, R³ (when R³ does not form a ring with R⁴ or R⁶) and R¹¹ (when R¹¹ does not form a ring with R¹²) can be joined together to form a bond;

alternatively, (a) R³ (when R³ does not form a ring with R⁶ or a bond with R¹¹) and R⁴ can be joined together to form a C₃-C₈ spirocycloalkyl, C₄-C₈ spirocycloalkenyl, 5-8 membered spiroheterocyclyl or 5-8 membered spiroheterocyclenyl moiety, with each of said spirocycloalkyl or spiroheterocyclyl or spirocycloalkenyl moiety being unsubstituted or optionally substituted with 1-5 independently selected R²¹ substituents, or (b) R¹¹ and R¹² can be joined together to form a C₃-C₈ spirocycloalkyl, C₄-C₈ spirocycloalkenyl, 5-8 membered spiroheterocyclyl or 5-8 membered spiroheterocyclenyl moiety, with each of said spirocycloalkyl or spiroheterocyclyl or spirocycloalkenyl moiety being unsubstituted or optionally substituted with 1-5 independently selected R²¹ substituents, and (c) with the proviso that ring A can have only one C₃-C₈ spirocycloalkyl, C₄-C₈ spirocycloalkenyl, 5-8 membered spiroheterocyclyl or 5-8 membered spiroheterocyclenyl moiety;

provided that when one of R³ or R⁴ is selected from the group consisting of: —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R¹⁵)(R¹⁶), —S(O)₂N(R¹⁵)(R¹), —S(O)R¹⁵, —S(O)₂R^{15A}, —P(O)(OR¹⁵)(OR¹⁶), ═NOR¹⁵, and —N₃, then the other is not selected from the group consisting of: —OR¹⁵, —CN, —SR¹⁵, and —NR—N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, and —N₃ (i.e., if one of R³ or R⁴ is —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, or —N₃, then the other one is not —OR¹⁵, —CN, —SR¹⁵, and —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷)^{, —N(R}¹⁵)^{C}(O)^{N}(R¹⁶)(R¹⁷), —N(R¹⁵)C(O)OR¹⁶, —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, or —N₃);

provided that when one of R¹¹ or R¹² is selected from the group consisting of: —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, and —N₃, then the other is not selected from the group consisting of: —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R₁₅)S(O)N(R¹⁶)(R¹⁷), —N(R¹⁵)C(O)N(R¹⁶)(R¹⁷), —N(R¹⁵)C(O)OR¹⁶), —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, and —N₃ (i.e., if one of R¹¹ or R¹² is −OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R₁₅)(R₁₆), —S(O)R¹⁵, —S(O)₂R^{15A}, —P(O)(OR¹⁵)(OR¹⁶), ═NOR¹⁵, or —N₃, then the other is not —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁶, or —N₃);

R⁸ (when R¹ is not joined to R⁸) is selected from the group consisting of H, alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl-, with each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- being unsubstituted or optionally substituted with 1-3 independently selected R²¹ substituents;

R⁹ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each R⁹ group is optionally substituted with 1-3 independently selected R²¹ substituents;

R¹⁰ is selected from the group consisting of a bond, alkyl-, alkenyl-, alkynyl-, aryl-, arylalkyl-, alkylaryl-, cycloalkyl-, cycloalkylalkyl-, heteroaryl-, heteroarylalkyl-, heterocyclyl-, heterocyclylalkyl- and the moieties:

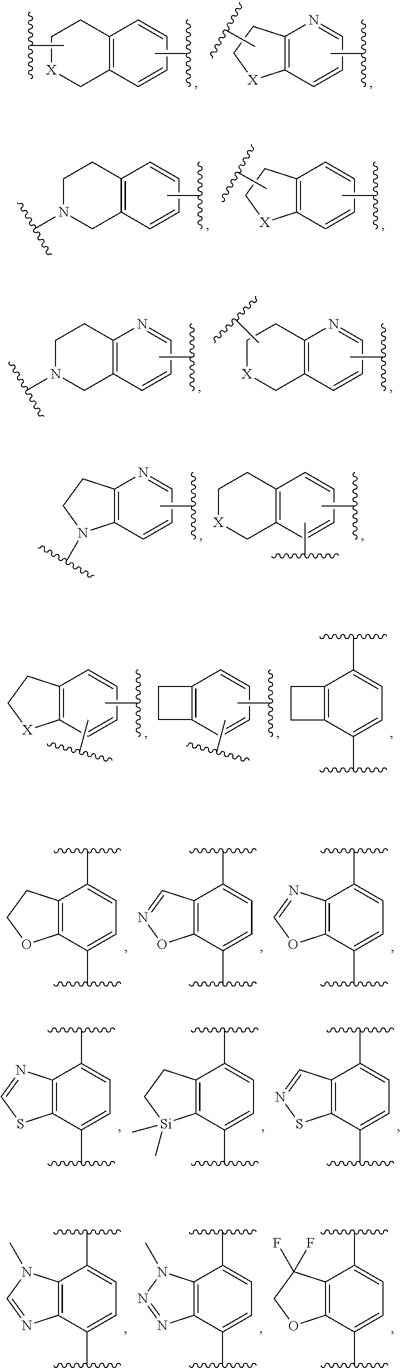

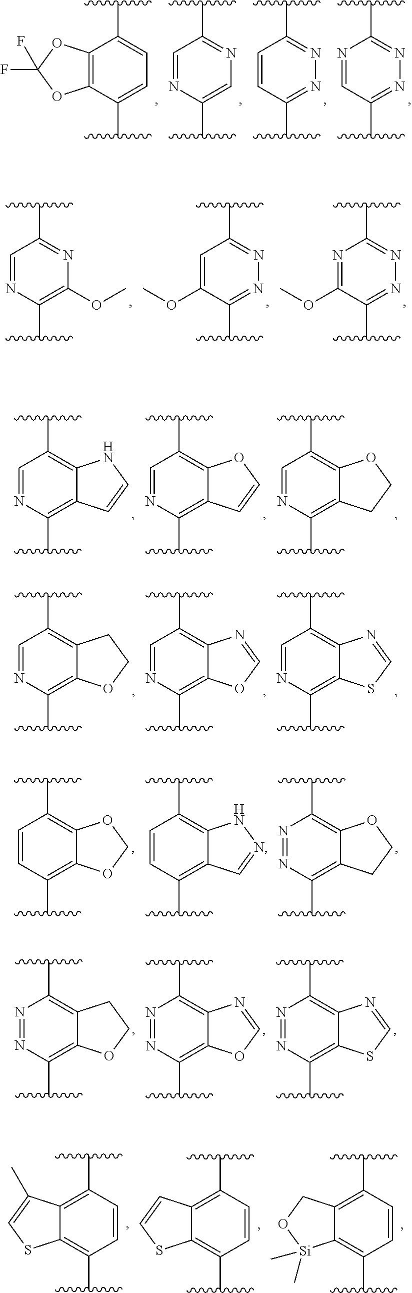

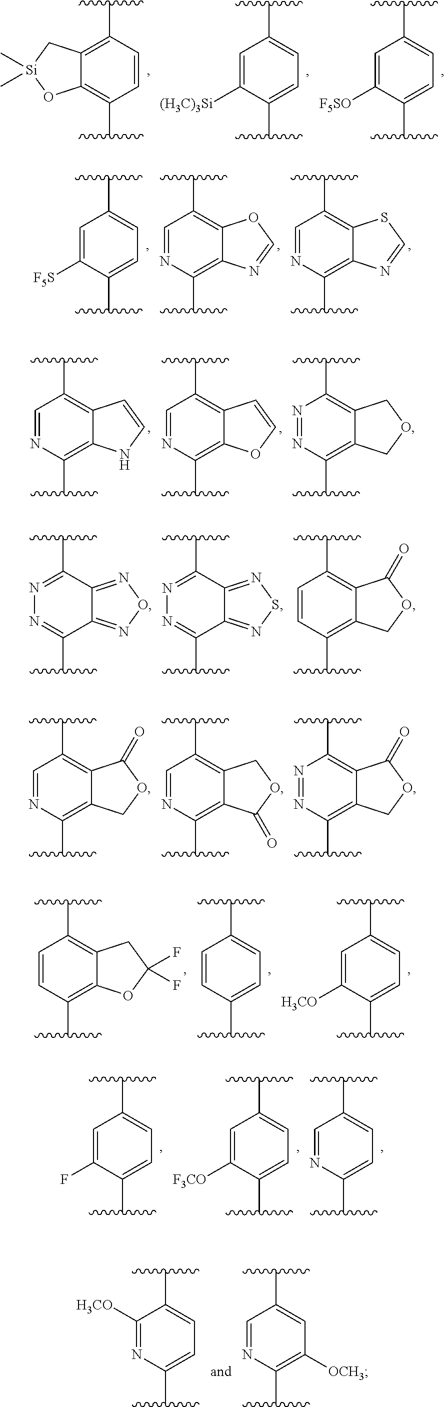

wherein X is O, N(R¹⁴) or S and wherein each R¹⁰ group (except for the bond) is optionally substituted with 1-3 independently selected R²¹ substituents;

R¹⁴ is selected from the group consisting of H, alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁶)(OR¹⁶), wherein each of said alkyl, alkenyl, alkynyl, cycloalkyl, cycloalkylalkyl-, cycloalkenyl, heterocyclyl, heterocyclylalkyl-, aryl, arylalkyl-, heteroaryl, and heteroarylalkyl- is optionally substituted with 1-5 independently selected R²¹ substitutents;

R¹⁵, R¹⁶ and R¹⁷ can be the same or different and are each independently selected from the group consisting of H, alkyl, alkenyl, alkynyl, cycloalkyl, cycloalkylalkyl-, heterocyclyl, heterocyclylalkyl-, aryl, arylalkyl-, heteroaryl, heteroarylalkyl-, arylcycloalkyl-, arylheterocyclyl-, (R¹⁸)ᵣ-alkyl, (R¹⁸)ᵣ-cycloalkyl, (R¹⁸)ᵣ-cycloalkylalkyl-, (R¹⁸)ᵣ-heterocyclyl, (R¹⁸)ᵣ-heterocyclylalkyl-, (R¹⁸)ᵣ-aryl, (R¹⁸)ᵣ-arylalkyl-, (R¹⁸)ᵣ-heteroaryl and (R¹⁸)ᵣ-heteroarylalkyl-; wherein r is 1-5;

each R¹⁸ is independently selected from the group consisting of alkyl, alkenyl, alkynyl, aryl, arylalkyl-, arylalkenyl-, arylalkynyl-, —NO₂, halo, heteroaryl, HO-alkyoxyalkyl-, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

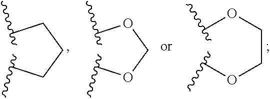

R¹⁹ is selected from the group consisting of: alkyl, cycloalkyl, aryl, arylalkyl- and heteroarylalkyl-;

R²⁰ is selected from the group consisting of: alkyl, cycloalkyl, aryl, halo substituted aryl, arylalkyl-, heteroaryl and heteroarylalkyl-;

each R²¹ group is independently selected from the group consisting of alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, halo, —CN, —OR¹⁵, —C(O)R¹⁵, —C(O)OR¹⁵, —C(O)N(R¹⁵)(R¹⁶), —SF₅, —OSF₅, —Si(R^{15A})₃, —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁸)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁶, —N₃, —NO₂ and —S(O)₂R^{15A}; and wherein each of the R²¹ alkyl, cycloalkenyl, cycloalkyl, cycloalkylalkyl-, heterocyclyl, heterocyclylalkyl-, aryl, arylalkyl-, heteroaryl, heteroarylalkyl-, alkenyl and alkynyl groups is optionally substituted with 1 to 5 independently selected R²² groups; and

each R²² is independently selected from the group consisting of: alkyl, cycloalkyl, cycloalkenyl, heterocyclyl, aryl, heteroaryl, halo, —CF₃, —CN, —OR¹⁸, —C(O)R¹⁵, —C(O)OR¹⁵, -alkyl-C(O)OR¹⁵, C(O)N(R¹⁵)(R¹⁶), —SF₅, —OSF₅, —Si(R^{15A})₃, —SR¹⁵, —S(O)N(R¹⁵)(R¹⁶), —S(O)²N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁶, —NO₂, —S(O)R¹⁵ and —S(O)₂R^{15A}.

Preferably, in the embodiment described immediately above, R⁹ is selected from the group consisting of alkyl-, alkenyl-, alkynyl-, aryl-, arylalkyl-, alkyla cycloalkyl-, cycloalkylalkyl-, heteroaryl-, heteroarylalkyl-, heterocyclyl- and heterocyclylalkyl-, wherein each R⁹ group is optionally substituted with 1-3 independently selected R²¹ substituents.

In another embodiment, the present application discloses a compound, or pharmaceutically acceptable salts, solvates, esters or prodrugs of said compound, said compound having the general structure shown in Formula I:

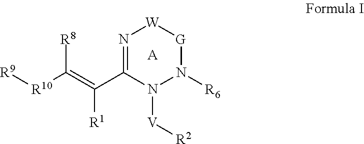

wherein:

R² and R⁶ are joined together to form 5-8 membered heterocyclyl or 5-8 membered heterocyclenyl moiety, wherein: (a) said heterocyclyl or heterocyclenyl moiety is optionally substituted with 1-5 independently selected R²¹ substituents, and (b) said heterocyclyl or heterocyclenyl moiety is optionally fused with an aryl or heteroaryl ring, and the ring moiety resulting from the fusion is optionally substituted with 1-5 independently selected R²¹ substituents;

R¹ (when R¹ does not together with R⁸ form a bond) is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- can be unsubstituted or optionally substituted with 1-5 independently selected R²¹ substituents;

or, alternatively, R¹ and R⁸ can be taken together to form a bond (i.e., there is a triple bond between the carbon atom to which R¹ was bonded to and the carbon to which R⁸ was bonded to, i.e., the compound of formula I is a compound of formula II:

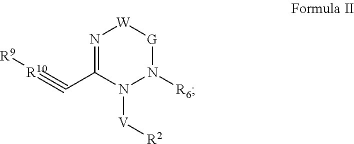

W is selected from the group consisting of a bond, —C(O)—, —S(O)—, —S(O₂)— and —(CR¹¹R¹²)_{1 or 2}—, e.g., —CR¹¹R¹², —CR¹¹R¹²—CH₂—, —CR¹¹R¹²—CR¹¹R¹²—, and —CH₂—C(R¹¹)(R¹²)—, with the proviso that ring A is a 5-, 6- or 7-membered ring;

G is selected from the group consisting of —C(O)—, —S(O)—, —S(O₂)— and —(CR³R⁴)_{1 or 2}—, e.g., —CR³R⁴—, —CR³R⁴—CH₂—, —CR³R⁴— CR³R⁴—, and —CH₂—CR³R⁴—, with the provisos that ring A is a 5-, 6- or 7-membered ring and that no combination of W and G can be —C(O)—S(O)—, C(O)—S(O)₂—, —S(O)—C(O)—, —S(O)₂—C(O)—, —S(O)—S(O)—, S(O)—S(O)₂—, —S(O)₂—S(O)— or S(O)₂—S(O)₂—;

V is selected from the group consisting of a bond and —C(O)—;

each R³ (when R³ does not form a ring with R⁴) can be the same or different and is independently selected from the group consisting of H, halo (and in one example, F), —OR¹⁵ (and in one example R¹⁵ is H), —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —C(O)R¹⁵, —C(O)OR¹⁵, —C(═NOR¹⁵)R¹⁶, —C(O)N(R¹⁵)(R¹⁶), —S(O)N(R¹⁵)(R¹⁶), S(O)₂N(R¹⁵)(R¹⁶), —S(O)R¹⁵, —S(O)₂R^{15A}, —P(O)(OR¹⁵)(OR¹⁶), ═NOR¹⁵, —N₃, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- can be unsubstituted or optionally substituted with 1-5 independently selected R²¹ substituents; or

each R⁴ (when R⁴ does not form a ring with R³), R¹¹ (when R¹¹ does not from a ring with R¹²) and R¹² (when R¹² does not for a ring with R¹¹) can be the same or different and is independently selected from the group consisting of H, halo (and in one example, F), —OR¹⁵ (and in one example R¹⁵ is H), —CN, —SR¹⁵, —NR¹⁵R¹⁶, —N(R¹⁵)C(O)R¹⁶, —N(R¹⁵)S(O)R¹⁶)(R¹⁷), —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —C(O)R¹⁵, —C(O)OR¹⁵, —C(═NOR¹⁵)R¹⁶, —C(O)N(R¹⁵)(R¹⁶), —S(O)N(R¹⁵)(R₁₆), —S(O)₂N(R₁₅)(R¹⁶), —S(O)R¹⁵, —S(O)₂R^{15A}, —P(P)(OR₁₅)(OR¹⁶), ═NOR¹⁵, —N₃, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- can be unsubstituted or optionally substituted with 1-5 independently selected R²¹ substituents;

alternatively, when W is —CR¹¹R¹²— and G is —CR³R⁴—, R³ (when R³ does not form a ring with R⁶) and R¹¹ (when R¹¹ does not form a ring with R¹²) can be joined together to form a bond;

alternatively, (a) R³ (when R³ does not form or a bond with R¹¹) and R⁴ can be joined together to form a C₃-C₈ spirocycloalkyl, C₄-C₈ spirocycloalkenyl, 5-8 membered spiroheterocyclyl or 5-8 membered spiroheterocyclenyl moiety, with each of said spirocycloalkyl or spiroheterocyclyl or spirocycloalkenyl moiety being unsubstituted or optionally substituted with 1-5 independently selected R²¹ substituents, or (b) R¹¹ and R¹² can be joined together to form a C₃-C₈ spirocycloalkyl, C₄-C₈ spirocycloalkenyl, 5-8 membered spiroheterocyclyl or 5-8 membered spiroheterocyclenyl moiety, with each of said spirocycloalkyl or spiroheterocyclyl or spirocycloalkenyl moiety being unsubstituted or optionally substituted with 1-5 independently selected R²¹ substituents, and (c) with the proviso that ring A can have only one C₃-C₈ spirocycloalkyl, C₄-C₈ spirocycloalkenyl, 5-8 membered spiroheterocyclyl or 5-8 membered spiroheterocyclenyl moiety;

provided that when one of R³ or R⁴ is selected from the group consisting of: —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R₁₅)S(O)₂N(R¹⁶)(R¹⁷), —N(R¹⁵)S(C)N(R¹⁶)(R¹⁷), C(O)N(R¹⁶)(R¹⁷), —N(R¹⁵)C(O)OR¹⁶, —S(O)N(R¹⁵)(R¹⁶), —S(O)²N(R¹⁵)(R¹⁶), —S(O)R¹⁵, —S(O)₂R^{15A}—P(O)(OR¹⁵)(OR¹⁶), ═NOR¹⁵, and —N₃, then the other is not selected from the group consisting of: —OR¹⁵, —CN, —SR¹⁵, and —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, and —N₃ (i.e., if one of R³ or R⁴ is —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R¹⁵)(R¹⁶), —S(O)R¹⁵, —S(O)₂R^{15A}—P(O)(OR¹⁵)(OR¹⁶), ═NOR¹⁵, or —N₃, then the other one is not —OR¹⁵, —CN, —SR¹⁵, and —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵C(O)N(R¹⁶)(R¹⁷), —N(R¹⁵)C(O)OR¹⁶, —S(O)N(R¹⁵)(R¹⁶), —S(O)²N(R¹⁵)(R¹⁶), —S(O)R¹⁵, —S(O)₂R^{15A}—P(O)(OR¹⁵)(CR¹⁶), ═NOR¹⁵, or —N₃);

provided that when one of R¹¹ or R¹² is selected from the group consisting of: —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, and —N₃, then the other is not selected from the group consisting of: —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)²N(R¹⁵)(R¹⁶), —S(O)R¹⁵, —S(O)₂R^{15A}, —P(O)(OR¹⁵)(OR¹⁶), ═NOR¹⁵, and —N₃ (i.e., if one of R¹¹ or R¹² is —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)²N(R¹⁵)(R¹⁶), —S(O)R¹⁵, —S(O)₂R^{15A}, —P(O)(OR¹⁵)(OR¹⁶), NOR¹⁵, or —N₃, then the other is not —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R₁₅)(R₁₆), —S(O)R¹⁵, —S(O)₂R^{15A}, —P(O)(OR¹⁵)(OR¹⁶), NOR¹⁵, or —N₃);

R⁸ (when R¹ is not joined to R⁸) is selected from the group consisting of H, alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl-, with each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- being unsubstituted or optionally substituted with 1-3 independently selected R²¹ substituents;

R⁹ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each R⁹ group is optionally substituted with 1-3 independently selected R²¹ substituents;

R¹⁰ is selected from the group consisting of a bond, alkyl-, alkenyl-, alkynyl-, aryl-, arylalkyl-, alkylaryl-, cycloalkyl-, cycloalkylalkyl-, heteroaryl-, heteroarylalkyl-, heterocyclyl-, heterocyclylalkyl- and the moieties:

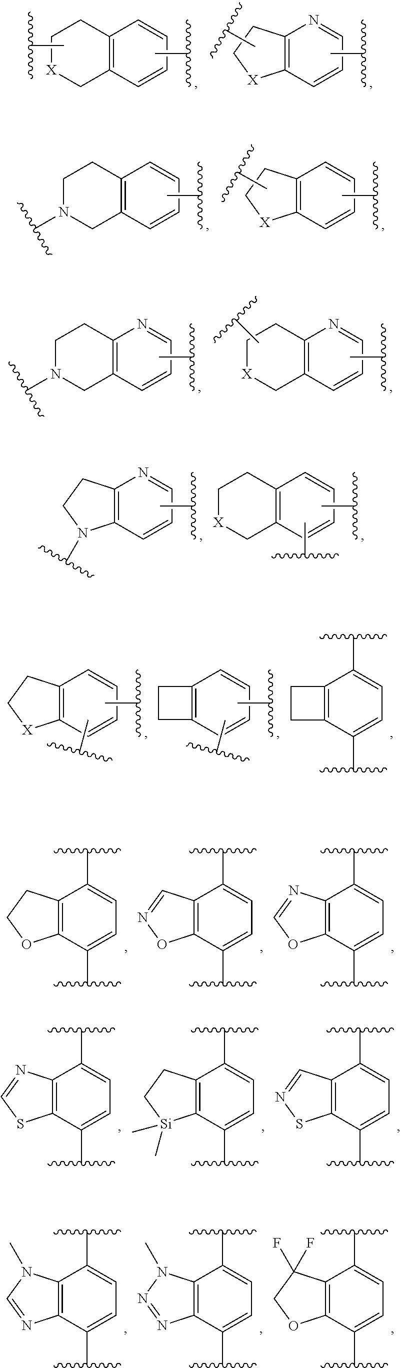

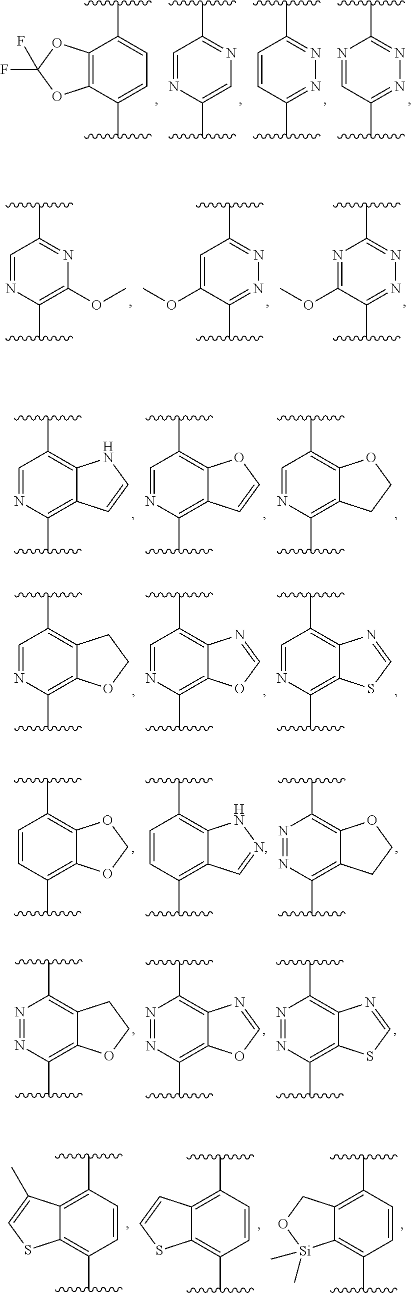

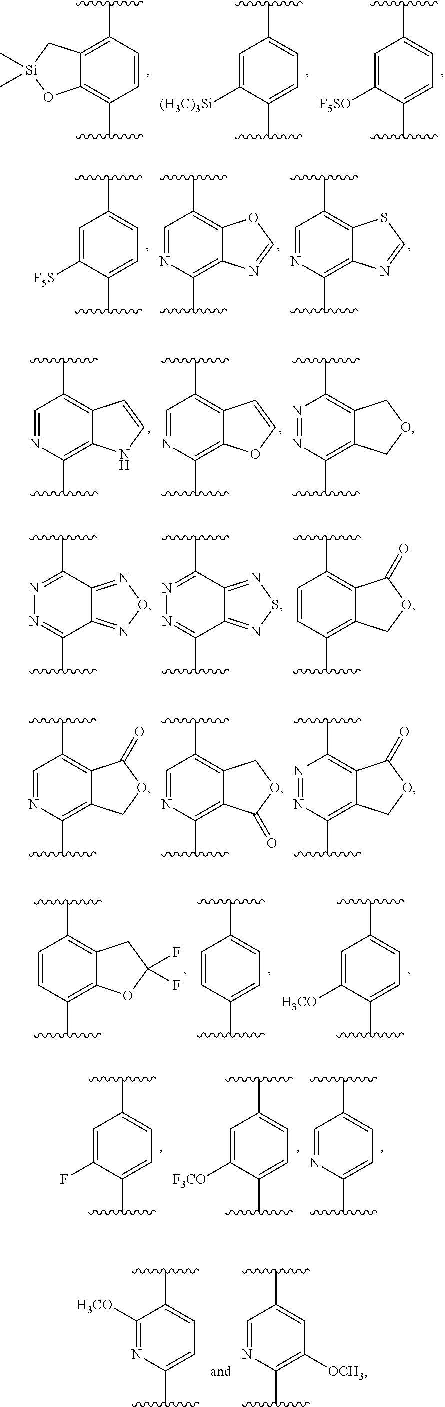

wherein X is O, N(R¹⁴) or S and wherein each R¹⁰ group (except for the bond) is optionally substituted with 1-3 independently selected R²¹ substituents;

R¹⁴ is selected from the group consisting of H, alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶), wherein each of said alkyl, alkenyl, alkynyl, cycloalkyl, cycloalkylalkyl-, cycloalkenyl, heterocyclyl, heterocyclylalkyl-, aryl, arylalkyl-, heteroaryl, and heteroarylalkyl- is optionally substituted with 1-5 independently selected A²¹ substitutents;

R¹⁵, R¹⁶ and R¹⁷ can be the same or different and are each independently selected from the group consisting of H, alkyl, alkenyl, alkynyl, cycloalkyl, cycloalkylalkyl-, heterocyclyl, heterocyclylalkyl-, aryl, arylalkyl-, heteroaryl, heteroarylalkyl-, arylcycloalkyl-, arylheterocyclyl-, (R¹⁵)ᵣ-alkyl, (R¹⁸)ᵣ-cycloalkyl, (R¹⁸)ᵣ (R¹⁸)ᵣ-heterocyclyl, (R¹⁸)ᵣ-heterocyclylalkyl-, (R¹⁸)ᵣ-aryl, (R¹⁸)ᵣ-arylalkyl-, (R¹⁸)ᵣ-heteroaryland (R¹⁸)ᵣ-heteroarylalkyl-; wherein r is 1-5;

each R¹⁸ is independently selected from the group consisting of alkyl, alkenyl, alkynyl, aryl, arylalkyl-, arylalkenyl-, arylalkynyl-, —NO₂, halo, heteroaryl, HO-alkyoxyalkyl-, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

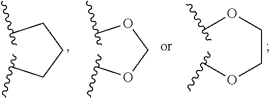

R¹⁹ is selected from the group consisting of: alkyl, cycloalkyl, aryl, arylalkyl- and heteroarylalkyl-;

R²⁰ is selected from the group consisting of: alkyl, cycloalkyl, aryl, halo substituted aryl, arylalkyl-, heteroaryl and heteroarylalkyl-;

each R²¹ group is independently selected from the group consisting of alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, halo, —CN, —OR¹⁸, —C(O)R¹⁵, —C(O)OR¹⁶, —C(O)N(R¹⁵)(R¹⁶), —SF₅, —OSF₅, —Si(R^{15A})₃, —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁶)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁶)C(O)N(R¹⁶)(R¹⁷), —CH₂—R¹⁵; —CH₂N(R¹⁵)(R¹⁶), —N(R¹⁵)S(O)R¹⁶, —N(R¹⁶)S(O)₂R¹⁶, —CH₂—N(R¹⁶)S(O)₂R¹⁶, —N(R¹⁵)S(O)₂N(R¹⁶)(R¹⁷), —N(R¹⁵)S(O)N(R¹⁶)(R¹⁷), —N(R¹⁶)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁸, —N₃, —NO₂ and —S(O)₂R^{15A}; and wherein each of the R²¹ alkyl, cycloalkenyl, cycloalkyl, cycloalkylalkyl-, heterocyclyl, heterocyclylalkyl-, aryl, arylalkyl-, heteroaryl, heteroarylalkyl-, alkenyl and alkynyl groups is optionally substituted with 1 to 5 independently selected R²² groups; and

each R²² is independently selected from the group consisting of: alkyl, cycloalkyl, cycloalkenyl, heterocyclyl, aryl, heteroaryl, halo, —CF₃, —CN, —OR¹⁶, —C(O)R¹⁵, —C(O)OR¹⁶, -alkyl-C(O)OR¹⁶, —C(O)^{N}(R¹⁵)(R¹⁶), —SF₅, —OSF₅, —Si(R^{15A})₃, —SR¹⁵, —S(O)N(R¹⁵)(R¹⁶), —S(O)₂N(R₁₅)(R₁₆), —C(═NOR¹⁵)R¹⁶, —P(O)(OR¹⁵)(OR¹⁶), —N(R¹⁵)S(O)R¹⁶, —N(R¹⁵)S(O)₂R¹⁶, —CH₂—N(R₁₅)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R^{15A}.

Preferably, in the embodiment described immediately above, R⁹ is selected from the group consisting of alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each R⁹ group is optionally substituted with 1-3 independently selected R²¹ substituents.

In another embodiment, the present application discloses a compound, or pharmaceutically acceptable salts, solvates, esters or prodrugs of said compound, said compound having the general structure shown in Formula I:

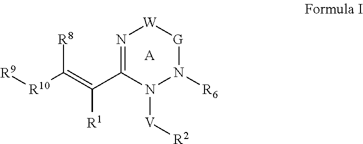

wherein:

(a) R¹ and R² are joined together to form 5-8 membered heterocyclyl or 5-8 membered heterocyclenyl moiety, wherein said heterocyclyl or heterocyclenyl moiety is optionally substituted with 1-5 independently selected R²¹ substituents, and

(b) R² and R⁶ are joined together to form 5-8 membered heterocyclyl or 5-8 membered heterocyclenyl moiety, wherein: said heterocyclyl or heterocyclenyl moiety is optionally substituted with 1-5 independently selected R²¹ substituents; and

(c) said R² and R⁶ heterocyclyl or heterocyclenyl moiety is optionally fused with an aryl or heteroaryl ring, and the ring moiety resulting from the fusion is optionally substituted with 1-5 independently selected R²¹ substituents; and

W is selected from the group consisting of a bond, —C(O)—, —S(O)—, —S(O₂)— and —(CR¹¹R¹²)_{1 or 2}—, e.g., —CR¹¹R¹²—, —CR¹¹R¹²—CH₂—, —CR¹¹R¹²— CR¹¹R¹²—, and —CH₂—C(R¹¹)(R¹²)—, with the proviso that ring A is a 5-, 6- or 7-membered ring;

G is selected from the group consisting of —C(O)—, —S(O)—, —S(O₂)— and —(CR³R⁴)_{1 or 2}—, e.g., —CR³R⁴—, —CR³R⁴—CH₂—, —CR³R⁴— CR³R⁴—, and —CH₂—CR³R⁴—, with the provisos that ring A is a 5-, 6- or 7-membered ring and that no combination of W and G can be —C(O)—S(O)—, C(O)—S(O)₂—, —S(O)—C(O)—, —S(O)₂—C(O)—, —S(O)—S(O)—, S(O)—S(O)₂—, —S(O)₂—S(O)— or S(O)₂—S(O)₂—;

V is selected from the group consisting of a bond and —C(O)—;

each R³ (when R³ does not form a ring with R⁴) can be the same or different and is independently selected from the group consisting of H, halo (and in one example, F), —OR¹⁵ (and in one example R¹⁵ is H), —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —C(O)R¹⁵, —C(O)OR¹⁵, —C(═NOR¹⁵)R¹⁶, —C(O)N(R¹⁵)(R¹⁶), —S(O)N(R¹⁵)(R¹⁶), S(O)₂N(R¹⁵)(R¹⁶), —S(O)R¹⁵, —S(O)₂R^{15A}, —P(O)(OR¹⁵)(OR¹⁶), ═NOR¹⁵, —N₃, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each of said alkyl-, alkenyl- and alkynyl-, aryl-, arylalkyl-, alkylaryl, cycloalkyl-, cycloalkylalkyl-, heteroaryl-, heteroarylalkyl-, heterocyclyl- and heterocyclylalkyl- can be unsubstituted or optionally substituted with 1-5 independently selected R²¹ substituents; or

each R⁴ (when R⁴ does not form a ring with R³), R¹¹ (when R¹¹ does not from a ring with R¹²) and R¹² (when R¹² does not for a ring with R¹¹) can be the same or different and is independently selected from the group consisting of H, halo (and in one example, F), —OR¹⁵ (and in one example R¹⁵ is H), —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —C(O)R¹⁵, —C(O)OR¹⁵, —C(═NOR¹⁵)R¹⁶—C(O)N(R¹⁵)(R¹⁶), —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, —N₃, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- can be unsubstituted or optionally substituted with 1-5 independently selected R²¹ substituents;

alternatively, when W is —CR¹¹R¹²— and G is —CR³R⁴—, R³ (when R³ does not form a ring with R⁴) and R¹¹ (when R¹¹ does not form a ring with R¹²) can be joined together to form a bond;

alternatively, (a) R³ (when R³ does not form a bond with R¹¹) and R⁴ can be joined together to form a C3-C8 spirocycloalkyl, C4-C8 spirocycloalkenyl, 5-8 membered spiroheterocyclyl or 5-8 membered spiroheterocyclenyl moiety, with each of said spirocycloalkyl or spiroheterocyclyl or spirocycloalkenyl moiety being unsubstituted or optionally substituted with 1-5 independently selected R²¹ substituents, or (b) R¹¹ and R¹² can be joined together to form a C3-C8 spirocycloalkyl, C4-C8 spirocycloalkenyl, 5-8 membered spiroheterocyclyl or 5-8 membered spiroheterocyclenyl moiety, with each of said spirocycloalkyl or spiroheterocyclyl or spirocycloalkenyl moiety being unsubstituted or optionally substituted with 1-5 independently selected R²¹ substituents, and (c) with the proviso that ring A can have only one C3-C8 spirocycloalkyl, C4-C8 spirocycloalkenyl, 5-8 membered spiroheterocyclyl or 5-8 membered spiroheterocyclenyl moiety;

provided that when one of R³ or R⁴ is selected from the group consisting of: N(R¹⁵)S(O)₂R¹⁶, —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, and —N₃, then the other is not selected from the group consisting of: —OR¹⁵, —CN, —SR¹⁵, and —NR¹⁵R¹⁶, —N(R¹⁵)^{C}(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)²N(R¹⁵)(R¹⁶), —S(O)R¹⁵, —S(O)₂R^{15A}, —P(O)(OR¹⁵)(OR¹⁶), ═NOR¹⁸, and —N₃ (i.e., if one of R³ or R⁴ is —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, or —N₃, then the other one is not —OR¹⁵, —CN, —SR¹⁵, and —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)C(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)²N(R¹⁵)(R¹⁶), —S(O)R¹⁵, —S(O)₂R^{15A}, —P(O)(OR¹⁵)(OR¹⁶), ═NOR¹⁵, or —N₃);

provided that when one of R¹¹ or R¹² is selected from the group consisting of: —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁸)(R¹⁸), —S(O)₂N(R¹⁵)(R¹⁶), —S(O)R¹⁵, —S(O)₂R^{15A}, —P(O)(OR¹⁵)(OR¹⁶), ═NOR¹⁵, and —N₃, then the other is not selected from the group consisting of: —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁸)C(O)OR¹⁸, —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, and —N₃ (i.e., if one of R¹¹ or R¹² is —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁸)C(O)N(R¹⁶)(R¹⁷), —N(R¹⁵)C(O)OR¹⁶, —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, or —N₃, then the other is not —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, or —N₃);

R⁸ (when R¹ is not joined to R⁸) is selected from the group consisting of H, alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl-, with each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- being unsubstituted or optionally substituted with 1-3 independently selected R²¹ substituents;

R⁹ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each R⁹ group is optionally substituted with 1-3 independently selected R²¹ substituents;

R¹⁰ is selected from the group consisting of a bond, alkyl-, alkenyl-, alkynyl-, aryl-, arylalkyl-, alkylaryl-, cycloalkyl-, cycloalkylalkyl-, heteroaryl-, heteroarylalkyl-, heterocyclyl-, heterocyclylalkyl- and the moieties:

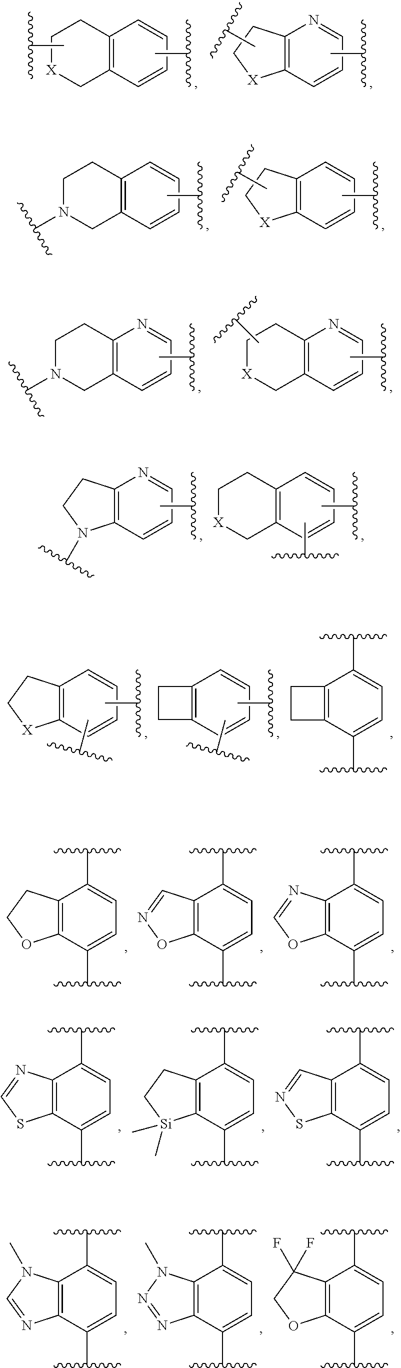

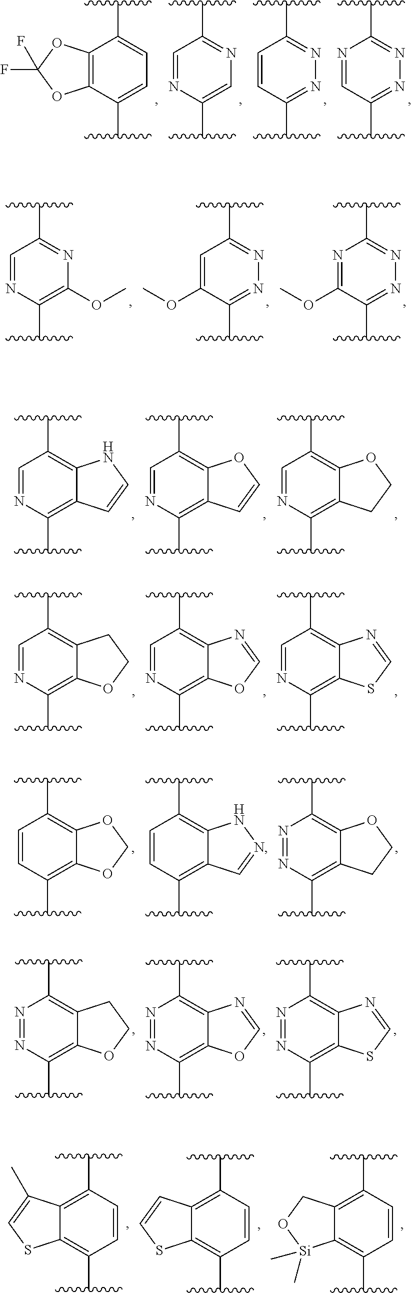

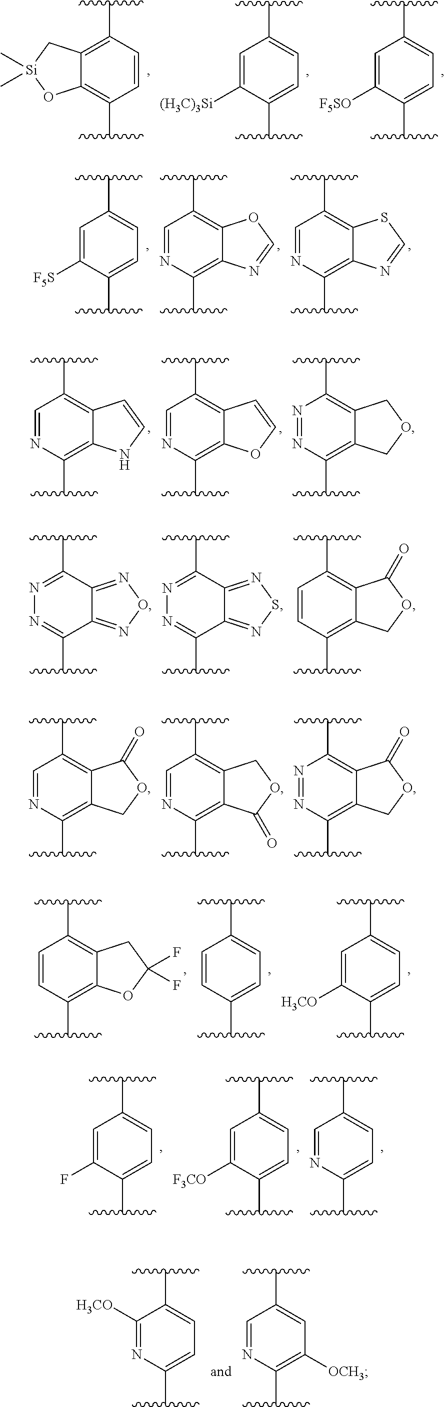

wherein X is O, N(R¹⁴) or S and wherein each R¹⁰ group (except for the bond) is optionally substituted with 1-3 independently selected R²¹ substituents;

R¹⁴ is selected from the group consisting of H, alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁸, and —P(O)(OR¹⁵)(OR¹⁶), wherein each of said alkyl, alkenyl, alkynyl, cycloalkyl, cycloalkylalkyl-, cycloalkenyl, heterocyclyl, heterocyclylalkyl-, aryl, arylalkyl-, heteroaryl, and heteroarylalkyl- is optionally substituted with 1-5 independently selected R²¹ substitutents;

R¹⁵, R¹⁶ and R¹⁷ can be the same or different and are each independently selected from the group consisting of H, alkyl, alkenyl, alkynyl, cycloalkyl, cycloalkylalkyl-, heterocyclyl, heterocyclylalkyl-, aryl, arylalkyl-, heteroaryl, heteroarylalkyl-, arylcycloalkyl-, arylheterocyclyl-, (R¹⁸)ᵣ-alkyl, (R¹⁸)ᵣ-cycloalkyl, (R¹⁸)ᵣ-cycloalkylalkyl-, (R¹⁸)ᵣ-heterocyclyl, (R¹⁸)ᵣ-heterocyclylalkyl-, (R¹⁸)ᵣ-aryl, (R¹⁸)ᵣ-arylalkyl-, (R¹⁸)ᵣ-heteroaryl and (R¹⁸)ᵣ-heteroarylalkyl-; wherein r is 1-5;

each R¹⁸ is independently selected from the group consisting of alkyl, alkenyl, alkynyl, aryl, arylalkyl-, arylalkenyl-, arylalkynyl-, —NO₂, halo, heteroaryl, HO-alkyoxyalkyl-, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

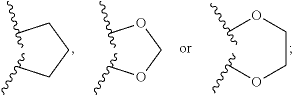

R¹⁹ is selected from the group consisting of: alkyl, cycloalkyl, aryl, arylalkyl- and heteroarylalkyl-;

R²⁰ is selected from the group consisting of: alkyl, cycloalkyl, aryl, halo substituted aryl, arylalkyl-, heteroaryl and heteroarylalkyl-;

each R²¹ group is independently selected from the group consisting of alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, halo, —CN, —OR¹⁵, —C(O)R¹⁵, —C(O)OR¹⁵, —C(O)N(R¹⁵)(R¹⁶), —SF₅, —OSF₅, —Si(R^{15A})₃, —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R^{15A}; and wherein each of the R²¹ alkyl, cycloalkenyl, cycloalkyl, cycloalkylalkyl-, heterocyclyl, heterocyclylalkyl-, aryl, arylalkyl-, heteroaryl, heteroarylalkyl-, alkenyl and alkynyl groups is optionally substituted with 1 to 5 independently selected R²² groups; and

each R²² is independently selected from the group consisting of: alkyl, cycloalkyl, cycloalkenyl, heterocyclyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SF₅, —OSF₅, —Si(R^{15A})₃, —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷) —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R^{15A}.

Preferably, in the embodiment described immediately above, R⁹ is selected from the group consisting of alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each R⁹ group is optionally substituted with 1-3 independently selected R²¹ substituents.

In another embodiment, the present application discloses a compound, or pharmaceutically acceptable salts, solvates, esters or prodrugs of said compound, said compound having the general structure shown in Formula I:

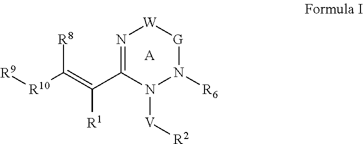

wherein:

R⁶ and one R³ of the —(CR³R⁴)_{1 or 2}-G moiety, are joined together to form 5-8 membered heterocyclyl or 5-8 membered heterocyclenyl moiety, wherein: (a) said heterocyclyl or heterocyclenyl moiety is optionally substituted with 1-5 independently selected R²¹ substituents, and (b) said heterocyclyl or heterocyclenyl moiety is optionally fused with an aryl or heteroaryl ring, and the ring moiety resulting from the fusion is optionally substituted with 1-5 independently selected R²¹ substituents;

R¹ and R² can be the same or different, each being independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- can be unsubstituted or optionally substituted with 1-5 independently selected R²¹ substituents;

or, alternatively, R¹ and R⁸ can be taken together to form a bond (i.e., there is a triple bond between the carbon atom to which R¹ was bonded to and the carbon to which R⁸ was bonded to, i.e., the compound of formula I is a compound of formula II:

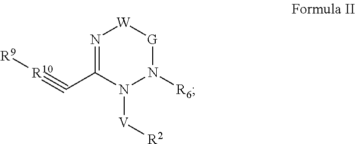

W is selected from the group consisting of a bond, —C(O)—, —S(O)—, —S(O₂)— and —(CR¹¹R¹²)_{1 or 2}—, e.g., —CR¹¹R¹²—, —CR¹¹R¹²—CH₂—, —CR¹¹R¹²—CR¹¹R¹²—, and —CH₂—C(R¹¹)(R¹²)—, with the proviso that ring A is a 5-, 6- or 7-membered ring;

G is selected from the group consisting of —C(O)—, —S(O)—, —S(O₂)— and —(CR³R⁴)_{1 or 2}—, e.g., —CR³R⁴—, —CR³R⁴—CH₂—, —CR³R⁴— CR³R⁴—, and —CH₂—CR³R⁴—, with the provisos that ring A is a 5-, 6- or 7-membered ring and that no combination of W and G can be —C(O)—S(O)—, C(O)—S(O)₂—, —S(O)—C(O)—, —S(O)₂—C(O)—, —S(O)—S(O)—, S(O)—S(O)₂—, —S(O)₂—S(O)— or S(O)₂—S(O)₂—;

V is selected from the group consisting of a bond and —C(O)—;

each R⁴ (when R⁴ does not form a ring with R³), R¹¹ (when R¹¹ does not from a ring with R¹²) and R¹² (when R¹² does not for a ring with R¹¹) can be the same or different and is independently selected from the group consisting of H, halo (and in one example, F), —OR¹⁶ (and in one example R¹⁵ is H), —CN, —SR¹⁵, —NR¹⁶R¹⁶, —N(R¹⁵)C(O)R¹⁶, —N(R¹⁵)S(O)R¹⁶, —N(R¹⁶)S(O)₂R¹⁶, —N(R¹⁶)S(O)₂N(R¹⁶)(R¹⁷), —N(R¹⁶)S(O)N(R¹⁶)(R¹⁷), —N(R¹⁵)C(O)N(R¹⁶)(R¹⁷), —N(R¹⁵)C(O)OR¹⁶, —C(O)R¹⁵, —C(O)OR¹⁶, —C(═NOR¹⁵)R¹⁶, —C(O)N(R¹⁵)(R¹⁶), —S(O)N(R¹⁵)(R¹⁶), —S(O)₂N(R¹⁵)(R¹⁶), —S(O)R¹⁶, —S(O)₂R^{15A}, —P(O)(OR¹⁵)(OR¹⁶), ═NOR¹⁶, —N₃, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- can be unsubstituted or optionally substituted with 1-5 independently selected R²¹ substituents;

provided that when one of R³ or R⁴ is selected from the group consisting of: —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R₁₅)S(O)N(R₁₆)(R₁₇), —N(R¹⁵)C(O)N(R¹⁶)(R¹⁷), —N(R¹⁵)C(O)OR¹⁶, —S(O)N(R¹⁵)(R¹⁶), —S(O)²N(R¹⁵)(R¹⁶), —S(O)R¹⁵, —S(O)₂R^{15A}, —P(O)(OR¹⁵)(OR¹⁶), ═NOR¹⁵, and —N₃, then the other is not selected from the group consisting of: —OR¹⁵, —CN, —SR¹⁵, and —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)²N(R¹⁵)(R¹⁶), —S(O)R¹⁵, —S(O)₂R^{15A}, —P(O)(OR¹⁵)(OR¹⁶), ═NOR¹⁵, and —N₃ (i.e., if one of R³ or R⁴ is −OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)²N(R¹⁵)(R¹⁶), —S(O)R¹⁵, —S(O)₂R^{15A}, —P(O)(OR¹⁵)(OR¹⁶), ═NOR¹⁵, or —N₃, then the other one is not —OR¹⁵, —CN, —SR¹⁵, and —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R₁₅)S(O)N(R₁₆)(R₁₇), —N(R¹⁵)C(O)N(R¹⁶)(R¹⁷), —N(R¹⁵)C(O)OR¹⁶, —S(O)N(R¹⁵)(R¹⁶), —S(O)²N(R¹⁵)(R¹⁶), —S(O)R¹⁵, —S(O)₂R^{15A}, —P(O)(OR¹⁵)(OR¹⁶), ═NOR¹⁵, or —N₃);

provided that when one of R¹¹ or R¹² is selected from the group consisting of: —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶)—S(O)₂N(R¹⁵)(R¹⁶), —S(O)R¹⁵, —S(O)₂R^{15A}, —P(O)(OR¹⁵)(OR¹⁶), ═NOR¹⁵, and —N₃, then the other is not selected from the group consisting of: —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)²N(R¹⁵)(R¹⁶), —S(O)R¹⁵, —S(O)₂R^{15A}, —P(O)(OR¹⁵)(OR¹⁶), ═NOR¹⁵, and —N₃ (i.e., if one of R¹¹ or R¹² is —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)^{C}(O)^{N}(R¹⁶)(R¹⁷), —N(R¹⁵)C(O)OR¹⁶, —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, or —N₃, then the other is not —OR¹⁵, —CN, —SR¹⁵, —NR¹⁵R¹⁶, —N(R¹⁵)C(O)R¹⁶, —N(R¹⁵)S(O)R¹⁶, —N(R¹⁶)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, or —N₃);

R⁸ (when R¹ is not joined to R⁸) is selected from the group consisting of H, alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl-, with each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- being unsubstituted or optionally substituted with 1-3 independently selected R²¹ substituents;

R⁹ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each R⁹ group is optionally substituted with 1-3 independently selected R²¹ substituents;

R¹⁰ is selected from the group consisting of a bond, alkyl-, alkenyl-, alkynyl-, aryl-, arylalkyl-, alkylaryl-, cycloalkyl-, cycloalkylalkyl-, heteroaryl-, heteroarylalkyl-, heterocyclyl-, heterocyclylalkyl- and the moieties:

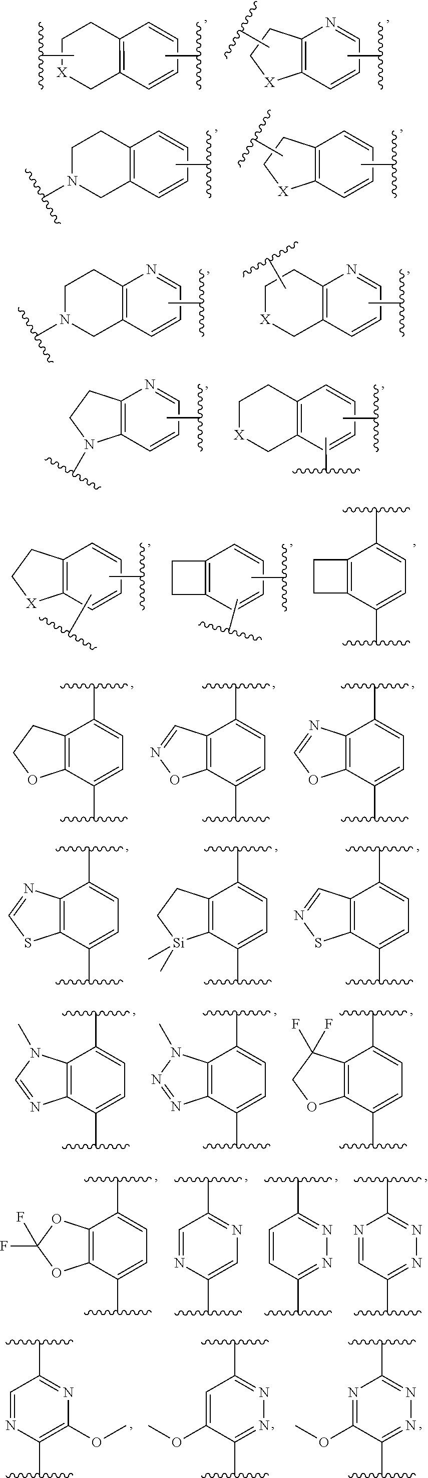

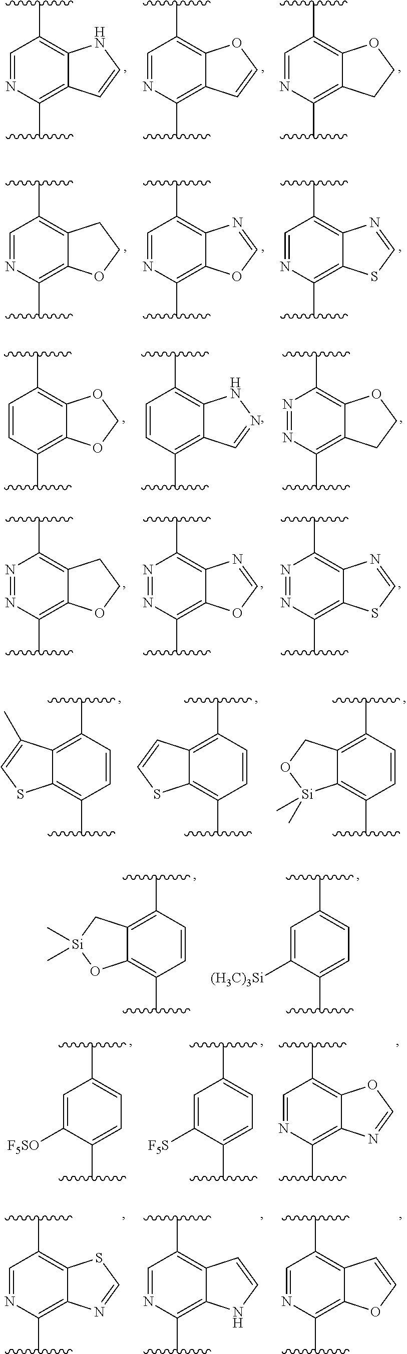

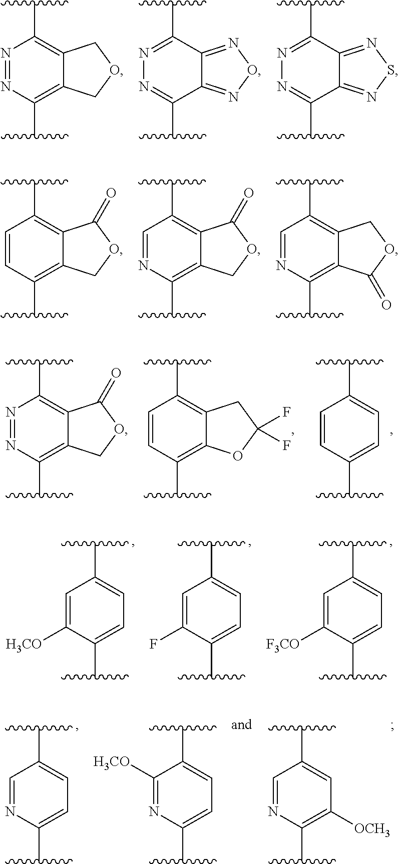

wherein X is O, N(R¹⁴) or S and wherein each R¹⁰ group (except for the bond) is optionally substituted with 1-3 independently selected R²¹ substituents;

R¹⁴ is selected from the group consisting of H, alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶), wherein each of said alkyl, alkenyl, alkynyl, cycloalkyl, cycloalkylalkyl-, cycloalkenyl, heterocyclyl, heterocyclylalkyl-, aryl, arylalkyl-, heteroaryl, and heteroarylalkyl- is optionally substituted with 1-5 independently selected R²¹ substitutents;

R¹⁵, R¹⁶ and R¹⁷ can be the same or different and are each independently selected from the group consisting of H, alkyl, alkenyl, alkynyl, cycloalkyl, cycloalkylalkyl-, heterocyclyl, heterocyclylalkyl-, aryl, arylalkyl-, heteroaryl, heteroarylalkyl-, arylcycloalkyl-, arylheterocyclyl-, (R¹⁸)ᵣ-alkyl, (R¹⁸)ᵣ-cycloalkyl, (R¹⁸)ᵣ-cycloalkylalkyl-, (R¹⁸)ᵣ-heterocyclyl, (R¹⁸)ᵣ-heterocyclylalkyl-, (R¹⁸)ᵣ-aryl, (R¹⁸)ᵣ-arylalkyl-, (R¹⁸)ᵣ-heteroaryl and (R¹⁸)ᵣ-heteroarylalkyl-; wherein r is 1-5;

each R¹⁸ is independently selected from the group consisting of alkyl, alkenyl, alkynyl, aryl, arylalkyl-, arylalkenyl-, arylalkynyl-, —NO₂, halo, heteroaryl, HO-alkyoxyalkyl-, —CF₃, —ON, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

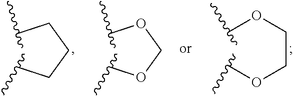

R¹⁹ is selected from the group consisting of: alkyl, cycloalkyl, aryl, arylalkyl- and heteroarylalkyl-;

R²⁰ is selected from the group consisting of: alkyl, cycloalkyl, aryl, halo substituted aryl, arylalkyl-, heteroaryl and heteroarylalkyl-;

each R²¹ group is independently selected from the group consisting of alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, halo, —CN, —OR¹⁵, —C(O)R¹⁵, —C(O)OR¹⁵, —C(O)N(R¹⁵)(R¹⁶), —SF₅, —OSF₅, —Si(R^{15A})₃, —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R^{15A}; and wherein each of the R²¹ alkyl, cycloalkenyl, cycloalkyl, cycloalkylalkyl-, heterocyclyl, heterocyclylalkyl-, aryl, arylalkyl-, heteroaryl, heteroarylalkyl-, alkenyl and alkynyl groups is optionally substituted with 1 to 5 independently selected R²² groups; and each R²² is independently selected from the group consisting of: alkyl, cycloalkyl, cycloalkenyl, heterocyclyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SF₅, —OSF₅, —Si(R^{15A})₃, —SR¹⁵, —S(O)N(R¹⁵)(R¹⁶), —S(O)₂N(R¹⁵)(R¹⁶), —C(═NOR¹⁵)R¹⁸, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁶)S(O)R¹⁶, —N(R¹⁵)S(O)₂R¹⁶, —CH₂—N(R¹⁵)S(O)₂R¹⁶, —N(R¹⁵)S(O)₂M(R¹⁶)(R¹⁷), —N(R¹⁶)S(O)N(R¹⁶)(R¹⁷), —N(R¹⁶)C(O)N(R¹⁶)(R¹⁷), —CH₂—N(R¹⁶)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R^{15A}.

Preferably, in the embodiment described immediately above, R⁹ is selected from the group consisting of alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each R⁹ group is optionally substituted with 1-3 independently selected R²¹ substituents.

In another embodiment, the present application discloses a compound, or pharmaceutically acceptable salts, solvates, esters or prodrugs of said compound, said compound having the general structure shown in Formula I:

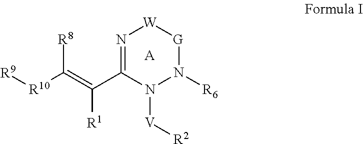

wherein:

R¹, R² and R⁶ can be the same or different, each being independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- can be unsubstituted or optionally substituted with 1-5 independently selected R²¹ substituents;

or, alternatively, R¹ and R⁸ can be taken together to form a bond (i.e., there is a triple bond between the carbon atom to which R¹ was bonded to and the carbon to which R⁸ was bonded to, i.e., the compound of formula I is a compound of formula II:

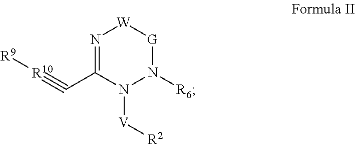

W is selected from the group consisting of a bond, —C(O)—, —S(O)—, —S(O₂)— and —(CR¹¹R¹²)_{1 or 2}—, e.g., —CR¹¹R¹²—, —CR—CH₂—, —CR—CR¹¹R¹²—, and —CH₂—C(R¹¹)(R¹²)—, with the proviso that ring A is a 5-, 6- or 7-membered ring;

G is selected from the group consisting of —C(O)—, —S(O)—, —S(O₂)— and —(CR³R⁴)_{1 or 2}—, e.g., —CR³R⁴—, —CR³R⁴—CH₂—, —CR³R⁴— CR³R⁴—, and —CH₂—CR³R⁴—, with the provisos that ring A is a 5-, 6- or 7-membered ring and that no combination of W and G can be —C(O)—S(O)—, C(O)—S(O)₂—, —S(O)—C(O)—, —S(O)₂—C(O)—, —S(O)—S(O)—, S(O)—S(O)₂—, —S(O)₂—S(O)— or S(O)₂—S(O)₂—;

V is selected from the group consisting of a bond and —C(O)—;

each R⁴ (when R⁴ does not form a ring with R³), R¹¹ (when R¹¹ does not from a ring with R¹²) and R¹² (when R¹² does not for a ring with R¹¹) can be the same or different and is independently selected from the group consisting of H, halo (and in one example, F), —OR¹⁵ (and in one example R¹⁵ is H), —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —C(O)R¹⁵, —C(O)OR¹⁵, —C(═NOR¹⁵)R¹⁶—C(O)N(R¹⁵)(R¹⁶), —S(O)N(R¹⁵)(R¹⁶), —S(O)²N(R¹⁵)(R¹⁶), —S(O)R¹⁵, —S(O)₂R^{15A}, —P(O)(OR¹⁵)(OR¹⁶), ═NOR¹⁵, —N₃, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- can be unsubstituted or optionally substituted with 1-5 independently selected R²¹ substituents;

provided that when one of R³ or R⁴ is selected from the group consisting of: —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, and —N₃, then the other is not selected from the group consisting of: —OR¹⁵, —CN, —SR¹⁵, and —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)²N(R¹⁵)(R¹⁶), —S(O)R¹⁵, —S(O)₂R^{15A}, —P(O)(OR¹⁵)(OR¹⁶), ═NOR¹⁵, and —N₃ (i.e., if one of R³ or R⁴ is —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R¹⁵)(R¹⁶), —S(O)R¹⁵, —S(O)₂R^{15A}—P(O)(OR¹⁵)(OR¹⁶), ═NOR¹⁵, or —N₃, then the other one is not —OR¹⁵, —CN, —SR¹⁵, and —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R¹⁵)(R¹⁶), —S(O)R¹⁵, —S(O)₂R^{15A}—P(O)(OR¹⁵)(OR¹⁶), ═NOR¹⁵, or —N₃);

provided that when one of R¹¹ or R¹² is selected from the group consisting of: —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, and —N₃, then the other is not selected from the group consisting of: —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)²N(R¹⁵)(R¹⁶), —S(O)R¹⁵, —S(O)₂R^{15A}, —P(O)(OR¹⁵)(OR¹⁶), ═NOR¹⁵, and —N₃ (i.e., if one of R¹¹ or R¹² is —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)²N(R¹⁵)(R¹⁶), —S(O)R¹⁵, —S(O)₂R^{15A}, —P(O)(OR¹⁵)(OR¹⁶)═NOR¹⁵, or —N₃, then the other is not —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R₁₅)(R₁₆), —S(O)R¹⁵, —S(O)₂R^{15A}, —P(O)(OR¹⁵)(OR¹⁶), ═NOR¹⁵, or —N₃);

R⁸ (when R¹ is not joined to R⁸) is selected from the group consisting of H, alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl-, with each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- being unsubstituted or optionally substituted with 1-3 independently selected R²¹ substituents;

R⁹ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each R⁹ group is optionally substituted with 1-3 independently selected R²¹ substituents;

R¹⁶ is selected from the group consisting of a bond, alkyl-, alkenyl-, alkynyl-, aryl-, arylalkyl-, alkylaryl-, cycloalkyl-, cycloalkylalkyl-, heteroaryl-, heteroarylalkyl-, heterocyclyl-, heterocyclylalkyl- and the moieties:

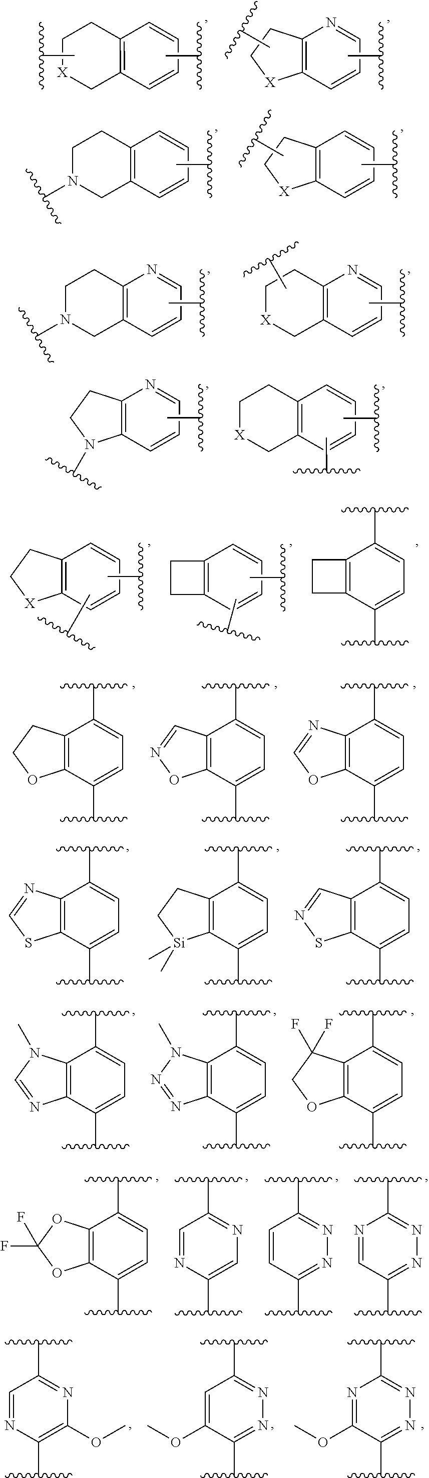

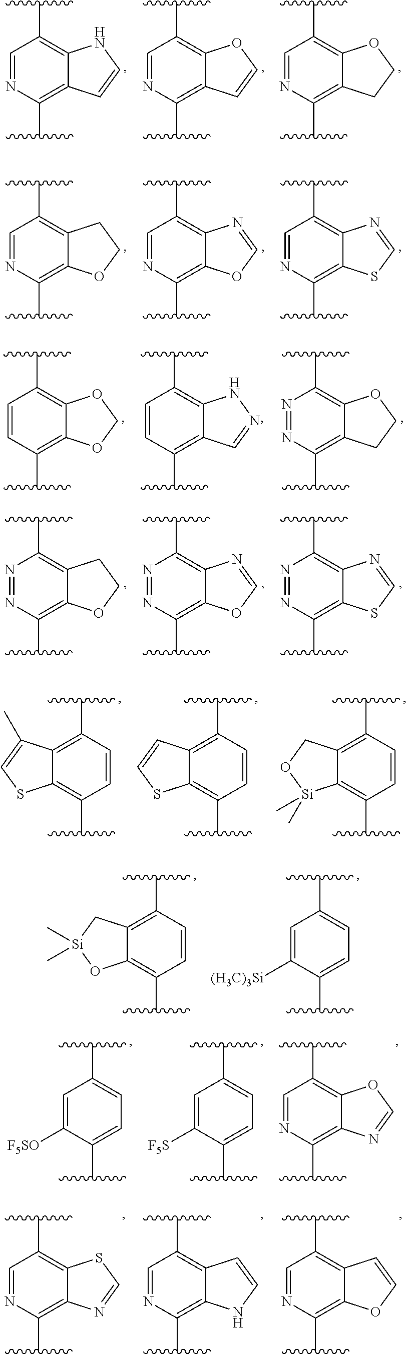

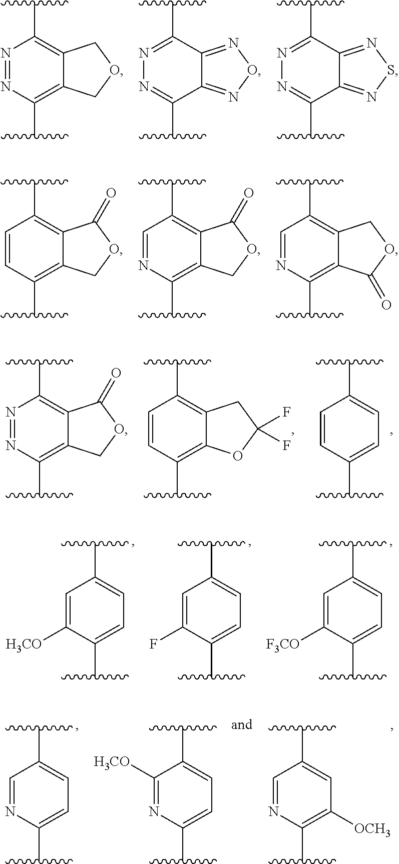

wherein X is O, N(R¹⁴) or S and wherein each R¹⁰ group (except for the bond) is optionally substituted with 1-3 independently selected R²¹ substituents;

R¹⁴ is selected from the group consisting of H, alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶), wherein each of said alkyl, alkenyl, alkynyl, cycloalkyl, cycloalkylalkyl-, cycloalkenyl, heterocyclyl, heterocyclylalkyl-, aryl, arylalkyl-, heteroaryl, and heteroarylalkyl- is optionally substituted with 1-5 independently selected R²¹ substitutents;

R¹⁵, R¹⁶ and R¹⁷ can be the same or different and are each independently selected from the group consisting of H, alkyl, alkenyl, alkynyl, cycloalkyl, cycloalkylalkyl-, heterocyclyl, heterocyclylalkyl-, aryl, arylalkyl-, heteroaryl, heteroarylalkyl-, arylcycloalkyl-, arylheterocyclyl-, (R¹⁸)ᵣ-alkyl, (R¹⁸)ᵣ-cycloalkyl, (R¹⁸)ᵣ-cycloalkylalkyl-, (R¹⁸)ᵣ-heterocyclyl, (R¹⁸)ᵣ-heterocyclylalkyl-, (R¹⁸)ᵣ-aryl, (R¹⁸)ᵣ-arylalkyl-, (R¹⁸)ᵣ-heteroaryl and (R¹⁸)ᵣ-heteroarylalkyl-; wherein r is 1-5;

each R¹⁸ is independently selected from the group consisting of alkyl, alkenyl, alkynyl, aryl, arylalkyl-, arylalkenyl-, arylalkynyl-, —NO₂, halo, heteroaryl, HO-alkyoxyalkyl-, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

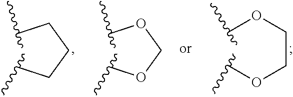

R¹⁹ is selected from the group consisting of: alkyl, cycloalkyl, aryl, arylalkyl- and heteroarylalkyl-;

R²⁰ is selected from the group consisting of: alkyl, cycloalkyl, aryl, halo substituted aryl, arylalkyl-, heteroaryl and heteroarylalkyl-;

each R²¹ group is independently selected from the group consisting of alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, halo, —CN, —OR¹⁵—C(O)R¹⁵, —C(O)OR¹⁵, —C(O)N(R¹⁵)(R¹⁶), —SF₅, —OSF₅, —Si(R^{15A})₃, —SR¹⁵, —S(O)N(R¹⁵)(R¹⁶), —CH(R¹⁵)(R¹⁶), —S(O)₂N(R¹⁵)(R¹⁶), —C(═NOR¹⁸)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁸)C(O)R¹⁶, —CH₂—N(R¹⁸)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁸)C(O)N(R¹⁶)(R¹⁷), —CH₂—N(R¹⁵)C(O)N(R¹⁶)(R¹⁷), —N(R¹⁸)C(O)OR¹⁶, —CH₂—N(R¹⁸)C(O)OR¹⁶, —S(O)R¹⁵, ═NOR¹⁸, —N₃, —NO₂ and —S(O)₂R^{15A}; and wherein each of the R²¹ alkyl, cycloalkenyl, cycloalkyl, cycloalkylalkyl-, heterocyclyl, heterocyclylalkyl-, aryl, arylalkyl-, heteroaryl, heteroarylalkyl-, alkenyl and alkynyl groups is optionally substituted with 1 to 5 independently selected R²² groups; and each R²² is independently selected from the group consisting of: alkyl, cycloalkyl, cycloalkenyl, heterocyclyl, aryl, heteroaryl, halo, —CF₃, —CN, —OR¹⁵, —C(O)R¹⁵, —C(O)OR¹⁵, -alkyl-C(O)OR¹⁵, C(O)^{N}(R¹⁵)(R¹⁶), —SF₅, —OSF₅, —Si(R^{15A})₃, —SR¹⁵, —S(O)N(R¹⁵)(R¹⁶), —S(O)₂N(R¹⁵)(R¹⁶), —C(═NOR¹⁸)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R^{15A}.

Preferably, in the embodiment described immediately above, R⁹ is selected from the group consisting of alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each R⁹ group is optionally substituted with 1-3 independently selected R²¹ substituents.

In another embodiment, R² is H.

In another embodiment, R² is alkyl.

In another embodiment, R² is methyl.

In another embodiment, R² is alkoxyalkyl-.

In another embodiment, R² is 3-methoxypropyl-.

In another embodiment, W is a bond.

In another embodiment, W is —C(O)—.

In another embodiment, W is —S(O)—.

In another embodiment, W is —S(O₂)—.

In another embodiment, W is —C(R¹¹)(R¹²)—.

In another embodiment, —W-G- is —C(R¹¹R¹²)—C(O)—.

In another embodiment, V is a bond.

In another embodiment, G is —C(R³)(R⁴)—.

In another embodiment, G is —C(O)—.

In another embodiment, R² is arylalkyl-.

In another embodiment, R² is phenylmethyl-.

In another embodiment, R² is (4-alkoxy)phenylmethyl-.

In another embodiment, R² is (4-methoxy)phenylmethyl-.

In another embodiment, R¹ is H.

In another embodiment, R¹ is alkyl.

In another embodiment, R¹ is methyl.

In another embodiment R¹ and R² are joined together to form a ring optionally substituted with 1 to 5 independently selected R²¹ substitutents, and said ring is fused with an aryl or heteroaryl ring, and said resulting fused ring is optionally substituted with 1 to 5 independently selected R²¹ substitutents.

In another embodiment R¹ and R² are joined together to form a ring substituted with 1 to 5 independently selected R²¹ substitutents, and said ring is fused with an aryl or heteroaryl ring, and said resulting fused ring is optionally substituted with 1 to 5 independently selected R²¹ substitutents.

In another embodiment R¹ and R² are joined together to form a ring optionally substituted with 1 to 5 independently selected R²¹ substitutents.

In another embodiment R¹ and R² are joined together to form a ring.

In another embodiment R¹ and R² are joined together to form a heterocyclyl ring optionally substituted with 1 to 5 independently selected R²¹ substitutents.

In another embodiment R¹ and R² are joined together to form a ring, and said ring is fused with an aryl or heteroaryl ring, and said resulting fused ring is optionally substituted with 1 to 5 independently selected R²¹ substitutents.

In another embodiment R¹ and R² are joined together to form a heterocyclyl ring.

In another embodiment R¹ and R² are joined together to form a piperidinyl ring optionally substituted with 1 to 5 independently selected R²¹ substitutents.

In another embodiment R¹ and R² are joined together to form a piperidinyl ring substituted with 1 to 5 independently selected R²¹ substitutents.

In another embodiment R¹ and R² are joined together to form a piperidinyl ring optionally substituted with a ═O moiety.

In another embodiment R¹ and R² are joined together to form a piperidinyl.

In another embodiment R¹ and R² are joined together to form a piperidinyl ring substituted with a ═O moiety.

In another embodiment, R⁶ is aryl.

In another embodiment, R⁶ is an unsubstituted phenyl.

In another embodiment, R⁶ is a phenyl which is substituted with 1-4 substituents which can be the same or different, each substituent being independently selected from the group consisting of halo, alkyl, —CN, —NH₂, —NH(alkyl), —N(alkyl)₂, hydroxy, alkoxy, aryl and heteroaryl groups.

In another embodiment, R⁶ is unsubstituted naphthyl.

In another embodiment, R⁶ is naphthyl which is substituted with 1-4 substituents which can be the same or different, each substituent being independently selected from the group consisting of halo, alkyl, —CN, —NH₂, —NH(alkyl), —N(alkyl)₂, hydroxy, alkoxy, aryl and heteroaryl groups.

In another embodiment, R⁶ is unsubstituted biphenyl.

In another embodiment, R⁶ is biphenyl which is substituted with 1-4 substituents which can be the same or different, each substituent being independently selected from the group consisting of halo, alkyl, —CN, —NH₂, —NH(alkyl), —N(alkyl)₂, hydroxy and alkoxy groups.

In another embodiment, R⁶ is 3-(1,1′-biphenyl)-yl.

In another embodiment, R⁶ is 4-(1,1′-biphenyl)-yl.

In another embodiment of this invention R⁶ is an unsubstituted or substituted aryl (e.g., phenyl) group.

In another embodiment of this invention R⁶ is an unsubstituted aryl (e.g., phenyl) or aryl (e.g., phenyl) substituted with one or more independently selected R²¹ groups.

In another embodiment of this invention R⁶ is an aryl or arylalkyl-group.

In another embodiment of this invention R⁶ is an aryl or arylalkyl-group, and said aryl group is substituted with one or more independently selected R²¹ groups.

In another embodiment of this invention R⁶ is an aryl or arylalkyl-group, and said aryl group is substituted with 1 to 3 independently selected R²¹ groups.

In another embodiment of this invention R⁶ is an aryl or arylalkyl-group, and said aryl group is substituted with one or more R²¹ groups, and each R²¹ group is the same or different halo.

In another embodiment of this invention R⁶ is an aryl or arylalkyl-group, and said aryl group is substituted with 1 to 3 R²¹ groups, and each R²¹ group is the same or different halo.

In another embodiment of this invention R⁶ is an aryl or arylalkyl-group, and said aryl group is substituted with three R²¹ halo groups, and each R²¹ group is the same or different halo.

In another embodiment of this invention R⁶ is an aryl or arylalkyl-group, and said aryl group is substituted with two R²¹ halo groups, and each R²¹ group is the same or different halo.

In another embodiment of this invention R⁶ is an aryl or arylalkyl-group, and said aryl group is substituted with one R²¹ halo group.

In another embodiment of this invention R⁶ is an aryl or arylalkyl-group, and said aryl group is substituted with one R²¹ halo groups, and each R²¹ group is the same or different halo.

In another embodiment of this invention R⁶ is an aryl or arylalkyl-group, and said aryl group is substituted with one F (i.e., said aryl is substituted with one R²¹ group, and said R²¹ group is halo, and said halo is F).

In another embodiment of this invention R⁶ is an aryl or arylalkyl-group, and said aryl group is substituted with two F atoms (i.e., said aryl is substituted with two R²¹ groups, and said R²¹ groups are halo, and said halo is F).

In another embodiment of this invention R⁶ is an aryl or arylalkyl-group, and said aryl group is substituted with three F atoms (i.e., said aryl is substituted with three R²¹ groups, and said R²¹ groups are halo, and said halo is F).

In another embodiment of this invention R⁶ is phenyl.

In another embodiment of this invention R⁶ is phenyl or phenylalkyl-group, and said phenyl is substituted with one or more independently selected R²¹ groups.

In another embodiment of this invention R⁶ is phenyl or phenylalkyl-group, and said phenyl is substituted with 1 to 3 independently selected R²¹ groups.

In another embodiment of this invention R⁶ is phenyl or phenylalkyl-group, and said phenyl is substituted with one or more R²¹ groups, and each R²¹ group is the same or different halo.

In another embodiment of this invention R⁶ is phenyl or phenylalkyl-group, and said phenyl is substituted with 1 to 3 R²¹ groups, and each R²¹ group is the same or different halo.

In another embodiment of this invention R⁶ is phenyl or phenylalkyl-group, and said phenyl is substituted with three R²¹ halo groups, and each R²¹ group is the same or different halo.

In another embodiment of this invention R⁶ is phenyl or phenylalkyl-group, and said phenyl is substituted with two R²¹ halo groups, and each R²¹ group is the same or different halo.

In another embodiment of this invention R⁶ is phenyl or phenylalkyl-group, and said phenyl is substituted with one R²¹ halo group.

In another embodiment of this invention R⁶ is phenyl or phenylalkyl-group, and said phenyl is substituted with one R²¹ halo group.

In another embodiment of this invention R⁶ is phenyl or phenylalkyl-group, and said phenyl is substituted with one F (i.e., said aryl is substituted with one R²¹ group, and said R²¹ group is halo, and said halo is F).

In another embodiment of this invention R⁶ is phenyl or phenylalkyl-group, and said phenyl is substituted with two F atoms (i.e., said aryl is substituted with two R²¹ groups, and said R²¹ groups are halo, and said halo is F).

In another embodiment of this invention R⁶ is phenyl or phenylalkyl-group, and said phenyl is substituted with three F atoms (i.e., said aryl is substituted with three R²¹ groups, and said R²¹ groups are halo, and said halo is F).

In another embodiment of this invention R⁶ is selected from the group consisting of:

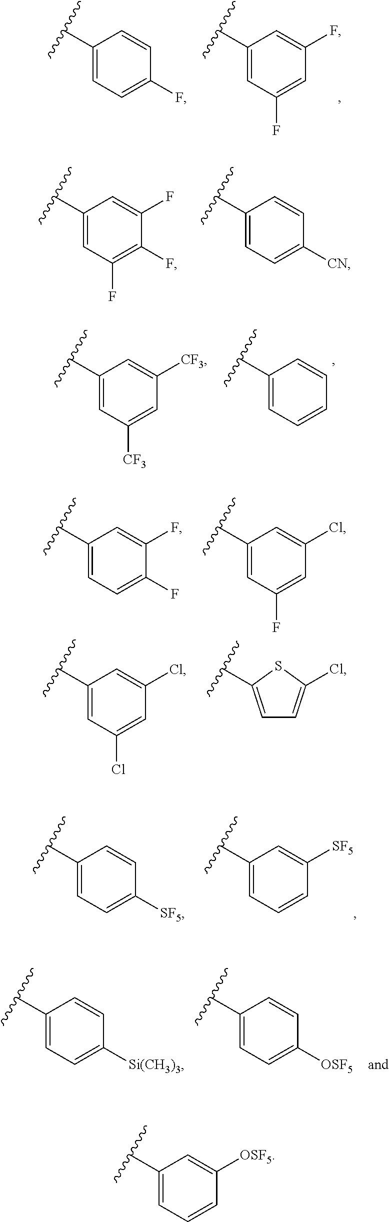

In another embodiment of this invention R⁶ is:

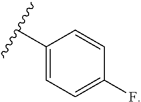

In another embodiment of this invention R⁶ is:

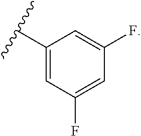

In another embodiment of this invention R⁶ is:

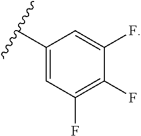

In another embodiment of this invention R⁶ is:

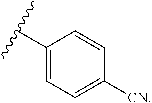

In another embodiment of this invention R⁶ is:

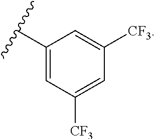

In another embodiment of this invention R⁶ is:

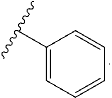

In another embodiment of this invention R⁶ is:

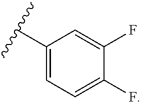

In another embodiment of this invention R⁶ is:

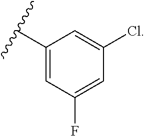

In another embodiment of this invention R⁶ is:

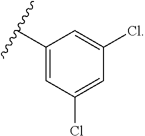

In another embodiment of this invention R⁶ is:

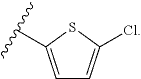

In another embodiment of this invention R⁶ is:

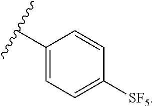

In another embodiment of this invention R⁶ is:

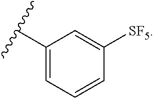

In another embodiment of this invention R⁶ is:

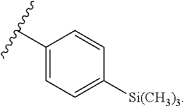

In another embodiment of this invention R⁶ is:

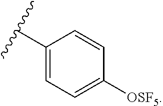

In another embodiment of this invention R⁶ is:

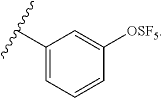

In another embodiment of this invention R⁶ is aryl substituted with R²¹ groups, and at least one (e.g. 1 to 2) of the R²¹ groups is selected from the group consisting of: —SF₅, —OSF₅ and —Si(R^{15A})³, wherein each R^{15A} is independently selected.

In another embodiment of this invention R⁶ is aryl substituted with R²¹ groups, and at least one (e.g. 1 to 2) of the R²¹ groups is selected from the group consisting of: —SF₅, —OSF₅ and —Si(R^{15A})₃, and each R^{15A} is the same or different alkyl group.

In another embodiment of this invention R⁶ is aryl substituted with R²¹ groups, and at least one (e.g. 1 to 2) of the R²¹ groups is selected from the group consisting of: —SF₅, —OSF₅ and —Si(CH₃)₃.

In another embodiment of this invention R⁶ is aryl substituted with R²¹ groups, and one R²¹ group is selected from the group consisting of: —SF₅, —OSF₅ and —Si(R^{15A})₃, wherein each R^{15A} is independently selected.

In another embodiment of this invention R⁶ is aryl substituted with R²¹ groups, and one R²¹ group is selected from the group consisting of: —SF₅, —OSF₅ and —Si(R^{15A})₃, and each R^{15A} is the same or different alkyl group.

In another embodiment of this invention R⁶ is aryl substituted with R²¹ groups, and one R²¹ group is selected from the group consisting of: —SF₅, OSF₅ and —Si(CH₃)₃.

In another embodiment of this invention R⁶ is aryl substituted with R²¹ groups, and two R²¹ groups are selected from the group consisting of: —SF₅, —OSF₅ and —Si(R^{15A})₃, wherein each R^{15A} is independently selected.

In another embodiment of this invention R⁶ is aryl substituted with R²¹ groups, and two R²¹ groups are selected from the group consisting of: —SF₅, —OSF₅ and —Si(R^{15A})₃, and each R^{15A} is the same or different alkyl group.

In another embodiment of this invention R⁶ is aryl substituted with R²¹ groups, and two R²¹ groups are selected from the group consisting of: —SF₅, —OSF₅ and —Si(CH₃)₃.

In another embodiment of this invention R⁶ is aryl substituted with R²¹ groups, and one R²¹ group is —SF₅.

In another embodiment of this invention R⁶ is aryl substituted with R²¹ groups, and two R²¹ groups are —SF₅.

In another embodiment of this invention R⁶ is aryl substituted with R²¹ groups, and one R²¹ group is —OSF₅.

In another embodiment of this invention R⁶ is aryl substituted with R²¹ groups, and two R²¹ groups are —OSF₅.

In another embodiment of this invention R⁶ is aryl substituted with R²¹ groups, and one R²¹ group is —Si(R^{15A})₃, wherein each R^{15A} is independently selected.

In another embodiment of this invention R⁶ is aryl substituted with R²¹ groups, and one R²¹ group is —Si(R^{15A})₃ and each R^{15A} is the same or different alkyl group.

In another embodiment of this invention R⁶ is aryl substituted with R²¹ groups, and one R²¹ group is —Si(CH₃)₃.

In another embodiment of this invention R⁶ is aryl substituted substituted with R²¹ groups, and two of the R²¹ groups are the same or different —Si(R^{15A})₃, wherein each R^{15A} is independently selected.

In another embodiment of this invention R⁶ is aryl substituted with R²¹ groups, and two of the R²¹ groups are the same or different —Si(R^{15A})₃ group, and each R^{15A} is the same or different alkyl group.

In another embodiment of this invention R⁶ is aryl substituted with R²¹ groups, and two of the R²¹ group are —Si(CH₃)₃.

In another embodiment of this invention R⁶ is an alkyl group substituted with one R²¹ group, and said R²¹ group is an aryl group, and said aryl group is substituted with one or more R²² groups, and at least one (e.g., 1 to 2) R²² group is selected from the group consisting of: —SF₅, —OSF₅ and —Si(R^{15A})₃, wherein each R^{15A} is independently selected.

In another embodiment of this invention R⁶ is an alkyl group substituted with one R²¹ group, and said R²¹ group is an aryl group, and said aryl group is substituted with one or more R²² groups, and at least one (e.g., 1 to 2) R²² group is selected from the group consisting of: —SF₅, —OSF₅ and —Si(R^{15A})₃, and each R^{15A} is the same or different alkyl group.

In another embodiment of this invention R⁶ is an alkyl group substituted with one R²¹ group, and said R²¹ group is an aryl group, and said aryl group is substituted with one or more R²² groups, and at least one (e.g., 1 to 2) R²² group is selected from the group consisting of: —SF₅, —OSF₅ and —Si(CH₃)₃.

In another embodiment of this invention R⁶ is an alkyl group substituted with one R²¹ group, and said R²¹ group is phenyl, and said phenyl is substituted with one or more R²² groups, and at least one (e.g., 1 to 2) R²² is selected from the group consisting of: —SF₅, —OSF₅ and —Si(R^{15A})₃, wherein each R^{15A} is independently selected.

In another embodiment of this invention R⁶ is an alkyl group substituted with one R²¹ group, and said R²¹ group is phenyl, and said phenyl is substituted with one or more R²² groups, and at least one (e.g., 1 to 2) R²² is selected from the group consisting of: —SF₅, —OSF₅ and —Si(R^{15A})₃, and each R^{15A} is the same or different alkyl group.

In another embodiment of this invention R⁶ is an alkyl group substituted with one R²¹ group, and said R²¹ group is phenyl, and said phenyl is substituted with one or more R²² groups, and at least one (e.g., 1 to 2) R²² is selected from the group consisting of: —SF₅, —OSF₅ and —Si(CH₃)₃.

In another embodiment of this invention R⁶ is an alkyl group substituted with one R²¹ group, and said R²¹ group is phenyl, and said phenyl is substituted with one or more R²² groups, and one of the R²² groups is selected from the group consisting of: —SF₅, —OSF₅ and —Si(R^{15A})₃.

In another embodiment of this invention R⁶ is an alkyl group substituted with one R²¹ group, and said R²¹ group is phenyl, and said phenyl is substituted with one or more R²² groups, and one of the R²² groups is selected from the group consisting of: —SF₅, —OSF₅ and —Si(R^{15A})₃, and each R^{15A} is the same or different alkyl group.

In another embodiment of this invention R⁶ is an alkyl group substituted with one R²¹ group, and said R²¹ group is phenyl, and said phenyl is substituted with one or more R²² groups, and one of the R²² groups is selected from the group consisting of: —SF₅, —OSF₅ and —Si(CH₃)₃.

In another embodiment of this invention R⁶ is an alkyl group substituted with one R²¹ group, and said R²¹ group is phenyl, and said phenyl is substituted with one or more R²² groups, and two of the R²² groups are selected from the group consisting of: —SF₅, —OSF₅ and —Si(R^{15A})₃.

In another embodiment of this invention R⁶ is an alkyl group substituted with one R²¹ group, and said R²¹ group is phenyl, and said phenyl is substituted with one or more R²² groups, and two of the R²² groups are selected from the group consisting of: —SF₅, —OSF₅ and —Si(R^{15A})₃, and each R^{15A} is the same or different alkyl group.

In another embodiment of this invention R⁶ is an alkyl group substituted with one R²¹ group, and said R²¹ group is phenyl, and said phenyl is substituted with one or more R²² groups, and two of the R²² groups are selected from the group consisting of: —SF₅, —OSF₅ and —Si(CH₃)₃.

In another embodiment of this invention R⁶ is an alkyl group substituted with one R²¹ group, and said R²¹ group is phenyl, and said phenyl is substituted with one or more R²² groups, and one of the R²² groups is —SF₅.

In another embodiment of this invention R⁶ is an alkyl group substituted with one R²¹ group, and said R²¹ group is phenyl, and said phenyl is substituted with one or more R²² groups, and two of the R²² groups are —SF₅.

In another embodiment of this invention R⁶ is an alkyl group substituted with one R²¹ group, and said R²¹ group is phenyl, and said phenyl is substituted with one or more R²² groups, and one of the R²² groups is —OSF₅.

In another embodiment of this invention R⁶ is an alkyl group substituted with one R²¹ group, and said R²¹ group is phenyl, and said phenyl is substituted with one or more R²² groups, and two of the R²² groups are —OSF₅.

In another embodiment of this invention R⁶ is an alkyl group substituted with one R²¹ group, and said R²¹ group is phenyl, and said phenyl is substituted with one or more R²² groups, and one of the R²² groups is —Si(R^{15A})₃.

In another embodiment of this invention R⁶ is an alkyl group substituted with one R²¹ group, and said R²¹ group is phenyl, and said phenyl is substituted with one or more R²² groups, and one of the R²² groups is —Si(R^{15A})₃, and each R^{15A} is the same or different alkyl group.

In another embodiment of this invention R⁶ is an alkyl group substituted with one R²¹ group, and said R²¹ group is phenyl, and said phenyl is substituted with one or more R²² groups, and one of the R²² groups is —Si(CH₃)₃.

In another embodiment of this invention R⁶ is an alkyl group substituted with one R²¹ group, and said R²¹ group is phenyl, and said phenyl is substituted with one or more R²² groups, and two of the R²² groups are —Si(R^{15A})₃.

In another embodiment of this invention R⁶ is an alkyl group substituted with one R²¹ group, and said R²¹ group is phenyl, and said phenyl is substituted with one or more R²² groups, and two of the R²² groups are —Si(R^{15A})₃, and each R^{15A} is the same or different alkyl group.

In another embodiment of this invention R⁶ is an alkyl group substituted with one R²¹ group, and said R²¹ group is phenyl, and said phenyl is substituted with one or more R²² groups, and two of the R²² groups are —Si(CH₃)₃.

In another embodiment of this invention R⁶ is an arylalkyl-group substituted with R²¹ groups, and at least one (e.g., 1 to 2) R²¹ group is selected from the group consisting of: —SF₅, —OSF₅ and —Si(R^{15A})₃, wherein each R^{15A} is independently selected.

In another embodiment of this invention R⁶ is an arylalkyl-group substituted with R²¹ groups, and at least one (e.g., 1 to 2) R²¹ group is selected from the group consisting of: —SF₅, —OSF₅ and —Si(R^{15A})₃, and each R^{15A} is the same or different alkyl group.

In another embodiment of this invention R⁶ is an arylalkyl-group substituted with R²¹ groups, and at least one (e.g., 1 to 2) R²¹ group is selected from the group consisting of: —SF₅, —OSF₅ and —Si(CH₃)₃.

In another embodiment of this invention R⁶ is an arylalkyl-group substituted with R²¹ groups, and said aryl moiety is phenyl, and at least one (e.g., 1 to 2) R²¹ group is selected from the group consisting of: —SF₅, —OSF₅ and —Si(R^{15A})₃, wherein each R^{15A} is independently selected.

In another embodiment of this invention R⁶ is an arylalkyl-group substituted with R²¹ groups, and said aryl moiety is phenyl, and at least one (e.g., 1 to 2) R²¹ group is selected from the group consisting of: —SF₅, —OSF₅ and —Si(R^{15A})₃, and each R^{15A} is the same or different alkyl group.

In another embodiment of this invention R⁶ is an arylalkyl-group substituted with R²¹ groups, and said aryl moiety is phenyl, and at least one (e.g., 1 to 2) R²¹ group is selected from the group consisting of: —SF₅, —OSF₅ and —Si(CH₃)₃.

In another embodiment of this invention R⁶ is an arylalkyl-group substituted with R²¹ groups, and said aryl moiety is phenyl, and said phenyl is substituted with at least one (e.g., 1 to 3, or 1 to 2) R²¹ group, and at least one (e.g., 1 or 2) R²¹ group on said phenyl is selected from the group consisting of: —SF₅, —OSF₅ and —Si(R^{15A})₃, wherein each R^{15A} is independently selected.

In another embodiment of this invention R⁶ is an arylalkyl-group substituted with R²¹ groups, and said aryl moiety is phenyl, and said phenyl is substituted with at least one (e.g., 1 to 3, or 1 to 2) R²¹ group, and at least one (e.g., 1 or 2) R²¹ group on said phenyl is selected from the group consisting of: —SF₅, —OSF₅ and —Si(R^{15A})₃, and each R^{15A} is the same or different alkyl group.

In another embodiment of this invention R⁶ is an arylalkyl-group substituted with R²¹ groups, and said aryl moiety is phenyl, and said phenyl is substituted with at least one (e.g., 1 to 3, or 1 to 2) R²¹ group, and at least one (e.g., 1 or 2) R²¹ group on said phenyl is selected from the group consisting of: —SF₅, —OSF₅ and —Si(CH₃)₃.

In another embodiment of this invention R⁶ is an arylalkyl-group substituted with R²¹ groups, and said aryl moiety is phenyl, and said phenyl is substituted with at least one (e.g., 1 to 3, or 1 to 2) R²¹ group, and one R²¹ group on said phenyl is selected from the group consisting of: —SF₅, —OSF₅ and —Si(R^{15A})₃, wherein each R^{15A} is independently selected.

In another embodiment of this invention R⁶ is an arylalkyl-group substituted with R²¹ groups, and said aryl moiety is phenyl, and said phenyl is substituted with at least one (e.g., 1 to 3, or 1 to 2) R²¹ group, and one R²¹ group on said phenyl is selected from the group consisting of: —SF₅, —OSF₅ and —Si(R^{15A})₃, and each R^{15A} is the same or different alkyl group.

In another embodiment of this invention R⁶ is an arylalkyl-group substituted with R²¹ groups, and said aryl moiety is phenyl, and said phenyl is substituted with at least one (e.g., 1 to 3, or 1 to 2) R²¹ group, and one R²¹ group on said phenyl is selected from the group consisting of: —SF₅, —OSF₅ and —Si(CH₃)₃.

In another embodiment of this invention R⁶ is an arylalkyl-group substituted with R²¹ groups, and said aryl moiety is phenyl, and said phenyl is substituted with at least two (e.g., 2 to 3, or 2, or 3) R²¹ groups, and two R²¹ groups on said phenyl is selected from the group consisting of: —SF₅, —OSF₅ and —Si(R^{15A})₃, wherein each R^{15A} is independently selected.

In another embodiment of this invention R⁶ is an arylalkyl-group substituted with R²¹ groups, and said aryl moiety is phenyl, and said phenyl is substituted with at least two (e.g., 2 to 3, or 2, or 3) R²¹ groups, and two R²¹ groups on said phenyl is selected from the group consisting of: —SF₅, —OSF₅ and —Si(R^{15A})₃, and each R^{15A} is the same or different alkyl group.

In another embodiment of this invention R⁶ is an arylalkyl-group substituted with R²¹ groups, and said aryl moiety is phenyl, and said phenyl is substituted with at least two (e.g., 2 to 3, or 2, or 3) R²¹ groups, and two R²¹ groups on said phenyl is selected from the group consisting of: —SF₅, —OSF₅ and —Si(CH₃)₃.

In another embodiment of this invention R⁶ is an arylalkyl-group substituted with R²¹ groups, and said aryl moiety is phenyl, and said phenyl is substituted with at least one (e.g., 1 to 3, or 1 to 2) R²¹ group, and one R²¹ group on said phenyl is —SF₅.

In another embodiment of this invention R⁶ is an arylalkyl-group substituted with R²¹ groups, and said aryl moiety is phenyl, and said phenyl is substituted with at least one (e.g., 1 to 3, or 1 to 2) R²¹ group, and one R²¹ group on said phenyl is —OSF₅.

In another embodiment of this invention R⁶ is an arylalkyl-group substituted with R²¹ groups, and said aryl moiety is phenyl, and said phenyl is substituted with at least one (e.g., 1 to 3, or 1 to 2) R²¹ group, and one R²¹ group on said phenyl is —Si(R^{15A})₃, wherein each R^{15A} is independently selected.

In another embodiment of this invention R⁶ is an arylalkyl-group substituted with R²¹ groups, and said aryl moiety is phenyl, and said phenyl is substituted with at least one (e.g., 1 to 3, or 1 to 2) R²¹ group, and one R²¹ group on said phenyl is —Si(R^{15A})₃, and each R^{15A} is the same or different alkyl group.

In another embodiment of this invention R⁶ is an arylalkyl-group substituted with R²¹ groups, and said aryl moiety is phenyl, and said phenyl is substituted with at least one (e.g., 1 to 3, or 1 to 2) R²¹ group, and one R²¹ group on said phenyl is —Si(CH₃)₃.

In another embodiment of this invention R⁶ is an arylalkyl-group substituted with R²¹ groups, and said aryl moiety is phenyl, and said phenyl is substituted with at least two (e.g., 2 to 3) R²¹ groups, and two of the R²¹ groups on said phenyl are —SF₅.

In another embodiment of this invention R⁶ is an arylalkyl-group substituted with R²¹ groups, and said aryl moiety is phenyl, and said phenyl is substituted with at least two (e.g., 2 to 3) R²¹ groups, and two of the R²¹ groups on said phenyl are —OSF₅.

In another embodiment of this invention R⁶ is an arylalkyl-group substituted with R²¹ groups, and said aryl moiety is phenyl, and said phenyl is substituted with at least two (e.g., 2 to 3) R²¹ groups, and two of the R²¹ groups on said phenyl are —Si(R^{15A})₃, wherein each R^{15A} is independently selected.

In another embodiment of this invention R⁶ is an arylalkyl-group substituted with R²¹ groups, and said aryl moiety is phenyl, and said phenyl is substituted with at least two (e.g., 2 to 3) R²¹ groups, and two of the R²¹ groups on said phenyl are —Si(R^{15A})₃, and each R^{15A} is the same or different alkyl group.

In another embodiment of this invention R⁶ is an arylalkyl-group substituted with R²¹ groups, and said aryl moiety is phenyl, and said phenyl is substituted with at least two (e.g., 2 to 3) R²¹ groups, and two of the R²¹ groups on said phenyl are —Si(CH₃)₃.

Other embodiments of the compounds of formula (I) are directed to any one of the embodiments directed to R¹ being an alkyl substituted with one R²¹ group, wherein said alkyl is

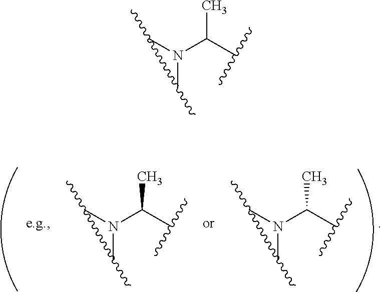

Other embodiments of the compounds of formula (I) are directed to any one of the embodiments directed to R¹ being an alkyl substituted with one R²¹ group, wherein said alkyl is

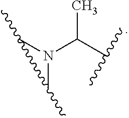

Other embodiments of the compounds of formula (I) are directed to any one of the embodiments directed to R¹ being an alkyl substituted with one R²¹ group, wherein said alkyl is

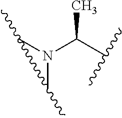

Other embodiments of the compounds of formula (I) are directed to any one of the embodiments directed to R¹ being an alkyl substituted with one R²¹ group, wherein said alkyl is

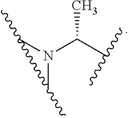

In another embodiment of the compounds of formula (I) R¹ is:

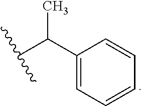

In another embodiment of the compounds of formula (I) R¹ is:

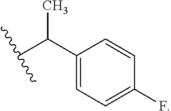

In another embodiment of the compounds of formula (I) R¹ is:

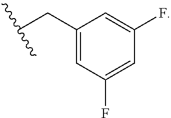

In another embodiment of the compounds of formula (I) R¹ is:

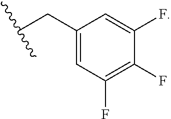

In another embodiment of the compounds of formula (I) R¹ is:

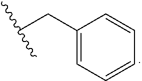

In another embodiment of the compounds of formula (I) R¹ is:

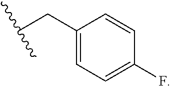

In another embodiment of the compounds of formula (I) R¹ is:

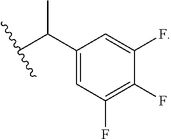

In another embodiment of the compounds of formula (I) R⁶ is:

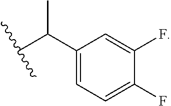

In another embodiment of the compounds of formula (I) R⁶ is:

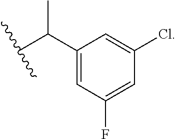

In another embodiment of the compounds of formula (I) R⁶ is:

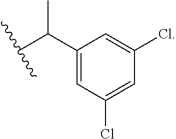

In another embodiment of the compounds of formula (I) R⁶ is:

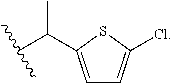

In another embodiment of the compounds of formula (I) R⁶ is:

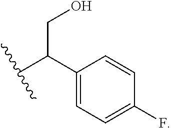

In another embodiment of the compounds of formula (I) R⁶ is:

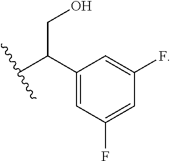

In another embodiment of the compounds of formula (I) R⁶ is:

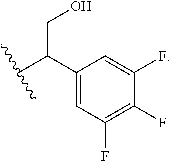

In another embodiment of the compounds of formula (I) R⁶ is:

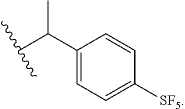

In another embodiment of the compounds of formula (I) R⁶ is:

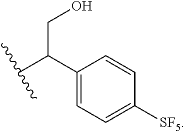

In another embodiment of the compounds of formula (I) R⁶ is:

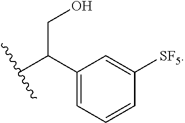

In another embodiment of the compounds of formula (I) R⁶ is:

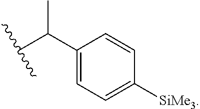

In another embodiment of the compounds of formula (I) R⁶ is:

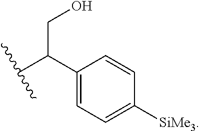

In another embodiment of the compounds of formula (I) R⁶ is:

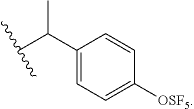

In another embodiment of the compounds of formula (I) R⁶ is:

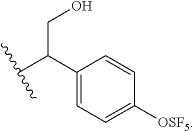

In another embodiment of the compounds of formula (I) R⁶ is:

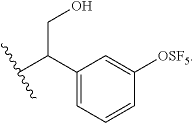

In another embodiment of this invention R⁶ is selected from the group consisting of:

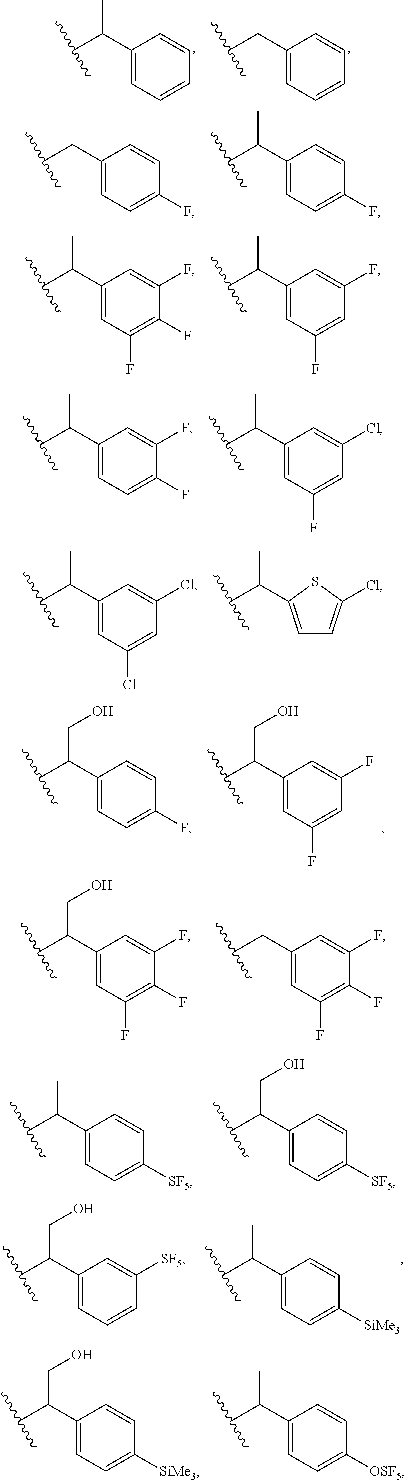

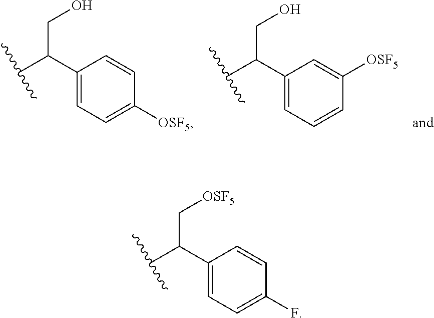

In another embodiment of this invention R⁶ is selected from the group consisting of:

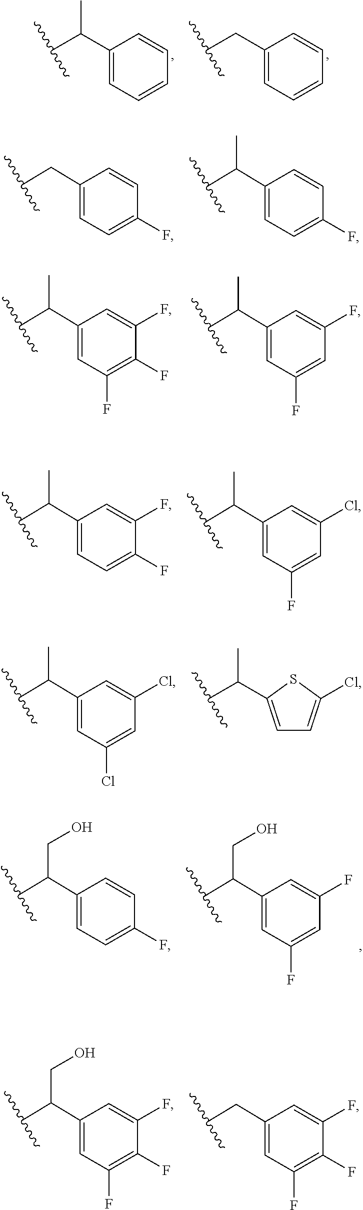

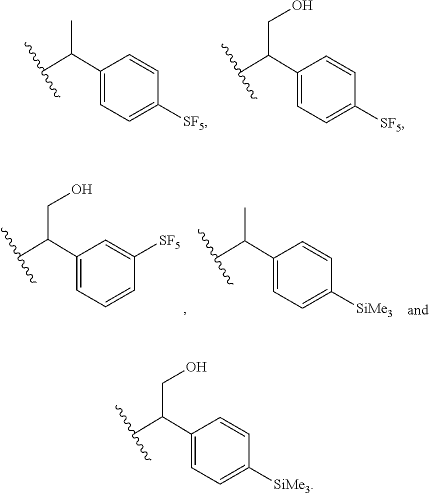

In another embodiment of this invention R⁶ is selected from the group consisting of:

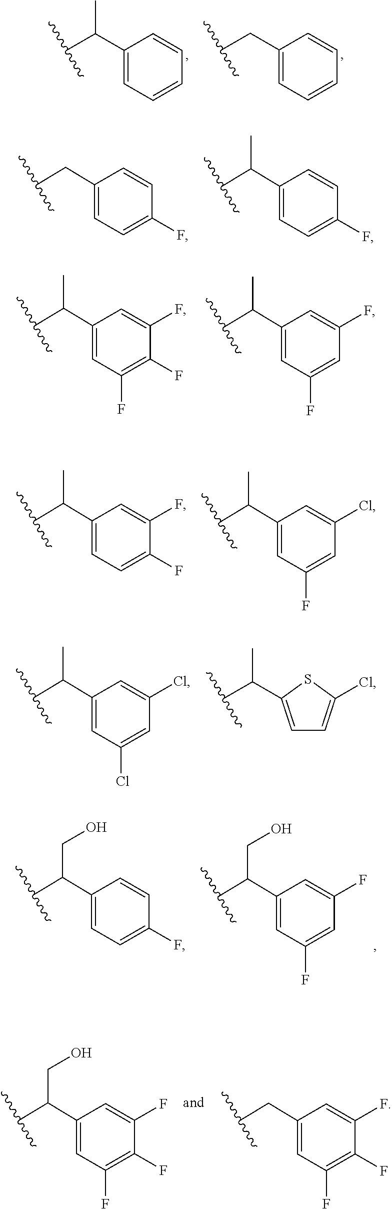

In another embodiment, R⁶ is selected from the group consisting of:

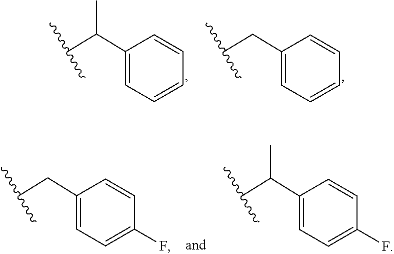

In another embodiment of this invention R⁶ is selected from the group consisting of:

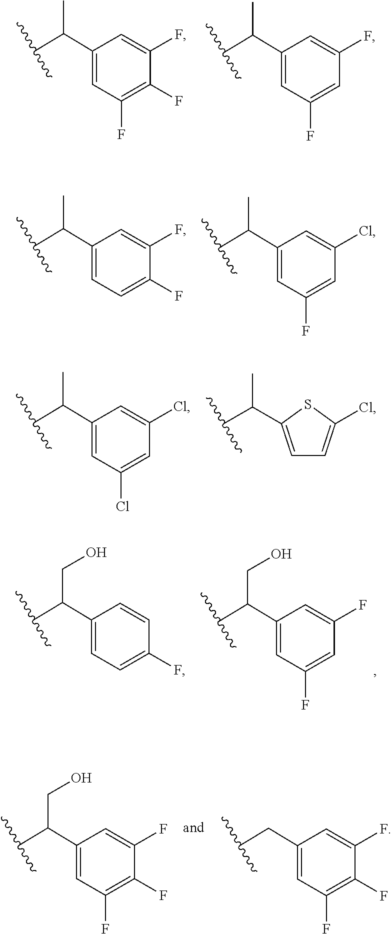

In another embodiment R⁶ is selected from the group consisting of:

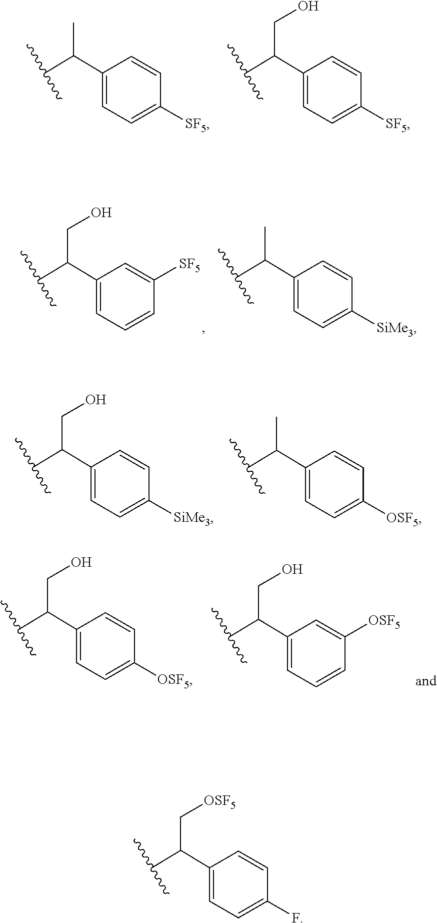

In another embodiment R⁶ is selected from the group consisting of:

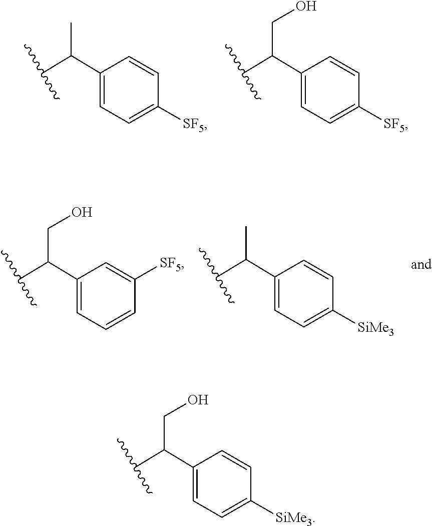

In another embodiment R⁶ is selected from the group consisting of:

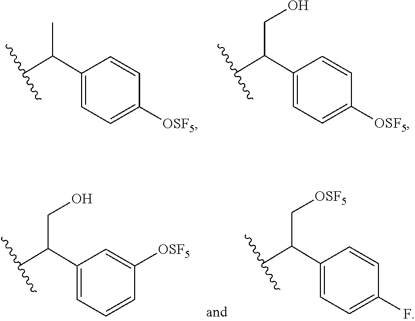

In another embodiment, R⁸ is H.

In another embodiment, R⁸ is alkyl.

In another embodiment, R⁸ is methyl.

In another embodiment of this invention R¹⁰ is selected from the group consisting of a bond, alkyl-, alkenyl-, alkynyl-, aryl-, arylalkyl-, alkylaryl-, cycloalkyl-, cycloalkylalkyl-, heteroaryl-, heteroarylalkyl-, heterocyclyl-, heterocyclylalkyl- and the moieties:

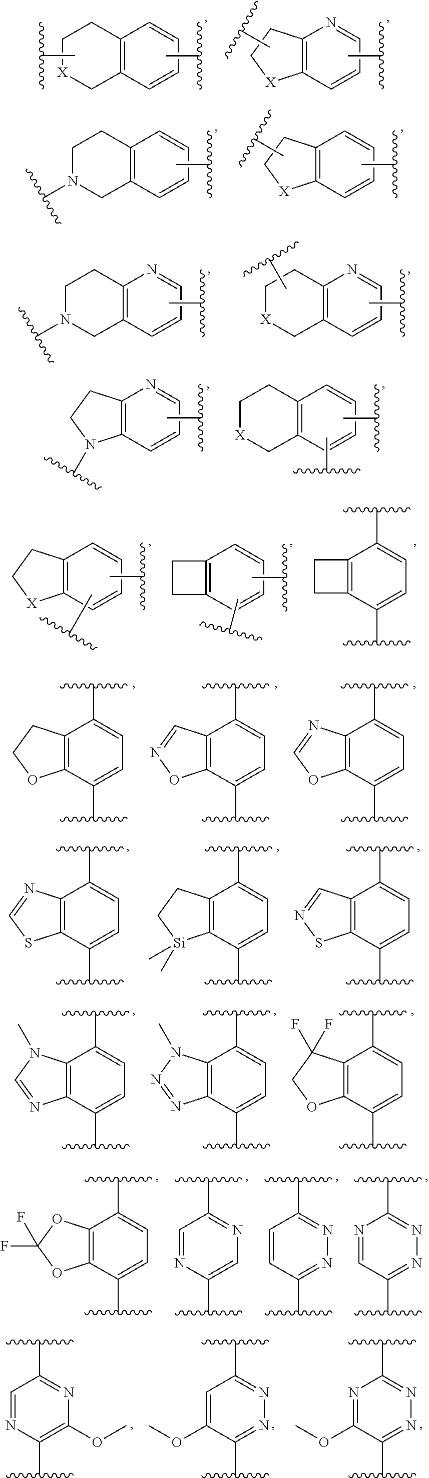

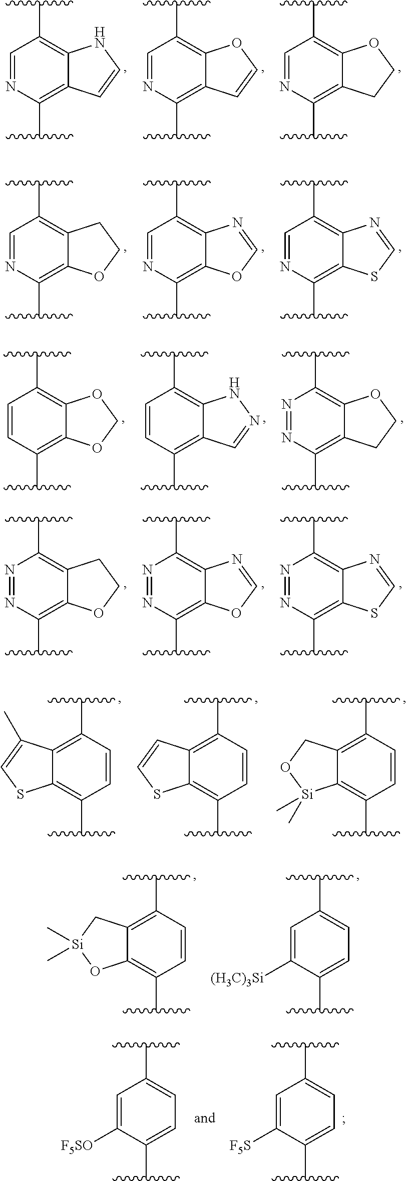

wherein X is O, N(R¹⁴) or S and wherein each R¹⁰ group (except for the bond) is optionally substituted with 1-3 independently selected R²¹ substituents.

In another embodiment of this invention R¹⁰ in formula I is selected from the group consisting of:

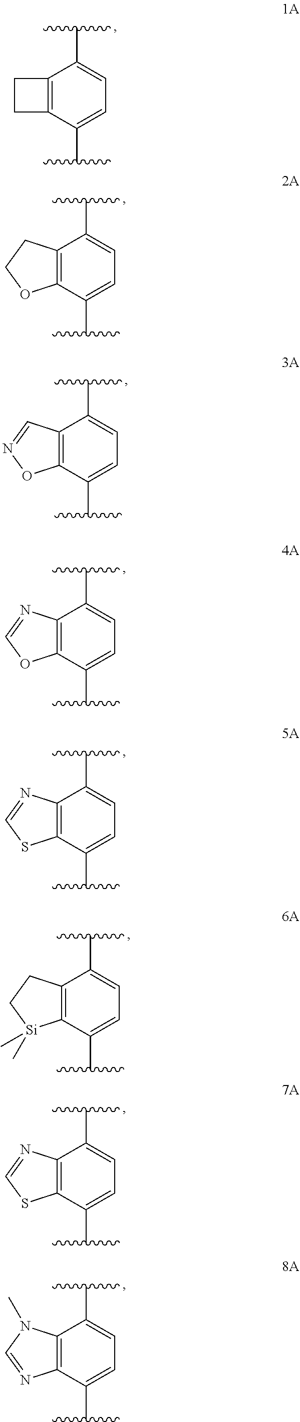

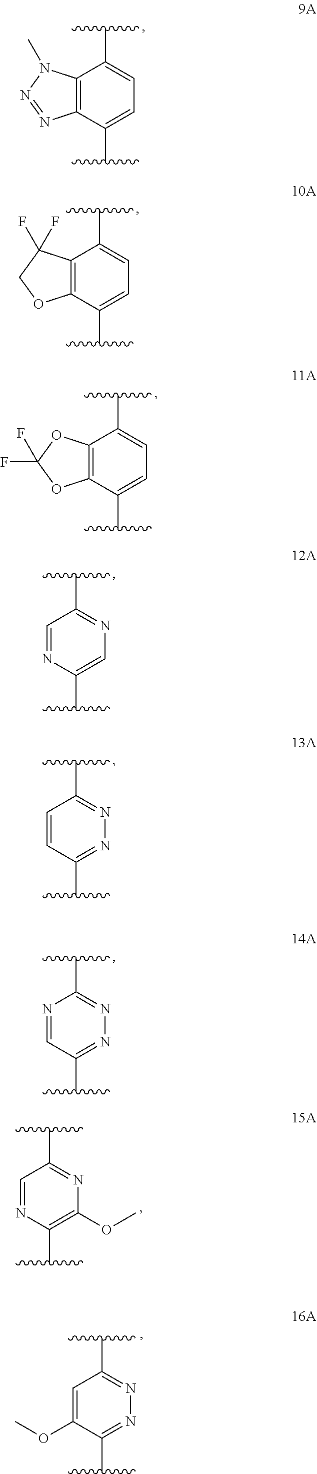

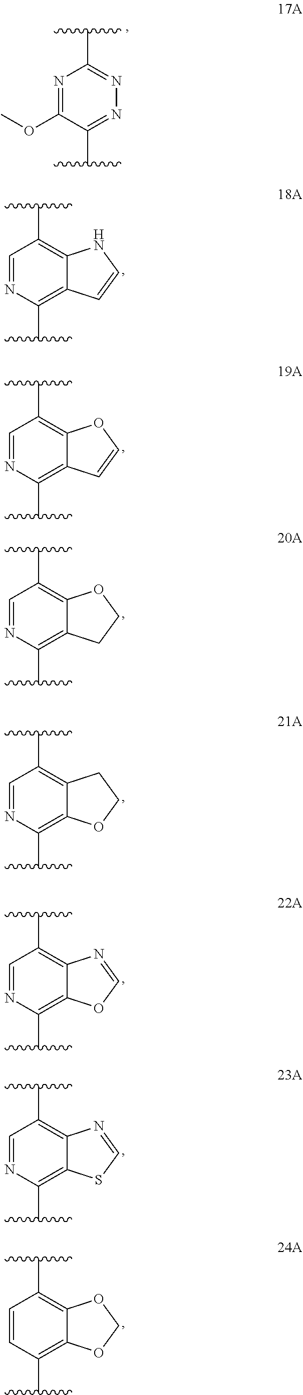

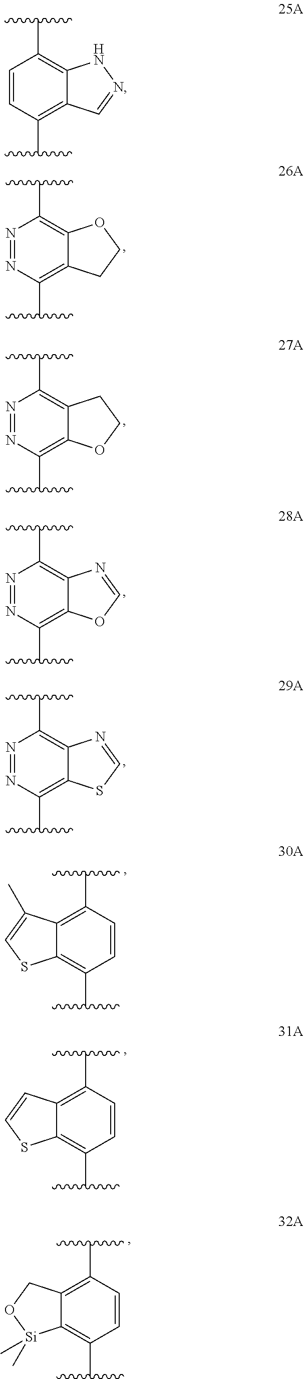

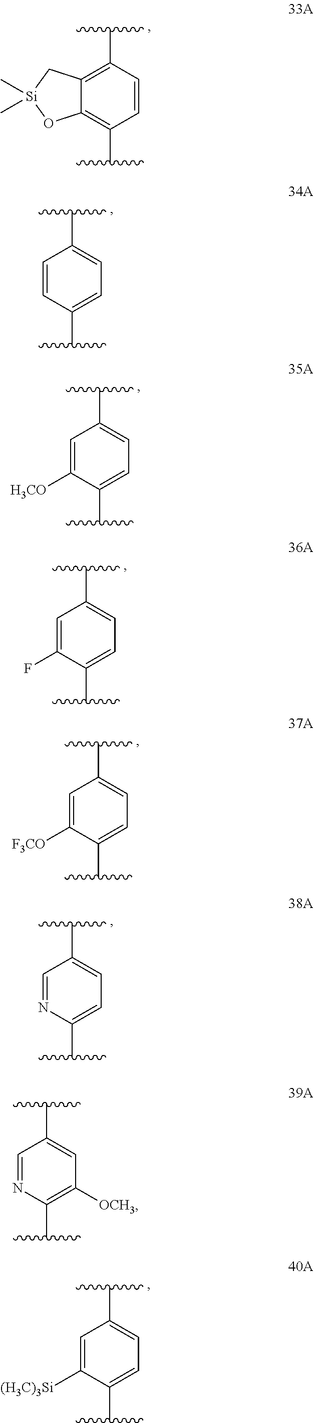

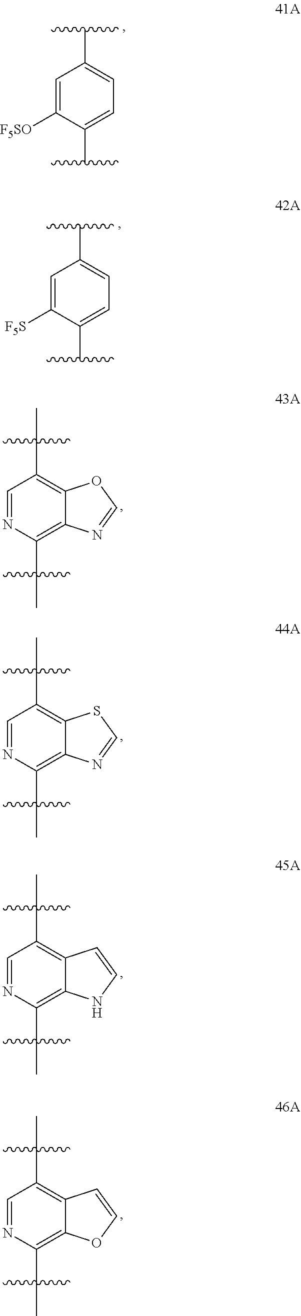

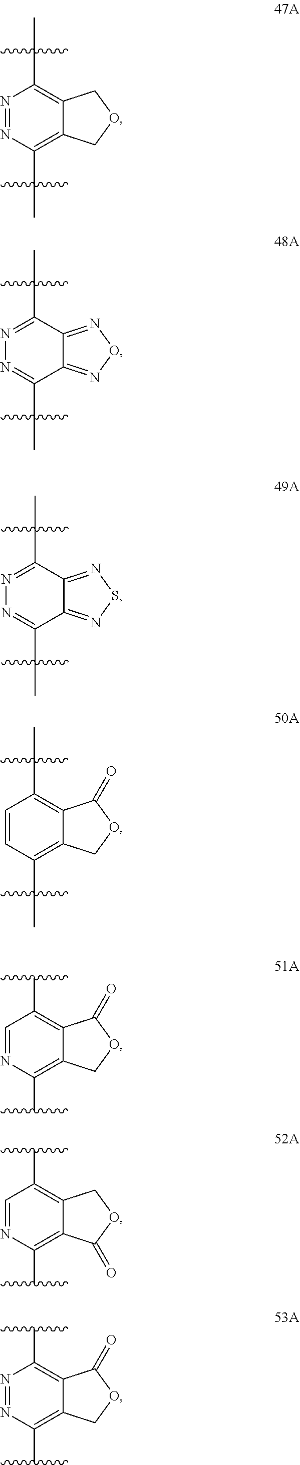

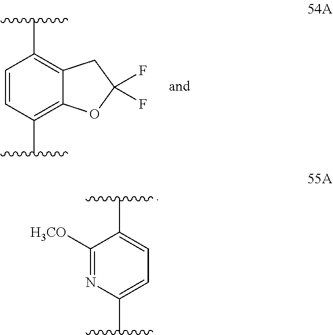

In another embodiment of this invention R¹⁰ in formula I is selected from the group consisting of:

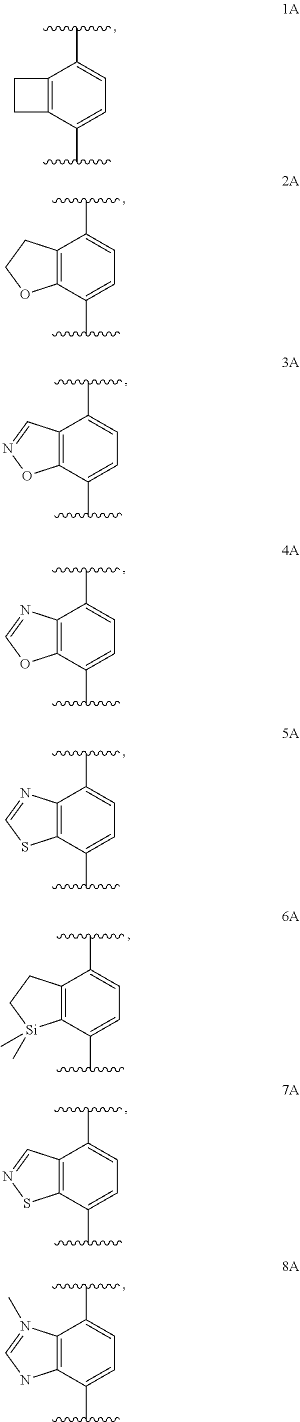

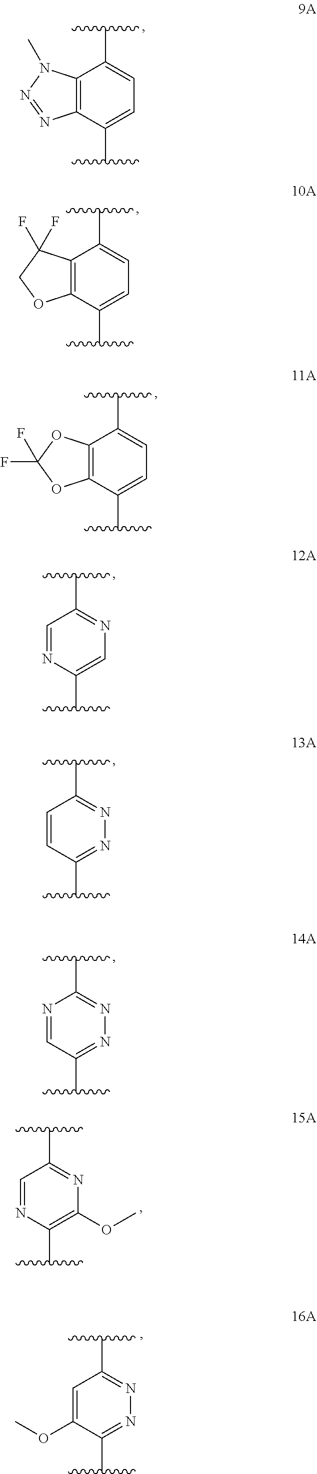

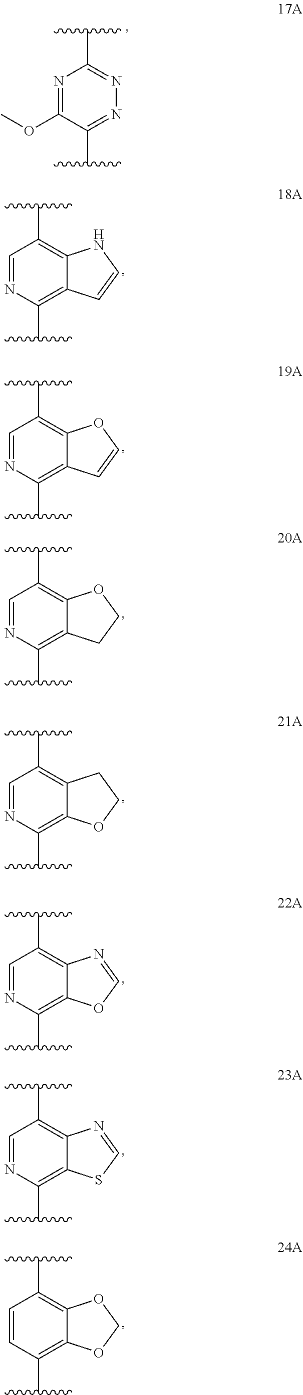

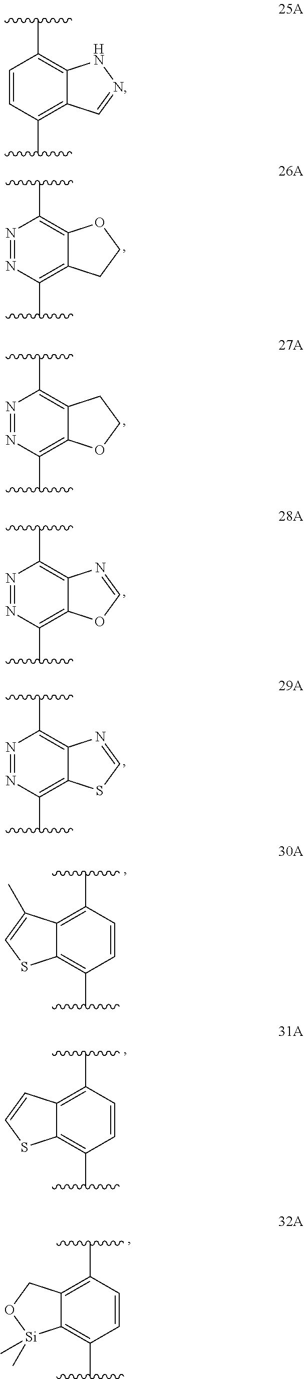

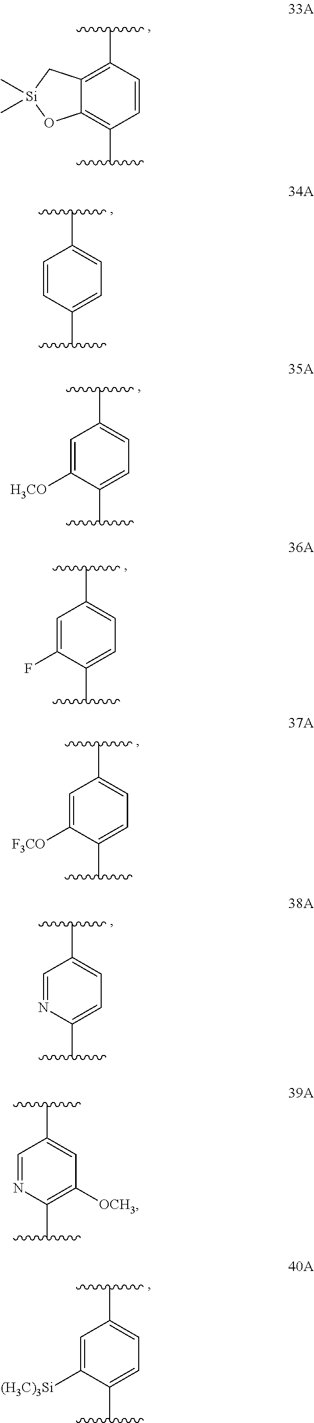

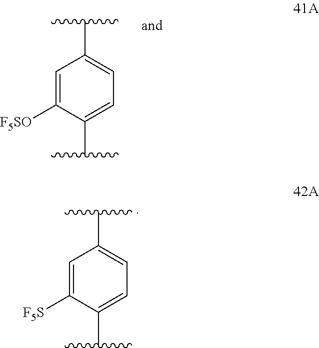

In another embodiment of this invention R¹⁰ is group 1A. In another embodiment of this invention R¹⁰ is group 2A. In another embodiment of this invention R¹⁰ is group 3A. In another embodiment of this invention R¹⁰ is group 4A. In another embodiment of this invention R¹⁰ is group 5A. In another embodiment of this invention R¹⁰ is group 6A. In another embodiment of this invention R¹⁰ is group 7A. In another embodiment of this invention R¹⁰ is group 8A. In another embodiment of this invention R¹⁰ is group 9A. In another embodiment of this invention R¹⁰ is group 10A. In another embodiment of this invention R¹⁰ is group 11A. In another embodiment of this invention R¹⁰ is group 12A. In another embodiment of this invention R¹⁰ is group 13A. In another embodiment of this invention R¹⁰ is group 14A. In another embodiment of this invention R¹⁰ is group 15A. In another embodiment of this invention R¹⁰ is group 16A. In another embodiment of this invention R¹⁰ is group 17A. In another embodiment of this invention R¹⁰ is group 18A. In another embodiment of this invention R¹⁰ is group 19A. In another embodiment of this invention R¹⁰ is group 20A. In another embodiment of this invention R¹⁰ is group 21A. In another embodiment of this invention R¹⁰ is group 22A. In another embodiment of this invention R¹⁰ is group 23A. In another embodiment of this invention R¹⁰ is group 24A. In another embodiment of this invention R¹⁰ is group 25A. In another embodiment of this invention R¹⁰ is group 26A. In another embodiment of this invention R¹⁰ is group 27A. In another embodiment of this invention R¹⁰ is group 28A. In another embodiment of this invention R¹⁰ is group 29A. In another embodiment of this invention R¹⁰ is group 30A. In another embodiment of this invention R¹⁰ is group 31A. In another embodiment of this invention R¹⁰ is group 32A. In another embodiment of this invention R¹⁰ is group 33A. In another embodiment of this invention R¹⁰ is group 34A. In another embodiment of this invention R¹⁰ is group 35A. In another embodiment of this invention R¹⁰ is group 36A. In another embodiment of this invention R¹⁰ is group 37A. In another embodiment of this invention R¹⁰ is group 38A. In another embodiment of this invention R¹⁰ is group 39A. In another embodiment of this invention R¹⁰ is group 40A. In another embodiment of this invention R¹⁰ is group 41A. In another embodiment of this invention R¹⁰ is group 42A. In another embodiment of this invention R¹⁰ is group 43A. In another embodiment of this invention R¹⁰ is group 44A. In another embodiment of this invention R¹⁰ is group 45A. In another embodiment of this invention R¹⁰ is group 46A. In another embodiment of this invention R¹⁰ is group 47A. In another embodiment of this invention R¹⁰ is group 48A. In another embodiment of this invention R¹⁰ is group 49A. In another embodiment of this invention R¹⁰ is group 50A. In another embodiment of this invention R¹⁰ is group 51A. In another embodiment of this invention R¹⁰ is group 52A. In another embodiment of this invention R¹⁰ is group 53A. In another embodiment of this invention R¹⁰ is group 54A. In another embodiment of this invention R¹⁰ is group 55A.

In another embodiment, R¹⁰ is aryl.

In another embodiment, R¹⁰ is phenyl.

In another embodiment R¹⁰ is aryl substituted with 1 halo.

In another embodiment R¹⁰ is aryl substituted with 1 halo, and said halo is F.

In another embodiment R¹⁹ is aryl substituted with 1 to 3 independently selected R²¹ moieties.

In another embodiment R¹⁹ is aryl substituted with 1 to 3 R²¹ moieties, wherein each R²¹ moiety is the same or different —OR¹⁵ group.

In another embodiment R¹⁹ is aryl substituted with 1 R²¹ moiety.

In another embodiment R¹⁹ is phenyl substituted with 1 halo.

In another embodiment R¹⁹ is phenyl substituted with 1 halo, and said halo is F.

In another embodiment R¹⁹ is 3-halo-phenyl:

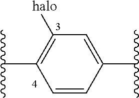

(wherein the bond from the carbon labeled as 4 is to the R⁹ group).

In another embodiment R¹⁹ is 3-F-phenyl:

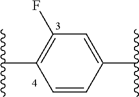

(wherein the bond from the carbon labeled as 4 is to the R⁹ group).

In another embodiment R¹⁹ is aryl substituted with one —OR¹⁵ group.

In another embodiment R¹⁹ is aryl substituted with one —OR¹⁵ group, and said R¹⁵ is alkyl (e.g., methyl).

In another embodiment R¹⁹ is phenyl substituted with 1 to 3 independently selected R²¹ moieties.

In another embodiment R¹⁹ is phenyl substituted with 1 to 3 R²¹ moieties, wherein each R²¹ moiety is the same or different —OR¹⁵ group.

In another embodiment R¹⁹ is phenyl substituted with 1 R²¹ moiety.

In another embodiment R¹⁹ is phenyl substituted with one —OR¹⁵ group.

In another embodiment R¹⁹ is phenyl substituted with one —OR¹⁵ group, and said R¹⁵ is alkyl (e.g., methyl).

In another embodiment R¹⁹ is 3-OR¹⁵-phenyl:

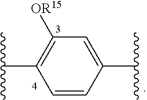

(wherein the bond from the carbon labeled as 4 is to the R⁹ group).

In another embodiment R¹⁰ is 3-OR¹⁵-phenyl:

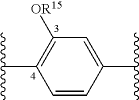

wherein R¹⁵ is alkyl (wherein the bond from the carbon labeled as 4 is to the R⁹ group).

In another embodiment R¹⁰ is 3-OR¹⁵-ohenyl:

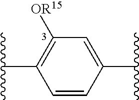

wherein R¹⁵ is methyl (i.e., R¹⁹ is 3-methoxy-phenyl).

In another embodiment, R¹⁹ is heteroaryl.

In another embodiment, R¹⁹ is unsubstituted heteroaryl.

In another embodiment R¹⁹ is unsubstituted heteroaryl wherein said heteroaryl is pyridyl.

In another embodiment R¹⁹ is:

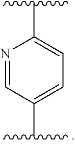

In another embodiment R¹⁰ is:

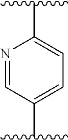

wherein the —R¹⁰-R⁹ moiety is:

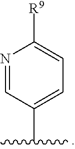

In another embodiment R¹⁰ is aryl substituted with 1 to 3 R²¹ moieties, wherein each R²¹ moiety is the same or different halo.

In another embodiment R¹⁰ is aryl substituted with 1 to 3 R²¹ moieties, wherein each R²¹ moiety is F.

In another embodiment R¹⁰ is aryl substituted with one R²¹ moiety, and said R²¹ moiety is halo.

In another embodiment R¹⁰ is aryl substituted with one R²¹ moiety, said R²¹ moiety is -halo, and said halo is F.

In another embodiment R¹⁰ is phenyl substituted with 1 to 3 R²¹ moieties, wherein each R²¹ moiety is the same or different halo.

In another embodiment R¹⁰ is phenyl substituted with 1 to 3 R²¹ moieties, wherein each R²¹ moiety is F.

In another embodiment R¹⁰ is selected from the group consisting of:

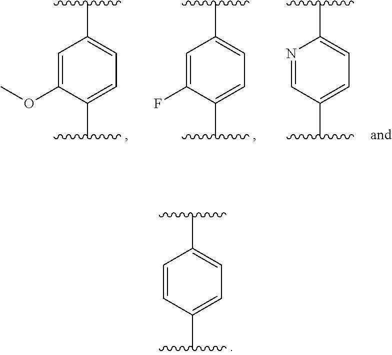

In another embodiment of this invention R⁹ is selected from the group consisting of heteroaryl and heteroaryl substituted with 1-3 R²¹ groups, and wherein each R²¹ is independently selected.

In another embodiment, R⁹ is unsubstituted heteroaryl.

In another embodiment, R⁹ is heteroaryl which is substituted with 1-3 substituents which can be the same or different, each substituent being independently selected from the group consisting of halo, alkyl, CN, NH₂, NH(alkyl), N(alkyl)₂, hydroxy and alkoxy groups.

In another embodiment, R⁹ is heteroaryl substituted with 1 to 3 independently selected alkyl groups.

In another embodiment of this invention R⁹ is selected from the group consisting of:

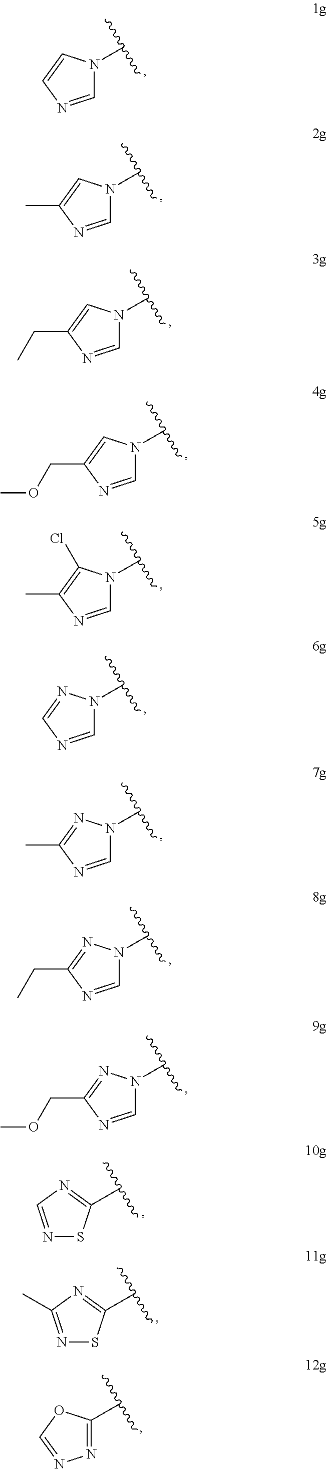

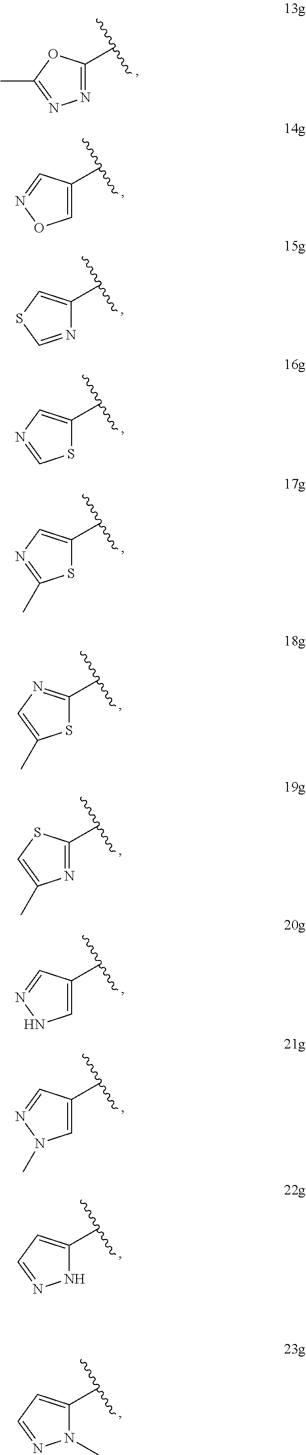

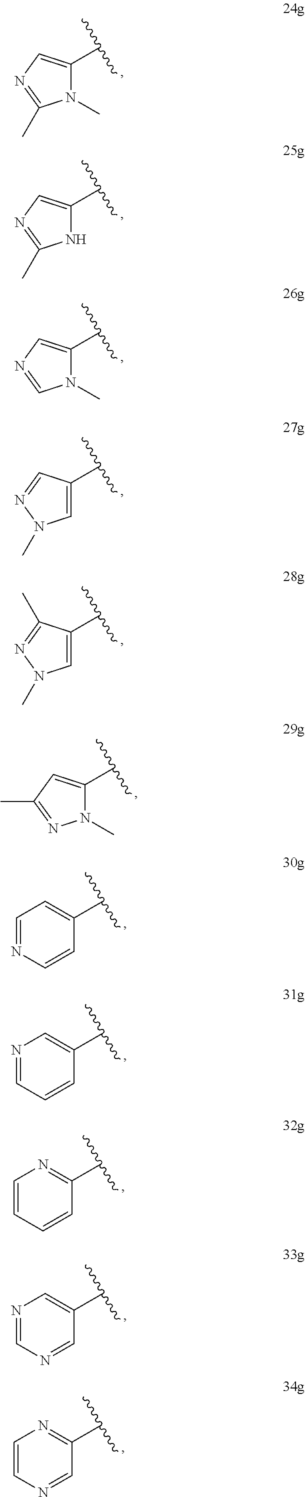

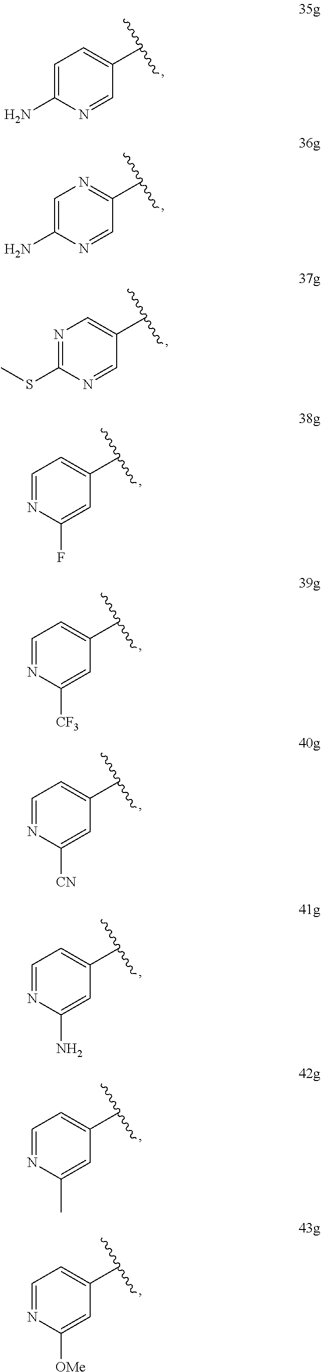

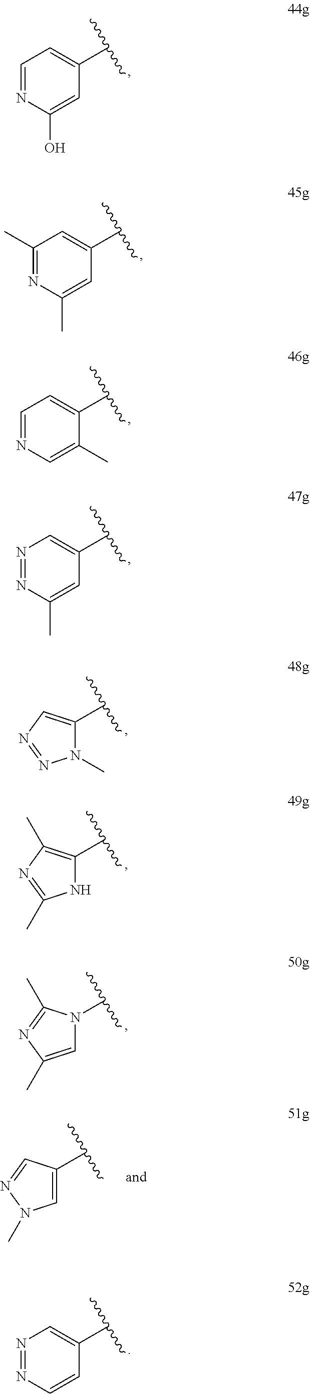

In another embodiment of this invention R⁹ is selected from the group consisting of:

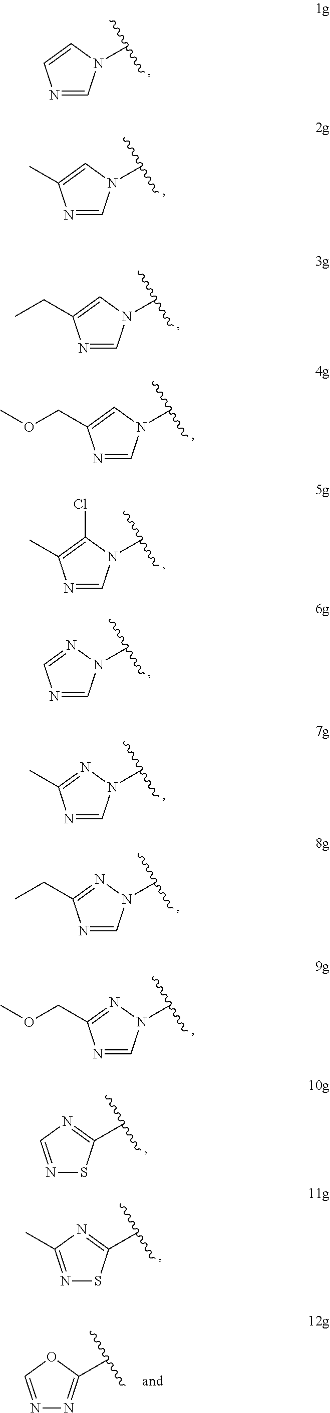

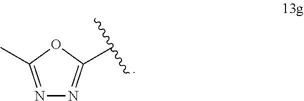

In another embodiment of this invention R⁹ is 1 g. In another embodiment of this invention R⁹ is:

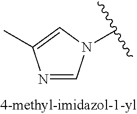

(i.e. 2 g). In another embodiment of this invention R⁹ is 3 g. In another embodiment of this invention R⁹ is 4 g. In another embodiment of this invention R⁹ is 5 g. In another embodiment of this invention R⁹ is 6 g. In another embodiment of this invention R⁹ is 7 g. In another embodiment of this invention R⁹ is 8 g. In another embodiment of this invention R⁹ is 9 g. In another embodiment of this invention R⁹ is 10 g. In another embodiment of this invention R⁹ is 11 g. In another embodiment of this invention R⁹ is 12 g. In another embodiment of this invention R⁹ is 13 g. In another embodiment of this invention R⁹ is 14 g. In another embodiment of this invention R⁹ is 15 g. In another embodiment of this invention R⁹ is 16 g. In another embodiment of this invention R⁹ is 17 g. In another embodiment of this invention R⁹ is 18 g. In another embodiment of this invention R⁹ is 19 g. In another embodiment of this invention R⁹ is 20 g. In another embodiment of this invention R⁹ is 21 g. In another embodiment of this invention R⁹ is 22 g. In another embodiment of this invention R⁹ is 23 g. In another embodiment of this invention R⁹ is 24 g. In another embodiment of this invention R⁹ is 25 g. In another embodiment of this invention R⁹ is 26 g. In another embodiment of this invention R⁹ is 27 g. In another embodiment of this invention R⁹ is 28 g. In another embodiment of this invention R⁹ is 29 g. In another embodiment of this invention R⁹ is 30 g. In another embodiment of this invention R⁹ is 31 g. In another embodiment of this invention R⁹ is 32 g. In another embodiment of this invention R⁹ is 33 g. In another embodiment of this invention R⁹ is 34 g. In another embodiment of this invention R⁹ is 35 g. In another embodiment of this invention R⁹ is 36 g. In another embodiment of this invention R⁹ is 37 g. In another embodiment of this invention R⁹ is 38 g. In another embodiment of this invention R⁹ is 39 g. In another embodiment of this invention R⁹ is 40 g. In another embodiment of this invention R⁹ is 41 g. In another embodiment of this invention R⁹ is 42 g. In another embodiment of this invention R⁹ is 43 g. In another embodiment of this invention R⁹ is 44 g. In another embodiment of this invention R⁹ is 45 g. In another embodiment of this invention R⁹ is 46 g. In another embodiment of this invention R⁹ is 47 g. In another embodiment of this invention R⁹ is 48 g. In another embodiment of this invention R⁹ is 49 g. In another embodiment of this invention R⁹ is 50 g. In another embodiment of this invention R⁹ is 51 g. In another embodiment of this invention R⁹ is 52 g.

In another embodiment, R⁹ is heteroaryl substituted with one is alkyl group (e.g., methyl).

In another embodiment of this invention R⁹ is selected from the group consisting of imidazolyl and imidazolyl substituted with 1-3 R²¹ groups, and wherein each R²¹ is independently selected.

In another embodiment of this invention R⁹ is imidazolyl substituted with 1-3 R²¹ groups, and wherein each R²¹ is independently selected.

In another embodiment, R⁹ is imidazolyl substituted with 1-3 substituents independently selected from the group consisting of halo, alkyl, CN, NH₂, NH(alkyl), N(alkyl)₂, hydroxy and alkoxy groups.

In another embodiment, R⁹ is imidazol-1-yl.

In another embodiment, R⁹ is 4-methyl-imidazol-1-yl:

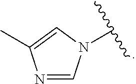

In another embodiment, R⁹ is 5-chloro-4-methyl-imidazol-1-yl.

In another embodiment R¹⁰ is selected from the group consisting of aryl and aryl substituted with one or more R²¹ groups, and R⁹ is selected from the group consisting of heteroaryl and heteroaryl substituted with one or more R²¹ groups, and wherein each R²¹ is independently selected.

In another embodiment R¹⁰ is selected from the group consisting of phenyl and phenyl substituted with 1-3 independently selected R²¹ groups, and R⁹ is selected from the group consisting of imidazolyl and imidazolyl substituted with 1-3 independently selected R²¹ groups.

In another embodiment R¹⁰ is phenyl substituted with 1-3 independently selected R²¹ groups, and R⁹ is selected from the group consisting of imidazolyl and imidazolyl substituted with 1-3 independently selected R²¹ groups.

In another embodiment R¹⁰ is selected from the group consisting of heteroaryl and heteroaryl substituted with 1-3 R²¹ groups, and the R⁹ group is selected from the group consisting of heteroaryl and heteroaryl substituted with 1-3 R²¹ groups, and wherein each R²¹ is independently selected.

In another embodiment R¹⁰ is selected from the group consisting of pyridyl and pyridyl substituted with 1-3 R²¹ groups, and the R⁹ group is selected from the group consisting of imidazolyl and imidazolyl substituted with 1-3 R²¹ groups, and wherein each R²¹ is independently selected.

In another embodiment, the R⁹—R¹⁰— moiety is:

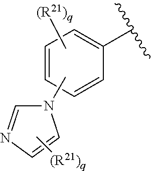

wherein q is 0, 1 or 2, such as, for example,

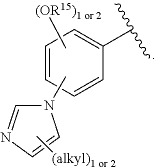

wherein R¹⁵ is alkyl (e.g., methyl), such as, for example

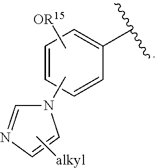

In another embodiment, the R⁹—R¹⁰— moiety is:

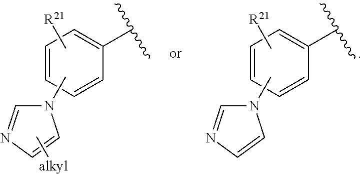

In another embodiment, the R⁹—R¹⁰— moiety is:

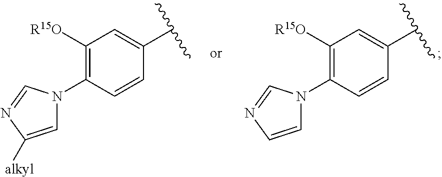

or wherein the R⁹—R¹⁰— moiety is:

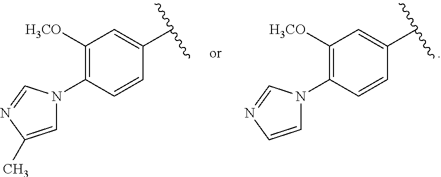

In another embodiment R¹⁰ is pyridyl, and the R⁹ group is imidazolyl substituted with 1-3 R²¹ groups, and wherein each R²¹ is independently selected.

In another embodiment the R⁹—R¹⁰— moiety is:

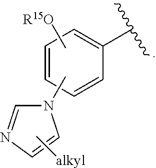

In another embodiment the R⁹—R¹⁰— moiety is:

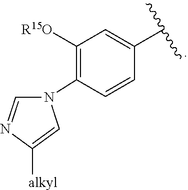

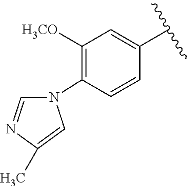

In another embodiment the R⁹—R¹⁰— moiety is:

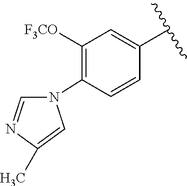

In another embodiment the R⁹—R¹⁰— moiety is:

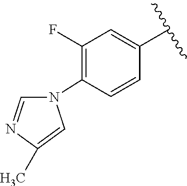

In another embodiment R⁹— R¹⁹— moiety is:

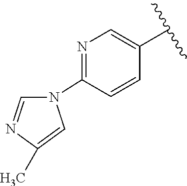

In another embodiment R⁹—R¹⁰— moiety is:

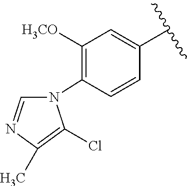

In another embodiment R⁹—R¹⁰— moiety is:

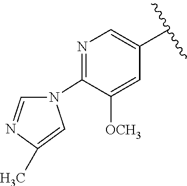

In another embodiment of this invention the R¹⁰-R⁹— moiety is selected from the group consisting of:

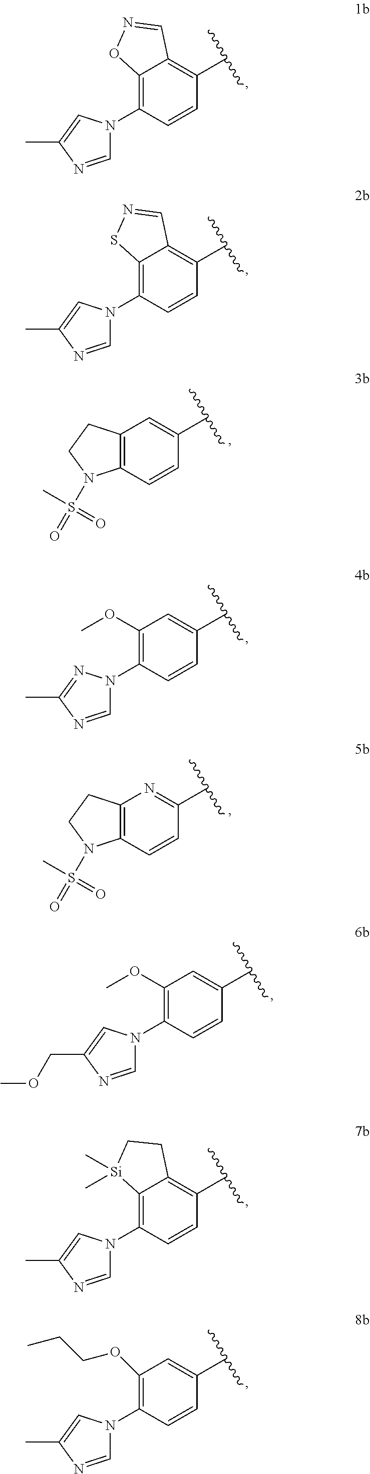

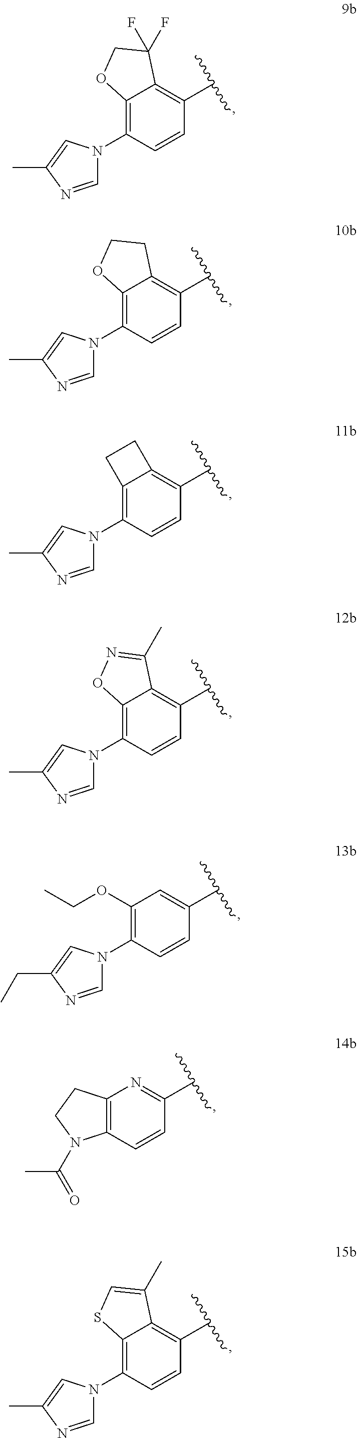

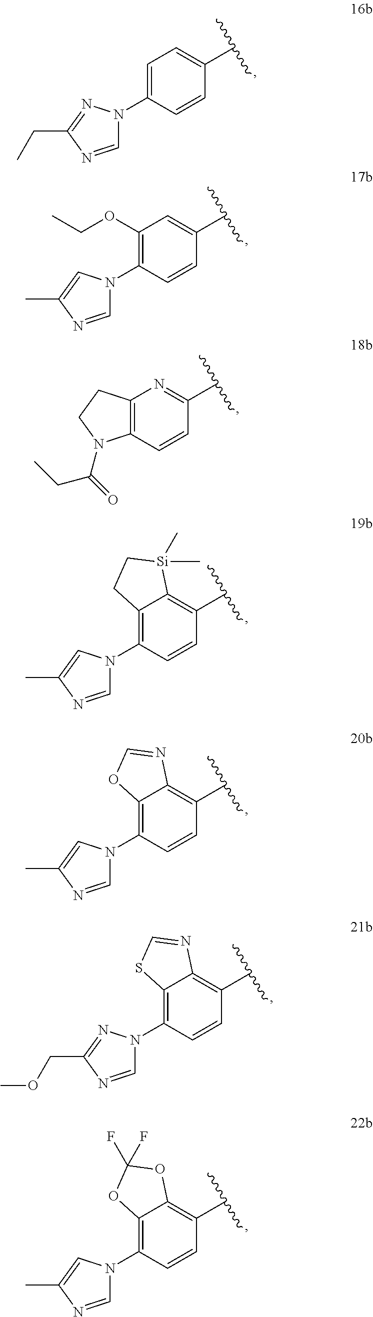

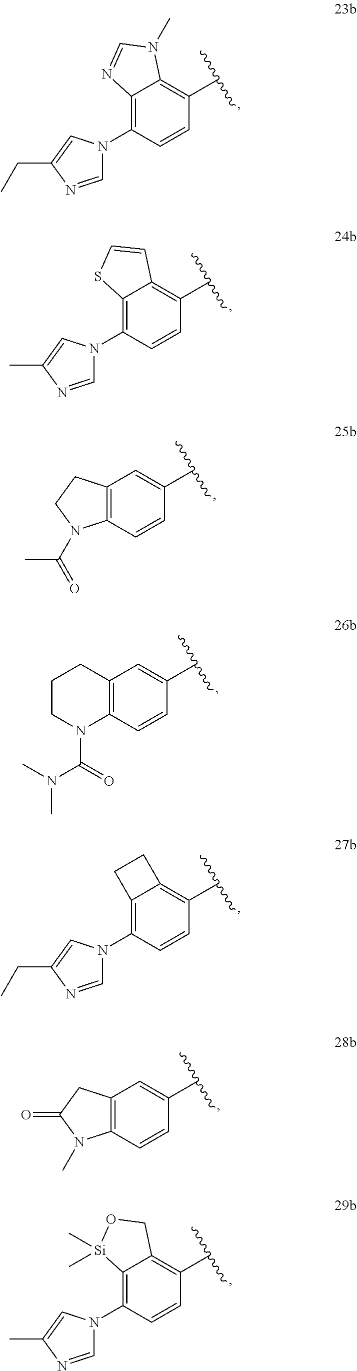

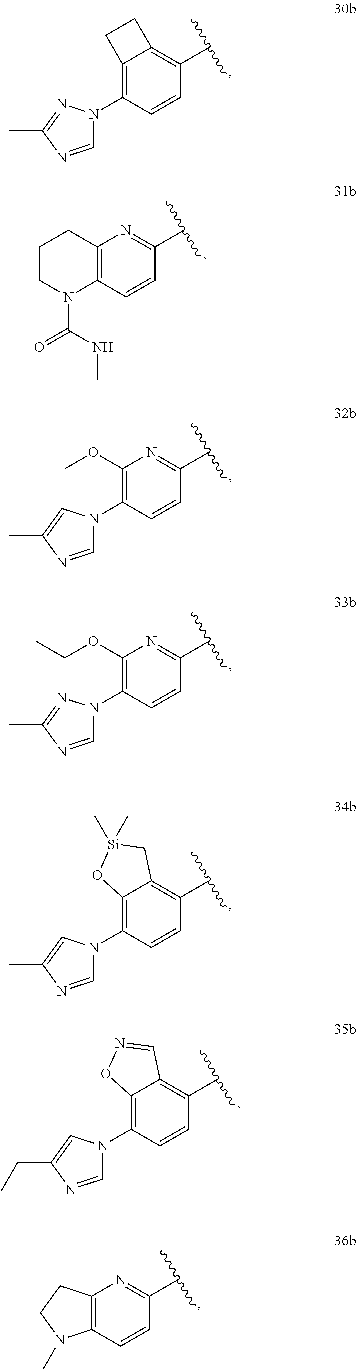

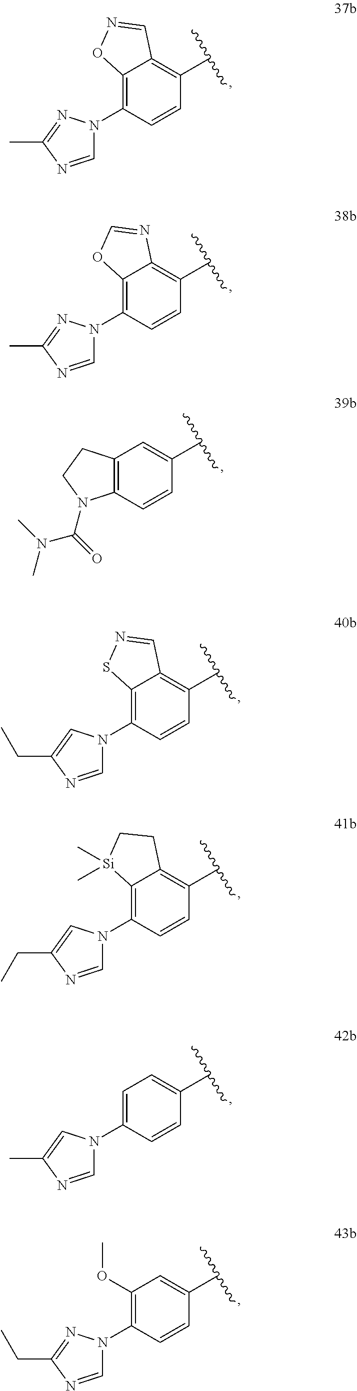

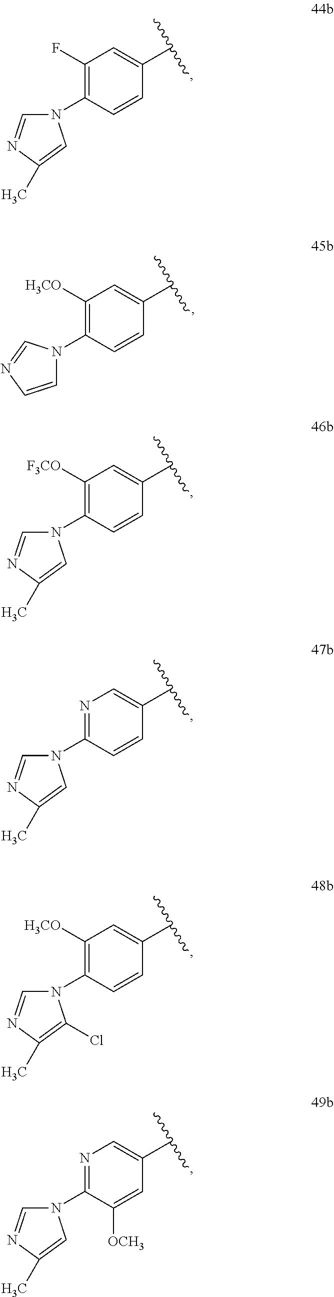

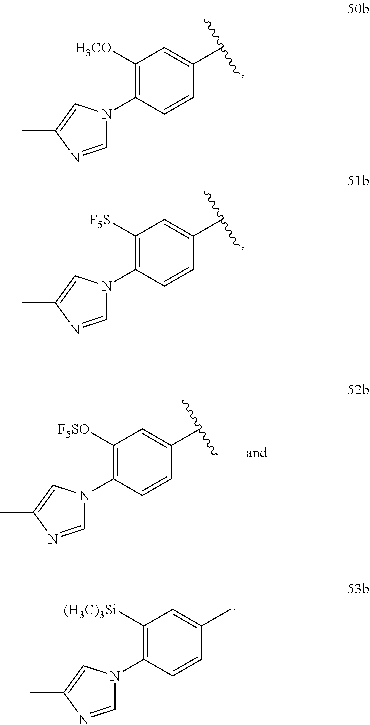

In another embodiment, this invention discloses a compound, or pharmaceutically acceptable salts, solvates, esters or prodrugs of said compound, said compound having the general structure shown in the formula 3:

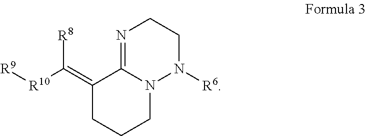

In another embodiment, this invention discloses a compound, or pharmaceutically acceptable salts, solvates, esters or prodrugs of said compound, said compound having the general structure shown in the formula 4:

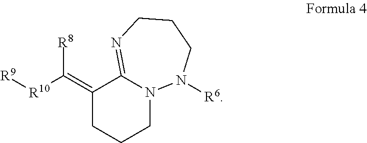

In another embodiment, this invention discloses a compound, or pharmaceutically acceptable salts, solvates, esters or prodrugs of said compound, said compound having the general structure shown in the formula 5:

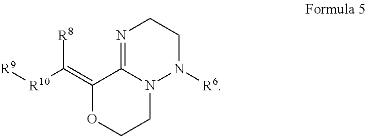

In another embodiment, this invention discloses a compound, or pharmaceutically acceptable salts, solvates, esters or prodrugs of said compound, said compound having the general structure shown in the formula 6:

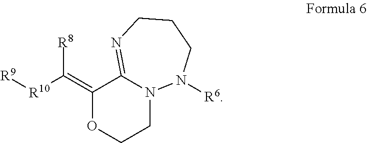

In another embodiment, this invention discloses a compound, or pharmaceutically acceptable salts, solvates, esters or prodrugs of said compound, said compound having the general structure shown in the formula 7:

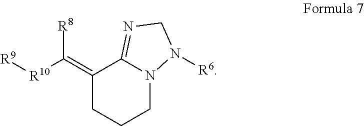

In another embodiment, this invention discloses a compound, or pharmaceutically acceptable salts, solvates, esters or prodrugs of said compound, said compound having the general structure shown in the formula 8:

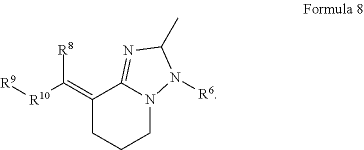

In another embodiment, this invention discloses a compound, or pharmaceutically acceptable salts, solvates, esters or prodrugs of said compound, said compound having the general structure shown in the formula 9:

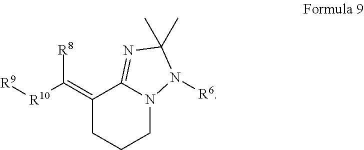

In another embodiment, this invention discloses a compound, or pharmaceutically acceptable salts, solvates, esters or prodrugs of said compound, said compound having the general structure shown in the formula 10:

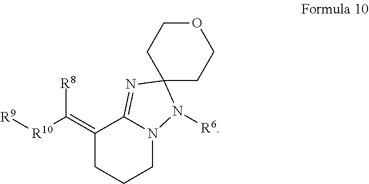

In another embodiment, this invention discloses a compound, or pharmaceutically acceptable salts, solvates, esters or prodrugs of said compound, said compound having the general structure shown in the formula II:

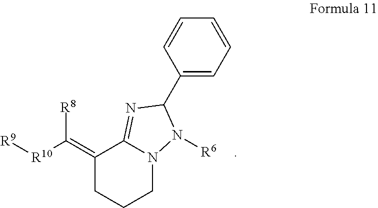

In another embodiment, this invention discloses a compound, or pharmaceutically acceptable salts, solvates, esters or prodrugs of said compound, said compound having the general structure shown in the formula 12:

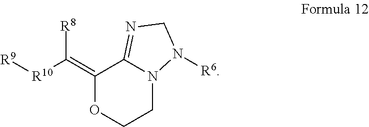

In another embodiment, this invention discloses a compound, or pharmaceutically acceptable salts, solvates, esters or prodrugs of said compound, said compound having the general structure shown in the formula 13:

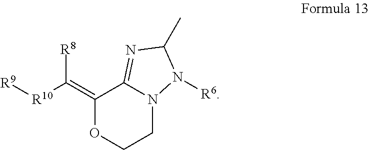

In another embodiment, this invention discloses a compound, or pharmaceutically acceptable salts, solvates, esters or prodrugs of said compound, said compound having the general structure shown in the formula 14:

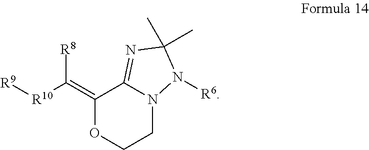

In another embodiment, this invention discloses a compound, or pharmaceutically acceptable salts, solvates, esters or prodrugs of said compound, said compound having the general structure shown in the formula 15:

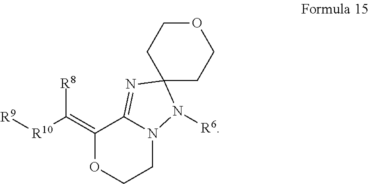

In another embodiment, this invention discloses a compound, or pharmaceutically acceptable salts, solvates, esters or prodrugs of said compound, said compound having the general structure shown in the formula 16:

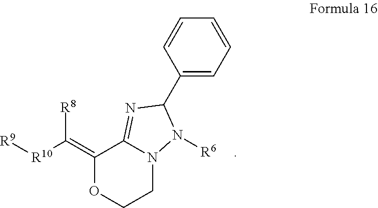

In another embodiment, this invention discloses a compound, or pharmaceutically acceptable salts, solvates, esters or prodrugs of said compound, said compound having the general structure shown in the formula 17:

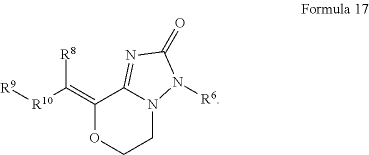

In another embodiment, this invention discloses a compound, or pharmaceutically acceptable salts, solvates, esters or prodrugs of said compound, said compound having the general structure shown in the formula 18:

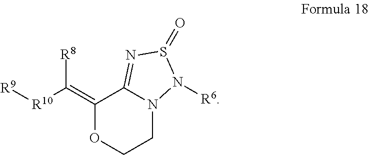

In another embodiment, this invention discloses a compound, or pharmaceutically acceptable salts, solvates, esters or prodrugs of said compound, said compound having the general structure shown in the formula 19:

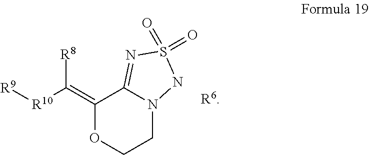

In another embodiment, this invention discloses a compound, or pharmaceutically acceptable salts, solvates, esters or prodrugs of said compound, said compound having the general structure shown in the formula 20:

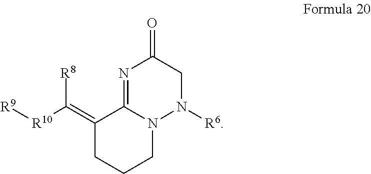

In another embodiment, this invention discloses a compound, or pharmaceutically acceptable salts, solvates, esters or prodrugs of said compound, said compound having the general structure shown in the formula 21:

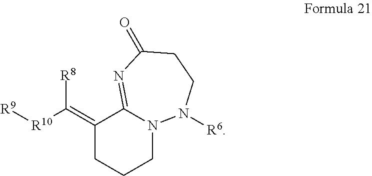

In another embodiment, this invention discloses a compound, or pharmaceutically acceptable salts, solvates, esters or prodrugs of said compound, said compound having the general structure shown in the formula 22:

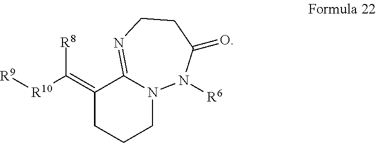

In another embodiment, this invention discloses a compound, or pharmaceutically acceptable salts, solvates, esters or prodrugs of said compound, said compound having the general structure shown in the formula 23:

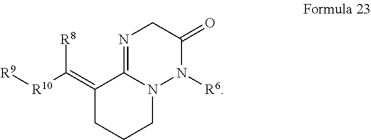

In another embodiment, this invention discloses a compound, or pharmaceutically acceptable salts, solvates, esters or prodrugs of said compound, said compound having the general structure shown in the formula 24:

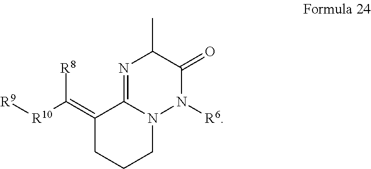

In another embodiment, this invention discloses a compound, or pharmaceutically acceptable salts, solvates, esters or prodrugs of said compound, said compound having the general structure shown in the formula 25:

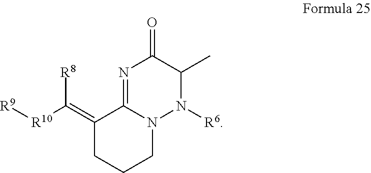

In another embodiment, this invention discloses a compound, or pharmaceutically acceptable salts, solvates, esters or prodrugs of said compound, said compound having the general structure shown in the formula 26:

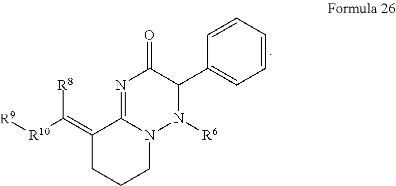

In another embodiment, this invention discloses a compound, or pharmaceutically acceptable salts, solvates, esters or prodrugs of said compound, said compound having the general structure shown in the formula 27:

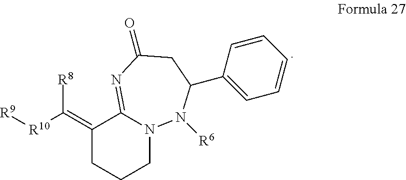

In another embodiment, this invention discloses a compound, or pharmaceutically acceptable salts, solvates, esters or prodrugs of said compound, said compound having the general structure shown in the formula 28:

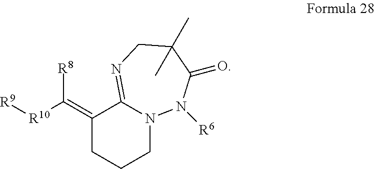

In another embodiment, this invention discloses a compound, or pharmaceutically acceptable salts, solvates, esters or prodrugs of said compound, said compound having the general structure shown in the formula 29:

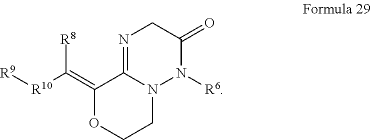

In another embodiment, this invention discloses a compound, or pharmaceutically acceptable salts, solvates, esters or prodrugs of said compound, said compound having the general structure shown in the formula 30:

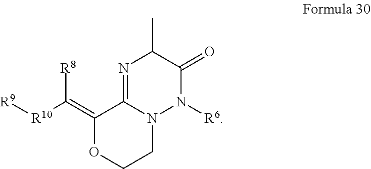

In another embodiment, this invention discloses a compound, or pharmaceutically acceptable salts, solvates, esters or prodrugs of said compound, said compound having the general structure shown in the formula 31:

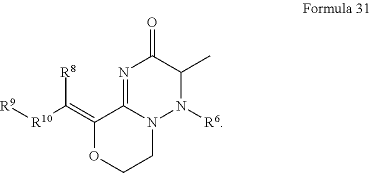

In another embodiment, this invention discloses a compound, or pharmaceutically acceptable salts, solvates, esters or prodrugs of said compound, said compound having the general structure shown in the formula 32:

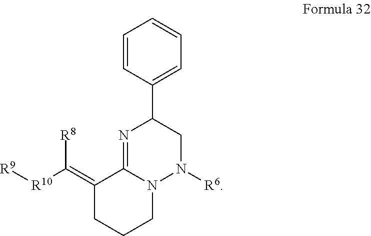

In another embodiment, this invention discloses a compound, or pharmaceutically acceptable salts, solvates, esters or prodrugs of said compound, said compound having the general structure shown in the formula 33:

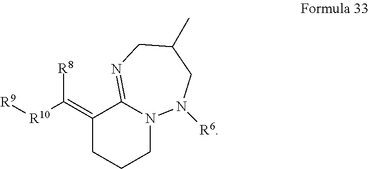

In another embodiment, this invention discloses a compound, or pharmaceutically acceptable salts, solvates, esters or prodrugs of said compound, said compound having the general structure shown in the formula 34:

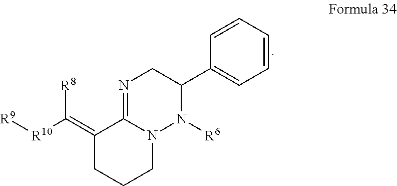

In another embodiment, this invention discloses a compound, or pharmaceutically acceptable salts, solvates, esters or prodrugs of said compound, said compound having the general structure shown in the formula 35:

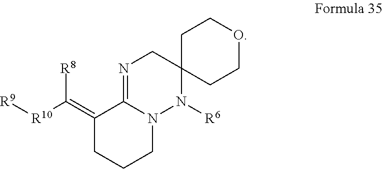

In another embodiment, this invention discloses a compound, or pharmaceutically acceptable salts, solvates, esters or prodrugs of said compound, said compound having the general structure shown in the formula 36:

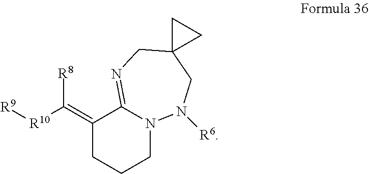

In another embodiment, this invention discloses a compound, or pharmaceutically acceptable salts, solvates, esters or prodrugs of said compound, said compound having the general structure shown in the formula 37:

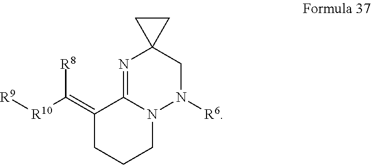

In another embodiment, this invention discloses a compound, or pharmaceutically acceptable salts, solvates, esters or prodrugs of said compound, said compound having the general structure shown in the formula 38:

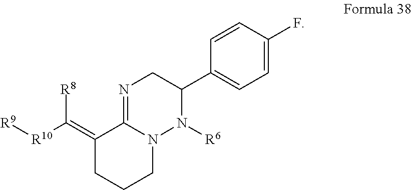

In another embodiment, this invention discloses a compound, or pharmaceutically acceptable salts, solvates, esters or prodrugs of said compound, said compound having the general structure shown in the formula 39:

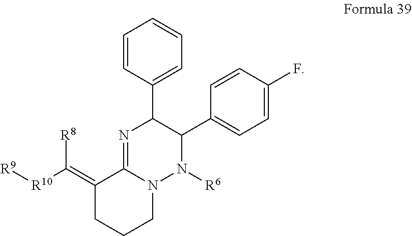

In another embodiment, this invention discloses a compound, or pharmaceutically acceptable salts, solvates, esters or prodrugs of said compound, said compound having the general structure shown in the formula 40:

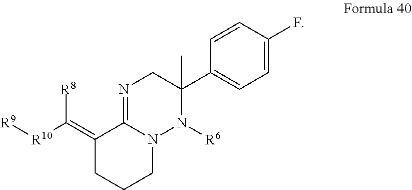

In another embodiment, this invention discloses a compound, or pharmaceutically acceptable salts, solvates, esters or prodrugs of said compound, said compound having the general structure shown in the formula 41:

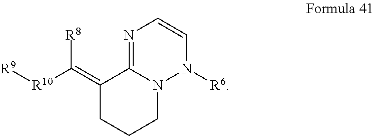

In another embodiment, this invention discloses a compound, or pharmaceutically acceptable salts, solvates, esters or prodrugs of said compound, said compound having the general structure shown in the formula 42:

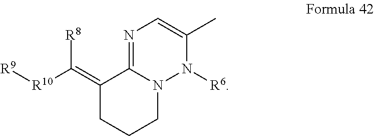

In another embodiment, this invention discloses a compound, or pharmaceutically acceptable salts, solvates, esters or prodrugs of said compound, said compound having the general structure shown in the formula 43:

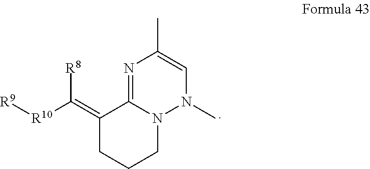

In another embodiment, this invention discloses a compound, or pharmaceutically acceptable salts, solvates, esters or prodrugs of said compound, said compound having the general structure shown in the formula 44:

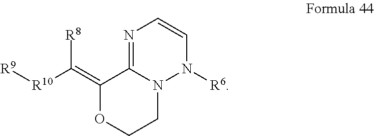

In another embodiment, this invention discloses a compound, or pharmaceutically acceptable salts, solvates, esters or prodrugs of said compound, said compound having the general structure shown in the formula 45:

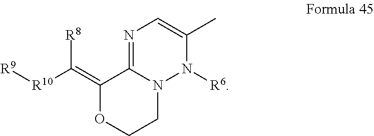

In another embodiment, this invention discloses a compound, or pharmaceutically acceptable salts, solvates, esters or prodrugs of said compound, said compound having the general structure shown in the formula 46:

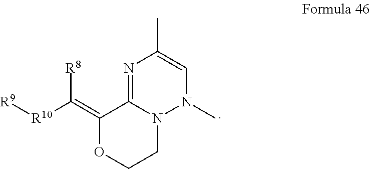

In another embodiment, this invention discloses a compound, or pharmaceutically acceptable salts, solvates, esters or prodrugs of said compound, said compound having the general structure shown in the formula 47:

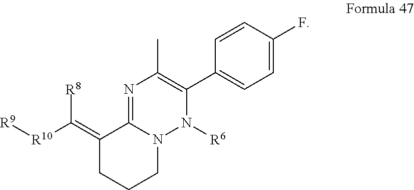

In another embodiment, this invention discloses a compound, or pharmaceutically acceptable salts, solvates, esters or prodrugs of said compound, said compound having the general structure shown in the formula 48:

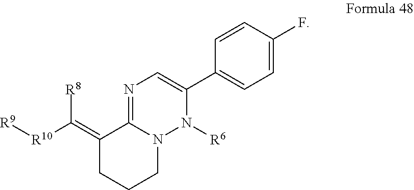

In another embodiment, this invention discloses a compound, or pharmaceutically acceptable salts, solvates, esters or prodrugs of said compound, said compound having the general structure shown in the formula 49:

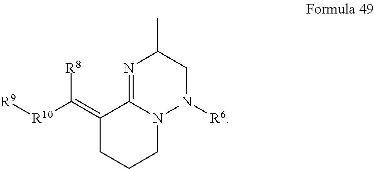

In another embodiment, this invention discloses a compound, or pharmaceutically acceptable salts, solvates, esters or prodrugs of said compound, said compound having the general structure shown in the formula 50:

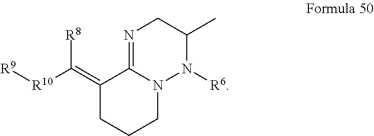

In each of embodiments of Formulas 3-50 above,

R⁶ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- can be unsubstituted or optionally substituted with 1-5 independently selected R²¹ substituents;

R⁸ is selected from the group consisting of H, alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl-, with each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- being unsubstituted or optionally substituted with 1-3 independently selected R²¹ substituents;

R⁹ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each R⁹ group is optionally substituted with 1-3 independently selected R²¹ substituents;

R¹⁰ is selected from the group consisting of a bond, alkyl-, alkenyl-, alkynyl-, aryl-, arylalkyl-, alkylaryl-, cycloalkyl-, cycloalkylalkyl-, heteroaryl-, heteroarylalkyl-, heterocyclyl-, heterocyclylalkyl- and the moieties:

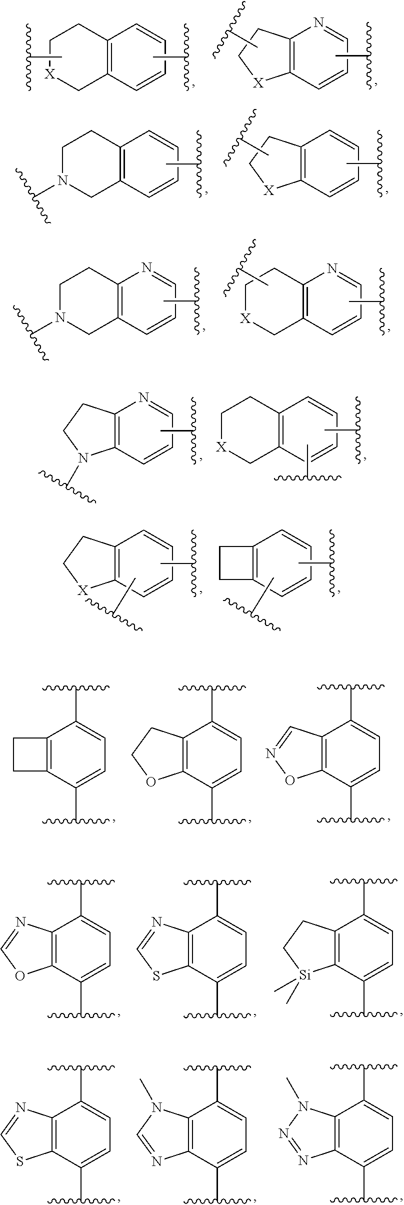

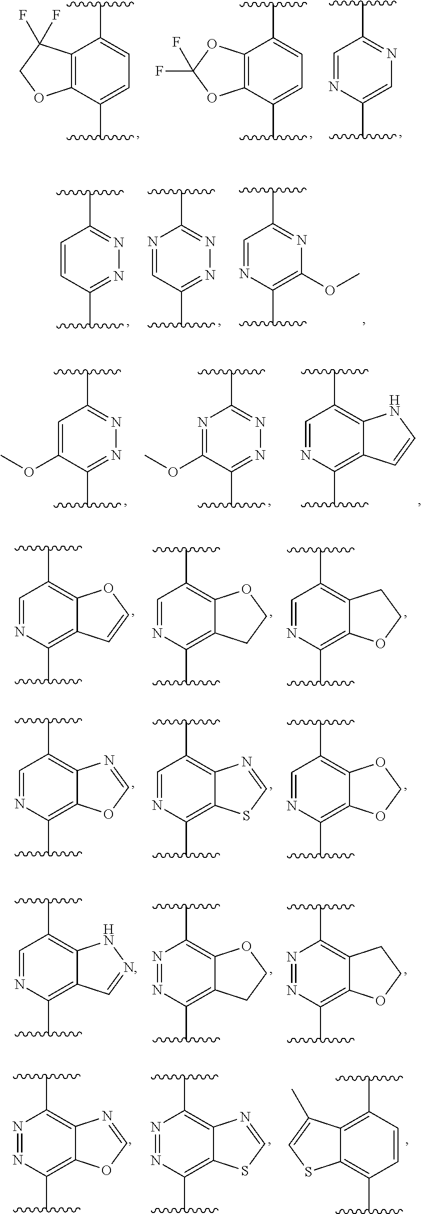

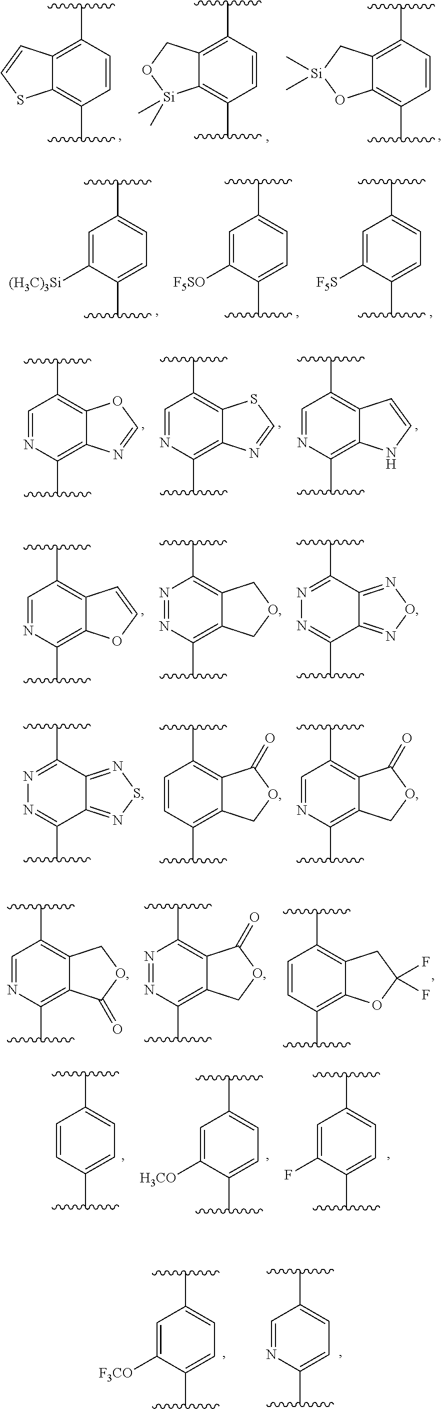

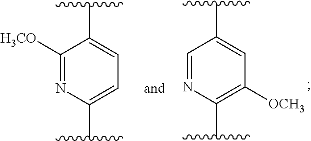

wherein X is O, N(R¹⁴) or S and wherein each R¹⁰ group (except for the bond) is optionally substituted with 1-3 independently selected R²¹ substituents;

each R²¹ group is independently selected from the group consisting of alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, halo, —CN, —OR¹⁵, —C(O)R¹⁵, —C(O)OR¹⁵, —C(O)N(R¹⁵)(R¹⁶), —SF₅, —OSF₅, —Si(R^{15A})₃, —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R^{15A}; and wherein each of the R²¹ alkyl, cycloalkenyl, cycloalkyl, cycloalkylalkyl-, heterocyclyl, heterocyclylalkyl-, aryl, arylalkyl-, heteroaryl, heteroarylalkyl-, alkenyl and alkynyl groups is optionally substituted with 1 to 5 independently selected R²² groups;

each R²² is independently selected from the group consisting of: alkyl, cycloalkyl, cycloalkenyl, heterocyclyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SF₅, —OSF₅, —Si(R^{15A})₃, —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R^{15A}; and

R¹⁴ is selected from the group consisting of H, alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, —CN, —C(O)R¹⁵, —C(O)OR¹⁵, —C(O)N(R¹⁵)(R¹⁶), —S(O)N(R¹⁵)(R¹⁵), —S(O)₂N(R¹⁵)(R¹⁶), —C(═NOR¹⁵)R¹⁶, and —P(O)(OR¹⁵)(OR¹⁶), wherein each of said alkyl, alkenyl, alkynyl, cycloalkyl, cycloalkylalkyl-, cycloalkenyl, heterocyclyl, heterocyclylalkyl-, aryl, arylalkyl-, heteroaryl, and heteroarylalkyl- is optionally substituted with 1-5 independently selected R²¹ substitutents;

R¹⁵, R¹⁶ and R¹⁷ can be the same or different and are each independently selected from the group consisting of H, alkyl, alkenyl, alkynyl, cycloalkyl, cycloalkylalkyl-, heterocyclyl, heterocyclylalkyl-, aryl, arylalkyl-, heteroaryl, heteroarylalkyl-, arylcycloalkyl-, arylheterocyclyl-, (R¹⁸)ᵣ-alkyl, (R¹⁸)ᵣ-cycloalkyl, (R¹⁸)ᵣ-cycloalkylalkyl-, (R¹⁸)ᵣ-heterocyclyl, (R¹⁸)ᵣ-heterocyclylalkyl-, (R¹⁸)ᵣ-aryl, (R¹⁸)ᵣ-arylalkyl-, (R¹⁸)ᵣ-heteroaryl and (R¹⁸)ᵣ-heteroarylalkyl-; wherein r is 1-5;

each R¹⁸ is independently selected from the group consisting of alkyl, alkenyl, alkynyl, aryl, arylalkyl-, arylalkenyl-, arylalkynyl-, —NO₂, halo, heteroaryl, HO-alkyoxyalkyl-, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

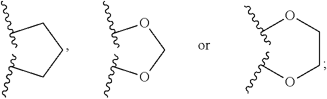

R¹⁹ is selected from the group consisting of: alkyl, cycloalkyl, aryl, arylalkyl- and heteroarylalkyl-;

R²⁰ is selected from the group consisting of: alkyl, cycloalkyl, aryl, halo substituted aryl, arylalkyl-, heteroaryl and heteroarylalkyl-.

Preferably, in each embodiment as described in formulas 3-50 above, R⁹ is selected from the group consisting of alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each R⁹ group is optionally substituted with 1-3 independently selected R²¹ substituents.

Preferably, in each embodiment as described in formulas 3-50 above, R⁶ is H, alkyl or aryl, which aryl can be unsubstituted or substituted with 1 to 3 independently selected R²¹ moieties; R⁸ is H, alkyl or aryl; R⁹ is selected from the group consisting of heteroaryl and heteroaryl substituted with 1-3 independently selected R²¹ groups; and R¹⁹ is aryl, which can be unsubstituted or substituted with 1 to 3 independently selected R²¹ moieties, heteroaryl and heteroaryl substituted with 1-3 independently selected R²¹ groups, or a fused aryl ring selected from

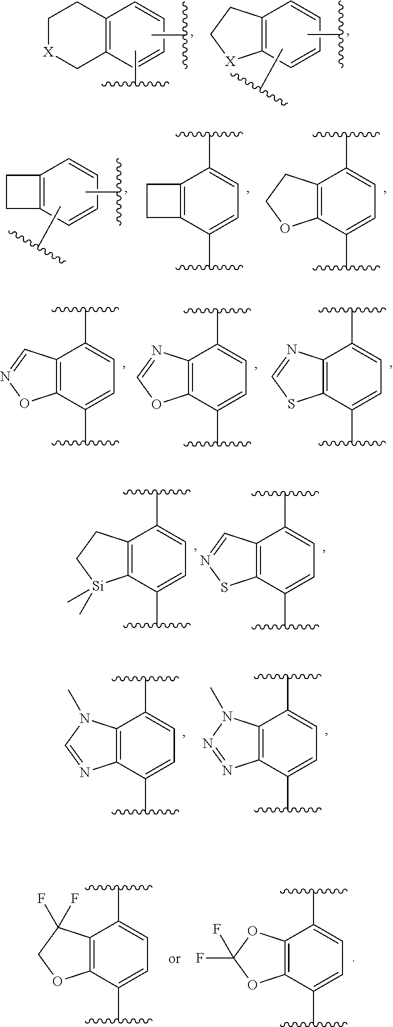

More preferably, in each embodiment as describe in formulas 3-50 above, R⁶ is H, methyl or phenyl, which phenyl can be unsubstituted or substituted with 1 to 3 independently selected R²¹ moieties; R⁸ is H or alkyl; and R⁹—R¹⁰— is selected from:

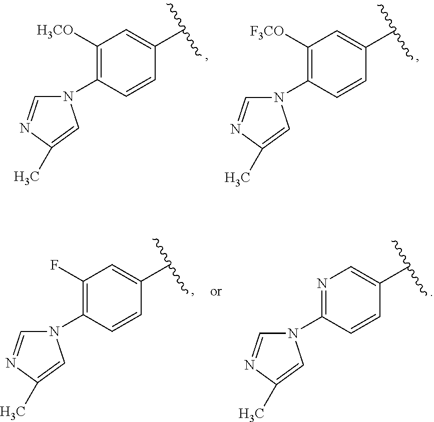

Most preferably, in each embodiment as described in formulas 3-50 above, R⁶ is phenyl, which can be unsubstituted or substituted with 1 to 3 R²¹ moieties which can be the same or different and are independently selected from halo (preferably fluoro), SF₅, OSF₅, and Si(Me)₃; R⁸ is H or alkyl; and R⁹—R¹⁰— is

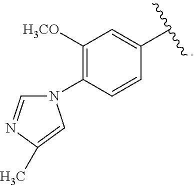

In another embodiment, this invention discloses a compound, or pharmaceutically acceptable salts, solvates, esters or prodrugs of said compound, said compound having the general structure shown in the formula 51:

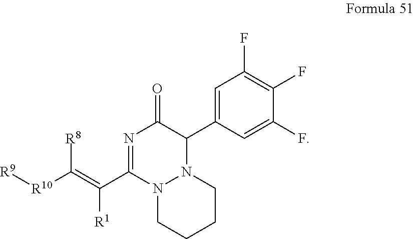

In another embodiment, this invention discloses a compound, or pharmaceutically acceptable salts, solvates, esters or prodrugs of said compound, said compound having the general structure shown in the formula 52:

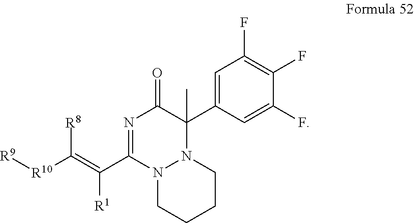

In another embodiment, this invention discloses a compound, or pharmaceutically acceptable salts, solvates, esters or prodrugs of said compound, said compound having the general structure shown in the formula 53:

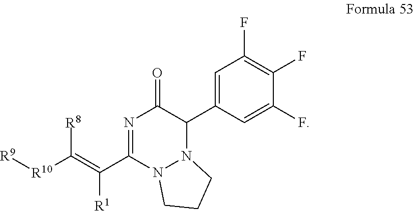

In each of embodiments of Formulas 51-53 above,

R¹ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- can be unsubstituted or optionally substituted with 1-5 independently selected R²¹ substituents;

R⁸ is selected from the group consisting of H, alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl-, with each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- being unsubstituted or optionally substituted with 1-3 independently selected R²¹ substituents;

R⁹ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each R⁹ group is optionally substituted with 1-3 independently selected R²¹ substituents;

R¹⁹ is selected from the group consisting of a bond, alkyl-, alkenyl-, alkynyl-, aryl-, arylalkyl-, alkylaryl-, cycloalkyl-, cycloalkylalkyl-, heteroaryl-, heteroarylalkyl-, heterocyclyl-, heterocyclylalkyl- and the moieties:

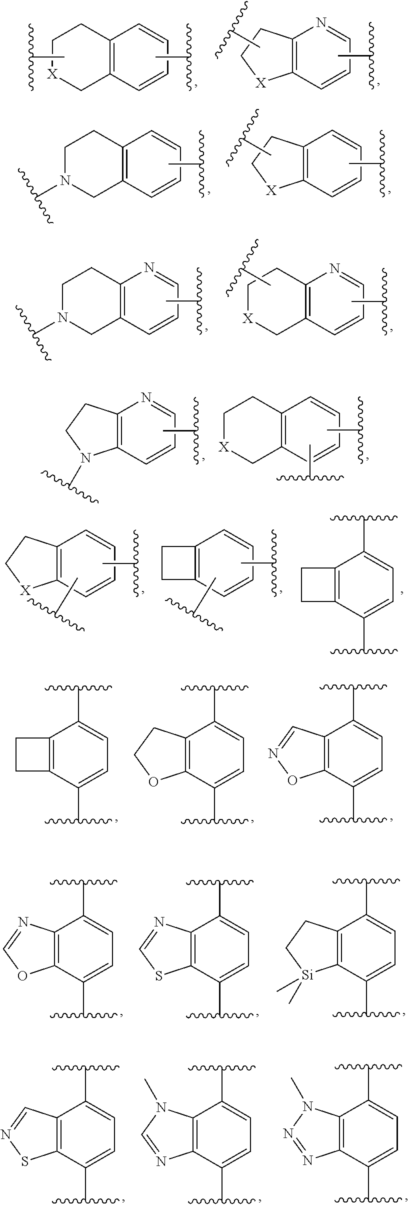

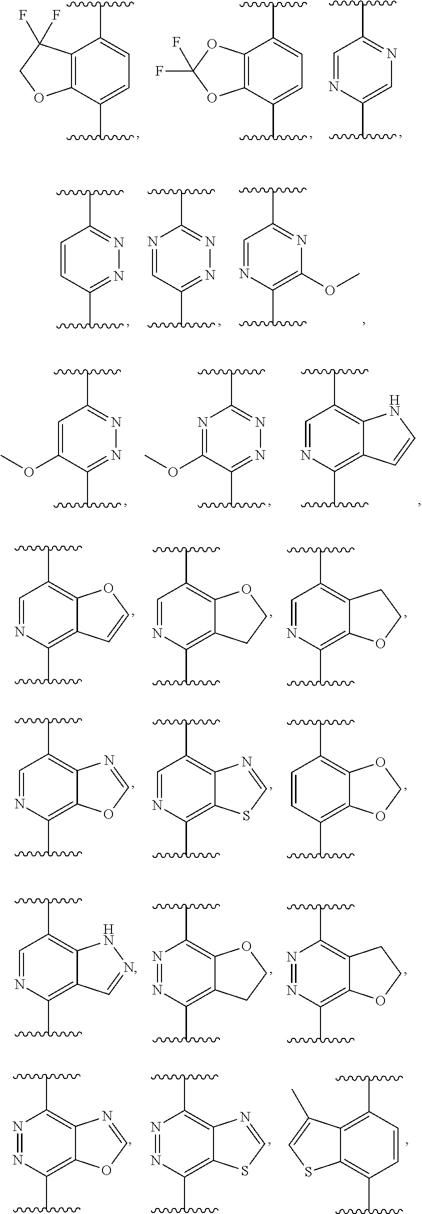

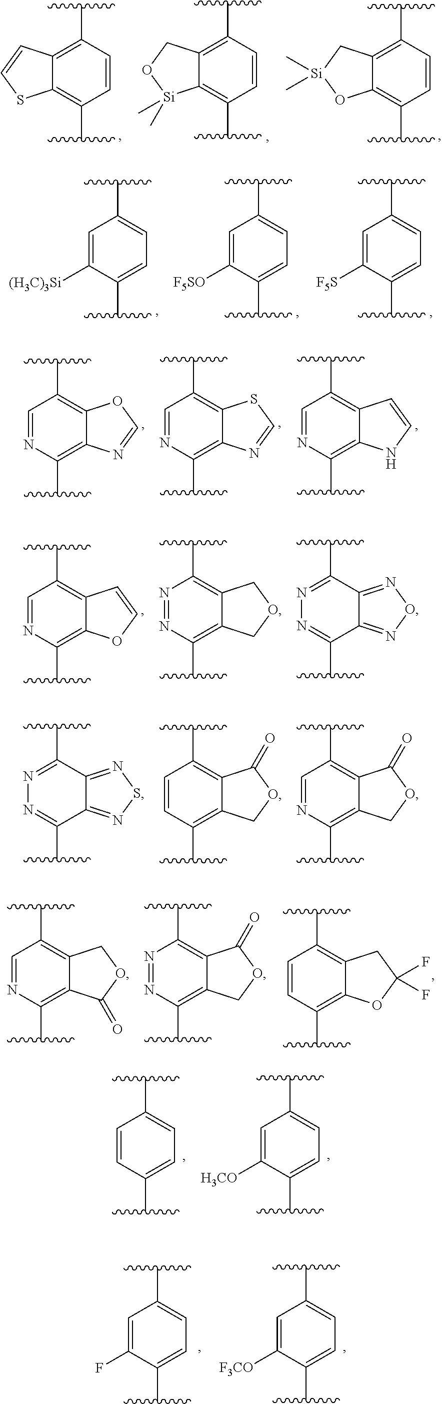

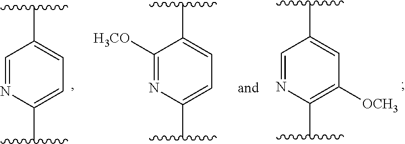

wherein X is O, N(R¹⁴) or S and wherein each R¹⁰ group (except for the bond) is optionally substituted with 1-3 independently selected R²¹ substituents;

each R²¹ group is independently selected from the group consisting of alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, halo, —CN, —OR¹⁵, —C(O)R¹⁵, —C(O)OR¹⁵, —C(O)N(R¹⁵)(R¹⁶), —SF₅, —OSF₅, —Si(R^{15A})₃, —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R^{15A}; and wherein each of the R²¹ alkyl, cycloalkenyl, cycloalkyl, cycloalkylalkyl-, heterocyclyl, heterocyclylalkyl-, aryl, arylalkyl-, heteroaryl, heteroarylalkyl-, alkenyl and alkynyl groups is optionally substituted with 1 to 5 independently selected R²² groups;

each R²² is independently selected from the group consisting of: alkyl, cycloalkyl, cycloalkenyl, heterocyclyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SF₅, —OSF₅, —Si(R^{15A})₃, —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R^{15A}; and

R¹⁴ is selected from the group consisting of H, alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶), wherein each of said alkyl, alkenyl, alkynyl, cycloalkyl, cycloalkylalkyl-, cycloalkenyl, heterocyclyl, heterocyclylalkyl-, aryl, arylalkyl-, heteroaryl, and heteroarylalkyl- is optionally substituted with 1-5 independently selected R²¹ substitutents;

R¹⁵, R¹⁶ and R¹⁷ can be the same or different and are each independently selected from the group consisting of H, alkyl, alkenyl, alkynyl, cycloalkyl, cycloalkylalkyl-, heterocyclyl, heterocyclylalkyl-, aryl, arylalkyl-, heteroaryl, heteroarylalkyl-, arylcycloalkyl-, arylheterocyclyl-, (R¹⁸)ᵣ-alkyl, (R¹⁸)ᵣ-cycloalkyl, (R¹⁸)ᵣ-cycloalkylalkyl-, (R¹⁸)ᵣ-heterocyclyl, (R¹⁸)ᵣ-heterocyclylalkyl-, (R¹⁸)ᵣ-aryl, (R¹⁸)ᵣ-arylalkyl-, (R¹⁸)ᵣ-heteroaryl and (R¹⁸)ᵣ-heteroarylalkyl-; wherein r is 1-5;

each R¹⁸ is independently selected from the group consisting of alkyl, alkenyl, alkynyl, aryl, arylalkyl-, arylalkenyl-, arylalkynyl-, —NO₂, halo, heteroaryl, HO-alkyoxyalkyl-, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

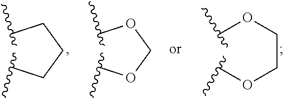

R¹⁹ is selected from the group consisting of: alkyl, cycloalkyl, aryl, arylalkyl- and heteroarylalkyl-;

R²⁰ is selected from the group consisting of: alkyl, cycloalkyl, aryl, halo substituted aryl, arylalkyl-, heteroaryl and heteroarylalkyl-.

Preferably, in the embodiment as described in formulas 51-53 above, R⁹ is selected from the group consisting of alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each R⁹ group is optionally substituted with 1-3 independently selected R²¹ substituents.

Preferably, in each embodiment as described in formulas 51-53 above, R¹ is H, alkyl or aryl, which aryl can be unsubstituted or substituted with 1 to 3 independently selected R²¹ moieties; R⁸ is H, alkyl or aryl; R⁹ is selected from the group consisting of heteroaryl and heteroaryl substituted with 1-3 independently selected R²¹ groups; and R¹⁰ is aryl, which can be unsubstituted or substituted with 1 to 3 independently selected R²¹ moieties, heteroaryl and heteroaryl substituted with 1-3 independently selected R²¹ groups, or a fused aryl ring selected from

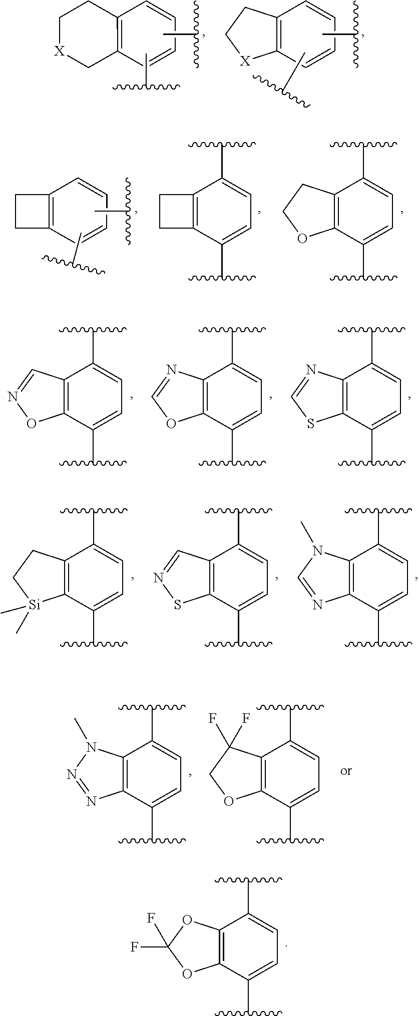

More preferably, in each embodiment as describe in formulas 51-53 above, R¹ is H, methyl or phenyl, which phenyl can be unsubstituted or substituted with 1 to 3 independently selected R²¹ moieties; R⁸ is H or alkyl; and R⁹—R¹⁰— is selected from:

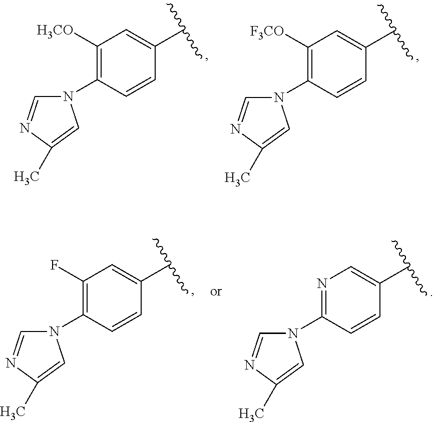

Most preferably, in each embodiment as described in formulas 51-53 above, R¹ is H, methyl or phenyl, which phenyl can be unsubstituted or substituted with 1 to 3 R²¹ moieties which can be the same or different and are independently selected from halo (preferably fluoro), SF₅, OSF₅, and Si(Me)₃; R⁸ is H or alkyl; and R⁹—R¹⁰— is

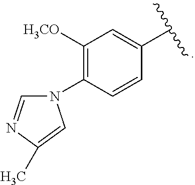

An illustrative group of compounds of the invention are shown in Table 1.

TABLE 1

| | |
|---|---|
| 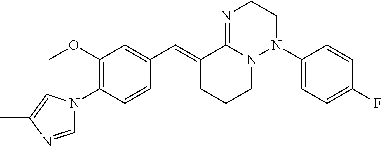 | A6 |
| 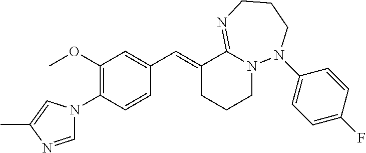 | A7 |
| 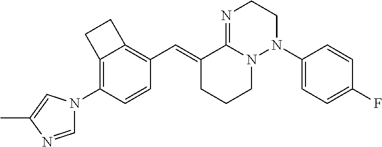 | A9 |
| 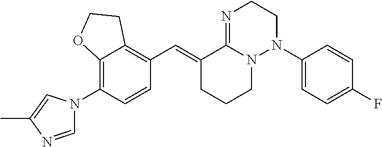 | A10 |
| 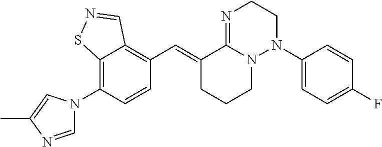 | A12 |
| 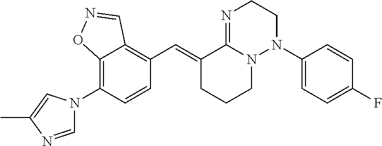 | A13 |
| 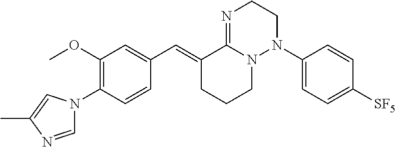 | A15 |
| 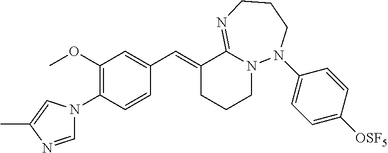 | A16 |
| 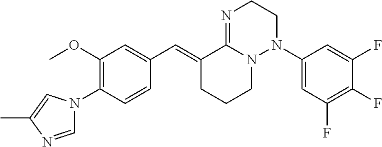 | A8 |
| 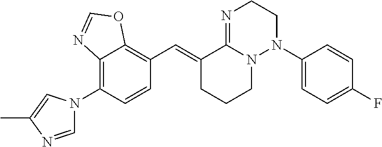 | A14 |
| 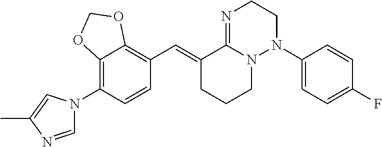 | A11 |
| 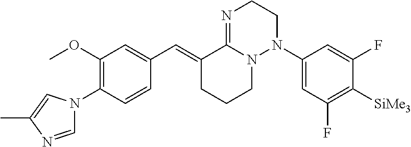 | A17 |
| 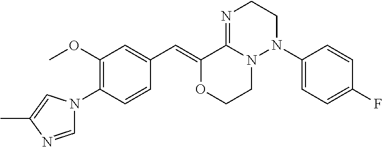 | D9 |
| 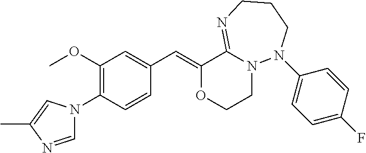 | D10 |
| 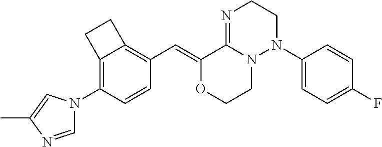 | D12 |
| 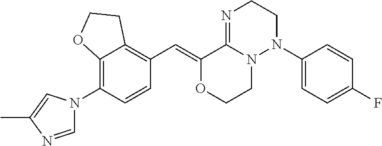 | D13 |
| 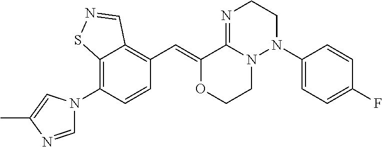 | D15 |
| 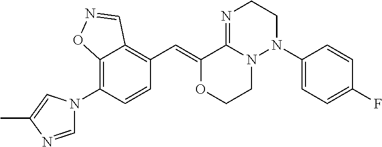 | D16 |
| 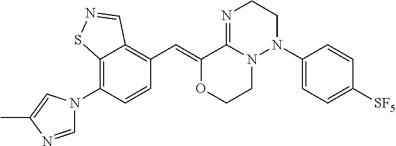 | D18 |
| 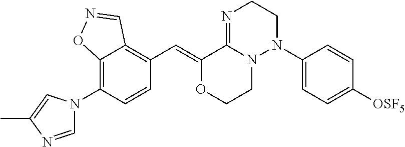 | D19 |
| 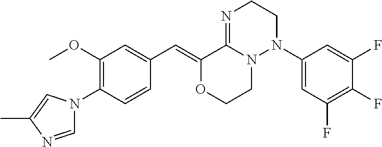 | D11 |
| 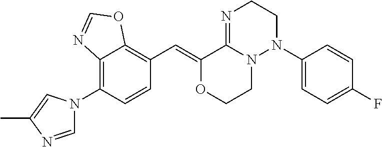 | D17 |
| 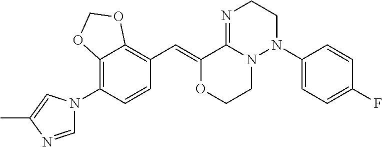 | D14 |
| 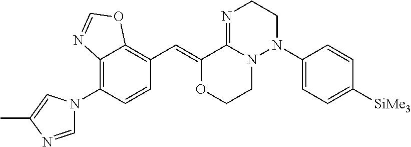 | D20 |
| 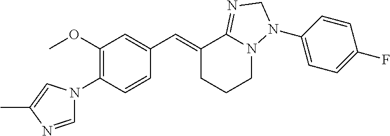 | E4 |
| 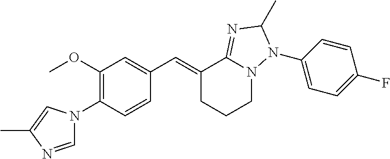 | E5 |
| 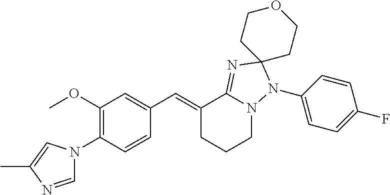 | E7 |
| 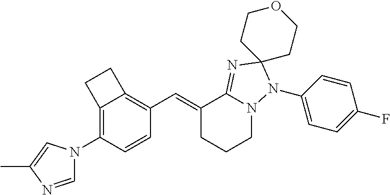 | E8 |
| 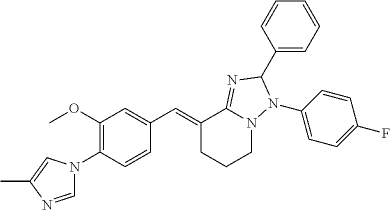 | E10 |
| 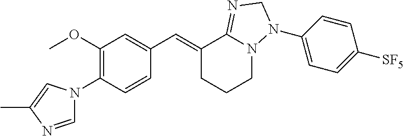 | E11 |
| 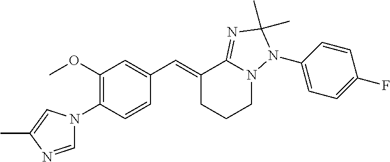 | E6 |
| 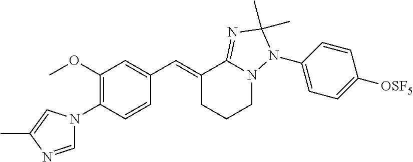 | E12 |
| 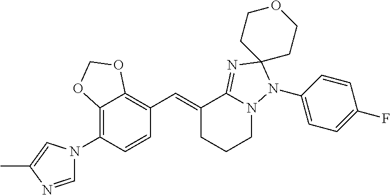 | E9 |
| 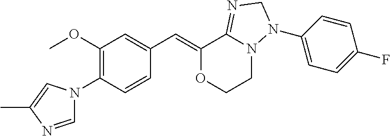 | E13 |
| 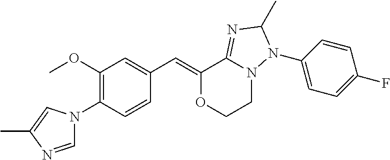 | E14 |
| 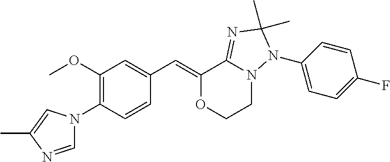 | E15 |
| 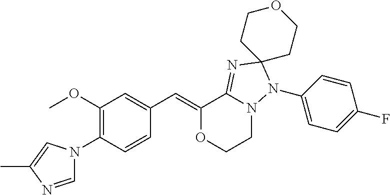 | E16 |
| 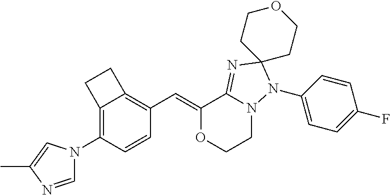 | E17 |
| 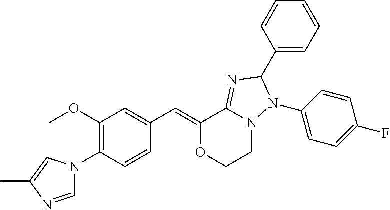 | E19 |
| 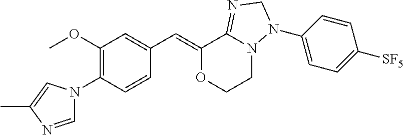 | E20 |
| 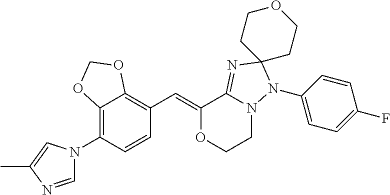 | E18 |
| 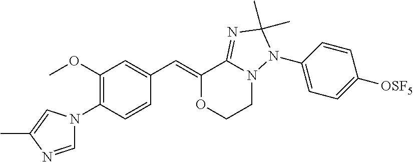 | E21 |
| 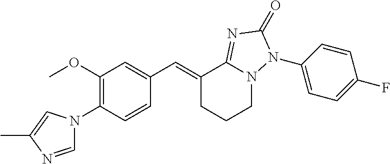 | F1 |
| 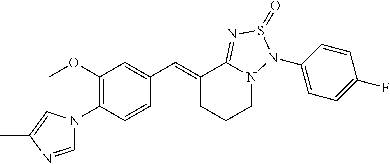 | F2 |
| 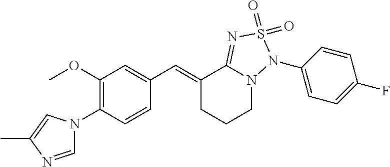 | F3 |
| 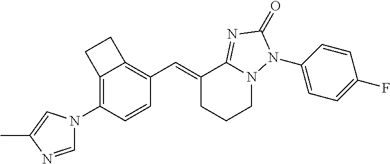 | F4 |
| 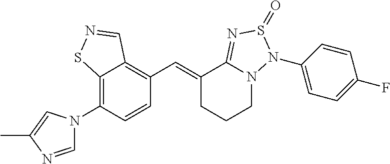 | F5 |
| 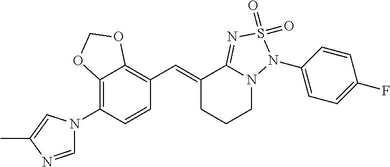 | F6 |
| 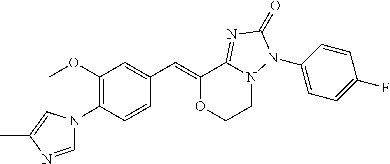 | F7 |
| 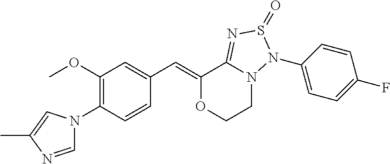 | F8 |
| 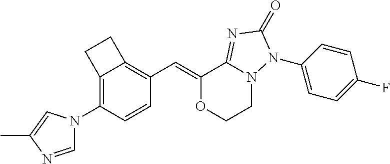 | F10 |
| 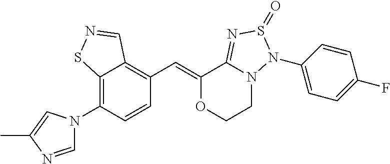 | F11 |
| 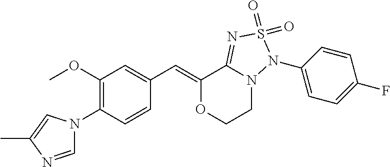 | F9 |
| 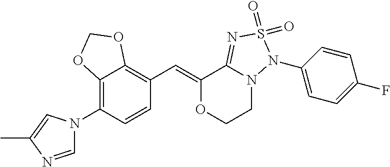 | F12 |
| 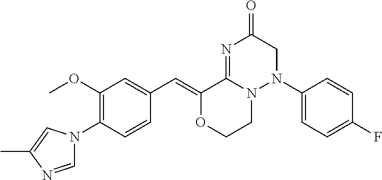 | G6 |
| 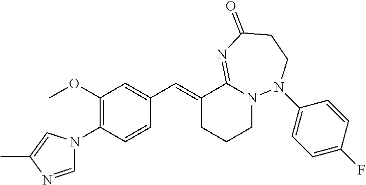 | G7 |
| 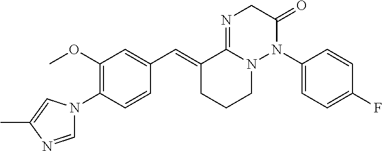 | G9 |
| 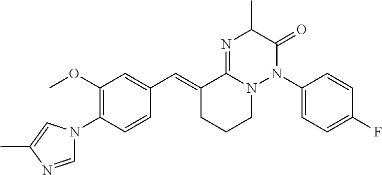 | G10 |
| 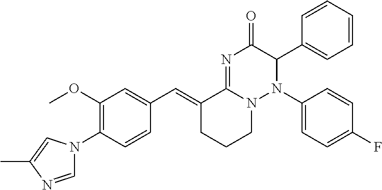 | G12 |
| 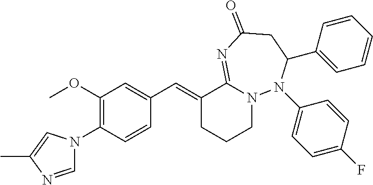 | G13 |
| 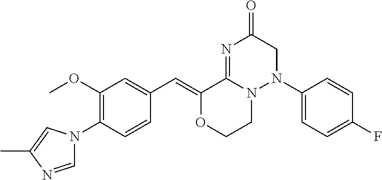 | G15 |
| 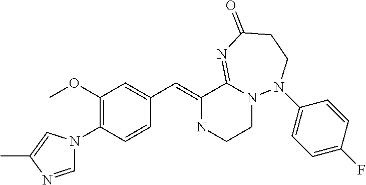 | G16 |
| 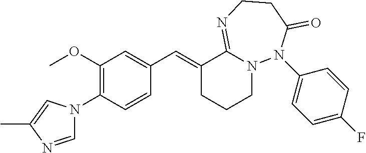 | G8 |
| 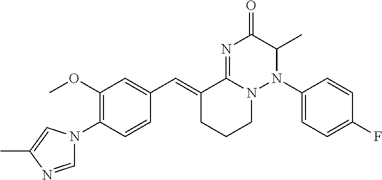 | G11 |
| 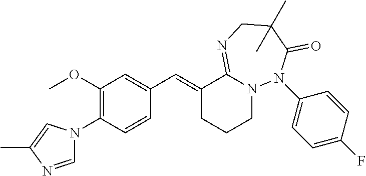 | G14 |
| 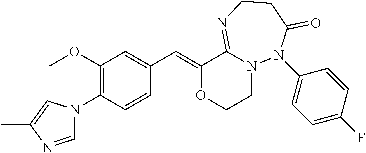 | G17 |
| 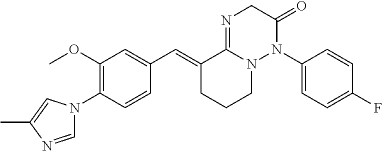 | G18 |
| 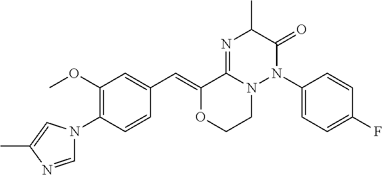 | G19 |
| 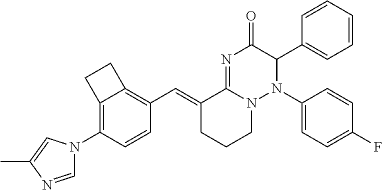 | G21 |
| 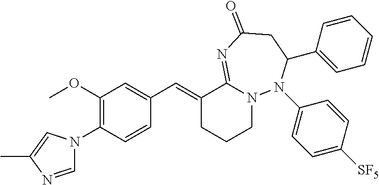 | G22 |
| 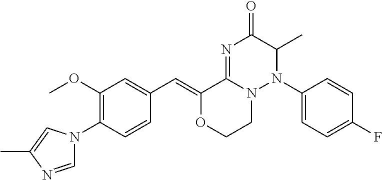 | G20 |
| 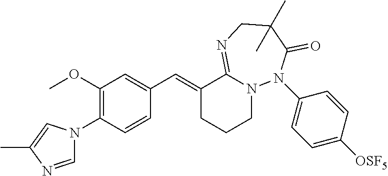 | G23 |
| 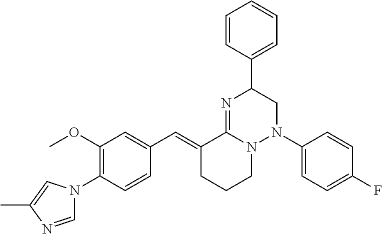 | H1 |
| 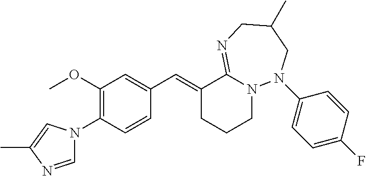 | H2 |
| 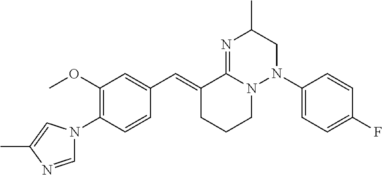 | H3 |
| 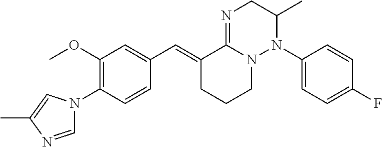 | H4 |
| 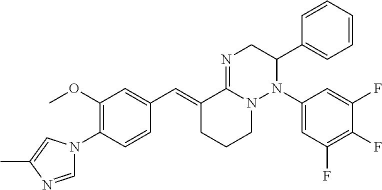 | H5 |
| 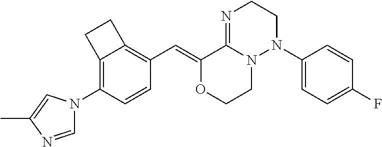 | H6 |
| 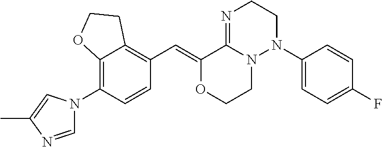 | H7 |
| 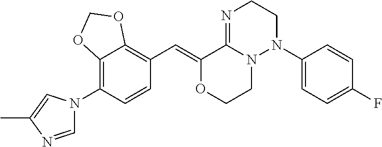 | H8 |
| 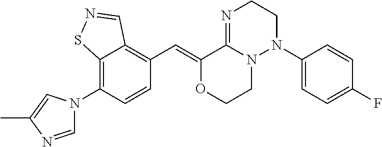 | H9 |
| 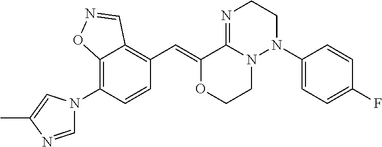 | H10 |
| 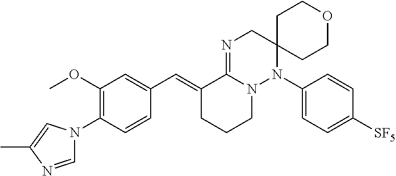 | H11 |
| 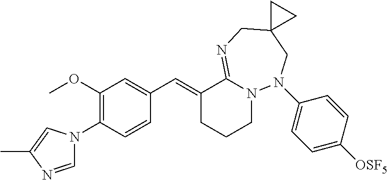 | H12 |
| 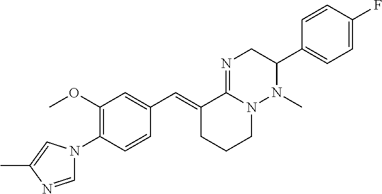 | H13 |
| 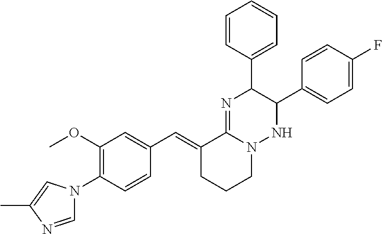 | H14 |
| 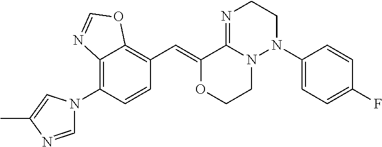 | H15 |
| 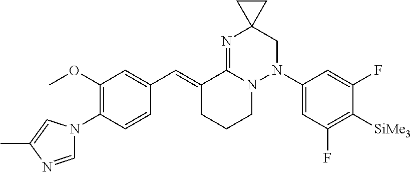 | H16 |
| 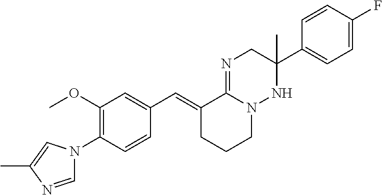 | H17 |
| 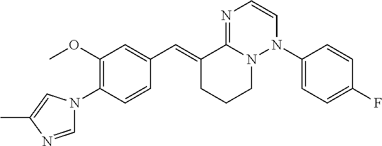 | I4 |
| 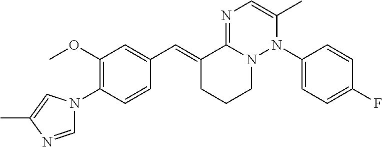 | I5 |
| 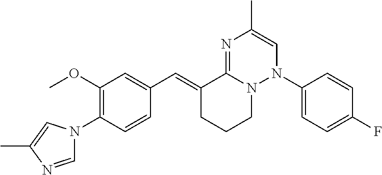 | I6 |
| 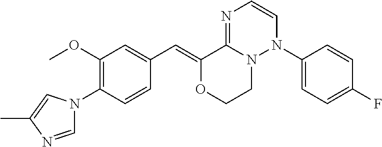 | I7 |
| 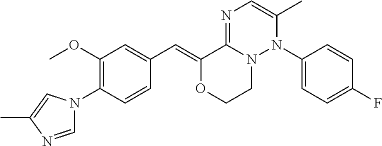 | I8 |
| 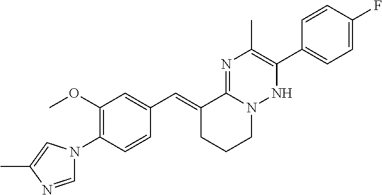 | I10 |
| 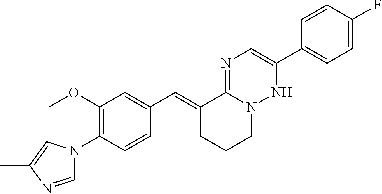 | I11 |
| 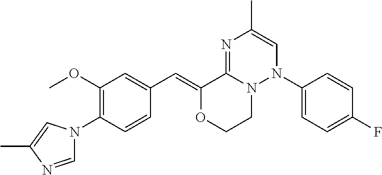 | I9 |
| 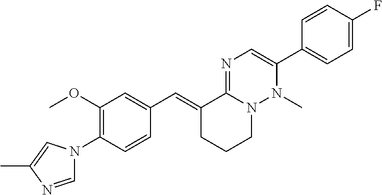 | I12 |
| 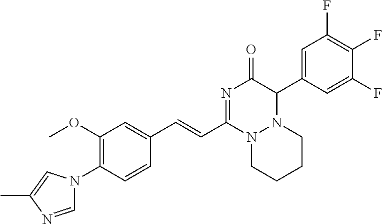 | J5 |
| 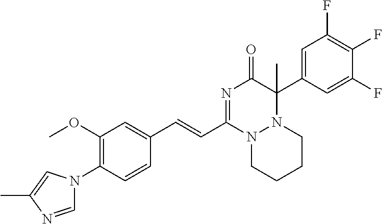 | J6 |
| 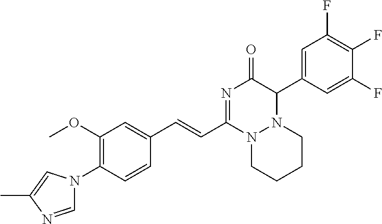 | J7 |
| 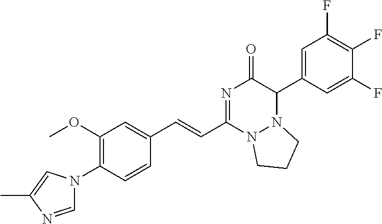 | J8 |
| 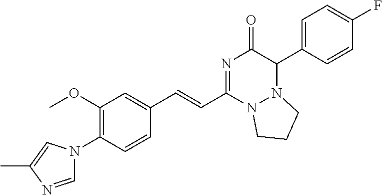 | J9 |
| 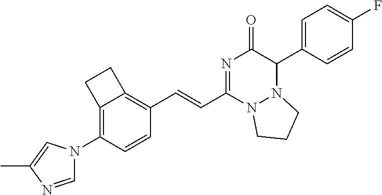 | J10 |
| 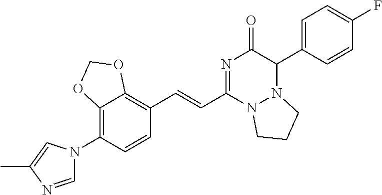 | J11 |
| 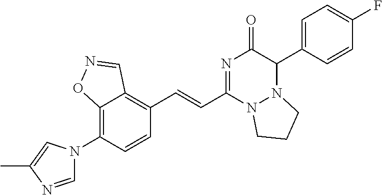 | J12 |
| 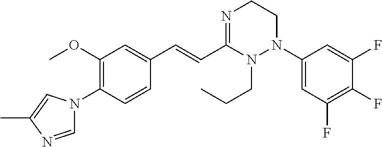 | M7 |
| 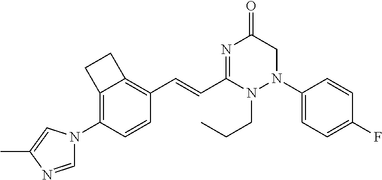 | M8 |
| 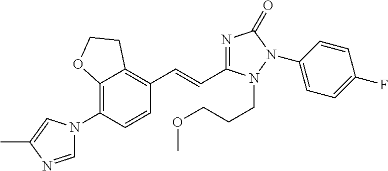 | M9 |
| 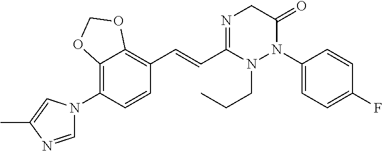 | M10 |
| 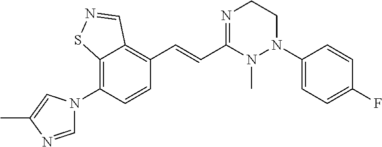 | M11 |
| 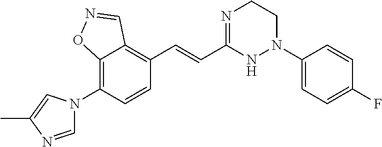 | M12 |
| 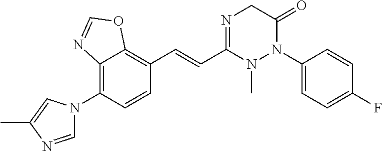 | M13 |
| 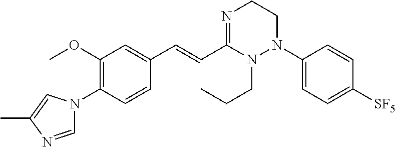 | M14 |
| 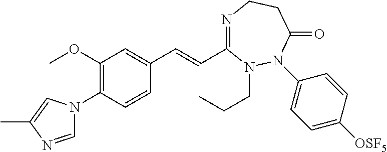 | M15 |
| 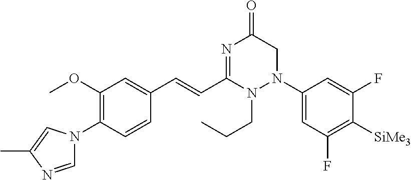 | M16 |

One embodiment of this invention is directed to compound A6.

Another embodiment of this invention is directed to compound A7.

Another embodiment of this invention is directed to compound A8.

Another embodiment of this invention is directed to compound A9.

Another embodiment of this invention is directed to compound A10.

Another embodiment of this invention is directed to compound A11.

Another embodiment of this invention is directed to compound A12.

Another embodiment of this invention is directed to compound A13.

Another embodiment of this invention is directed to compound A14.

Another embodiment of this invention is directed to compound A15.

Another embodiment of this invention is directed to compound A16.

Another embodiment of this invention is directed to compound A17.

Another embodiment of this invention is directed to compound D9.

Another embodiment of this invention is directed to compound D10.

Another embodiment of this invention is directed to compound D11.

Another embodiment of this invention is directed to compound D12.

Another embodiment of this invention is directed to compound D13.

Another embodiment of this invention is directed to compound D14.

Another embodiment of this invention is directed to compound D15.

Another embodiment of this invention is directed to compound D16.

Another embodiment of this invention is directed to compound D17.

Another embodiment of this invention is directed to compound D18.

Another embodiment of this invention is directed to compound D19.

Another embodiment of this invention is directed to compound D20.

Another embodiment of this invention is directed to compound E4.

Another embodiment of this invention is directed to compound E5.

Another embodiment of this invention is directed to compound E6.

Another embodiment of this invention is directed to compound E7.

Another embodiment of this invention is directed to compound E8.

Another embodiment of this invention is directed to compound E9.

Another embodiment of this invention is directed to compound E10.

Another embodiment of this invention is directed to compound E11.

Another embodiment of this invention is directed to compound E12.

Another embodiment of this invention is directed to compound E13.

Another embodiment of this invention is directed to compound E14.

Another embodiment of this invention is directed to compound E15.

Another embodiment of this invention is directed to compound E16.

Another embodiment of this invention is directed to compound E17. Another embodiment of this invention is directed to compound E18.

Another embodiment of this invention is directed to compound E19.

Another embodiment of this invention is directed to compound E20.

Another embodiment of this invention is directed to compound E21.

Another embodiment of this invention is directed to compound F7.

Another embodiment of this invention is directed to compound F8.

Another embodiment of this invention is directed to compound F9.

Another embodiment of this invention is directed to compound F10.

Another embodiment of this invention is directed to compound F11.

Another embodiment of this invention is directed to compound F12.

Another embodiment of this invention is directed to compound G6.

Another embodiment of this invention is directed to compound G7.

Another embodiment of this invention is directed to compound G8.

Another embodiment of this invention is directed to compound G9.

Another embodiment of this invention is directed to compound G10.

Another embodiment of this invention is directed to compound G11.

Another embodiment of this invention is directed to compound G12.

Another embodiment of this invention is directed to compound G13.

Another embodiment of this invention is directed to compound G14.

Another embodiment of this invention is directed to compound G15.

Another embodiment of this invention is directed to compound G16.

Another embodiment of this invention is directed to compound G17.

Another embodiment of this invention is directed to compound G18.

Another embodiment of this invention is directed to compound G19.

Another embodiment of this invention is directed to compound G20.

Another embodiment of this invention is directed to compound G21.

Another embodiment of this invention is directed to compound G22.

Another embodiment of this invention is directed to compound G23.

Another embodiment of this invention is directed to compound H1.

Another embodiment of this invention is directed to compound H2.

Another embodiment of this invention is directed to compound H3.

Another embodiment of this invention is directed to compound H4.

Another embodiment of this invention is directed to compound H5.

Another embodiment of this invention is directed to compound H6.

Another embodiment of this invention is directed to compound H7.

Another embodiment of this invention is directed to compound H8.

Another embodiment of this invention is directed to compound H9.

Another embodiment of this invention is directed to compound H10.

Another embodiment of this invention is directed to compound H11.

Another embodiment of this invention is directed to compound H12.

Another embodiment of this invention is directed to compound H13.

Another embodiment of this invention is directed to compound H14.

Another embodiment of this invention is directed to compound H15.

Another embodiment of this invention is directed to compound H16.

Another embodiment of this invention is directed to compound H17.

Another embodiment of this invention is directed to compound 15.

Another embodiment of this invention is directed to compound 16.

Another embodiment of this invention is directed to compound 17.

Another embodiment of this invention is directed to compound 18.

Another embodiment of this invention is directed to compound 19.

Another embodiment of this invention is directed to compound 110.

Another embodiment of this invention is directed to compound 111.

Another embodiment of this invention is directed to compound 112.

Another embodiment of this invention is directed to compound J5.

Another embodiment of this invention is directed to compound J6.

Another embodiment of this invention is directed to compound J7.

Another embodiment of this invention is directed to compound J8.

Another embodiment of this invention is directed to compound J9.

Another embodiment of this invention is directed to compound J10.

Another embodiment of this invention is directed to compound J11.

Another embodiment of this invention is directed to compound J12.

Another embodiment of this invention is directed to compound M6.

Another embodiment of this invention is directed to compound M7.

Another embodiment of this invention is directed to compound M8.

Another embodiment of this invention is directed to compound M9.

Another embodiment of this invention is directed to compound M10.

Another embodiment of this invention is directed to compound M11.

Another embodiment of this invention is directed to compound M12.

Another embodiment of this invention is directed to compound M13.

Another embodiment of this invention is directed to compound M14.

Another embodiment of this invention is directed to compound M15.

Another embodiment of this invention is directed to compound M16.

Another embodiment of this invention is directed to compound O11.

Another embodiment of this invention is directed to compound N6.

As used above, and throughout this disclosure, the following terms, unless otherwise indicated, shall be understood to have the following meanings:

“ADDP” means 1,1′-(azodicarbonyl)dipiperidine.

“AIBN” means 2,2′-azobis(2-methylpropionitrile).

“CAN” means ammonium cerium (IV) nitrate.

“DCC” means N,N′-dicyclohexylcarbodiimide.

“DCM” means dichloromethane.

“DMF” means dimethylformamide.

“HOBT” means 1-hydroxylbenzotriazole.

“LDA” means lithium diisopropylamide.

“TBAF” means tetra-N-butylammonium fluoride.

“TBSO” means tert-butyldimethylsilyloxy.

“TfO” means trifluoromethylsulfonyloxy.

“At least one” means one or more than one, for example, 1, 2 or 3, or in another example, 1 or 2, or in another example 1.

“One or more” with reference to the use of the compounds of this invention means that one or more than one compound is used, for example, 1, 2 or 3, or in another example, 1 or 2, or in another example 1.

“Patient” includes both human and animals.

“Mammal” means humans and other mammalian animals.

It is noted that the carbons of formula I and other formulas herein may be replaced with 1 to 3 silicon atoms so long as all valency requirements are satisfied.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being independently selected from the group consisting of halo, alkyl, aryl, cycloalkyl, cyano, hydroxy, alkoxy, alkylthio, amino, oxime (e.g., ═N—OH), —NH(alkyl), —NH(cycloalkyl), —N(alkyl)₂, —O—C(O)-alkyl, —O—C(O)-aryl, —O—C(O)-cycloalkyl, carboxy and —C(O)O-alkyl. Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. “Alkenyl” may be unsubstituted or optionally substituted by one or more substituents which may be the same or different, each substituent being independently selected from the group consisting of halo, alkyl, aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkylene” means a difunctional group obtained by removal of a hydrogen atom from an alkyl group that is defined above. Non-limiting examples of alkylene include methylene, ethylene and propylene.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. “Alkynyl” may be unsubstituted or optionally substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl-group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl-group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Cycloalkylalkyl” means a cycloalkyl moiety as defined above linked via an alkyl moiety (defined above) to a parent core. Non-limiting examples of suitable cycloalkylalkyls include cyclohexyl methyl, adamantylmethyl and the like.

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl.

“Cycloalkenylalkyl” means a cycloalkenyl moiety as defined above linked via an alkyl moiety (defined above) to a parent core. Non-limiting examples of suitable cycloalkenylalkyls include cyclopentenylmethyl, cyclohexenylmethyl and the like.

“Halogen” means fluorine, chlorine, bromine, or iodine. Preferred are fluorine, chlorine and bromine. “Halo” refers to fluoro, chloro, bromo or iodo.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —O—C(O)-alkyl, —O—C(O)-aryl, —O—C(O)-cycloalkyl, —C(═N—CN)—NH₂, —C(═NH)—NH₂, —C(═NH)—NH(alkyl), oxime (e.g., ═N—OH), Y₁Y₂N—, Y₁Y₂N-alkyl-, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moiety are methylene dioxy, ethylenedioxy, —C(CH₃)₂— and the like which form moieties such

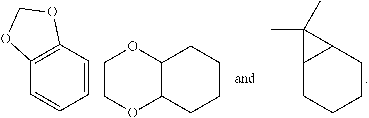

“Heteroarylalkyl” means a heteroaryl moiety as defined above linked via an alkyl moiety (defined above) to a parent core. Non-limiting examples of suitable heteroaryls include 2-pyridinylmethyl, quinolinylmethyl and the like.

“Heterocyclyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. “Heterocyclyl” may also mean a heterocyclyl ring wherein a single moiety (e.g ═O) simultaneously replaces two available hydrogens on the same carbon atom on a ring system. An example of

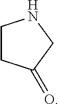

“Heterocyclylalkyl” means a heterocyclyl moiety as defined above linked via an alkyl moiety (defined above) to a parent core. Non-limiting examples of suitable heterocyclylalkyls include piperidinylmethyl, piperazinylmethyl and the like.

“Heterocyclenyl” means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” may also mean a single moiety (e.g., carbonyl) which simultaneously replaces two available hydrogens on the same carbon atom on a ring system. Example of such moiety is pyrrolidinone:

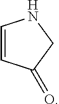

“Heterocyclenylalkyl” means a heterocyclenyl moiety as defined above linked via an alkyl moiety (defined above) to a parent core.

It should be noted that in hetero-atom containing ring systems of this invention, there are no hydroxyl groups on carbon atoms adjacent to a N, O or S, as well as there are no N or S groups on carbon adjacent to another heteroatom.

Thus, for example, in the ring:

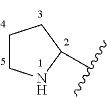

there is no —OH attached directly to carbons marked 2 and 5.

It should also be noted that tautomeric forms such as, for example, the moieties:

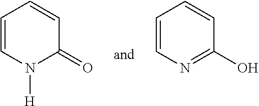

are considered equivalent in certain embodiments of this invention.

“Alkynylalkyl” means an alkynyl-alkyl-group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl-group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl-group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “purified”, “in purified form” or “in isolated and purified form” for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or in Formula I, its definition on each occurrence is independent of its definition at every other occurrence, unless otherwise indicated.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to yield a compound of Formula (I) or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of Formula (I) or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as 6-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl, and the like.

Similarly, if a compound of Formula (I) contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-(C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula (I) incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄)alkyl and Y³ is (C₁-C₆)alkyl, carboxy(C₁-C₆)alkyl, amino(C₁-C₄)alkyl or mono-N- or di-N,N—(C₁-C₆)alkylaminoalkyl, —C(Y⁴)Y⁵ wherein Y⁴ is H or methyl and Y⁵ is mono-N—or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds of Formula I can form salts which are also within the scope of this invention. Reference to a compound of Formula I herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula I may be formed, for example, by reacting a compound of Formula I with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

Compounds of Formula I, and salts, solvates, esters and prodrugs thereof, may exist in their tautomeric form (for example, as an amide, enol, keto or imino ether). All such tautomeric forms are contemplated herein as part of the present invention.

The compounds of Formula (I) may contain asymmetric or chiral centers, and, therefore, exist in different stereoisomeric forms. It is intended that all stereoisomeric forms of the compounds of Formula (I) as well as mixtures thereof, including racemic mixtures, form part of the present invention. In addition, the present invention embraces all geometric and positional isomers. For example, if a compound of Formula (I) incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this invention. Enantiomers can also be separated by use of chiral HPLC column.

It is also possible that the compounds of Formula (I) may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of Formula (I) incorporates a double bond or a fused ring, both the cis- and trans-forms, as well as mixtures, are embraced within the scope of the invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl and ¹²³I, respectively.

Certain isotopically-labelled compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Certain isotopically-labelled compounds of Formula (I) can be useful for medical imaging purposes. E.g., those labeled with positron-emitting isotopes like ¹¹C or ¹⁸F can be useful for application in Positron Emission Tomography (PET) and those labeled with gamma ray emitting isotopes like ¹²³I can be useful for application in Single photon emission computed tomography (SPECT). Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Additionally, isotopic substitution at a site where epimerization occurs may slow or reduce the epimerization process and thereby retain the more active or efficacious form of the compound for a longer period of time. Isotopically labeled compounds of Formula (I), in particular those containing isotopes with longer half lives (T1/2>1 day), can generally be prepared by following procedures analogous to those disclosed in the Schemes and/or in the Examples herein below, by substituting an appropriate isotopically labeled reagent for a non-isotopically labeled reagent.

Polymorphic forms of the compounds of Formula I, and of the salts, solvates, esters and prodrugs of the compounds of Formula I, are intended to be included in the present invention.

The compounds according to the invention can have pharmacological properties; in particular, the compounds of Formula I can be modulators of gamma secretase (including inhibitors, antagonists and the like).

More specifically, the compounds of Formula I can be useful in the treatment of a variety of disorders of the central nervous system including, for example, including, but not limited to, Alzheimer's disease, AIDS-related dementia, Parkinson's disease, amyotrophic lateral sclerosis, retinitis pigmentosa, spinal muscular atrophy and cerebellar degeneration and the like.

Another aspect of this invention is a method of treating a mammal (e.g., human) having a disease or condition of the central nervous system by administering a therapeutically effective amount of at least one compound of Formula I, or a pharmaceutically acceptable salt, solvate, ester or prodrug of said compound to the mammal.

A preferred dosage is about 0.001 to 500 mg/kg of body weight/day of the compound of Formula I. An especially preferred dosage is about 0.01 to 25 mg/kg of body weight/day of a compound of Formula I, or a pharmaceutically acceptable salt or solvate of said compound.

The compounds of this invention may also be useful in combination (administered together or sequentially) with one or more additional agents listed above and below.

The compounds of this invention may also be useful in combination (administered together or sequentially) with one or more compounds selected from the group consisting of Aβ antibody inhibitors, gamma secretase inhibitors and beta secretase inhibitors.

If formulated as a fixed dose, such combination products employ the compounds of this invention within the dosage range described herein and the other pharmaceutically active agent or treatment within its dosage range.

Accordingly, in an aspect, this invention includes combinations comprising an amount of at least one compound of Formula I, or a pharmaceutically acceptable salt, solvate, ester or prodrug thereof, and an amount of one or more additional agents listed above wherein the amounts of the compounds/treatments result in desired therapeutic effect.

The pharmacological properties of the compounds of this invention may be confirmed by a number of pharmacological assays. Certain assays are exemplified later in this document.

This invention is also directed to pharmaceutical compositions which comprise at least one compound of Formula I, or a pharmaceutically acceptable salt, solvate, ester or prodrug of said compound and at least one pharmaceutically acceptable carrier.

Other embodiments of this invention are directed to pharmaceutically acceptable salts of any one of compounds formulas 3-50 as defined herein, formulas 51-53 as defined herein, A6-A17, D9-D20, E4-E21, F1-12, G6-G23, H1-H17, I4-I12, J5-J12, M6-M16, O11 and N6.

Other embodiments of this invention are directed to pharmaceutically acceptable esters of any one of compounds formulas 3-50 as defined herein, formulas 51-53 as defined herein, A6-A17, D9-D20, E4-E21, F1-12, G6-G23, H1-H17, I4-I12, J5-J12, M6-M16, O11 and N6.

Other embodiments of this invention are directed to solvates of any one of compounds formulas 3-50 as defined herein, formulas 51-53 as defined herein, A6-A17, D9-D20, E4-E21, F1-12, G6-G23, H1-H17, I4-I12, J5-J12, M6-M16, O11 and N6.

Other embodiments of this invention are directed to any one of compounds formulas 3-50 as defined herein, formulas 51-53 as defined herein, A6-A17, D9-D20, E4-E21, F1-12, G6-G23, H1-H17, I4-I12, J5-J12, M6-M16, O11 and N6 in pure and isolated form.

Another embodiment of this invention is directed to a pharmaceutically acceptable salt of a compound of formula I.

Another embodiment of this invention is directed to a pharmaceutically acceptable ester of a compound of formula I.

Another embodiment of this invention is directed to a solvate of a compound of formula I.

Another embodiment of this invention is directed to a compound of formula I in isolated form.

Another embodiment of this invention is directed to a compound of formula I in pure form.

Another embodiment of this invention is directed to a compound of formula I in pure and isolated form.

Another embodiment of this invention is directed to a pharmaceutical composition comprising an effective amount of one or more (e.g., one) compounds of formula I and a pharmaceutically acceptable carrier.

Another embodiment of this invention is directed to a pharmaceutical composition comprising an effective amount of a pharmaceutically acceptable salt of one or more (e.g., one) compounds of formula I and a pharmaceutically acceptable carrier.

Another embodiment of this invention is directed to a pharmaceutical composition comprising an effective amount of a pharmaceutically acceptable ester of one or more (e.g., one) compounds of formula I and a pharmaceutically acceptable carrier.

Another embodiment of this invention is directed to a pharmaceutical composition comprising an effective amount of a solvate of one or more (e.g., one) compounds of formula I and a pharmaceutically acceptable carrier.

Another embodiment of this invention is directed to a pharmaceutical composition comprising an effective amount of one or more (e.g., one) compounds of formula I, and an effective amount of one or more (e.g., one) other pharmaceutically active ingredients (e.g.,) drugs, and a pharmaceutically acceptable carrier. Examples of the other pharmaceutically active ingredients include, but are not limited to drugs selected form the group consisting of: (a) drugs useful for the treatment of Alzheimer's disease, (b) drugs useful for inhibiting the deposition of amyloid protein (e.g., amyloid beta protein) in, on or around neurological tissue (e.g., the brain), (c) drugs useful for treating neurodegenerative diseases, and (d) drugs useful for inhibiting gamma-secretase.

Another embodiment of this invention is directed to a pharmaceutical composition comprising a therapeutically effective amount of at least one compound of Formula (I), or a pharmaceutically acceptable salt, solvate, or ester thereof, and at least one pharmaceutically acceptable carrier, and a therapeutically effective amount of one or more compounds selected from the group consisting of cholinesterase inhibitors, Aβ antibody inhibitors, gamma secretase inhibitors and beta secretase inhibitors.

Another embodiment of this invention is directed to a pharmaceutical composition comprising an effective amount of one or more (e.g., one) compounds of formula I, and effective amount of one or more BACE inhibitors, and a pharmaceutically acceptable carrier.

Another embodiment of this invention is directed to a pharmaceutical composition comprising an effective amount of one or more (e.g., one) compounds of formula I, and effective amount of one or more cholinesterase inhibitors (e.g., acetyl- and/or butyrylchlolinesterase inhibitors), and a pharmaceutically acceptable carrier.

Another embodiment of this invention is directed to a pharmaceutical composition comprising an effective amount of one or more (e.g., one) compounds of formula (I), and effective amount of one or more muscarinic antagonists (e.g., m₁ or m₂ antagonists), and a pharmaceutically acceptable carrier.

Another embodiment of this invention is directed to a pharmaceutical composition comprising an effective amount of one or more (e.g., one) compounds of formula I, and effective amount of Exelon (rivastigmine), and a pharmaceutically acceptable carrier.

Another embodiment of this invention is directed to a pharmaceutical composition comprising an effective amount of one or more (e.g., one) compounds of formula I, and effective amount of Cognex (tacrine), and a pharmaceutically acceptable carrier.

Another embodiment of this invention is directed to a pharmaceutical composition comprising an effective amount of one or more (e.g., one) compounds of formula I, and effective amount of a Tau kinase inhibitor, and a pharmaceutically acceptable carrier.

Another embodiment of this invention is directed to a pharmaceutical composition comprising an effective amount of one or more (e.g., one) compounds of formula I, and effective amount of one or more Tau kinase inhibitor (e.g., GSK3beta inhibitor, cdk5 inhibitor, ERK inhibitor), and a pharmaceutically acceptable carrier.

Another embodiment of this invention is directed to a pharmaceutical composition comprising an effective amount of one or more (e.g., one) compounds of formula I, and effective amount of one anti-Abeta vaccine (active immunization), and a pharmaceutically acceptable carrier.

Another embodiment of this invention is directed to a pharmaceutical composition comprising an effective amount of one or more (e.g., one) compounds of formula I, and effective amount of one or more APP ligands, and a pharmaceutically acceptable carrier.

Another embodiment of this invention is directed to a pharmaceutical composition comprising an effective amount of one or more (e.g., one) compounds of formula I, and effective amount of one or more agents that upregulate insulin degrading enzyme and/or neprilysin, and a pharmaceutically acceptable carrier.

Another embodiment of this invention is directed to a pharmaceutical composition comprising an effective amount of one or more (e.g., one) compounds of formula I, and effective amount of one or more cholesterol lowering agents (for example, statins such as Atorvastatin, Fluvastatin, Lovastatin, Mevastatin, Pitavastatin, Pravastatin, Rosuvastatin, Simvastatin, and cholesterol absorption inhibitor such as Ezetimibe), and a pharmaceutically acceptable carrier.

Another embodiment of this invention is directed to a pharmaceutical composition comprising an effective amount of one or more (e.g., one) compounds of formula I, and effective amount of one or more fibrates (for example, clofibrate, Clofibride, Etofibrate, Aluminium Clofibrate), and a pharmaceutically acceptable carrier

Another embodiment of this invention is directed to a pharmaceutical composition comprising an effective amount of one or more (e.g., one) compounds of formula I, and effective amount of one or more LXR agonists, and a pharmaceutically acceptable carrier.

Another embodiment of this invention is directed to a pharmaceutical composition comprising an effective amount of one or more (e.g., one) compounds of formula I, and effective amount of one or more LRP mimics, and a pharmaceutically acceptable carrier.

Another embodiment of this invention is directed to a pharmaceutical composition comprising an effective amount of one or more (e.g., one) compounds of formula I, and effective amount of one or more 5-HT6 receptor antagonists, and a pharmaceutically acceptable carrier.

Another embodiment of this invention is directed to a pharmaceutical composition comprising an effective amount of one or more (e.g., one) compounds of formula I, and effective amount of one or more nicotinic receptor agonists, and a pharmaceutically acceptable carrier.

Another embodiment of this invention is directed to a pharmaceutical composition comprising an effective amount of one or more (e.g., one) compounds of formula I, and effective amount of one or more H3 receptor antagonists, and a pharmaceutically acceptable carrier.

Another embodiment of this invention is directed to a pharmaceutical composition comprising an effective amount of one or more (e.g., one) compounds of formula I, and effective amount of one or more histone deacetylase inhibitors, and a pharmaceutically acceptable carrier.

Another embodiment of this invention is directed to a pharmaceutical composition comprising an effective amount of one or more (e.g., one) compounds of formula I, and effective amount of one or more hsp90 inhibitors, and a pharmaceutically acceptable carrier.

Another embodiment of this invention is directed to a pharmaceutical composition comprising an effective amount of one or more (e.g., one) compounds of formula I, and effective amount of one or more m1 muscarinic receptor agonists, and a pharmaceutically acceptable carrier.

Another embodiment of this invention is directed to combinations, i.e., a pharmaceutical composition, comprising a pharmaceutically acceptable carrier, an effective (i.e., therapeutically effective) amount of one or more compounds of formula (I), in combination with an effective (i.e., therapeutically effective) amount of one or more compounds selected from the group consisting of cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), Aβ antibody inhibitors, gamma secretase inhibitors and beta secretase inhibitors.

Another embodiment of this invention is directed to a pharmaceutical composition comprising an effective amount of one or more (e.g., one) compounds of formula I, and effective amount of one or more 5-HT6 receptor antagonists mGluR1 or mGluR5 positive allosteric modulators or agonists, and a pharmaceutically acceptable carrier.

Another embodiment of this invention is directed to a pharmaceutical composition comprising an effective amount of one or more (e.g., one) compounds of formula I, and effective amount of one or more one mGluR2/3 antagonists, and a pharmaceutically acceptable carrier.

Another embodiment of this invention is directed to a pharmaceutical composition comprising an effective amount of one or more (e.g., one) compounds of formula I, and effective amount of one or more anti-inflammatory agents that can reduce neuroinflammation, and a pharmaceutically acceptable carrier.

Another embodiment of this invention is directed to a pharmaceutical composition comprising an effective amount of one or more (e.g., one) compounds of formula I, and effective amount of one or more Prostaglandin EP2 receptor antagonists, and a pharmaceutically acceptable carrier.

Another embodiment of this invention is directed to a pharmaceutical composition comprising an effective amount of one or more (e.g., one) compounds of formula I, and effective amount of one or more PAI-1 inhibitors, and a pharmaceutically acceptable carrier.

Another embodiment of this invention is directed to a pharmaceutical composition comprising an effective amount of one or more (e.g., one) compounds of formula I, and effective amount of one or more agents that can induce Abeta efflux such as gelsolin, and a pharmaceutically acceptable carrier.

The compounds of formula (I) can be useful as gamma secretase modulators and can be useful in the treatment and prevention of diseases such as, for example, central nervous system disorders (such as Alzheimers disease and Downs Syndrome), mild cognitive impairment, glaucoma, cerebral amyloid angiopathy, stroke, dementia, microgliosis, brain inflammation, and olfactory function loss.

The compounds of formula I can be useful as gamma secretase modulators and can be useful in the treatment and prevention of diseases such as, for example, central nervous system disorders such as Alzheimers disease and Downs Syndrome.

Thus, another embodiment of this invention is directed to a method for modulating (including inhibiting, antagonizing and the like) gamma-secretase comprising administering an effective amount of one or more (e.g., one) compounds of formula I to a patient in need of such treatment.

Another embodiment of this invention is directed to a method for modulating (including inhibiting, antagonizing and the like) gamma-secretase, comprising administering an effective amount of a compound of formula I to a patient in need of treatment.

Another embodiment of this invention is directed to a method of treating one or more neurodegenerative diseases, comprising administering an effective amount of one or more (e.g., one) compounds of formula I to a patient in need of treatment.

Another embodiment of this invention is directed to a method of treating one or more neurodegenerative diseases, comprising administering an effective amount of a compound of formula I to a patient in need of treatment.

Another embodiment of this invention is directed to a method of inhibiting the deposition of amyloid protein (e.g., amyloid beta protein) in, on or around neurological tissue (e.g., the brain), comprising administering an effective amount of one or more (e.g., one) compounds of formula I to a patient in need of treatment.

Another embodiment of this invention is directed to a method of inhibiting the deposition of amyloid protein (e.g., amyloid beta protein) in, on or around neurological tissue (e.g., the brain), comprising administering an effective amount of a compound of formula I to a patient in need of treatment.

Another embodiment of this invention is directed to a method of treating Alzheimer's disease, comprising administering an effective amount of one or more (e.g., one) compounds of formula I to a patient in need of treatment.

Another embodiment of this invention is directed to a method of treating Alzheimer's disease, comprising administering an effective amount of a compound of formula I to a patient in need of treatment.

Another embodiment of this invention is directed to a method of treating mild cognitive impairment, glaucoma, cerebral amyloid angiopathy, stroke, dementia, microgliosis, brain inflammation, or olfactory function loss, comprising administering an effective (i.e., therapeutically effective) amount of one or more (e.g., one) compounds of formula (I) to a patient in need of treatment.

Another embodiment of this invention is directed to a method of treating mild cognitive impairment, glaucoma, cerebral amyloid angiopathy, stroke, dementia, microgliosis, brain inflammation, or olfactory function loss, comprising administering an effective (i.e., therapeutically effective) amount of a compound of formula (I) to a patient in need of treatment.

Another embodiment of this invention is directed to a method of treating mild cognitive impairment, comprising administering an effective amount of one or more (e.g., one) compounds of formula (I) to a patient in need of treatment.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of an effective amount of one or more (e.g. one) compounds of formula I and the administration of an effective amount of one or more (e.g., one) other pharmaceutical active ingredients (e.g., drugs). The compounds of formula I and the other drugs can be administered separately (i.e., each is in its own separate dosage form), or the compounds of formula I can be combined with the other drugs in the same dosage form.

Thus, other embodiments of this invention are directed to any one of the methods of treatment, or methods of inhibiting, described herein, wherein an effective amount of the compound of formula (I) is used in combination with an effective amount of one or more other pharmaceutically active ingredients (e.g., drugs). The other pharmaceutically active ingredients (i.e., drugs) are selected from the group consisting of: BACE inhibitors (beta secretase inhibitors), muscarinic antagonists (e.g., m₁ agonists or m₂ antagonists), cholinesterase inhibitors (e.g., acetyl- and/or butyrylchlolinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors; Exelon (rivastigmine); Cognex (tacrine); Tau kinase inhibitors (e.g., GSK3beta inhibitors, cdk5 inhibitors, or ERK inhibitors); anti-Abeta vaccine; APP ligands; agents that upregulate insulin cholesterol lowering agents (for example, statins such as Atorvastatin, Fluvastatin, Lovastatin, Mevastatin, Pitavastatin, Pravastatin, Rosuvastatin, Simvastatin); cholesterol absorption inhibitors (such as Ezetimibe); fibrates (such as, for example, for example, clofibrate, Clofibride, Etofibrate, and Aluminium Clofibrate); LXR agonists; LRP mimics; nicotinic receptor agonists; H3 receptor antagonists; histone deacetylase inhibitors; hsp90 inhibitors; m1 muscarinic receptor agonists; 5-HT6 receptor antagonists; mGluR1; mGluR5; positive allosteric modulators or agonists; mGluR2/3 antagonists; anti-inflammatory agents that can reduce neuroinflammation; Prostaglandin EP2 receptor antagonists; PAI-1 inhibitors; and agents that can induce Abeta efflux such as gelsolin.

Another embodiment of this invention is directed to a method of treating Alzheimer's disease, comprising administering an effective amount of one or more (e.g., one) compounds of formula I, in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Alzheimer's disease, comprising administering an effective amount of a compound of formula I, in combination with an effective amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Alzheimer's disease, comprising administering an effective amount of one or more (e.g., one) compounds of formula I, in combination with an effective amount of one or more compounds selected from the group consisting of Aβ antibody inhibitors, gamma secretase inhibitors and beta secretase inhibitors.

Another embodiment of this invention is directed to a method of treating Alzheimer's disease, comprising administering an effective amount of one or more (e.g., one) compounds of formula I, in combination with an effective amount of one or more BACE inhibitors.

Another embodiment of this invention is directed to a method of treating Alzheimer's disease, comprising administering an effective amount of one or more compounds of formula I, in combination with an effective amount of Exelon (rivastigmine).

Another embodiment of this invention is directed to a method of treating Alzheimer's disease, comprising administering an effective amount of one or more compounds of formula I, in combination with an effective amount of Cognex (tacrine).

Another embodiment of this invention is directed to a method of treating Alzheimer's disease, comprising administering an effective amount of one or more compounds of formula I, in combination with an effective amount of a Tau kinase inhibitor.

Another embodiment of this invention is directed to a method of treating Alzheimer's disease, comprising administering an effective amount of one or more compounds of formula I, in combination with an effective amount of one or more Tau kinase inhibitor (e.g., GSK3beta inhibitor, cdk5 inhibitor. ERK inhibitor).

This invention also provides a method of treating Alzheimer's disease, comprising administering an effective amount of one or more compounds of formula I. in combination with an effective amount of one anti-Abeta vaccination (active immunization).

Another embodiment of this invention is directed to a method of treating Alzheimer's disease, comprising administering an effective amount of one or more compounds of formula I, in combination with an effective amount of one or more APP ligands.

Another embodiment of this invention is directed to a method of treating Alzheimer's disease, comprising administering an effective amount of one or more compounds of formula I, in combination with an effective amount of one or more agents that upregulate insulin degrading enzyme and/or neprilysin.

Another embodiment of this invention is directed to a method of treating Alzheimer's disease, comprising administering an effective amount of one or more compounds of formula I, in combination with an effective amount of one or more cholesterol lowering agents (for example, statins such as Atorvastatin, Fluvastatin, Lovastatin, Mevastatin, Pitavastatin, Pravastatin, Rosuvastatin, Simvastatin, and cholesterol absorption inhibitor such as Ezetimibe).

This invention also provides a method of treating Alzheimer's disease, comprising administering an effective amount of one or more compounds of formula I, in combination with an effective amount of one or more fibrates (for example, clofibrate, Clofibride, Etofibrate, Aluminium Clofibrate).

Another embodiment of this invention is directed to a method of treating Alzheimer's disease, comprising administering an effective amount of one or more compounds of formula I, in combination with an effective amount of one or more LXR agonists.

Another embodiment of this invention is directed to a method of treating Alzheimer's disease, comprising administering an effective amount of one or more compounds of formula I, in combination with an effective amount of one or more

LRP mimics.

Another embodiment of this invention is directed to a method of treating Alzheimer's disease, comprising administering an effective amount of one or more compounds of formula I, in combination with an effective amount of one or more 5-HT6 receptor antagonists.

Another embodiment of this invention is directed to a method of treating Alzheimer's disease, comprising administering an effective amount of one or more compounds of formula I, in combination with an effective amount of one or more nicotinic receptor agonists.

Another embodiment of this invention is directed to a method of treating Alzheimer's disease, comprising administering an effective amount of one or more compounds of formula I, in combination with an effective amount of one or more H3 receptor antagonists.

This invention also provides a method of treating Alzheimer's disease, comprising administering an effective amount of one or more compounds of formula I, in combination with an effective amount of one or more histone deacetylase inhibitors.

Another embodiment of this invention is directed to a method of treating Alzheimer's disease, comprising administering an effective amount of one or more compounds of formula I, in combination with an effective amount of one or more hsp90 inhibitors.

Another embodiment of this invention is directed to a method of treating Alzheimer's disease, comprising administering an effective amount of one or more compounds of formula I, in combination with an effective amount of one or more ml muscarinic receptor agonists.

Another embodiment of this invention is directed to a method of treating Alzheimer's disease, comprising administering an effective amount of one or more compounds of formula I, in combination with an effective amount of one or more 5-HT6 receptor antagonists mGluR1 or mGluR5 positive allosteric modulators or agonists.

Another embodiment of this invention is directed to a method of treating Alzheimer's disease, comprising administering an effective amount of one or more compounds of formula I, in combination with an effective amount of one or more mGluR2/3 antagonists.

Another embodiment of this invention is directed to a method of treating Alzheimer's disease, comprising administering an effective amount of one or more compounds of formula I, in combination with an effective amount of one or more anti-inflammatory agents that can reduce neuroinflammation.

Another embodiment of this invention is directed to a method of treating Alzheimer's disease, comprising administering an effective amount of one or more compounds of formula I, in combination with an effective amount of one or more Prostaglandin EP2 receptor antagonists.

Another embodiment of this invention is directed to a method of treating Alzheimer's disease, comprising administering an effective amount of one or more compounds of formula I, in combination with an effective amount of one or more PAI-1 inhibitors.

Another embodiment of this invention is directed to a method of treating Alzheimer's disease, comprising administering an effective amount of one or more compounds of formula I, in combination with an effective amount of one or more agents that can induce Abeta efflux such as gelsolin.

Other embodiments of this invention are directed to any one of the above embodiments directed to combination therapies (i.e., the above methods of treating wherein compounds of formula (I) are used in combination with other pharmaceutically active ingredients, i.e., drugs) wherein the compound of formula (I) is selected from the group consisting of the compounds formulas 3-50 as defined herein, formulas 51-53 as defined herein, A6-A17, D9-D20, E4-E21, F1-12, G6-G23, H1-H17, I4-I12, J5-J12, M6-M16, O11 and N6.

Another embodiment of this invention is directed to a pharmaceutical composition comprising an effective amount of one or more (e.g., one) compounds of formula I, and effective amount of one or more Tau kinase inhibitor (e.g., GSK3beta inhibitor, cdk5 inhibitor, ERK inhibitor), and a pharmaceutically acceptable carrier.

Another embodiment of this invention is directed to a pharmaceutical composition comprising an effective amount of one or more (e.g., one) compounds of formula I, and effective amount of one anti-Abeta vaccine (active immunization), and a pharmaceutically acceptable carrier.

Another embodiment of this invention is directed to a pharmaceutical composition comprising an effective amount of one or more (e.g., one) compounds of formula I, and effective amount of one or more APP ligands, and a pharmaceutically acceptable carrier.

Another embodiment of this invention is directed to a pharmaceutical composition comprising an effective amount of one or more (e.g., one) compounds of formula I, and effective amount of one or more agents that upregulate insulin degrading enzyme and/or neprilysin, and a pharmaceutically acceptable carrier.

Another embodiment of this invention is directed to a pharmaceutical composition comprising an effective amount of one or more (e.g., one) compounds of formula I, and effective amount of one or more cholesterol lowering agents (for example, statins such as Atorvastatin, Fluvastatin, Lovastatin, Mevastatin, Pitavastatin, Pravastatin, Rosuvastatin, Simvastatin, and cholesterol absorption inhibitor such as Ezetimibe), and a pharmaceutically acceptable carrier.

Another embodiment of this invention is directed to a pharmaceutical composition comprising an effective amount of one or more (e.g., one) compounds of formula I, and effective amount of one or more fibrates (for example, clofibrate, Clofibride, Etofibrate, Aluminium Clofibrate), and a pharmaceutically acceptable carrier

Another embodiment of this invention is directed to a pharmaceutical composition comprising an effective amount of one or more (e.g., one) compounds of formula I, and effective amount of one or more LXR agonists, and a pharmaceutically acceptable carrier.

Another embodiment of this invention is directed to a pharmaceutical composition comprising an effective amount of one or more (e.g., one) compounds of formula I, and effective amount of one or more LRP mimics, and a pharmaceutically acceptable carrier.

Another embodiment of this invention is directed to a pharmaceutical composition comprising an effective amount of one or more (e.g., one) compounds of formula I, and effective amount of one or more 5-HT6 receptor antagonists, and a pharmaceutically acceptable carrier.

Another embodiment of this invention is directed to a pharmaceutical composition comprising an effective amount of one or more (e.g., one) compounds of formula I, and effective amount of one or more nicotinic receptor agonists, and a pharmaceutically acceptable carrier.

Another embodiment of this invention is directed to a pharmaceutical composition comprising an effective amount of one or more (e.g., one) compounds of formula I, and effective amount of one or more H3 receptor antagonists, and a pharmaceutically acceptable carrier.

Another embodiment of this invention is directed to a pharmaceutical composition comprising an effective amount of one or more (e.g., one) compounds of formula I, and effective amount of one or more histone deacetylase inhibitors, and a pharmaceutically acceptable carrier.

Another embodiment of this invention is directed to a pharmaceutical composition comprising an effective amount of one or more (e.g., one) compounds of formula I, and effective amount of one or more hsp90 inhibitors, and a pharmaceutically acceptable carrier.

Another embodiment of this invention is directed to a pharmaceutical composition comprising an effective amount of one or more (e.g., one) compounds of formula I, and effective amount of one or more m1 muscarinic receptor agonists, and a pharmaceutically acceptable carrier.

Another embodiment of this invention is directed to combinations, i.e., a pharmaceutical composition, comprising a pharmaceutically acceptable carrier, an effective (i.e., therapeutically effective) amount of one or more compounds of formula (I), in combination with an effective (i.e., therapeutically effective) amount of one or more compounds selected from the group consisting of cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), Aβ antibody inhibitors, gamma secretase inhibitors and beta secretase inhibitors.

Another embodiment of this invention is directed to a pharmaceutical composition comprising an effective amount of one or more (e.g., one) compounds of formula I, and effective amount of one or more 5-HT6 receptor antagonists mGluR1 or mGluR5 positive allosteric modulators or agonists, and a pharmaceutically acceptable carrier.

Another embodiment of this invention is directed to a pharmaceutical composition comprising an effective amount of one or more (e.g., one) compounds of formula I, and effective amount of one or more one mGluR2/3 antagonists, and a pharmaceutically acceptable carrier.

Another embodiment of this invention is directed to a pharmaceutical composition comprising an effective amount of one or more (e.g., one) compounds of formula I, and effective amount of one or more anti-inflammatory agents that can reduce neuroinflammation, and a pharmaceutically acceptable carrier.

Another embodiment of this invention is directed to a pharmaceutical composition comprising an effective amount of one or more (e.g., one) compounds of formula I, and effective amount of one or more Prostaglandin EP2 receptor antagonists, and a pharmaceutically acceptable carrier.

Another embodiment of this invention is directed to a pharmaceutical composition comprising an effective amount of one or more (e.g., one) compounds of formula I, and effective amount of one or more PAI-1 inhibitors, and a pharmaceutically acceptable carrier.

Another embodiment of this invention is directed to a pharmaceutical composition comprising an effective amount of one or more (e.g., one) compounds of formula I, and effective amount of one or more agents that can induce Abeta efflux such as gelsolin, and a pharmaceutically acceptable carrier.

This invention also provides a method of treating Downs syndrome, comprising administering an effective amount of one or more (e.g., one) compounds of formula I to a patient in need of treatment.

This invention also provides a method of treating Downs syndrome, comprising administering an effective amount of a compound of formula I to a patient in need of treatment.

This invention also provides a method of treating Downs syndrome, comprising administering an effective amount of one or more (e.g., one) compounds of formula I, in combination with an effective amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

This invention also provides a method of treating Downs syndrome, comprising administering an effective amount of a compound of formula I, in combination with an effective amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

This invention also provides a method of treating mild cognitive impairment, comprising administering an effective amount of one or more (e.g., one) compounds of formula I to a patient in need of treatment.

This invention also provides a method of treating glaucoma, comprising administering an effective amount of one or more (e.g., one) compounds of formula I to a patient in need of treatment.

This invention also provides a method of treating cerebral amyloid angiopathy, comprising administering an effective amount of one or more (e.g., one) compounds of formula I to a patient in need of treatment.

This invention also provides a method of treating stroke, comprising administering an effective amount of one or more (e.g., one) compounds of formula I to a patient in need of treatment.

This invention also provides a method of treating dementia, comprising administering an effective amount of one or more (e.g., one) compounds of formula I to a patient in need of treatment.

This invention also provides a method of treating microgliosis, comprising administering an effective amount of one or more (e.g., one) compounds of formula I to a patient in need of treatment.

This invention also provides a method of treating brain inflammation, comprising administering an effective amount of one or more (e.g., one) compounds of formula I to a patient in need of treatment.

This invention also provides a method of treating olfactory function loss, comprising administering an effective amount of one or more (e.g., one) compounds of formula I to a patient in need of treatment.

This invention also provides combinations (i.e., pharmaceutical compositions) comprising an effective amount of one or more (e.g., one) compounds of formula I, in combination with an effective amount of one or more compounds selected from the group consisting of cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), Aβ antibody inhibitors, gamma secretase inhibitors and beta secretase inhibitors. The pharmaceutical compositions also comprise a pharmaceutically acceptable carrier.

This invention also provides a kit comprising, in separate containers, in a single package, pharmaceutical compositions for use in combination, wherein one container comprises an effective amount of a compound of formula I in a pharmaceutically acceptable carrier, and another container (i.e., a second container) comprises an effective amount of another pharmaceutically active ingredient (as described above), the combined quantities of the compound of formula I and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase.

Another embodiment of this invention is directed to a pharmaceutically acceptable salt of a compound of formula (I), said compound of formula (I) being selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6.

Another embodiment of this invention is directed to a pharmaceutically acceptable ester of a compound of formula (I), said compound of formula (I) being selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6. Another embodiment of this invention is directed to a solvate of a compound of formula (I), said compound of formula (I) being selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6.

Another embodiment of this invention is directed to a compound of formula (I) in isolated form, said compound of formula (I) being selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6.

Another embodiment of this invention is directed to a compound of formula (I) in pure form, said compound of formula (I) being selected from the group consisting of: said compound of formula (I) being selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6.

Another embodiment of this invention is directed to a compound of formula (I) in pure and isolated form, said compound of formula (I) being selected from the group consisting of: said compound of formula (I) being selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6.

Another embodiment is directed to a pharmaceutical composition comprising an effective amount of one or more (e.g., one) compounds of formula (I) and a pharmaceutically acceptable carrier, said compound of formula (I) being selected from the group consisting of: said compound of formula (I) being selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6.

Another embodiment is directed to a pharmaceutical composition comprising an effective amount of a pharmaceutically acceptable salt of one or more (e.g., one) compounds of formula (I) and a pharmaceutically acceptable carrier, said compound of formula (I) being selected from the group consisting of: said compound of formula (I) being selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6.

Another embodiment is directed to a pharmaceutical composition comprising an effective amount of a pharmaceutically acceptable ester of one or more (e.g., one) compounds of formula (I) and a pharmaceutically acceptable carrier, said compound of formula (I) being selected from the group consisting of: said compound of formula (I) being selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6.

Another embodiment is directed to a pharmaceutical composition comprising an effective amount of a solvate of one or more (e.g., one) compounds of formula (I) and a pharmaceutically acceptable carrier, said compound of formula (I) being selected from the group consisting of: said compound of formula (I) being selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6.

Another embodiment of this invention is directed to a pharmaceutical composition comprising an effective amount of one or more (e.g., one) compounds of formula (I), and an effective amount of one or more (e.g., one) other pharmaceutically active ingredients (e.g., drugs), and a pharmaceutically acceptable carrier. Examples of the other pharmaceutically active ingredients include, but are not limited to drugs selected form the group consisting of: (a) drugs useful for the treatment of Alzheimer's disease, (b) drugs useful for inhibiting the deposition of amyloid protein (e.g., amyloid beta protein) in, on or around neurological tissue (e.g., the brain), (c) drugs useful for treating neurodegenerative diseases, and (d) drugs useful for inhibiting gamma-secretase, said compound of formula (I) being selected from the group consisting of: said compound of formula (I) being selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6.

Another embodiment of this invention is directed to a pharmaceutical composition comprising an effective amount of one or more (e.g., one) compounds of formula (I), and effective amount of one or more BACE inhibitors, and a pharmaceutically acceptable carrier, said compound of formula (I) being selected from the group consisting of: said compound of formula (I) being selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6.

Another embodiment of this invention is directed to a pharmaceutical composition comprising an effective amount of one or more (e.g., one) compounds selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, and an effective amount of one or more cholinesterase inhibitors (e.g., acetyl- and/or butyrylchlolinesterase inhibitors), and a pharmaceutically acceptable carrier.

Another embodiment of this invention is directed to a pharmaceutical composition comprising an effective amount of one or more (e.g., one) compounds selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, and effective amount of one or more muscarinic antagonists (e.g., m₁ agonists or m₂ antagonists), and a pharmaceutically acceptable carrier.

Another embodiment of this invention is directed to a pharmaceutical composition comprising an effective amount of one or more (e.g., one) compounds selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, and effective amount of Exelon (rivastigmine), and a pharmaceutically acceptable carrier.

Another embodiment of this invention is directed to a pharmaceutical composition comprising an effective amount of one or more (e.g., one) compounds selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, and effective amount of Cognex (tacrine), and a pharmaceutically acceptable carrier.

Another embodiment of this invention is directed to a pharmaceutical composition comprising an effective amount of one or more (e.g., one) compounds selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, and effective amount of a Tau kinase inhibitor, and a pharmaceutically acceptable carrier.

Another embodiment of this invention is directed to a pharmaceutical composition comprising an effective amount of one or more (e.g., one) compounds selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, and effective amount of one or more Tau kinase inhibitor (e.g., GSK3beta inhibitor, cdk5 inhibitor, ERK inhibitor), and a pharmaceutically acceptable carrier.

Another embodiment of this invention is directed to a pharmaceutical composition comprising an effective amount of one or more (e.g., one) compounds selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, and effective amount of one anti-Abeta vaccine (active immunization), and a pharmaceutically acceptable carrier.

Another embodiment of this invention is directed to a pharmaceutical composition comprising an effective amount of one or more (e.g., one) compounds selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, and effective amount of one or more APP ligands, and a pharmaceutically acceptable carrier.

Another embodiment of this invention is directed to a pharmaceutical composition comprising an effective amount of one or more (e.g., one) compounds selected from the group consisting of: consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, and effective amount of one or more agents that upregulate insulin degrading enzyme and/or neprilysin, and a pharmaceutically acceptable carrier.

Another embodiment of this invention is directed to a pharmaceutical composition comprising an effective amount of one or more (e.g., one) compounds selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, and effective amount of one or more cholesterol lowering agents (for example, statins such as Atorvastatin, Fluvastatin, Lovastatin, Mevastatin, Pitavastatin, Pravastatin, Rosuvastatin, Simvastatin, and cholesterol absorption inhibitor such as Ezetimibe), and a pharmaceutically acceptable carrier.

Another embodiment of this invention is directed to a pharmaceutical composition comprising an effective amount of one or more (e.g., one) compounds selected from the group consisting of: consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, and effective amount of one or more fibrates (for example, clofibrate, Clofibride, Etofibrate, Aluminium Clofibrate), and a pharmaceutically acceptable carrier.

Another embodiment of this invention is directed to a pharmaceutical composition comprising an effective amount of one or more (e.g., one) compounds selected from the group consisting of: consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, and effective amount of one or more LXR agonists, and a pharmaceutically acceptable carrier.

Another embodiment of this invention is directed to a pharmaceutical composition comprising an effective amount of one or more (e.g., one) compounds selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, and effective amount of one or more LRP mimics, and a pharmaceutically acceptable carrier.

Another embodiment of this invention is directed to a pharmaceutical composition comprising an effective amount of one or more (e.g., one) compounds selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, and effective amount of one or more 5-HT6 receptor antagonists, and a pharmaceutically acceptable carrier.

Another embodiment of this invention is directed to a pharmaceutical composition comprising an effective amount of one or more (e.g., one) compounds selected from the group consisting of: consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, and effective amount of one or more nicotinic receptor agonists, and a pharmaceutically acceptable carrier.

Another embodiment of this invention is directed to a pharmaceutical composition comprising an effective amount of one or more (e.g., one) compounds selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, and effective amount of one or more H3 receptor antagonists, and a pharmaceutically acceptable carrier.

Another embodiment of this invention is directed to a pharmaceutical composition comprising an effective amount of one or more (e.g., one) compounds selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, and effective amount of one or more histone deacetylase inhibitors, and a pharmaceutically acceptable carrier.

Another embodiment of this invention is directed to a pharmaceutical composition comprising an effective amount of one or more (e.g., one) compounds selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, and effective amount of one or more hsp90 inhibitors, and a pharmaceutically acceptable carrier.

Another embodiment of this invention is directed to a pharmaceutical composition comprising an effective amount of one or more (e.g., one) compounds selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, and effective amount of one or more m1 muscarinic receptor agonists, and a pharmaceutically acceptable carrier.

Another embodiment of this invention is directed to a pharmaceutical composition comprising an effective amount of one or more (e.g., one) compounds selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, and effective amount of one or more 5-HT6 receptor antagonists mGluR1 or mGluR5 positive allosteric modulators or agonists, and a pharmaceutically acceptable carrier.

Another embodiment of this invention is directed to a pharmaceutical composition comprising an effective amount of one or more (e.g., one) compounds selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, and effective amount of one or more one mGluR2/3 antagonists, and a pharmaceutically acceptable carrier.

Another embodiment of this invention is directed to a pharmaceutical composition comprising an effective amount of one or more (e.g., one) compounds selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, and effective amount of one or more anti-inflammatory agents that can reduce neuroinflammation, and a pharmaceutically acceptable carrier.

Another embodiment of this invention is directed to a pharmaceutical composition comprising an effective amount of one or more (e.g., one) compounds selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, and effective amount of one or more Prostaglandin EP2 receptor antagonists, and a pharmaceutically acceptable carrier.

Another embodiment of this invention is directed to a pharmaceutical composition comprising an effective amount of one or more (e.g., one) compounds selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, and effective amount of one or more PAH inhibitors, and a pharmaceutically acceptable carrier.

Another embodiment of this invention is directed to a pharmaceutical composition comprising an effective amount of one or more (e.g., one) compounds selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, and effective amount of one or more agents that can induce Abeta efflux such as gelsolin, and a pharmaceutically acceptable carrier.

The compounds of formula I selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6 can be useful as gamma secretase modulators and can be useful in the treatment and prevention of diseases such as, for example, central nervous system disorders (such as Alzheimers disease and Downs Syndrome), and treating mild cognitive impairment, glaucoma, cerebral amyloid angiopathy, stroke, dementia, microgliosis, brain inflammation, and olfactory function loss.

Thus, another embodiment of this invention is directed to a method for modulating (including inhibiting, antagonizing and the like) gamma-secretase comprising administering an effective (i.e., therapeutically effective) amount of one or more (e.g., one) compounds of formula (I) to a patient in need of such treatment, said compound of formula (I) being selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6.

Another embodiment of this invention is directed to a method for modulating (including inhibiting, antagonizing and the like) gamma-secretase, comprising administering an effective (i.e., therapeutically effective) amount of a compound of formula (I) to a patient in need of treatment, said compound of formula (I) being selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6.

Another embodiment of this invention is directed to a method of treating one or more neurodegenerative diseases, comprising administering an effective (i.e., therapeutically effective) amount of one or more (e.g., one) compounds of formula (I) to a patient in need of treatment, said compound of formula (I) being selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6.

Another embodiment of this invention is directed to a method of treating one or more neurodegenerative diseases, comprising administering an effective (i.e., therapeutically effective) amount of a compound of formula (I) to a patient in need of treatment, said compound of formula (I) being selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6.

Another embodiment of this invention is directed to a method of inhibiting the deposition of amyloid protein (e.g., amyloid beta protein) in, on or around neurological tissue (e.g., the brain), comprising administering an effective (i.e., therapeutically effective) amount of one or more (e.g., one) compounds of formula (I) to a patient in need of treatment, said compound of formula (I) being selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6.

Another embodiment of this invention is directed to a method of inhibiting the deposition of amyloid protein (e.g., amyloid beta protein) in, on or around neurological tissue (e.g., the brain), comprising administering an effective (i.e., therapeutically effective) amount of a compound of formula (I) to a patient in need of treatment, said compound of formula (I) being selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6.

Another embodiment of this invention is directed to a method of treating Alzheimer's disease, comprising administering an effective (i.e., therapeutically effective) amount of one or more (e.g., one) compounds of formula (I) to a patient in need of treatment, said compound of formula (I) being selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6.

Another embodiment of this invention is directed to a method of treating Alzheimer's disease, comprising administering an effective (i.e., therapeutically effective) amount of a compound of formula (I) to a patient in need of treatment, said compound of formula (I) being selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6.

Another embodiment of this invention is directed to a method of treating mild cognitive impairment, glaucoma, cerebral amyloid angiopathy, stroke, dementia, microgliosis, brain inflammation, or olfactory function loss, comprising administering an effective (i.e., therapeutically effective) amount of one or more (e.g., one) compounds of formula (I) to a patient in need of treatment, said compound of formula (I) being selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6.

Another embodiment of this invention is directed to a method of treating mild cognitive impairment, glaucoma, cerebral amyloid angiopathy, stroke, dementia, microgliosis, brain inflammation, or olfactory function loss, comprising administering an effective (i.e., therapeutically effective) amount of a compound of formula (I) to a patient in need of treatment, said compound of formula (I) being selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of one or more (e.g. one) compounds of formula (I), and the administration of one or more (e.g., one) other pharmaceutical active ingredients (e.g., drugs). The compounds of formula (I), and the other drugs can be administered separately (i.e., each is in its own separate dosage form), or the compounds of formula (I) can be combined with the other drugs in the same dosage form.

Thus, other embodiments of this invention are directed to any one of the methods of treatment, or methods of inhibiting, described herein, wherein the compounds of formula (I) are used in combination with an effective amount of one or more other pharmaceutically active ingredients selected from the group consisting of: BACE inhibitors (beta secretase inhibitors); muscarinic antagonists (e.g., m₁ agonists or m₂ antagonists); cholinesterase inhibitors (e.g., acetyl- and/or butyrylchlolinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors; Exelon (rivastigmine); Cognex (tacrine); Tau kinase inhibitors (e.g., GSK3beta inhibitors, cdk5 inhibitors, or ERK inhibitors); anti-Abeta vaccine; APP ligands; agents that upregulate insulin cholesterol lowering agents (for example, statins such as

Atorvastatin, Fluvastatin, Lovastatin, Mevastatin, Pitavastatin, Pravastatin, Rosuvastatin, Simvastatin); cholesterol absorption inhibitors (such as Ezetimibe); fibrates (such as, for example, for example, clofibrate, Clofibride, Etofibrate, and Aluminium Clofibrate); LXR agonists; LRP mimics; nicotinic receptor agonists; H3 receptor antagonists; histone deacetylase inhibitors; hsp90 inhibitors; m1 muscarinic receptor agonists; 5-HT6 receptor antagonists; mGluR1; mGluR5; positive allosteric modulators or agonists; mGluR2/3 antagonists; anti-inflammatory agents that can reduce neuroinflammation; Prostaglandin EP2 receptor antagonists; PAI-1 inhibitors; and agents that can induce Abeta efflux such as gelsolin.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of one or more (e.g. one) compounds of formula (I) selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, and the administration of one or more (e.g., one) other pharmaceutical active ingredients (e.g., drugs). The compounds of formula formulas 3-50 as defined herein, formulas 51-53 as defined herein, A6-A17, D9-D20, E4-E21, F1-12, G6-G23, H1-H17, I4-I12, J5-J12, M6-M16, O11 and N6, and the other drugs can be administered separately (i.e., each is in its own separate dosage form), or the compounds of formula formulas 3-50 as defined herein, formulas 51-53 as defined herein, A6-A17, D9-D20, E4-E21, F1-12, G6-G23, H1-H17, I4-I12, J5-J12, M6-M16, O11 and N6 can be combined with the other drugs in the same dosage form.

Thus, other embodiments of this invention are directed to any one of the methods of treatment, or methods of inhibiting, described herein, wherein the compounds of formula (I), selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6 are used in combination with an effective amount of one or more other pharmaceutically active ingredients selected from the group consisting of: BACE inhibitors (beta secretase inhibitors), muscarinic antagonists (e.g., m₁ agonists or m₂ antagonists), cholinesterase inhibitors (e.g., acetyl- and/or butyrylchlolinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors; Exelon (rivastigmine); Cognex (tacrine); Tau kinase inhibitors (e.g., GSK3beta inhibitors, cdk5 inhibitors, or ERK inhibitors); anti-Abeta vaccine; APP ligands; agents that upregulate insulin cholesterol lowering agents (for example, statins such as Atorvastatin, Fluvastatin, Lovastatin, Mevastatin, Pitavastatin, Pravastatin, Rosuvastatin, Simvastatin); cholesterol absorption inhibitors (such as Ezetimibe); fibrates (such as, for example, for example, clofibrate, Clofibride, Etofibrate, and Aluminium Clofibrate); LXR agonists; LRP mimics; nicotinic receptor agonists; H3 receptor antagonists; histone deacetylase inhibitors; hsp90 inhibitors; m1 muscarinic receptor agonists; 5-HT6 receptor antagonists; mGluR1; mGluR5; positive allosteric modulators or agonists; mGluR2/3 antagonists; anti-inflammatory agents that can reduce neuroinflammation; Prostaglandin EP2 receptor antagonists; PAI-1 inhibitors; and agents that can induce Abeta efflux such as gelsolin.

Other embodiments of this invention are directed to any one of the methods of treatment, or methods of inhibiting, described herein, wherein the compounds of formula (I), selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6 are used in combination with an effective amount of one or more other pharmaceutically active ingredients selected from the group consisting of: BACE inhibitors (beta secretase inhibitors), muscarinic antagonists (e.g., m₁ agonists or m₂ antagonists), cholinesterase inhibitors (e.g., acetyl- and/or butyrylchlolinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors; and cholesterol absorption inhibitors (e.g., ezetimibe).

Other embodiments of this invention are directed to any one of the methods of treatment, or methods of inhibiting, described herein, wherein the compounds of formula (I), selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6 are used in combination with an effective amount of one or more other pharmaceutically active ingredients selected from the group consisting of: Exelon (rivastigmine); Cognex (tacrine); Tau kinase inhibitors (e.g., GSK3beta inhibitors, cdk5 inhibitors, or ERK inhibitors); anti-Abeta vaccine; APP ligands; agents that upregulate insulin cholesterol lowering agents (for example, statins such as Atorvastatin, Fluvastatin, Lovastatin, Mevastatin, Pitavastatin, Pravastatin, Rosuvastatin, Simvastatin); cholesterol absorption inhibitors (such as Ezetimibe); fibrates (such as, for example, for example, clofibrate, Clofibride, Etofibrate, and Aluminium Clofibrate); LXR agonists; LRP mimics; nicotinic receptor agonists; H3 receptor antagonists; histone deacetylase inhibitors; hsp90 inhibitors; ml muscarinic receptor agonists; 5-HT6 receptor antagonists; mGluR1, mGluR5; positive allosteric modulators or agonists; mGluR2/3 antagonists; anti-inflammatory agents that can reduce neuroinflammation; Prostaglandin EP2 receptor antagonists; PAI-1 inhibitors; and agents that can induce Abeta efflux such as gelsolin.

Another embodiment of this invention is directed to a method of treating Alzheimer's disease, comprising administering an effective (i.e., therapeutically effective) amount of one or more (e.g., one) compounds of formula (I) selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Alzheimer's disease, comprising administering an effective (i.e., therapeutically effective) amount of a compound of formula (I) selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, in combination with an effective (i.e., therapeutically effective) amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Alzheimer's disease, comprising administering an effective (i.e., therapeutically effective) amount of one or more (e.g., one) compounds of formula (I) selected from the group consisting of: in combination with an effective (i.e., therapeutically effective) amount of one or more compounds selected from the group consisting of Aβ antibody inhibitors, gamma secretase inhibitors and beta secretase inhibitors.

Another embodiment of this invention is directed to a method of treating Alzheimer's disease, comprising administering an effective (i.e., therapeutically effective) amount of one or more (e.g., one) compounds of formula (I) selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, in combination with an effective (i.e., therapeutically effective) amount of one or more BACE inhibitors.

Another embodiment of this invention is directed to a method of treating Alzheimer's disease, comprising administering an effective (i.e., therapeutically effective) amount of a compound of formula (I) selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, in combination with an effective (i.e., therapeutically effective) amount of one or more BACE inhibitors.

Another embodiment of this invention is directed to a method of treating Downs syndrome, comprising administering an effective (i.e., therapeutically effective) amount of one or more (e.g., one) compounds of formula (I) selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6 to a patient in need of treatment.

Another embodiment of this invention is directed to a method of treating Downs syndrome, comprising administering an effective (i.e., therapeutically effective) amount of a compound of formula (I) selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, to a patient in need of treatment.

Another embodiment of this invention is directed to a method of treating Downs syndrome, comprising administering an effective (i.e., therapeutically effective) amount of one or more (e.g., one) compounds of formula (I) selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Downs syndrome, comprising administering an effective (i.e., therapeutically effective) amount of a compound of formula (I) selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6, in combination with an effective (i.e., therapeutically effective) amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to combinations (i.e., pharmaceutical compositions) comprising an effective (i.e., therapeutically effective) amount of one or more (e.g., one) compounds of formula (I) selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6 in combination with an effective (i.e., therapeutically effective) amount of one or more compounds selected from the group consisting of cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), A13 antibody inhibitors, gamma secretase inhibitors and beta secretase inhibitors. The pharmaceutical compositions also comprise a pharmaceutically acceptable carrier.

This invention also provides a kit comprising, in separate containers, in a single package, pharmaceutical compositions for use in combination, wherein one container comprises an effective amount of one or more (e.g., one) compounds of formula (I) (e.g., compounds selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6 in a pharmaceutically acceptable carrier, and another container (i.e., a second container) comprises an effective amount of another pharmaceutically active ingredient (as described above), the combined quantities of the compounds of formula (I) and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase.

This invention also provides a kit comprising, in separate containers, in a single package, pharmaceutical compositions for use in combination, wherein one container comprises an effective amount of a compound selected from the group consisting of the compounds of formulas (I) (e.g. the compounds selected from the group consisting of: formulas 3-50 as defined herein, formulas 51-53 as defined herein, A6-A17, D9-D20, E4-E21, F1-12, G6-G23, H1-H17, I4-I12, J5-J12, M6-M16, O11 and N6) in a pharmaceutically acceptable carrier, and another container (i.e., a second container) comprises an effective amount of another pharmaceutically active ingredient (as described above), the combined quantities of the compound of formulas (I) and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase.

Other embodiments of this invention are directed to any one of the above methods of treatment, pharmaceutical compositions, or kits wherein the compound of formula I is any one of the compounds formulas 3-50 as defined herein, formulas 51-53 as defined herein, A6-A17, D9-D20, E4-E21, F1-12, G6-G23, H1-H17, I4-I12, J5-J12, M6-M16, O11 and N6.

Examples of cholinesterase inhibitors are tacrine, donepezil, rivastigmine, galantamine, pyridostigmine and neostigmine, with tacrine, donepezil, rivastigmine and galantamine being preferred.

Examples of m₁ agonists are known in the art. Examples of m₂ antagonists are also known in the art; in particular, m₂ antagonists are disclosed in U.S. Pat. Nos. 5,883,096; 6,037,352; 5,889,006; 6,043,255; 5,952,349; 5,935,958; 6,066,636; 5,977,138; 6,294,554; 6,043,255; and 6,458,812; and in WO 03/031412, all of which are incorporated herein by reference.

Examples of BACE inhibitors include those described in: US200510119227 published Jun. 2, 2005 (see also WO2005/016876 published Feb. 24, 2005), US2005/0043290 published Feb. 24, 2005 (see also WO2005/014540 published Feb. 17, 2005), WO2005/058311 published Jun. 30, 2005 (see also US2007/0072852 published Mar. 29, 2007), US2006/0111370 published May 25, 2006 (see also WO2006/065277 published Jun. 22, 2006), U.S. application Ser. No. 11/710,582 filed Feb. 23, 2007, US2006/0040994 published Feb. 23, 2006 (see also WO2006/014762 published Feb. 9, 2006), WO2006/014944 published Feb. 9, 2006 (see also US2006/0040948 published Feb. 23, 2006), WO2006/138266 published Dec. 28, 2006 (see also US2007/0010667 published Jan. 11, 2007), WO2006/138265 published Dec. 28, 2006, WO2006/138230 published Dec. 28, 2006, WO2006/138195 published Dec. 28, 2006 (see also US2006/0281729 published Dec. 14, 2006), WO2006/138264 published Dec. 28, 2006 (see also US2007/0060575 published Mar. 15, 2007), WO2006/138192 published Dec. 28, 2006 (see also US2006/0281730 published Dec. 14, 2006), WO2006/138217 published Dec. 28, 2006 (see also US2006/0287294 published Dec. 21, 2006), US2007/0099898 published May 3, 200 (see also WO2007/050721 published May 3, 2007), WO2007/053506 published May 10, 2007 (see also US2007/099875 published May 3, 2007), U.S. application Ser. No. 11/759,336 filed Jun. 7, 2007, U.S. Application Ser. No. 60/874,362 filed Dec. 12, 2006, and U.S. Application Ser. No. 60/874,419 filed Dec. 12, 2006, the disclosures of each being incorporated incorporated herein by reference thereto.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The compounds of this invention may also be delivered subcutaneously.

Preferably the compound is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 mg to about 100 mg, preferably from about 1 mg to about 50 mg, more preferably from about 1 mg to about 25 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 200 mg/day, in two to four divided doses.

Another aspect of this invention is a kit comprising a therapeutically effective amount of at least one compound of Formula I, or a pharmaceutically acceptable salt, solvate, ester or prodrug of said compound and a pharmaceutically acceptable carrier, vehicle or diluent.

Yet another aspect of this invention is a kit comprising an amount of at least one compound of Formula I, or a pharmaceutically acceptable salt, solvate, ester or prodrug of said compound and an amount of at least one additional agent listed above, wherein the amounts of the two or more ingredients result in desired therapeutic effect.

The invention disclosed herein is exemplified by the following illustrative example which should not be construed to limit the scope of the disclosure. Alternative mechanistic pathways and analogous structures will be apparent to those skilled in the art.

The following methods A-N (except Method C, Step 1 and Methods K and N) are prophetic and may be used to prepare the indicated compounds:

### Method A

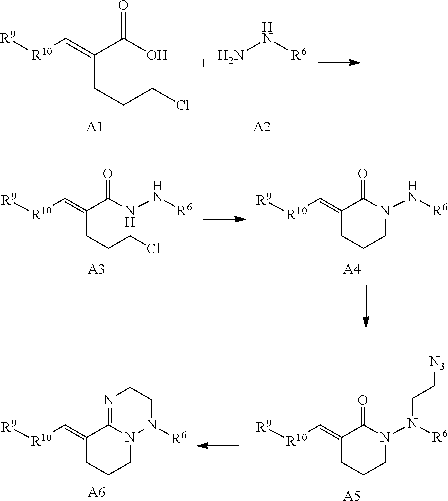

### Method A, Step 1;

To a DMF solution of compound A1 (R¹⁰=m-MeO-phenyl, R⁹=4-(4-methylimidazol-1-yl)) and compound A2 (R⁶=p-F-phenyl, 1 eq) will be added EDCl and the final compound A3 (R¹⁰=m-MeO-phenyl, R⁹=4-(4-methylimidazol-1-yl) and R⁶=p-F-phenyl) will be isolated from the reaction mixture after work-up.

### Method A, Step 2;

To a THF solution of A3 (R¹⁰=m-MeO-phenyl, R⁹=4-(4-methylimidazol-1-yl) and R⁶=p-F-phenyl) will be treated with NaH and product A4 (R¹⁰=m-MeO-phenyl, R⁹=4-(4-methylimidazol-1-yl) and R⁶=p-F-phenyl) will be isolated from the reaction mixture after work up.

### Method A, Step 3;

Compound A4 (R¹⁰=m-MeO-phenyl, R⁹=4-(4-methylimidazol-1-yl) and R⁶=p-F-phenyl) and 2-Bromoethylazide in THF will be treated with NaH and the production A5 (R¹⁰=m-MeO-phenyl, R⁹=4-(4-methylimidazol-1-yl) and R⁶=p-F-phenyl) will be isolated from the reaction mixture after work-up.

### Method A, Step 4;

Compound A5 (R¹⁰=m-MeO-phenyl, R⁹=4-(4-methylimidazol-1-yl) and R⁶=p-F-phenyl) will be treated with Ph3P under microwave conditions and product A6 (R¹⁰=m-MeO-phenyl, R⁹=4-(4-methylimidazol-1-yl) and R⁶=p-F-phenyl) will be isolated from the reaction mixture.

The following compounds may be synthesized using method similar to Method A.

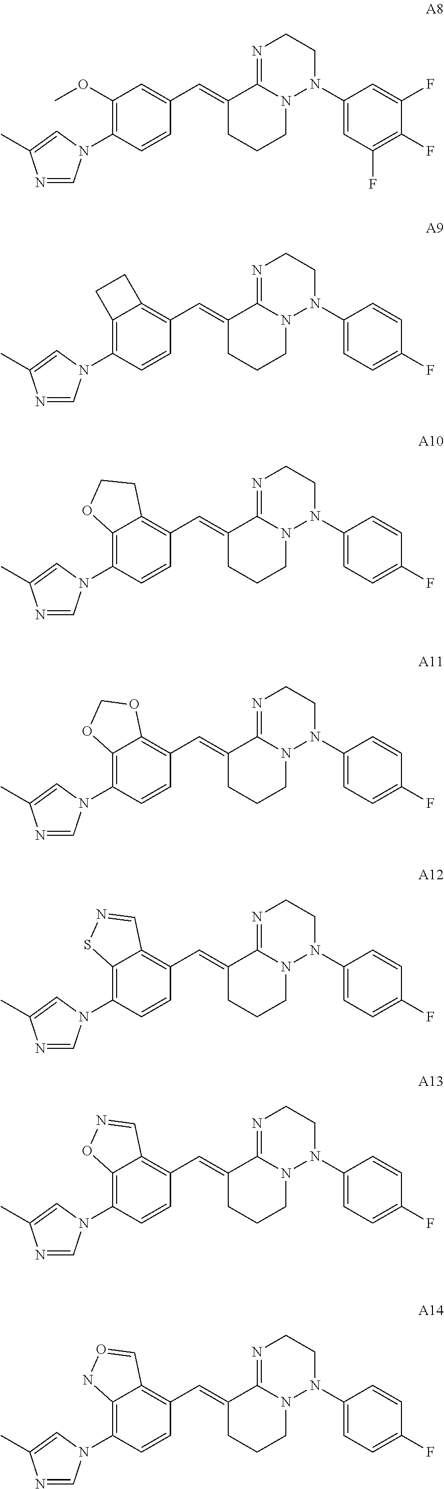

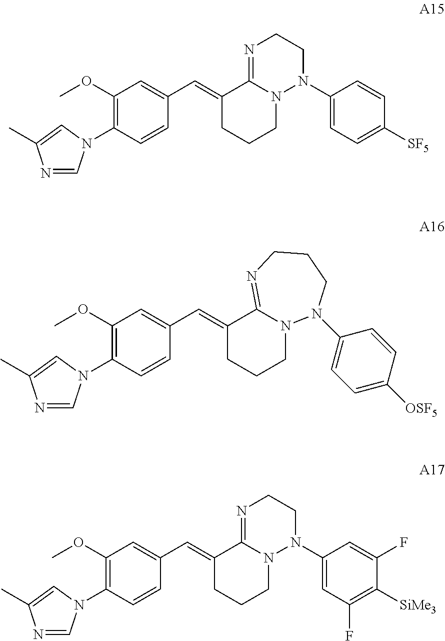

### Method B

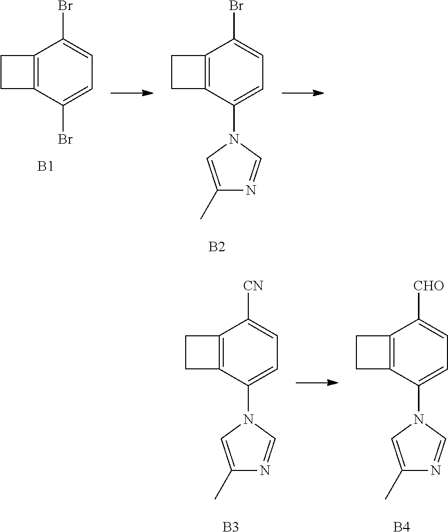

Compound B1 is obtained using a literature method by K. Walker, L., Markoski and J. Moore Synthesis, 1992, 1265.

### Method B, Step 1

To a solution of B1 (0.11 mmol) in dry 0.5 mL will be added 4-methyl imidazole (5 eq, 0.546 mmol, 44 mg), Cu₂O (0.4 equiv, 0.044 mmol, 6 mg), 4,7-dimethoxyl-1,8-phenanthracene (0.4 equiv, 0.044 mmol, 10 mg), Cs₂CO₃ (1.4 equiv, 0.154 mmol, 50 mg) and PEG (40 mg). The resulting solution will be degassed and heated at 110° C. for 40 h to give compound B1 after purification.

### Method B, Step 2

A procedure from P. Schirch and V. Bockclheide is adapted (J. Amer. Chem. Soc. 1981, 103, 6873). To a solution of 62 (1.5 g) will be added 5.0 eq of cuprous cyanide in 100 ml of N-methyl-2-pyrrolidinone. The mixture will be heated at 115° C. with stirring under nitrogen to give B3 after workup and purification.

### Method B, Step 3

To a 140 mg of B3 in ether will be added 1 eq of DiBAL in hexane. After 1 h, 5 mL of MeOH will be added and the mixture will be poured into ice water followed by acidification with 10% HCl and extraction with ether. The organic layers will be combined and solvent evaporated to give a residue which will be chromatographed to give compound B4.

The following intermediates may be synthesized using method similar to Method B for use in method C.

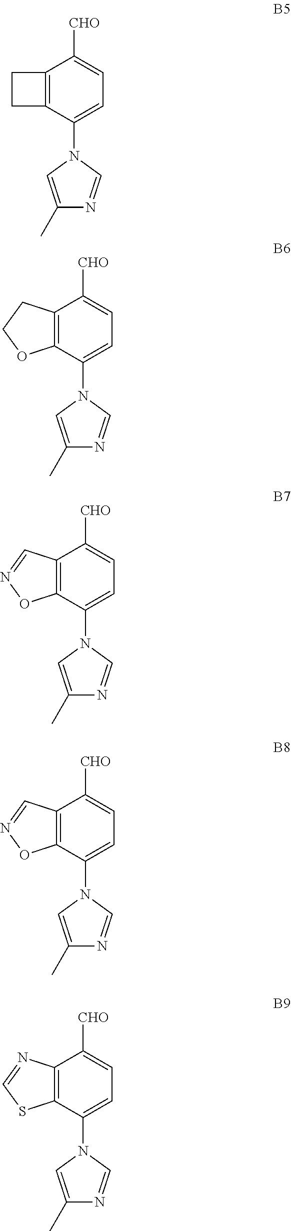

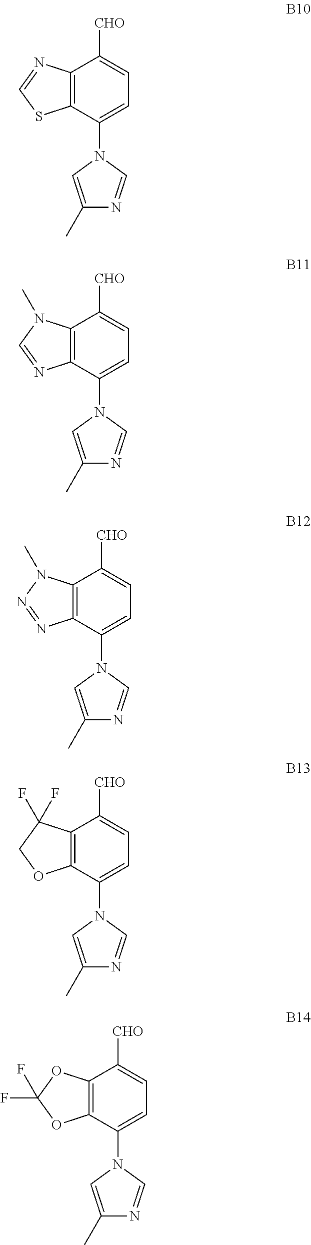

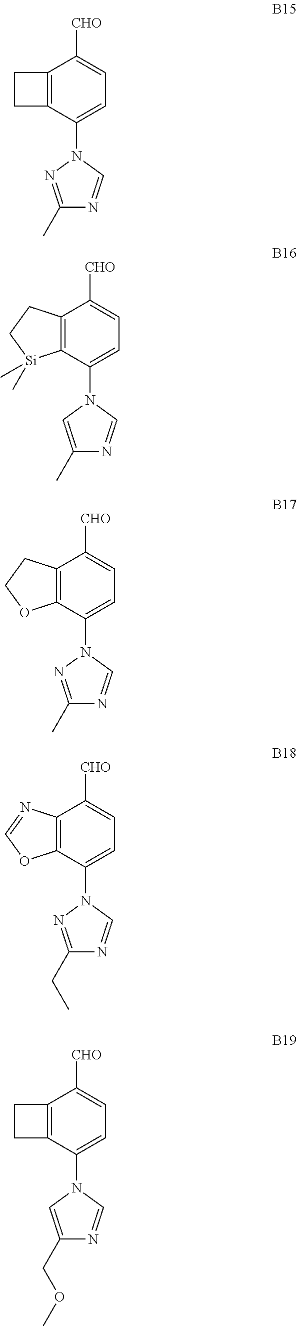

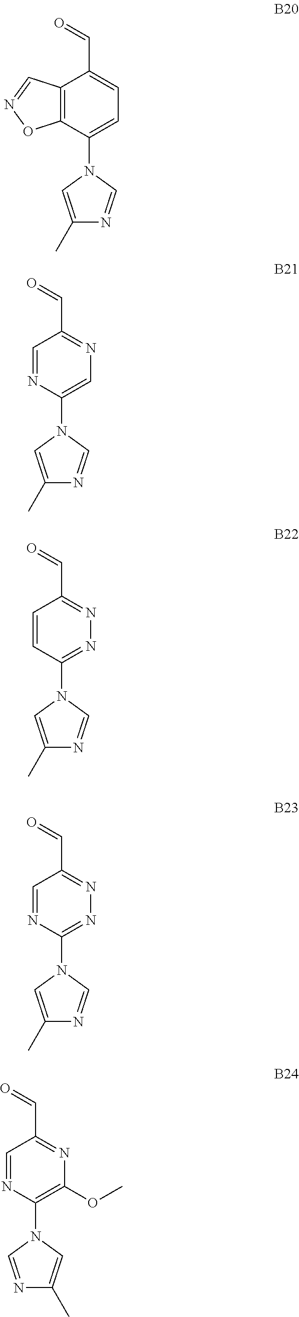

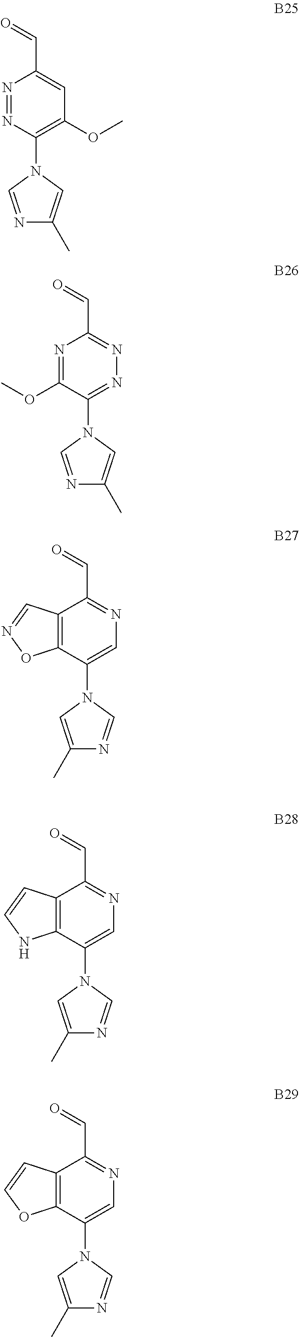

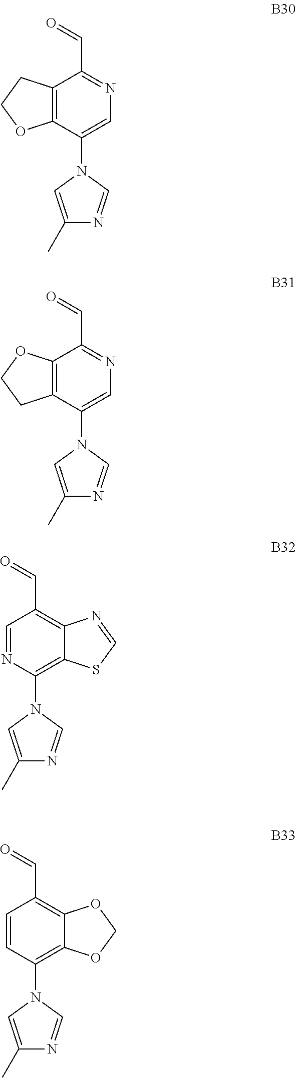

### Method C

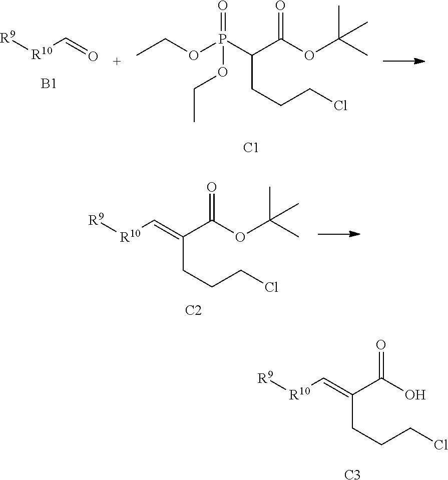

### Method C, Step 1

To a solution of B1 (R¹⁰=m-MeO-phenyl, R⁹=4-(4-methylimidazol-1-yl), 38.9 g) and C1 (1 eq) in THF (244 mL) and absolute EtOH (81.5 mL) was added LiOH.H₂O (11.5 g). The mixture was stirred for 1d followed by diluting with EtOAc (10V, 326 mL) and water (326 mL). The phases were spilt and the aqueous phase was extracted with EtOAc (326 mL). The combined organic phases were washed with brine (326 mL) and dried over MgSO₄. The crude solid was partially purified by column chromatography using 25-100% EtOAc in CH₂Cl₂ as the eluting solvent. The isolated solid (31 g) was dissolved in a hot (77° C.) 1:1 EtOAc/heptane solution (150 mL) and diluted with heptane (580 mL) while maintaining an internal temperature of 65-77° C. The resulting homogeneous solution was allowed to gradually cool to ambient temperature. The crystallized material was collected using a glass fritted funnel, slurry washed with heptane (150 mL) and air dried under house vacuum for 4 h which furnished compound C2 (R¹⁰=m-MeO-phenyl, R⁹=4-(4-methylimidazol-1-yl), 20.5 g) as an off white solid.

### Method C, Step 2

Compound C2 will be dissolved in Acetonitrile and conc HCl(20 eq) and the solution stirred until C2 (R¹⁰=m-MeO-phenyl, R⁹=4-(4-methylimidazol-1-yl) is completely converted to C3 (R¹⁰=m-MeO-phenyl, R⁹=4-(4-methylimidazol-1-yl)) upon workup and purification.

### Method D

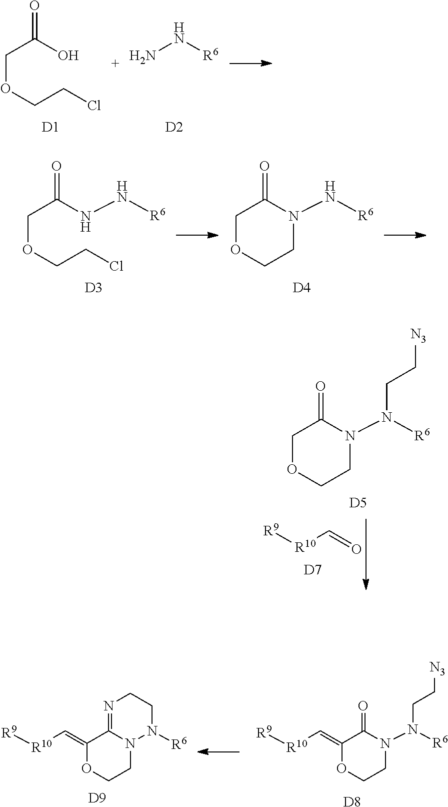

### Method D, Step 1,

To a DMF solution of compound D1 compound D2 (R⁶=p-F-phenyl, 1 eq) will be added EDCl and the final compound D3 (R⁶=p-F-phenyl) will be isolated from the reaction mixture after work-up.

### Method D, Step 2

To a THF solution of D3 (R⁶=p-F-phenyl) will be added NaH and the product D4 (R⁶=p-F-phenyl) will be isolated from the reaction mixture upon workup.

### Method D, Step 3

To a THF solution of D4 (R⁶=p-F-phenyl) and 2-bromoethylazide in THF will be added NaH and the product D5(R⁶=p-F-phenyl) will be isolated from the reaction mixture upon workup

### Method D, Step 4.

To a THF solution of compound D5 (R⁶=p-F-phenyl) will be added LDA (1 eq) followed by D7(R¹⁰=m-MeO-phenyl, R⁹=4-(4-methylimidazol-1-yl)) and the final reaction mixture will be treated with MsCVEt₃N before work up and purification to give compound D8.

### Method D, Step 5

A THF solution of D8 and triphenylphosphine (1 eq) will be heated in microwave to give compound D9 after workup and purification.

The following compounds may be generated using method similar to Method D:

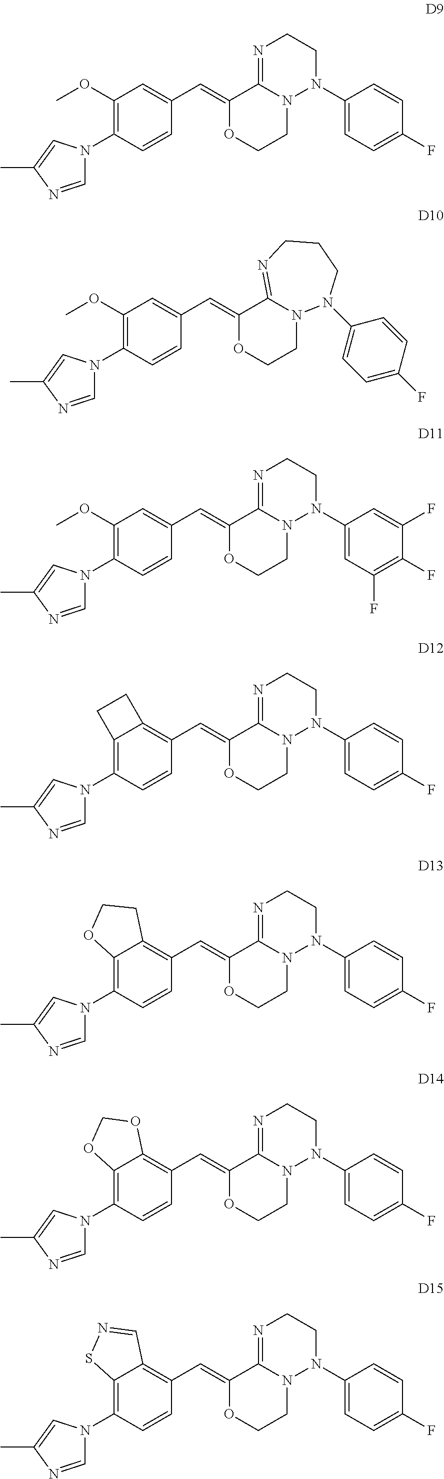

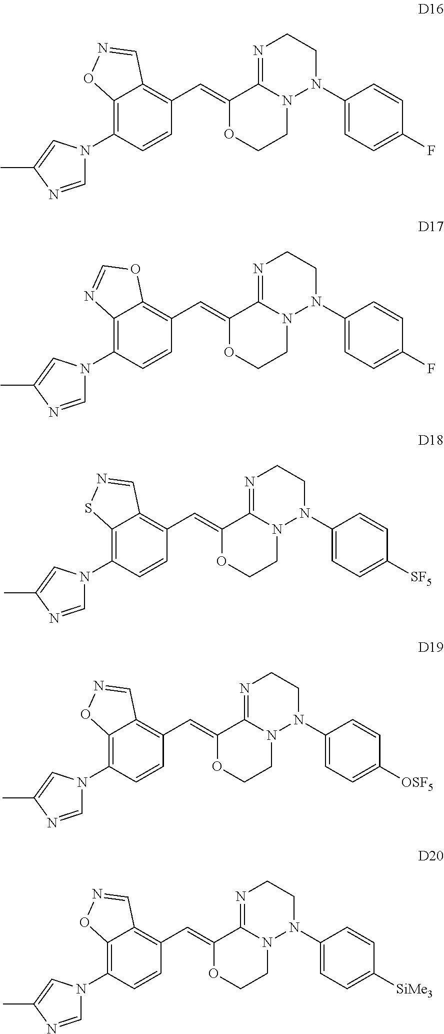

### Method E

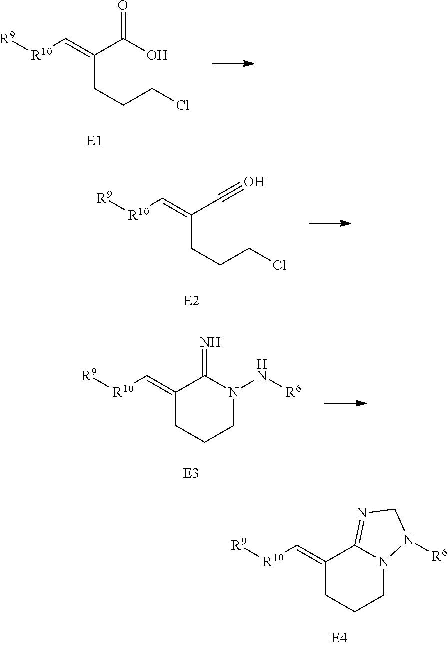

### Method E, Step 1

To a DMF solution of E1 and Ammonium Chloride will be added EDCl and triethylamine. The primary amide product after purification will be treated with TFAA (2 eq) in DCM with triethylamine (3 eq) to give compound E2 after purification.

### Method E, Step 2

A literature procedure will be adapted: Kisel, V. M.; Kostyrko, E. O.; Shishkin, O. V.; Shishkina, S. V.; Kovtunenko, V. A. Taras Shevchenko I (New York, N.Y., United States)(Translation of Khimiya Geterotsiklicheskikh Soedinenii) (2002), 38(10), 1253-1262.

A dioxane solution of compound E2 (R¹⁰=m-MeO-phenyl, R⁹=4-(4-methylimidazol-1-yl) and p-F-phenylhydrazine will be heat in microwave to give compound E3 (R¹⁰=m-MeO-phenyl, R⁹=4-(4-methylimidazol-1-yl) and R⁶=p-F-phenyl) after purification.

### Method E, Step 3

A THF solution of E3 (R¹⁰=m-MeO-phenyl, R⁹=4-(4-methylimidazol-1-yl) and R⁶=p-F-phenyl) will be refluxed with paraformaldehyde to give compound E4 (R¹⁰=m-MeO-phenyl, R⁹=4-(4-methylimidazol-1-yl) and R⁶=p-F-phenyl) after workup and purification.

The following compounds may be synthesized using method similar to Method E.

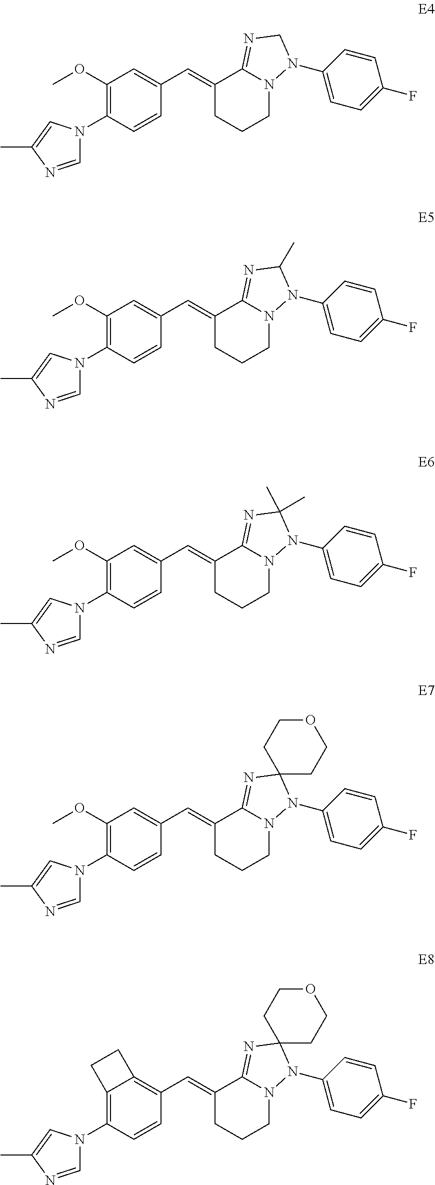

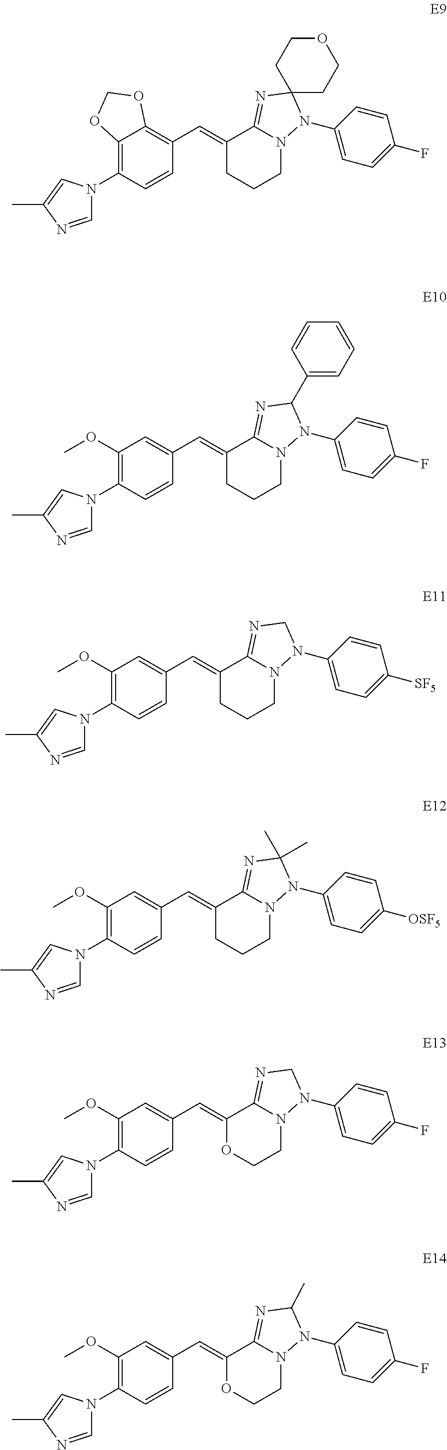

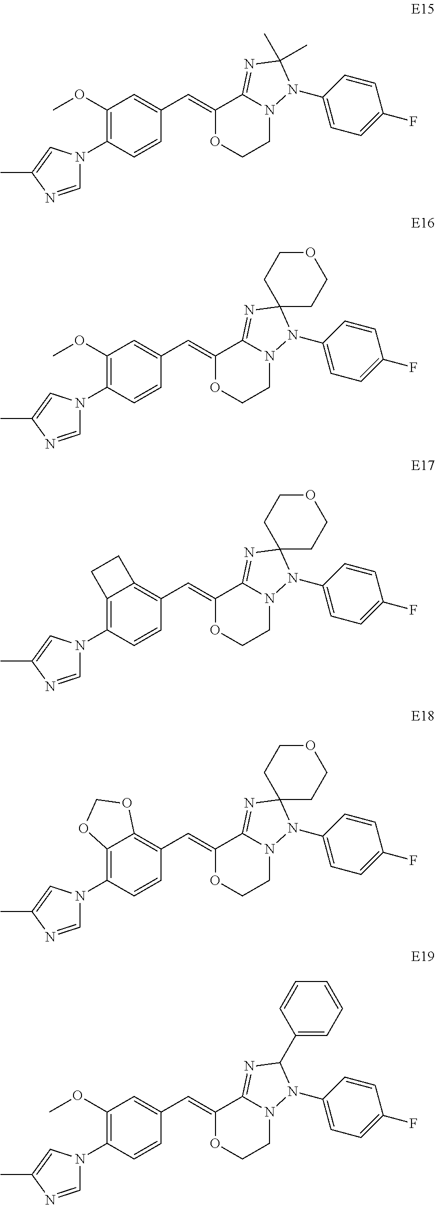

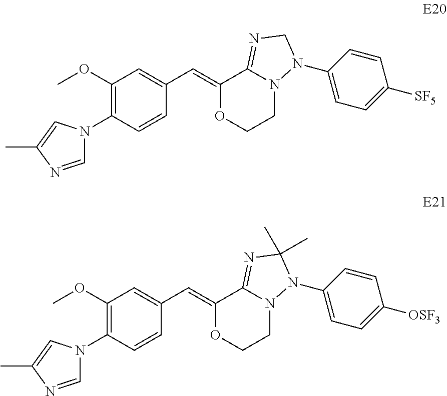

### Method F

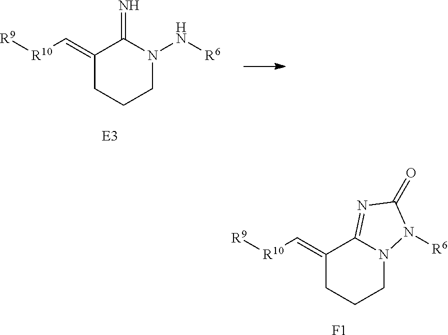

To a DCM solution of E3 (R¹⁰=m-MeO-phenyl, R⁹=4-(4-methylimidazol-1-yl) and R⁶=p-F-phenyl) will be added triphosgene to give F1 after workup and purification.

The following compounds may be synthesized using method similar to method F:

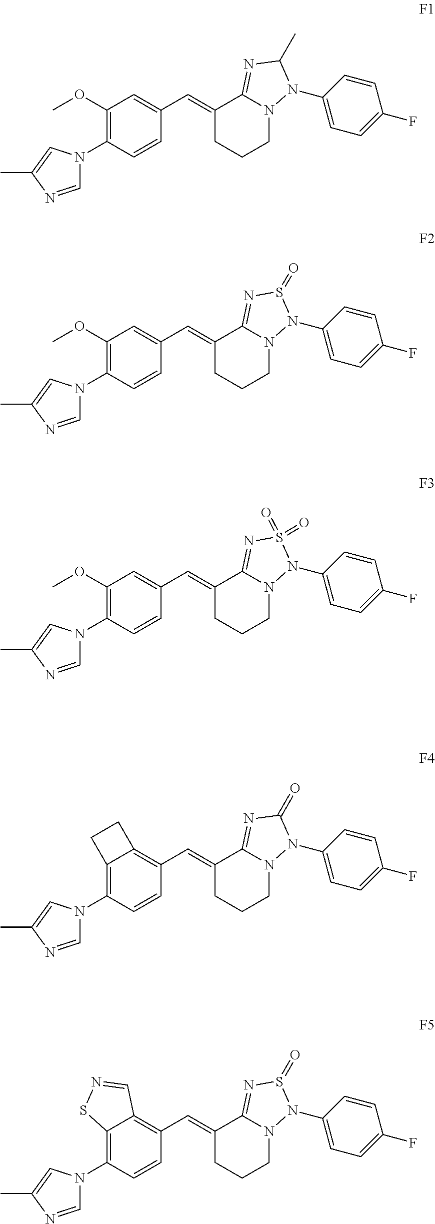

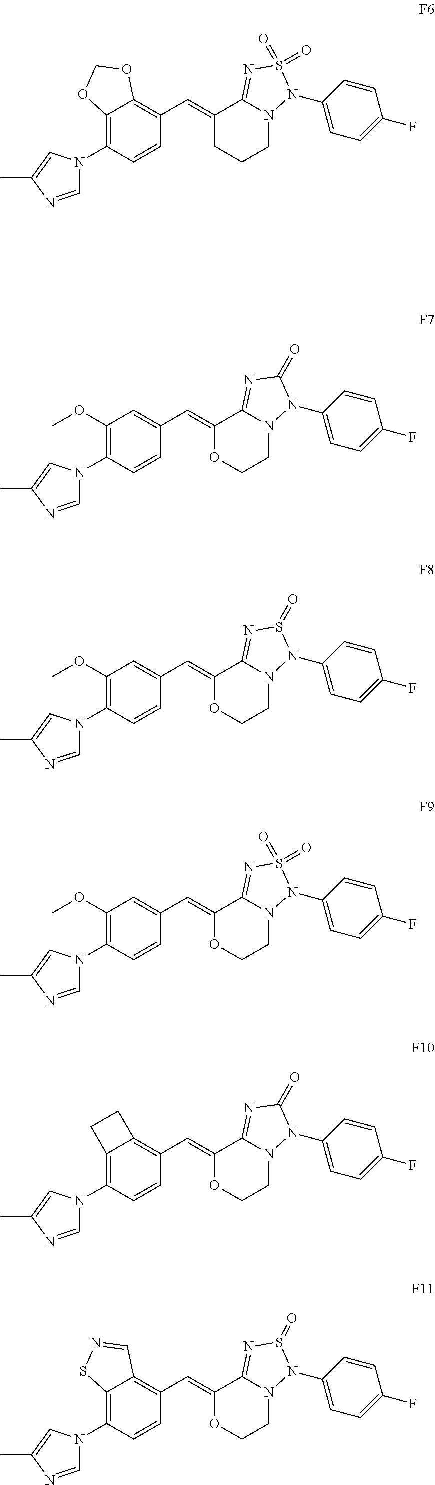

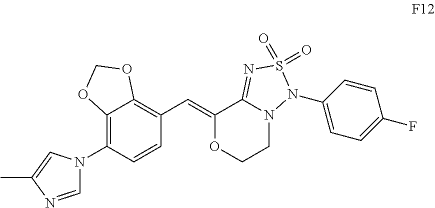

### Method G

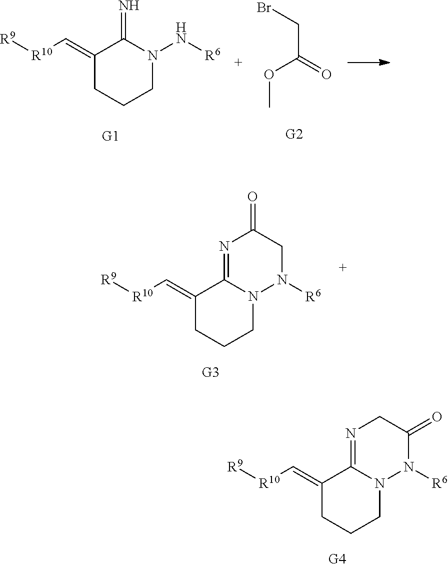

A DMF solution of G1 (R¹⁰=m-MeO-phenyl, R⁹=4-(4-methylimidazol-1-yl) and R⁶=p-F-phenyl) and G2 will be heated using a microwave oven to give G3 (R¹⁰=m-MeO-phenyl, R⁹=4-(4-methylimidazol-1-yl) and R⁶=p-F-phenyl) and G4(R¹⁰=m-MeO-phenyl, R⁹=4-(4-methylimidazol-1-yl) and R⁶=p-F-phenyl) after work-up and purification.

The following compounds may be generated using method similar to Method G.

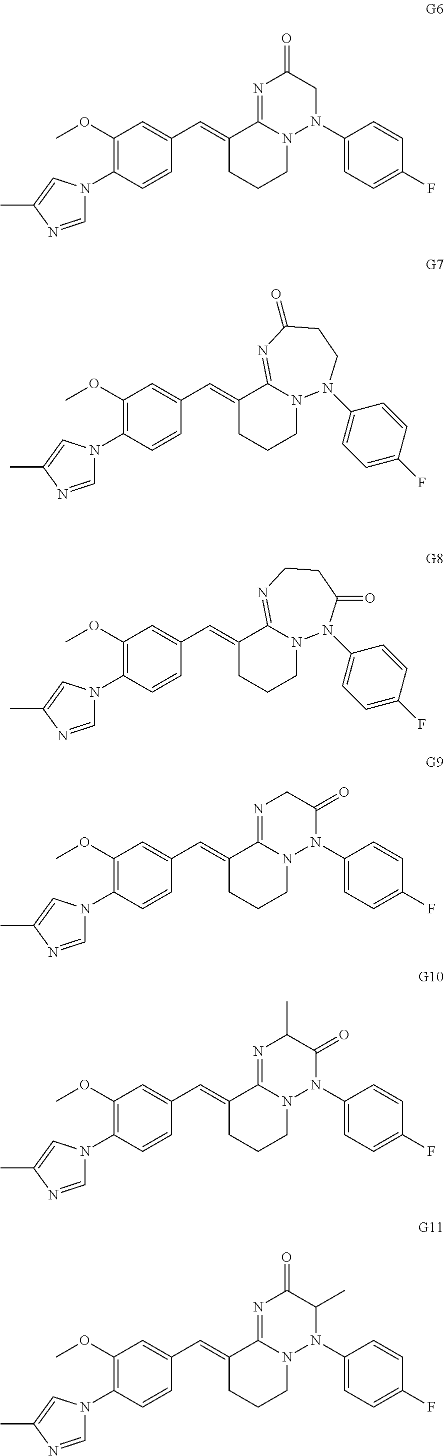

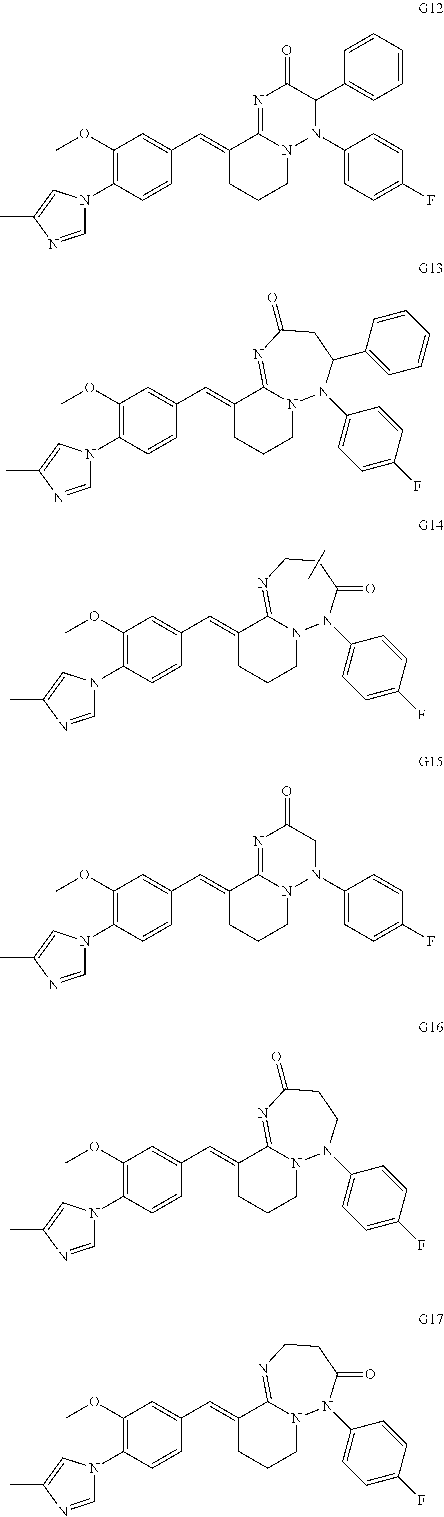

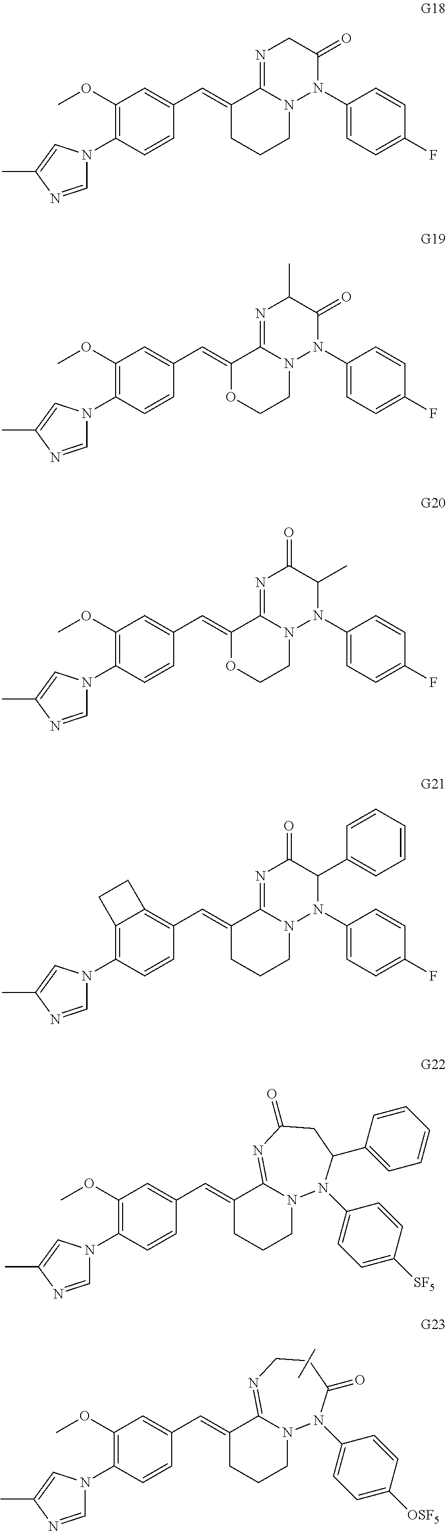

### Method H

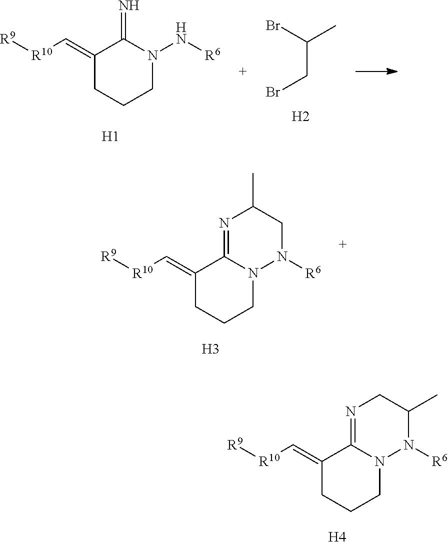

A DMF solution of H1 (R¹⁰=m-MeO-phenyl, R⁹=4-(4-methylimidazol-1-yl) and R⁶=p-F-phenyl) and H2 will be heated using a microwave oven to give H3 (R¹⁰=m-MeO-phenyl, R⁹=4-(4-methylimidazol-1-yl) and R⁶=p-F-phenyl) and H4(R¹⁰=m-MeO-phenyl, R⁹=4-(4-methylimidazol-1-yl) and R⁶=p-F-phenyl) after work-up and purification.

The following compounds may be generated using method similar to Method H.

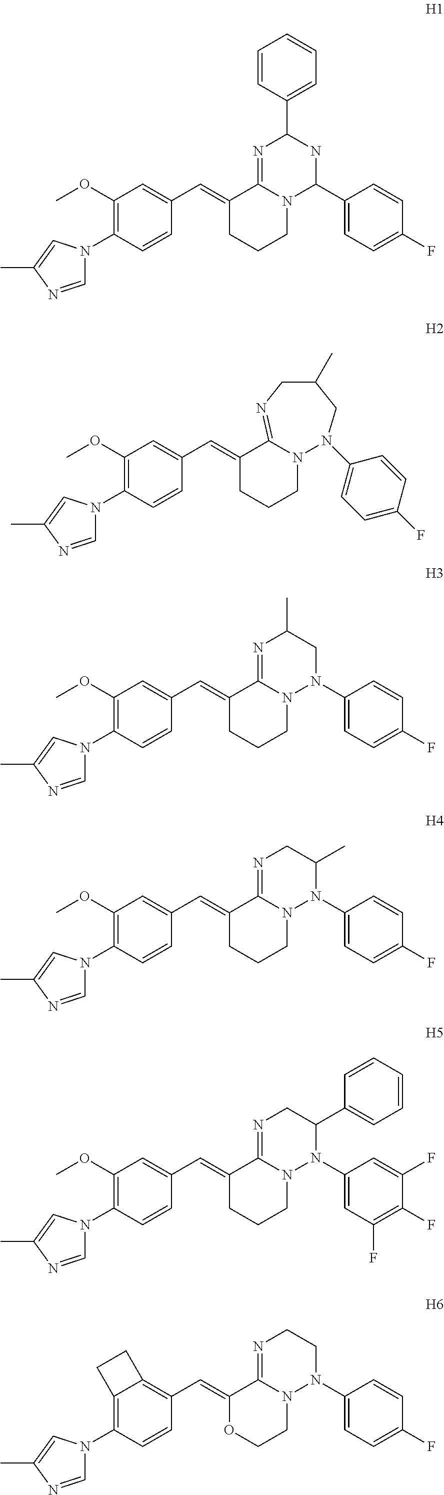

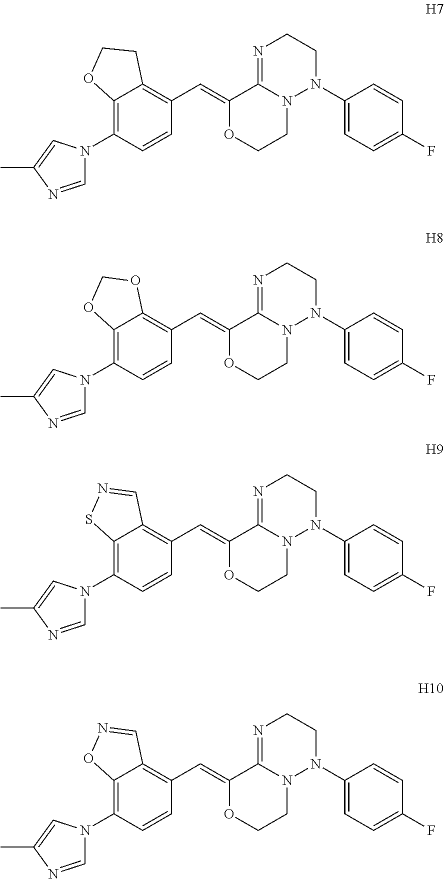

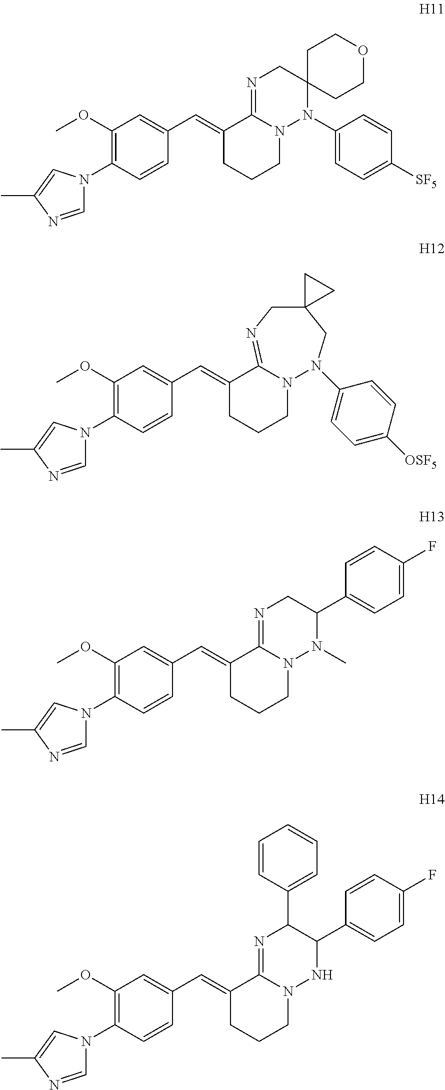

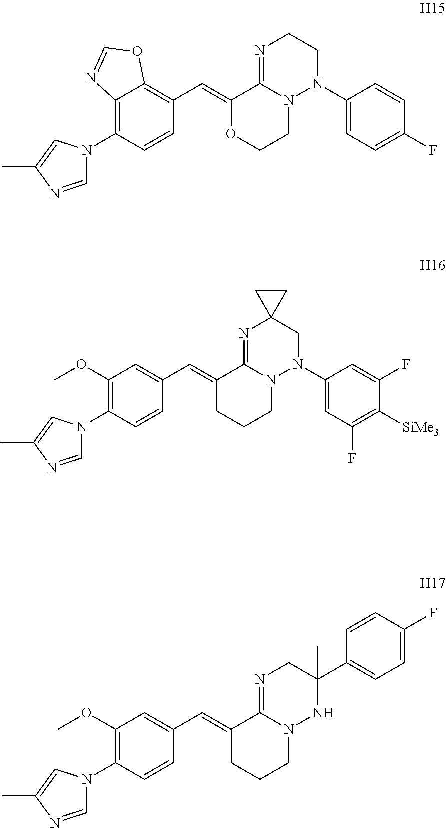

### Method I

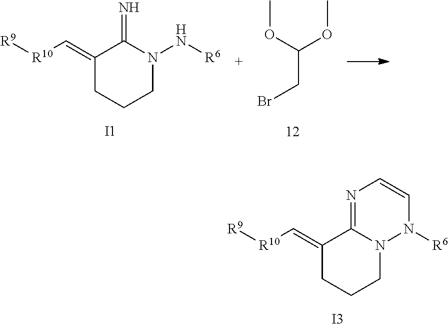

A DMF solution of I1 (R¹⁰=m-MeO-phenyl, R⁹=4-(4-methylimidazol-1-yl) and R⁶=p-F-phenyl) and I2 will be heated using a microwave oven to give I3 (R¹⁶=m-MeO-phenyl, R⁹=4-(4-methylimidazol-1-yl) and R⁶=p-F-phenyl) after work-up and purification.

The following compounds may be generated using method similar to Method I

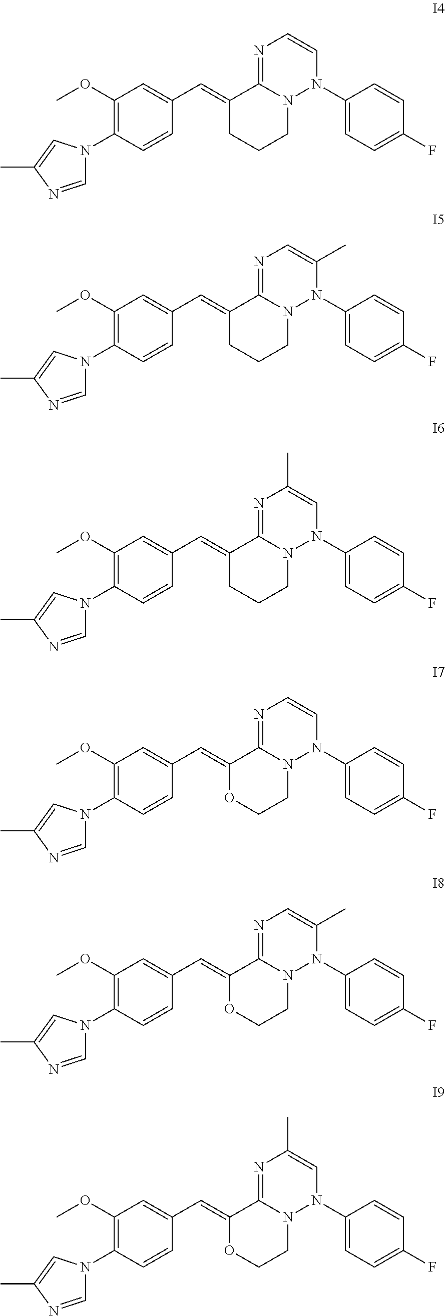

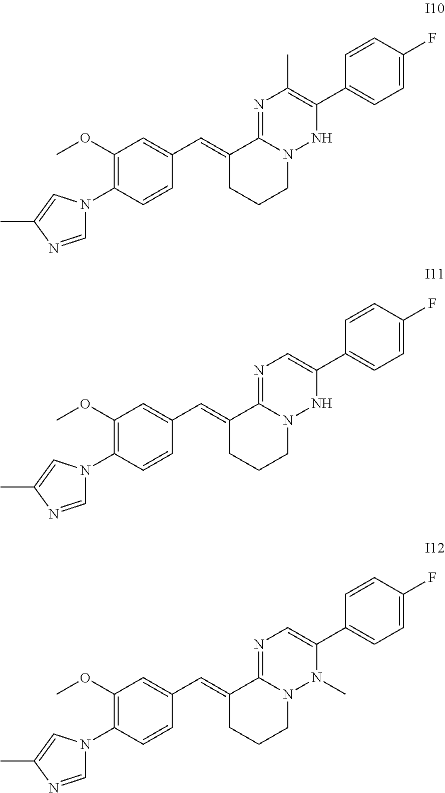

### Method J

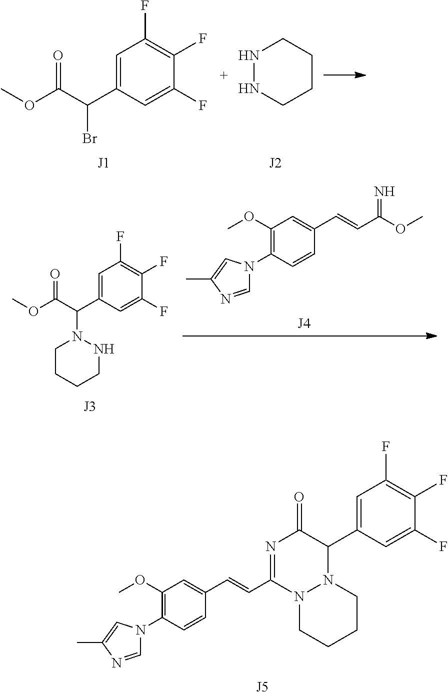

### Method J, Step 1

To a THF solution of compound J1 will be added compound J2 to give compound J3 after reaction, workup and purification.

### Method J, Step 2

To a DMF solution of J3 will be added compound J4 and triethylamine. The reaction will be heated in microwave ovens to give compound J5 after workup and purification.

The following compounds may be synthesized using method similar to Method J:

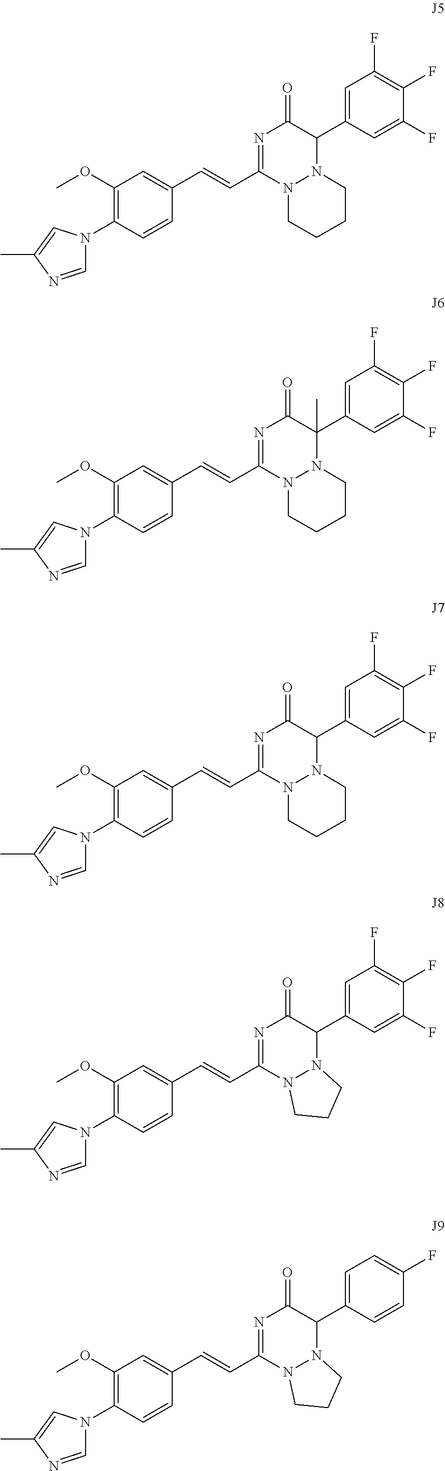

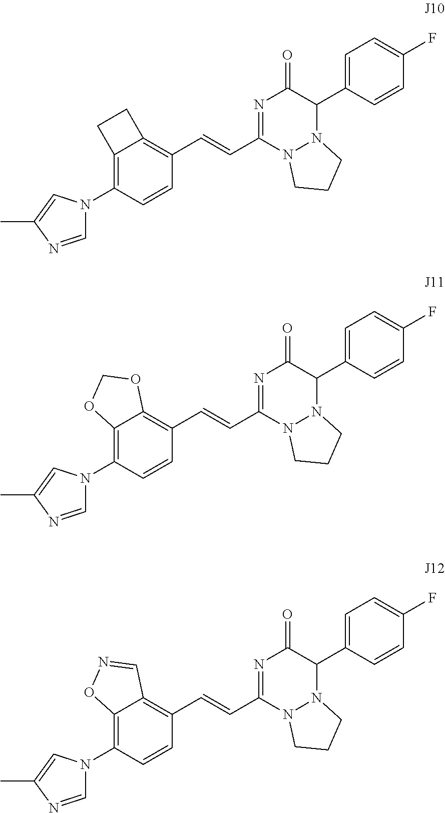

### Method K

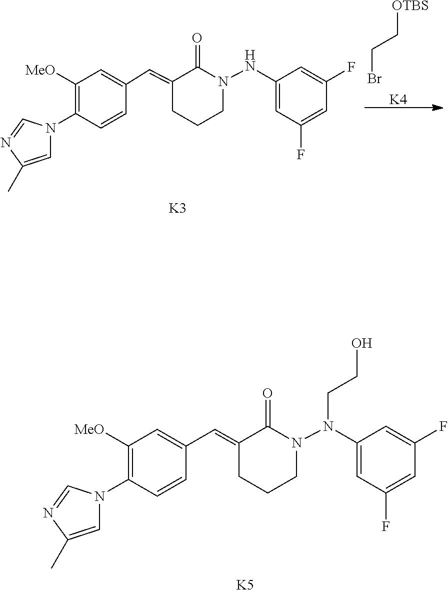

To a solution of K3 (1.85 gm, 4.36 mmol, 1 equiv.) and bromide K₄ (2.2 mL, 8.46 mmol, 2.2 equiv.) in a mixture THF and DMF (8 mL THF and 4 mL DMF) was added KMHDS (5.23 mmol, 1.2 equiv.) at 0° C. Ice bath was removed and the resulting solution was stirred at room temperature for 12 hours. The reaction mixture was quenched with saturated NH₄Cl, extracted with ethyl acetate, dried with MgSO₄, concentrated and purified using methanol in dichloromethane to obtain TBS protected alcohol in 80% yield. To this TBS protected alcohol in 5 mL THF was added 2 mL HF/pyridine (70%) at 0° C., and resulting solution was stirred 30 minutes. Upon completion of the reaction, the excess of HF was quenched with 2 mL triethyl amine, and the resulting solution was evaporated and directly loaded into column and purified using methanol in dichloromethane to provide the alcohol K5 in 80% yield. ¹H NMR δ 7.89 (s, 1H), 7.71 (s, 1H), 7.24 (m, 1H), 7.02 (m, 2H), 6.92 (br-s, 1H), 6.29-6.17 (m, 3H), 4.47 (br-s, 1H), 3.93-3.85 (m, 5H), 3.67-3.54 (m, 3H), 3.41-3.35 (m, 1H), 2.88 (m, 2H), 2.27 (s, 3H), 2.04 (m, 2H).

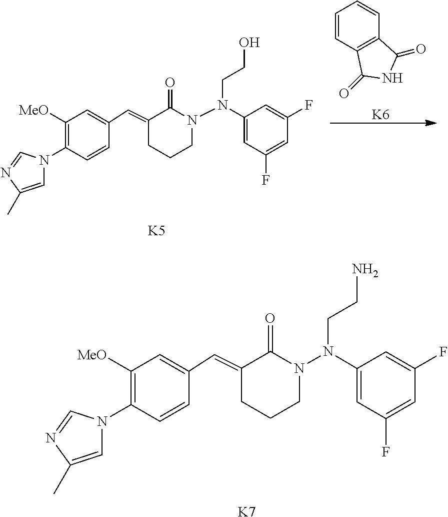

To a solution of alcohol K5 (100 mg, 0.213 mmol, 1 equiv.) in 0.8 mL THF were added ADDP (64 mg, 0.256 mmol, 1.2 equiv.), nBu₃P (51 mg, 0.256 mmol, 1.2 equiv.) and phthalimide (37 mg, 0.256 mmol, 1.2 equiv.), and the resulting solution was heated at 80° C. for 12 hours. Upon completion, the reaction mixture was evaporated to dryness, diluted with ethyl acetate, washed with saturated sodium bicarbonate, dried with MgSO4, concentrated and purified using ethyl acetate to obtained the phthalimide adduct in 85% yield. To this phthalimide adduct (125 mg, 0.209 mmo, 1.0 equiv.) in 0.5 mL ethanol was added hydrazine hydrate (0.1 mL, 2.09 mmol, 10 equiv.) and resulting solution was stirred for 12 hours. Upon completion of the reaction, the reaction mixture was diluted with water and extracted with ethyl acetate, dried with MgSO4, concentrated and purified using methanol in dichloromethane to obtain K7 in 60% yield. ¹H NMR δ 7.84 (s, 1H), 7.71 (s, 1H), 7.25 (m, 2H), 7.01 (m, 2H), 6.92 (m, 1H), 6.20 (m, 3H), 3.85 (s, 3H), 3.72-3.52 (m, 4H), 2.99 (m, 3H), 2.78 (m, 1H), 2.28 (s, 3H), 2.0 (m, 2H).

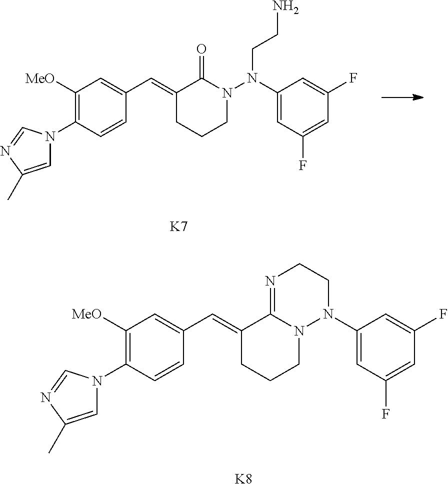

A solution of K7 in 0.5 mL POCl₃ was heated at 80° C. for 12 hours. Upon disappearance of starting material, the reaction mixture was evaporated to dryness, then diluted with water, neutralized with 1 N NaOH, extracted with ethyl acetate, dried with MgSO4, concentrated and purified using methanol in dichloromethane to obtain K8. ¹H NMP δ 7.69 (s, 1H), 7.45 (s, 1H), 7.22 (m, 1H), 6.97 (m, 2H), 6.91 (s, 1H), 3.82 (s, 3H), 3.49-3.36 (m, 6H), 2.79 (m, 2H), 2.28 (s, 3H), 1.97 (m, 2H).

### Method L

Intermediates useful for the preparation of compounds having —SF₅ and —OSF₅ groups can be prepared, for example, from the reactions below as well as other techniques well known in the art.

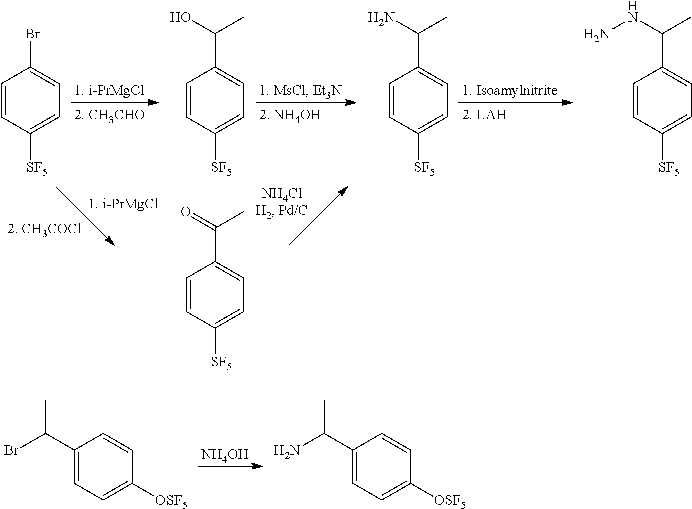

Compounds having —Si(R¹⁵)₃ (such as, for example, —Si(CH₃)₃) groups, or other —SF₅ substituted groups, or other —OSF₅ substituted groups can be prepared following procedures similar to those above, as well as techniques well known in the art.

### Method M

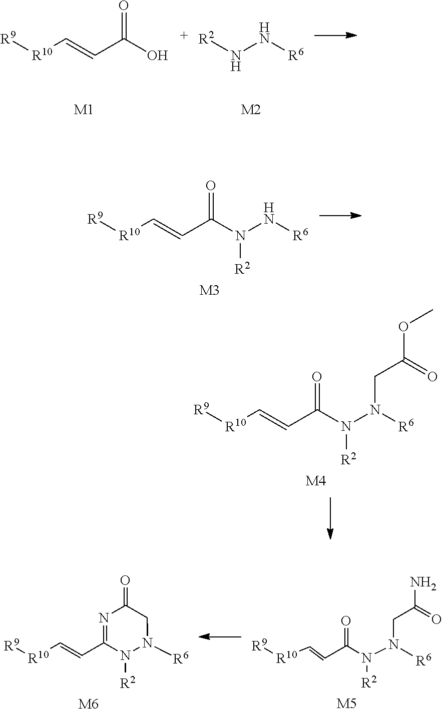

### Method M, Step 1;

To a DMF solution of compound M1 (R¹⁰=m-MeO-phenyl, R⁹=4-(4-methylimidazol-1-yl)) and compound M2 (R²=methyl, R⁶=p-F-phenyl, 1 eq) will be added EDCl/triethylamine and the final compound M3 (R¹⁰=m-MeO-phenyl, R⁹=4-(4-methylimidazol-1-yl, R²=methyl and R⁶=p-F-phenyl) will be isolated from the reaction mixture after work-up.

### Method M, Step 2;

To a THF solution of M3 (R¹⁰=m-MeO-phenyl, R⁹=4-(4-methylimidazol-1-yl, R²=methyl and R⁶=p-F-phenyl) and methyl 2-bromoacetate will be treated with NaH and product M4 (R¹⁰=m-MeO-phenyl, R⁹=4-(4-methylimidazol-1-yl, R²=methyl and R⁶=p-F-phenyl) will be isolated from the reaction mixture after work up.

### Method M, Step 3;

Compound M4 (R¹⁰=m-MeO-phenyl, R⁹=4-(4-methylimidazol-1-yl, R²=methyl and R⁶=p-F-phenyl will be hydrolyzed using LiOH in methanol to give the corresponding acid. The acid will be treated with ammonium chloride, DIEA, EDCl in DMF to give compound M5 (R¹⁰=m-MeO-phenyl, R⁹=4-(4-methylimidazol-1-yl, R²=methyl and R⁶=p-F-phenyl) after workup and purification.

### Method M, Step 4;

Compound M5 (R¹⁰=m-MeO-phenyl, R⁹=4-(4-methylimidazol-1-yl, R²=methyl and R⁶=p-F-phenyl) will be treated with P₂O₅ in trifluoromethylbenzene under microwave at 20° C. to give compound M6 (R¹⁰=m-MeO-phenyl, R⁹=4-(4-methylimidazol-1-yl, R²=methyl and R⁶=p-F-phenyl) after workup and purification.

The following compounds may be synthesized using method similar to Method M.

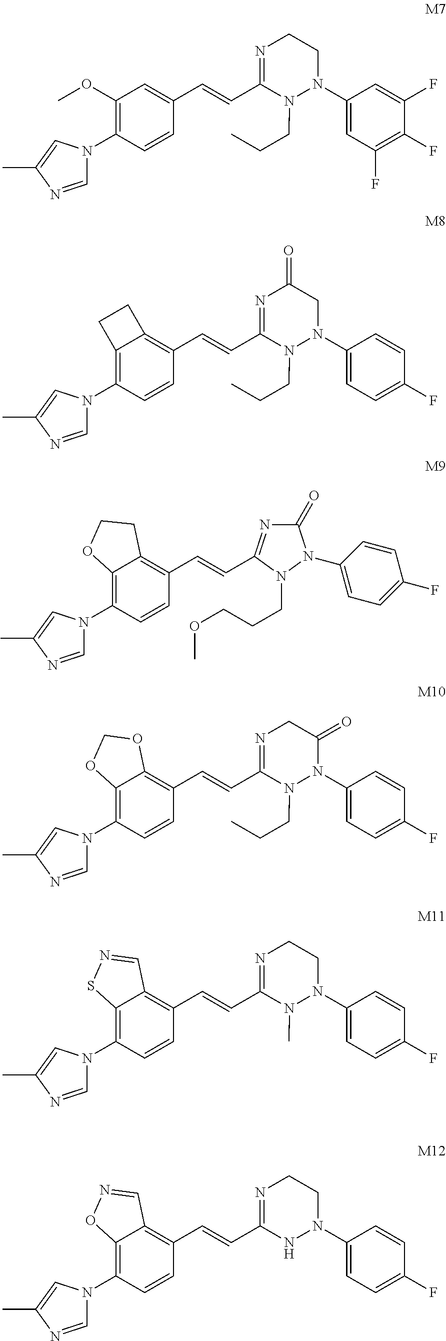

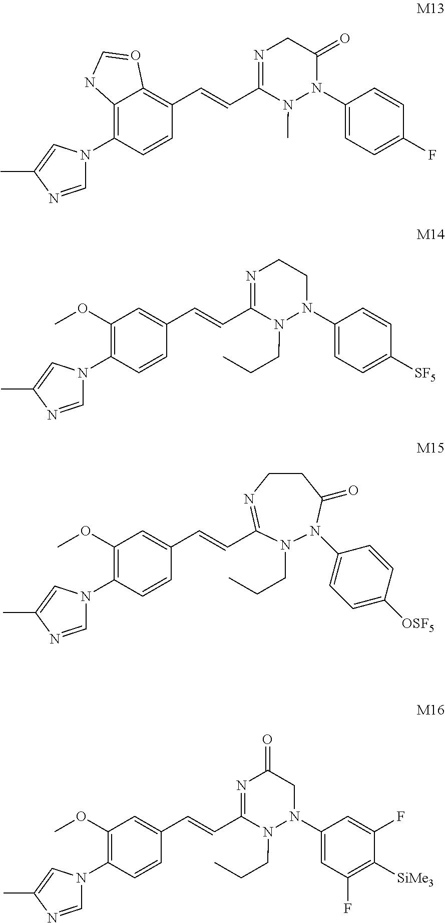

### Method N

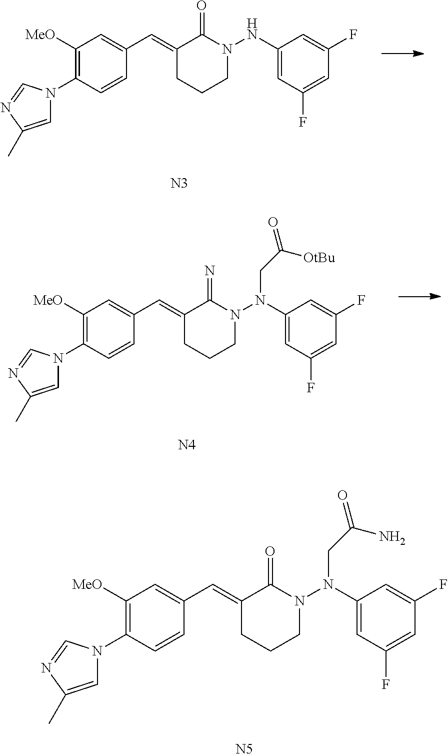

To a solution of N3 (100 mg, 0.235 mmol, 1 equiv.) in the mixture of THF (0.7 mL) and DMF (0.5 mL) was added tert-butyl bromoacetate (0.036 mL, 0.258 mmol, 1.1 equiv.), and the resulting mixture was cooled to 0° C. To this solution was added a solution of KHMDS in THF and the reaction mixture was stirred for 30 minutes before quenching with 2 mL saturated aqueous NH₄Cl. The resulting solution was diluted with water, extracted with ether, dried with MgSO₄, concentrated and purified using ethyl acetate in hexanes to provide N4 in 60% yield. To this tert-butyl ester, N4, was added 4N HCl (1 mL) in dioxane at rt, and the resulting solution was stirred for 30 minutes. The reaction mixture was evaporated to dryness and crude product was taken for primary amide formation.

To this crude acid in CH₃CN (0.5 mL) was added EDCl (18 mg), HOBt (12 mg) and DIEA (0.042 mL), and resulting mixture was stirred for 15 minutes before the addition of aqueous NH₄OH (1 mL). The resulting solution was stirred for 30 minutes, then evaporated to dryness and purified using C18 column (0.1% TFA in water and 0.1% TFA in MeCN was used as mobile phase) to yield the amide N5 in 50% yield. ¹H NMP: δ 7.96 (s, 1H), 7.83 (s, 1H), 7.41 (d, J. 8.0 Hz, 1H), 7.24 (s, 1H), 7.15 (m, 2H), 6.4 (m, 1H), 6.17 (m, 2H), 4.35 (d, J=18 Hz, 1H), 4.05 (d, J. 18 Hz, 1H), 3.90 (s, 3H), 3.80 (m, 1H), 3.65 (m, 1H), 2.98 (m, 1H), 2.88 (m, 1H), 2.24 (s, 3H), 2.06 (2H).

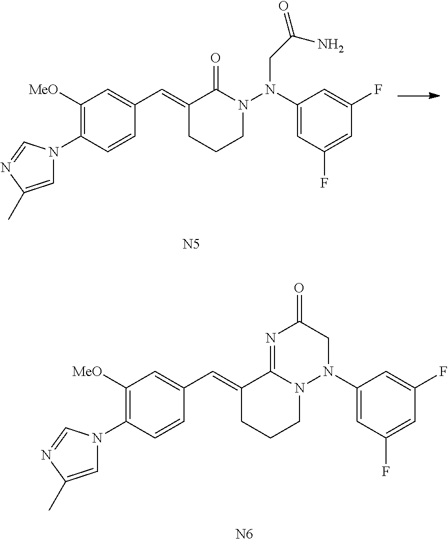

To a solution of N5 (10 mg) in trifluoro toluene (0.5 mL) was added P₂O₅ (10 equiv.) and heated at 125° C. overnight. The reaction mixture was evaporated to dryness and purified using C18 column (0.1% TFA in water and 0.1% TFA in acetonitrile was used as eluent) to provide N6 in 50% yield. ¹H NMP: δ 7.83 (s, 1H), 7.77 (s, 1H), 7.40 (d, J=8.0 Hz, 1H), 7.23 (s, 1H), 7.14 (m, 2H), 6.44 (m, 3H), 4.77 (m, 2H), 3.89 (m, 4H), 3.72 (m, 1H), 3.01 (m, 1H), 2.88 (m, 1H), 2.22 (s, 3H), 2.08 (m, 2H).

### Assay:

Secretase Reaction and Aβ Analysis in Whole Cells: HEK293 cells overexpressing APP with Swedish and London mutations were treated with the specified compounds for 5 hour at 37° C. in 100 ml of DMEM medium containing 10% fetal bovine serum. At the end of the incubation, total Aβ, Aβ40 and Aβ42 were measured using electrochemiluminescence (ECL) based sandwich immunoassays. Total Aβ was determined using a pair of antibodies TAG-WO2 and biotin-4G8, Aβ40 was identified with antibody pairs TAG-G2-10 and biotin-4G8, while Aβ42 was identified with TAG-G2-11 and biotin-4G8. The ECL signal was measured using Sector Imager 2400 (Meso Scale Discovery).

MS Analysis of Aβ Profile: Aβ profile in conditioned media was determined using surface enhanced laser desorption/ionization (SELDI) mass spectrometry. Conditioned media was incubated with antibody WO2 coated PS20 ProteinChip array. Mass spectra of Aβ captured on the array were read on SELDI ProteinChip Reader (Bio-Rad) according to manufacture's instructions.

CSF Aβ Analysis: Aβ in rat CSF was determined using MSD technology as described above. Aβ40 was measured using antibody pair Tag-G2-10 and biotin-4G8, while Aβ42 was measured using Tag-anti Aβ42 (Meso Scale Discovery) and biotin-4G8. The ECL signal was measured using Sector Imager 2400 (Meso Scale Discovery).

Matrix-assisted laser desorption/ionization mass spectrometric (MALDI MS) analysis of Aβ is performed on a Voyager-DE STR mass spectrometer (ABI, Framingham, Mass.). The instrument is equipped with a pulsed nitrogen laser (337 nm). Mass spectra are acquired in the linear mode with an acceleration voltage of 20 kV. Each spectrum presented in this work represents an average of 256 laser shots. To prepare the sample-matrix solution, 1 μL of immunoprecipitated Aβ sample is mixed with 3 μL of saturated α-cyano-4-hydroxycinnamic acid solution in 0.1% TFA/acetonitrile. The sample-matrix solution is then applied to the sample plate and dried at ambient temperature prior to mass spectrometric analysis. All the spectra are externally calibrated with a mixture of bovine insulin and ACTH (18-39 clip).

Certain compounds of the invention had an Aβ42 IC₅₀ in the range of about 218 nm to about 3686 nM, and an Aβtotal to Aβ42 ratio in the range of about 4 to about 92.

While the present invention has been described with in conjunction with the specific embodiments set forth above, many alternatives, modifications and other variations thereof will be apparent to those of ordinary skill in the art. All such alternatives, modifications and variations are intended to fall within the spirit and scope of the present invention.

1-20. (canceled)

21. A compound, or a pharmaceutically acceptable salt thereof, the compound having the general structure shown in the formula: 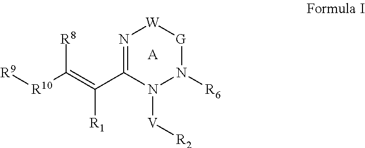
wherein:
either
(i) R¹ and R² are joined together to form 5-8 membered heterocyclyl or 5-8 membered heterocyclenyl moiety, wherein: (a) said heterocyclyl or heterocyclenyl moiety is optionally substituted with 1-5 independently selected R²¹ and (b) said heterocyclyl or heterocyclenyl moiety is optionally fused with an aryl or heteroaryl ring, and the ring moiety resulting from the fusion is optionally substituted with 1-5 independently selected R²¹ substituents; or
(ii) R² and R⁶ are joined together to form 5-8 membered heterocyclyl or 5-8 membered heterocyclenyl moiety, wherein: (a) said heterocyclyl or heterocyclenyl moiety is optionally substituted with 1-5 independently selected R²¹ substituents, and (b) said heterocyclyl or heterocyclenyl moiety is optionally fused with an aryl or heteroaryl ring, and the ring moiety resulting from the fusion is optionally substituted with 1-5 independently selected R²¹ substituents; or
(iii)
(a) R¹ and R² are joined together to form 5-8 membered heterocyclyl or 5-8 membered heterocyclenyl moiety, wherein: said heterocyclyl or heterocyclenyl moiety is optionally substituted with 1-5 independently selected R²¹ substituents, and
(b) R² and R⁶ are joined together to form 5-8 membered heterocyclyl or 5-8 membered heterocyclenyl moiety, wherein: said heterocyclyl or heterocyclenyl moiety is optionally substituted with 1-5 independently selected R²¹ substituents; and
(c) said R² and R⁶ heterocyclyl or heterocyclenyl moiety is optionally fused with an aryl or heteroaryl ring, and the ring moiety resulting from the fusion is optionally substituted with 1-5 independently selected R²¹ substituents; or
(iv) R⁶ and one R³ of the —(CR³R⁴)_{1 or 2}— G moiety are joined together to form 5-8 membered heterocyclyl or 5-8 membered heterocyclenyl moiety, wherein: (a) said heterocyclyl or heterocyclenyl moiety is optionally substituted with 1-5 independently selected R²¹ substituents, and (b) said heterocyclyl or heterocyclenyl moiety is optionally fused with an aryl or heteroaryl ring, and the ring moiety resulting from the fusion is optionally substituted with 1-5 independently selected R²¹ substituents; or
(v) R¹ and R² are not joined together to form 5-8 membered heterocyclyl or 5-8 membered heterocyclenyl moiety, R² and R⁶ are not joined together to form 5-8 membered heterocyclyl or 5-8 membered heterocyclenyl moiety, and R⁶ and one R³ of the —(CR³R⁴)_{1 or 2}— G moiety are not joined together to form 5-8 membered heterocyclyl or 5-8 membered heterocyclenyl moiety; and
R¹ (when R¹ is not joined to R² and when R¹ does not together with R⁸ form a bond), R² (when R² is not joined to R¹ or R⁶), and R⁶ (when R⁶ is not joined to R² or R³) can be the same or different, each being independently selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- can be unsubstituted or optionally substituted with 1-5 independently selected R²¹ substituents;
or, alternatively, R¹ (when R¹ is not joined to R²) and R⁸ can be taken together to form a bond (i.e., there is a triple bond between the carbon atom to which R¹ was bonded to and the carbon to which R⁸ was bonded to, i.e., the compound of formula I is a compound of formula II: 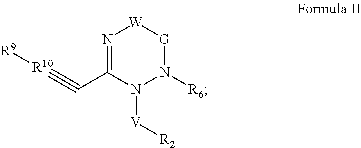
W is selected from the group consisting of a bond, —C(O)—, —S(O)—, —S(O₂)— and —(CR¹¹R¹²)_{1 or 2}—, e.g., —CR¹¹R¹² —, —CR¹¹R¹²—CR—, —CR¹¹CR¹²—, CR¹¹R¹²—, and —CH₂—C(R¹¹)(R¹²)—, with the proviso that ring A is a 5-, 6- or 7-membered ring;
G is selected from the group consisting of —C(O)—, —S(O)—, —S(O₂)— and —(CR³R⁴)_{1 or 2}—, e.g., —CR³R⁴—, —CR³R⁴—CH₂—, —CR³R⁴— CR³R⁴—, and —CH₂—CR³R⁴—, with the provisos that ring A is a 5-, 6- or 7-membered ring and that no combination of W and G can be —C(O)—S(O)—, C(O)—S(O)₂—, —S(O)—C(O)—, —S(O)₂—C(O)—, —S(O)—S(O)—, S(O)—S(O)₂—, —S(O)₂—S(O)— or S(O)₂—S(O)₂—;
V is selected from the group consisting of a bond and —C(O)—;
each R³ (when R³ does not form a ring with R⁶ or with R⁴) can be the same or different and is independently selected from the group consisting of H, halo (and in one example, F), —OR¹⁵ (and in one example R¹⁵ is H), —CN, —SR¹⁵, —NR¹⁵R¹⁶, —N(R¹⁵)C(O)R¹⁶, —N(R¹⁵)S(O)R¹⁶, —N(R¹⁶)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —C(O)R¹⁵, —C(O)OR¹⁵, —C(═NOR¹⁵)R¹⁶, —C(O)N(R¹⁵)(R¹⁶), —S(O)N(R¹⁵)(R¹⁶), S(O)₂N(R¹⁵)(R¹⁶), —S(O)R¹⁵, —S(O)₂R^{15A}, —P(O)(OR¹⁵)(OR¹⁵), ═NOR¹⁵, —N₃, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- can be unsubstituted or optionally substituted with 1-5 independently selected R²¹ substituents; or
each R⁴ (when R⁴ does not form a ring with R³), R¹¹ (when R¹¹ does not from a ring with R¹²) and R¹² (when R¹² does not for a ring with R¹¹) can be the same or different and is independently selected from the group consisting of H, halo (and in one example, F), OR¹⁵ (and in one example R¹⁵ is H), —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —C(O)R¹⁶, —C(O)OR¹⁶, —C(═NOR¹⁵)R¹⁶, —C(O)N(R¹⁵)(R¹⁶), —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, —N₃, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- can be unsubstituted or optionally substituted with 1-5 independently selected R²¹ substituents;
alternatively, when W is —CR¹¹R¹²— and G is —CR³R⁴—, R³ (when R³ does not form a ring with R⁴ or R⁶) and R¹¹ (when R¹¹ does not form a ring with R¹²) can be joined together to form a bond;
alternatively, (a) R³ (when R³ does not form a ring with R⁶ or a bond with R¹¹) and R⁴ can be joined together to form a C₃-C₈ spirocycloalkyl, C₄-C₈ spirocycloalkenyl, 5-8 membered spiroheterocyclyl or 5-8 membered spiroheterocyclenyl moiety, with each of said spirocycloalkyl or spiroheterocyclyl or spirocycloalkenyl moiety being unsubstituted or optionally substituted with 1-5 independently selected R²¹ substituents, or (b) R¹¹ and R¹² can be joined together to form a C₃-C₈ spirocycloalkyl, C₄-C₈ spirocycloalkenyl, 5-8 membered spiroheterocyclyl or 5-8 membered spiroheterocyclenyl moiety, with each of said spirocycloalkyl or spiroheterocyclyl or spirocycloalkenyl moiety being unsubstituted or optionally substituted with 1-5 independently selected R²¹ substituents, and (c) with the proviso that ring A can have only one C₃-C₈ spirocycloalkyl, C₄-C₈ spirocycloalkenyl, 5-8 membered spiroheterocyclyl or 5-8 membered spiroheterocyclenyl moiety;
provided that when one of R³ or R⁴ is selected from the group consisting of: —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶—N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶)S(O)₂N(R¹⁵)(R¹⁶), —S(O)R¹⁵, —S(O)₂R^{15A}, —P(O)(OR¹⁵)(OR¹⁶), ═NOR¹⁶, and —N₃, then the other is not selected from the group consisting of: —OR¹⁵, —CN, —SR¹⁵, and —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, and —N₃ (i.e., if one of R³ or R⁴ is —OR¹⁵, —CN, —SR¹⁵, —NR¹⁵R¹⁶, —N(R¹⁵)C(O)R¹⁶, —N(R¹⁵)S(O)R¹⁶—N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R¹⁵)(R¹⁶, —S(O)R¹⁵, —S(O)₂R^{15A}, —P(O)(OR¹⁵)(OR¹⁶), ═NOR¹⁵, or —N₃, then the other one is not —OR¹⁵, —CN, —SR¹⁵, and —NR¹⁵R¹⁶, —N(R¹⁵)C(O)R¹⁶—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, or —N₃);
provided that when one of R¹¹ or R¹² is selected from the group consisting of: —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, and —N₃, then the other is not selected from the group consisting of: —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, and —N₃ (i.e., if one of R¹¹ or R¹² is —OR¹⁵, —CN, —SR¹⁵, —NR¹⁵R¹⁶, —N(R¹⁵)C(O)R¹⁶, —N(R¹⁵)S(O)R¹⁶, —N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, or —N₃, then the other is not —OR¹⁵, —CN, —SR¹⁵, —NR¹⁵R¹⁶, —N(R¹⁵)C(O)R¹⁶—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —N(R¹⁵)C(O)OR¹⁶, —S(O)N(R¹⁵)(R¹⁶), —S(O)₂N(R¹⁵)(R¹⁶), —S(O)R¹⁵, —S(O)₂R^{15A}, —P(O)(OR¹⁵)(OR¹⁶), ═NOR¹⁵, or —N₃),
R⁸ (when R¹ is not joined to R⁸) is selected from the group consisting of H, alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl-, with each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- being unsubstituted or optionally substituted with 1-3 independently selected R²¹ substituents;
R⁹ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each R⁹ group is optionally substituted with 1-3 independently selected R²¹ substituents;
R¹⁰ is selected from the group consisting of a bond, alkyl-, alkenyl-, alkynyl-, aryl-, arylalkyl-, alkylaryl-, cycloalkyl-, cycloalkylalkyl-, heteroaryl-, heteroarylalkyl-, heterocyclyl-, heterocyclylalkyl- and the moieties: 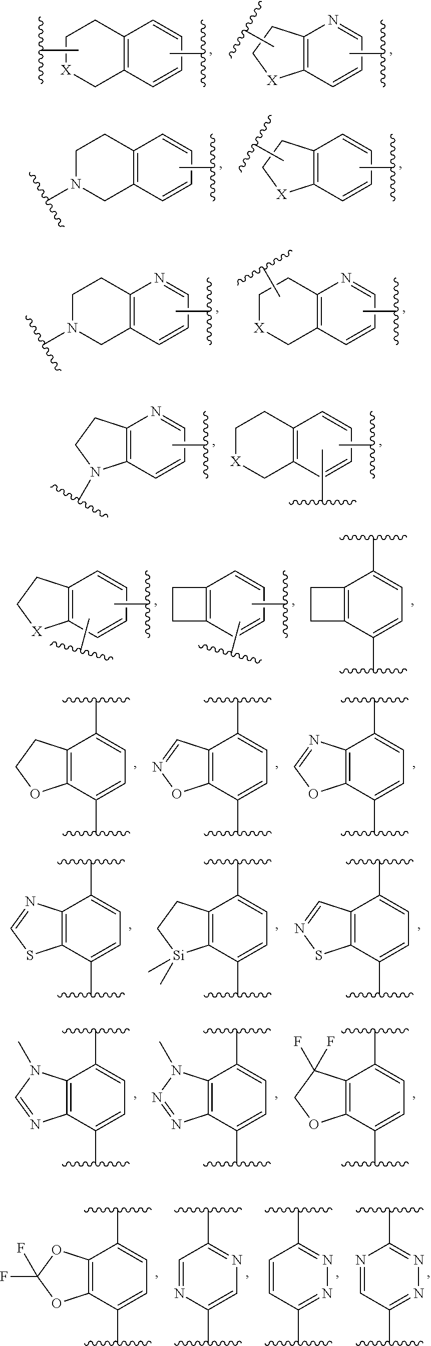 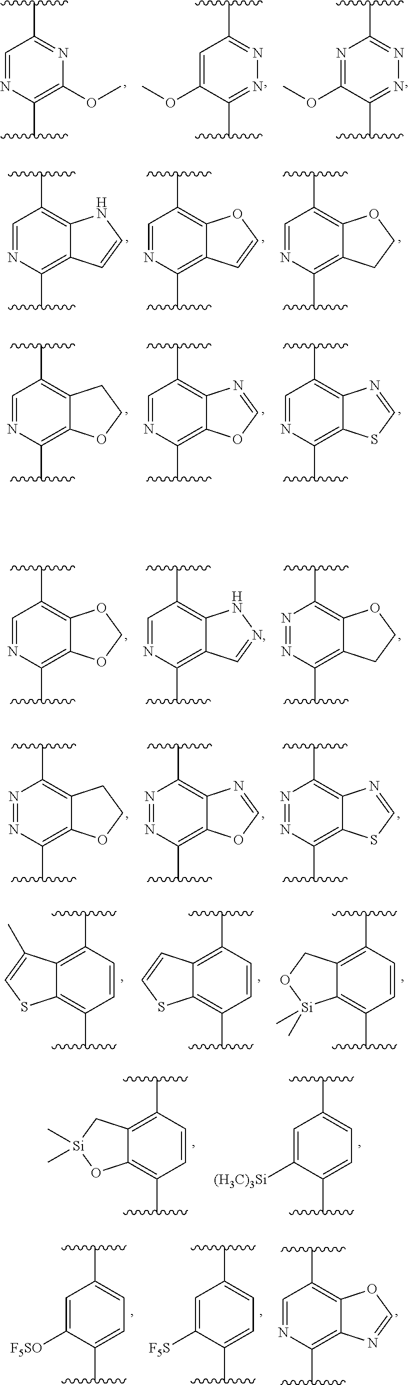 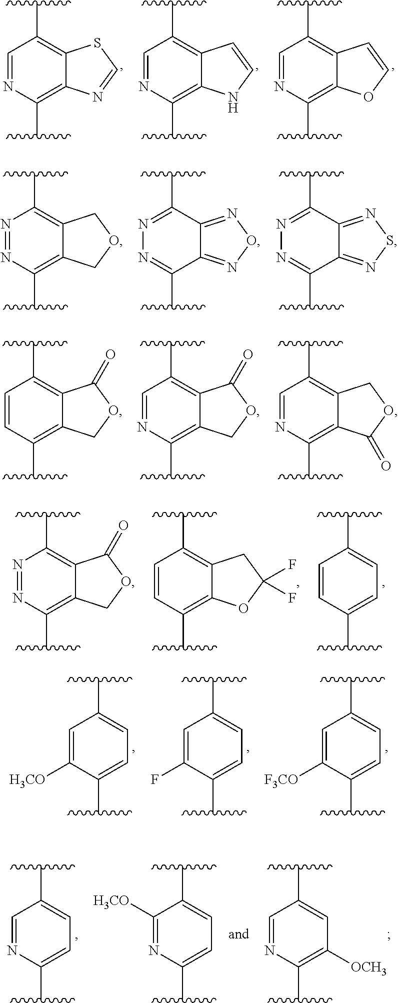
wherein X is O, N(R¹⁴) or S and wherein each R¹⁰ group (except for the bond) is optionally substituted with 1-3 independently selected R²¹ substituents;
R¹⁴ is selected from the group consisting of H, alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, —CN, —C(O)R¹⁸, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶), wherein each of said alkyl, alkenyl, alkynyl, cycloalkyl, cycloalkylalkyl-, cycloalkenyl, heterocyclyl, heterocyclylalkyl-, aryl, arylalkyl-, heteroaryl, and heteroarylalkyl- is optionally substituted with 1-5 independently selected R²¹ substitutents;
R¹⁵, R¹⁶ and R¹⁷ can be the same or different and are each independently selected from the group consisting of H, alkyl, alkenyl, alkynyl, cycloalkyl, cycloalkylalkyl-, heterocyclyl, heterocyclylalkyl-, aryl, arylalkyl-, heteroaryl, heteroarylalkyl-, arylcycloalkyl-, arylheterocyclyl-, (R¹⁸)ᵣ-alkyl, (R¹⁸)ᵣ-cycloalkyl, (R¹⁸)ᵣ-cycloalkylalkyl-, (R¹⁸)ᵣ-heterocyclyl, (R¹⁸)ᵣ-heterocyclylalkyl-, (R¹⁸)ᵣ-aryl, (R¹⁸)ᵣ-arylalkyl-, (R¹⁸)ᵣ-heteroaryl and (R¹⁸), heteroarylalkyl-; wherein r is 1-5;
each R¹⁸ is independently selected from the group consisting of alkyl, alkenyl, alkynyl, aryl, arylalkyl-, arylalkenyl-, arylalkynyl-, —NO₂, halo, heteroaryl, HO-alkyoxyalkyl-, —CF₃, —CN, alkyl-CN, —C(O)R¹⁹, —C(O)ON, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N (alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl)-OCF₃, —OH, —OR²⁰, —O-heterocyclyl, —O-cycloalkylalkyl, —O-heterocyclylalkyl, —NH₂, —NHR²⁰, —N(alkyl)₂, N(arylalkyl)₂: —N(arylalkyl)-(heteroarylalkyl), —NHC(O)R²⁰, —NHC(O)NH₂, —NHC(O)NH (alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);
or, alternately, two R¹⁸ moieties on adjacent carbons can be linked together to form: 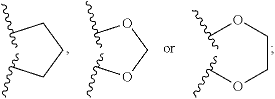
R¹⁹ is selected from the group consisting of: alkyl, cycloalkyl, aryl, arylalkyl- and heteroarylalkyl-;
R²⁰ is selected from the group consisting of: alkyl, cycloalkyl, aryl, halo substituted aryl, arylalkyl-, heteroaryl and heteroarylalkyl-;
each R²¹ group is independently selected from the group consisting of alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, halo, —CN, —OR¹⁵, —C(O)R¹⁵, —C(O)OR¹⁵, —C(O)N(R¹⁵)(R¹⁶), —SF₅, —OSF₅, —Si(R^{15A})₃, —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁶)S(O)₂N(R¹⁶)(R¹⁷), —N(R¹⁵)S(O)N(R¹⁶)(R¹⁷), —N(R¹⁸)C(O)N(R¹⁶)(R¹⁷), —CH₂—N(R¹⁸)C(O)N(R¹⁶)(R¹⁷), —N(R¹⁸)C(O)OR¹⁸, —CH₂—N(R¹⁸)C(O)OR¹⁸, —S(O)R¹⁵, ═NOR¹⁵, —N₃, —NO₂ and —S(O)₂R^{15A}; and wherein each of the R²¹ alkyl, cycloalkenyl, cycloalkyl, cycloalkylalkyl-, heterocyclyl, heterocyclylalkyl-, aryl, arylalkyl-, heteroaryl, heteroarylalkyl-, alkenyl and alkynyl groups is optionally substituted with 1 to 5 independently selected R²² groups; and
each R²² is independently selected from the group consisting of: alkyl, cycloalkyl, cycloalkenyl, heterocyclyl, aryl, heteroaryl, halo, —CF₃, —CN, —OR¹⁵, —C(O)R¹⁶, —C(O)OR¹⁶, -alkyl-C(O)OR¹⁶, C(O)N(R¹⁵)(R¹⁶), —SF₅, —OSF₅, —Si(R^{15A})₃, —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁶)C(O)R¹⁶, —CH₂—N(R¹⁸)C(O)R¹⁸, N(R¹⁶)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R^{15A}.

22. The compound of claim 21, wherein: (a) R¹ is H; or (b) R¹ is alkyl; or (c) R¹ is methyl.

23. The compound of claim 21, wherein: (a) W is a bond; or (b) W is —C(O)—; or (c)═N—W-G- is ═N—C(R¹¹R¹²)—C(O)—; or (d) W is —C(R¹¹)(R¹²)—.

24. The compound of claim 21, wherein:
(a) R⁶ is an aryl or arylalkyl-group, and said aryl group is substituted with one or more independently selected R²¹ groups; or
(b) R⁶ is aryl which is substituted with 1-4 substituents which can be the same or different, each substituent being independently selected from the group consisting of halo, alkyl, —CN, —NH₂, —NH(alkyl), —N(alkyl)₂, hydroxy, alkoxy, aryl and heteroaryl groups; or
(c) R⁶ is phenyl which is substituted with 1-4 substituents which can be the same or different, each substituent being independently selected from the group consisting of halo, alkyl, —CN, —NH₂, —NH(alkyl), —N(alkyl)₂, hydroxy, alkoxy, aryl and heteroaryl groups; or
(d) R⁶ is selected from the group consisting of: 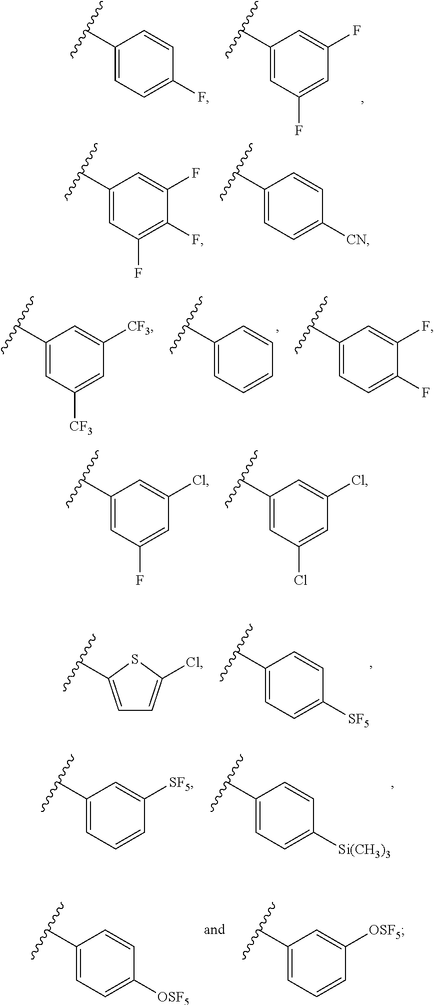
or
(e) R⁶ is 4-fluorophenyl.

25. The compound of claim 21, wherein: (a) R⁸ is H; or (b) R⁸ is alkyl; or (c) R⁸ is methyl.

26. The compound of claim 1, wherein(a) R¹⁹ is aryl; or (b) R¹⁹ is heteroaryl.

27. The compound of claim 21, wherein:
(a) R⁹ is unsubstituted heteroaryl; or
(b) R⁹ is heteroaryl which is substituted with 1-3 substituents which can be the same or different, each substituent being independently selected from the group consisting of halo, alkyl, CN, NH₂, NH(alkyl), N(alkyl)₂, hydroxy and alkoxy groups; or
(c) R⁹ is heteroaryl substituted with 1 to 3 independently selected alkyl groups; or
(d) R⁹ is heteroaryl substituted with one is alkyl group; or
(e) R⁹ is imidazol-1-yl; or
(f) R⁹ is 4-methyl-imidazol-1-yl; or
(g) R⁹ is 5-chloro-4-methyl-imidazol-1-yl.

28. The compound of claim 1 wherein:
(a) R¹⁰ is phenyl; or
(b) R¹⁰ is phenyl substituted with 1 halo; or
(c) R¹⁰ is phenyl substituted with 1 halo, and said halo is F; or
(d) R¹⁰ is: 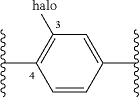
(wherein the bond from the carbon labeled as 4 is to the R⁹ group); or
(e) R¹⁰ is: 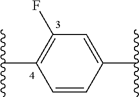
(wherein the bond from the carbon labeled as 4 is to the R⁹ group); or
(f) R¹⁰ is phenyl substituted with one —OR¹⁵ group; or
(g) R¹⁰ is phenyl substituted with one —OR¹⁵ group, and said R¹⁵ is alkyl; or
(h) R¹⁰ is: 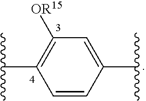
(wherein the bond from the carbon labeled as 4 is to the R⁹ group); or
(i) R¹⁰ is: 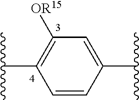
wherein R¹⁵ is alkyl (wherein the bond from the carbon labeled as 4 is to the R⁹ group); or
(j) R¹⁰ is: 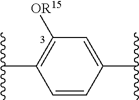
wherein R¹⁵ is methyl (i.e., R¹⁰ is 3-methoxy-phenyl).

29. The compound of claim 21 wherein:
(a) R¹⁰ is selected from the group consisting of aryl and aryl substituted with one or more R²¹ groups, and R⁹ is selected from the group consisting of heteroaryl and heteroaryl substituted with one or more R²¹ groups, and wherein each R²¹ is independently selected; or
(b) R¹⁰ is selected from the group consisting of phenyl and phenyl substituted with 1-3 independently selected R²¹ groups, and R⁹ is selected from the group consisting of imidazolyl and imidazolyl substituted with 1-3 independently selected R²¹ groups; or
(c) R¹⁰ is phenyl substituted with 1-3 independently selected R²¹ groups, and R⁹ is selected from the group consisting of imidazolyl and imidazolyl substituted with 1-3 independently selected R²¹ groups; or
(d) R¹⁰ is selected from the group consisting of heteroaryl and heteroaryl substituted with 1-3 R²¹ groups, and the R⁹ group is selected from the group consisting of heteroaryl and heteroaryl substituted with 1-3 R²¹ groups, and wherein each R²¹ is independently selected; or
(e) R¹⁰ is selected from the group consisting of pyridyl and pyridyl substituted with 1-3 R²¹ groups, and the R⁹ group is selected from the group consisting of imidazolyl and imidazolyl substituted with 1-3 R²¹ groups, and wherein each R²¹ is independently selected; or
(f) R¹⁰ is pyridyl, and the R⁹ group is imidazolyl substituted with 1-3 R²¹ groups, and wherein each R²¹ is independently selected; or
(g) the R⁹—R¹⁰— moiety is: 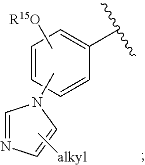
or
(h) R⁹—R¹⁰— moiety is: 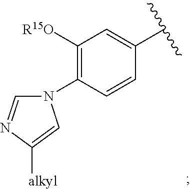
or
(i) the R⁹—R¹⁰— moiety is: 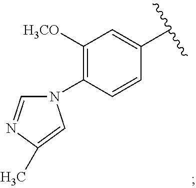
or
(j) the R⁹—R¹⁰— moiety is: 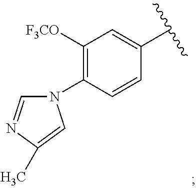
or
(k) the R⁹—R¹⁰— moiety is: 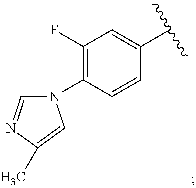
or
(l) R⁹—R¹⁰-moiety is: 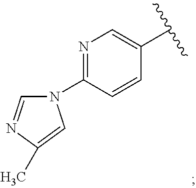
or
(m) the R⁹—R¹⁰— moiety is:

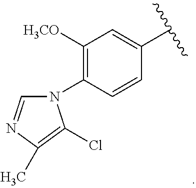

30. A compound according to claim 21 selected from the formulas 3-50: 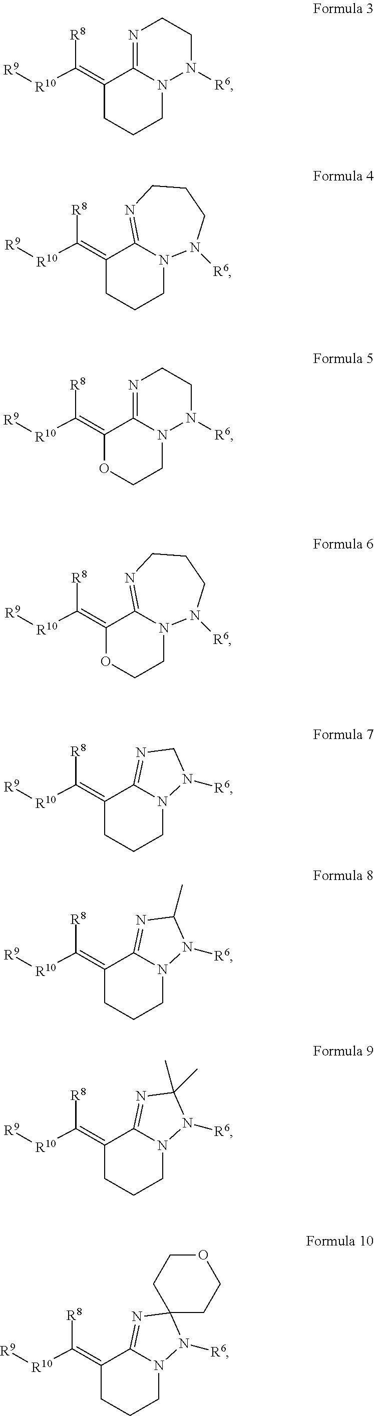 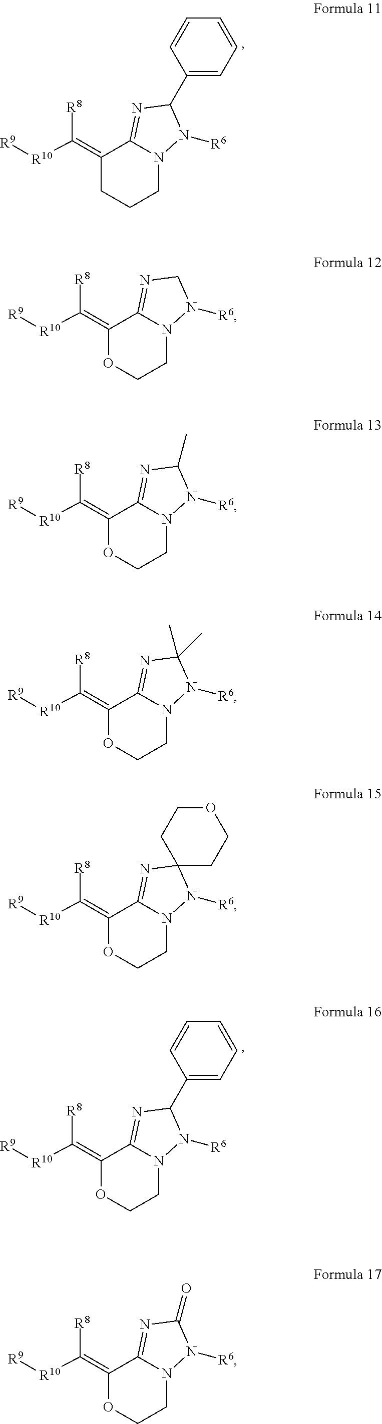 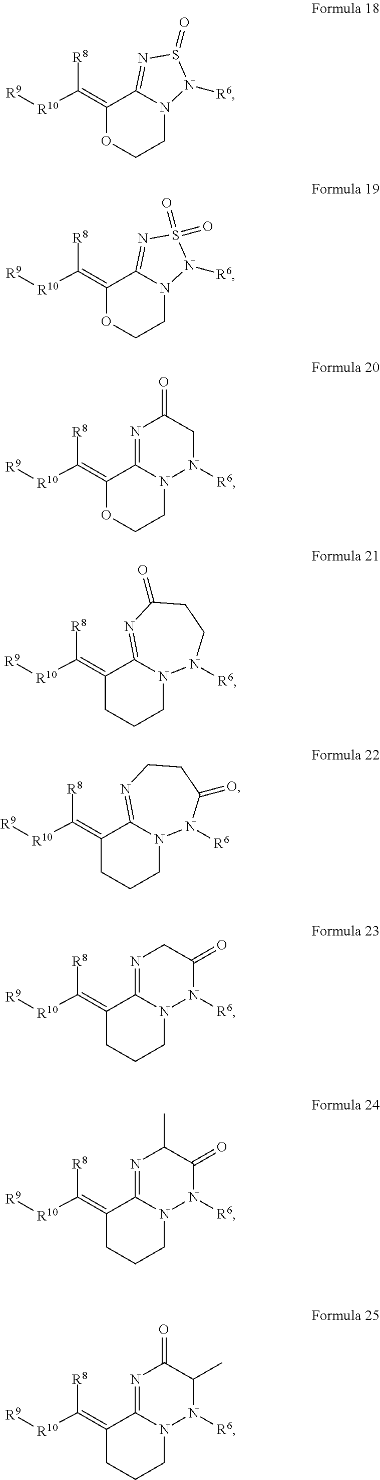 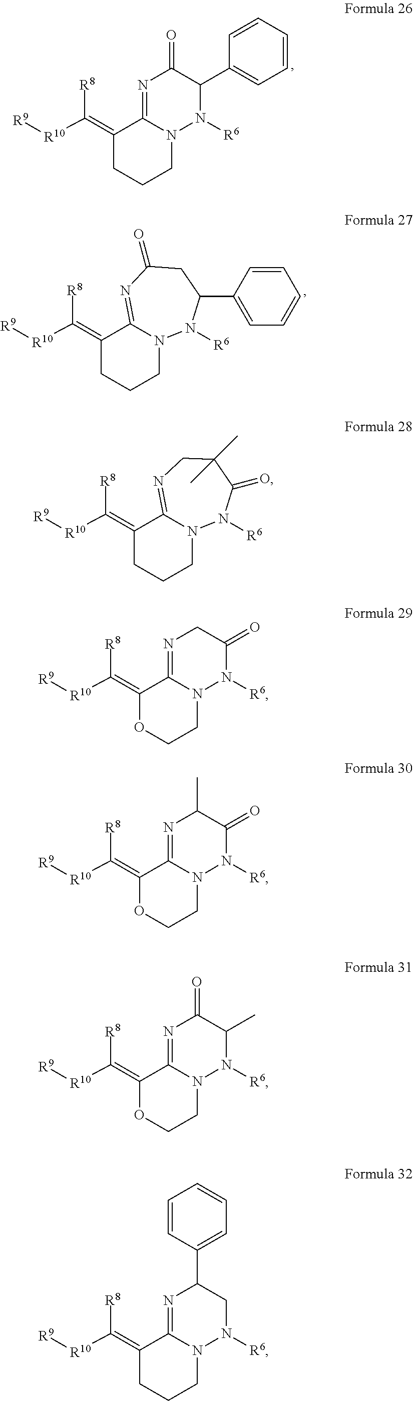 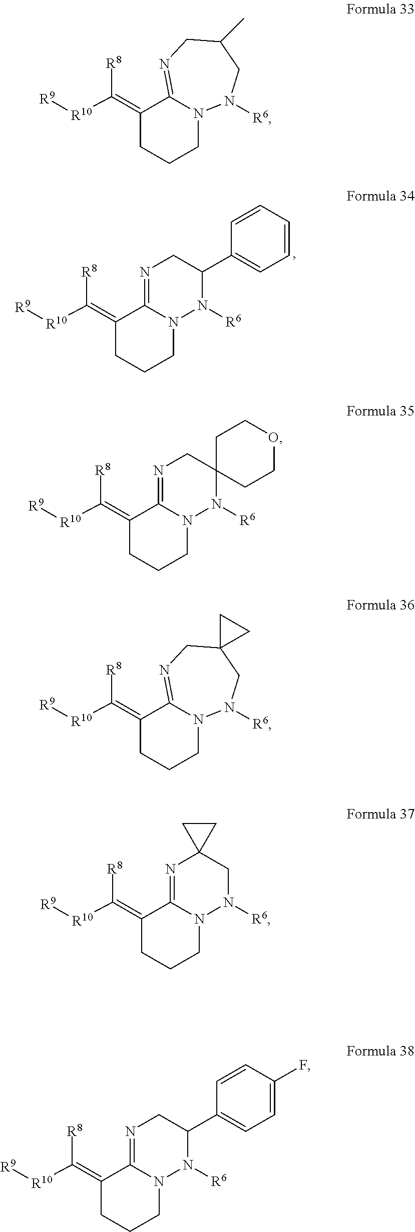 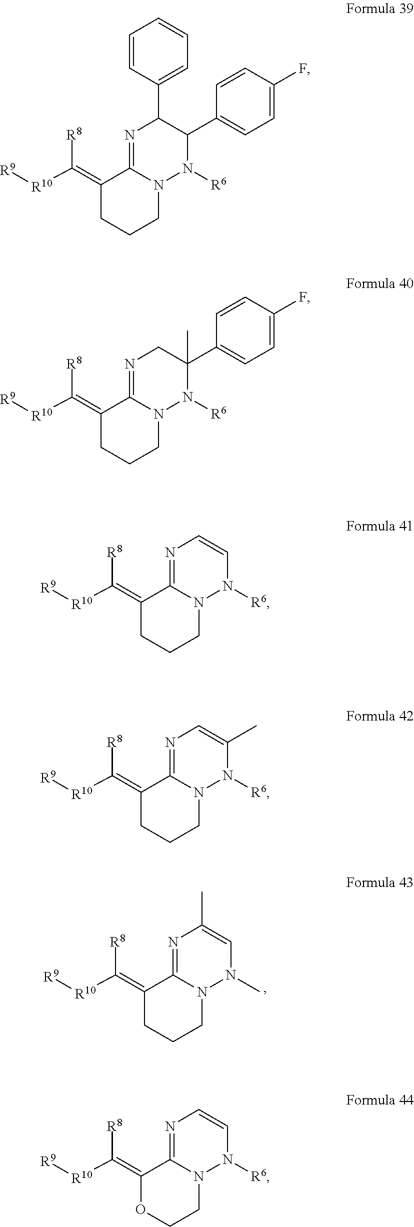 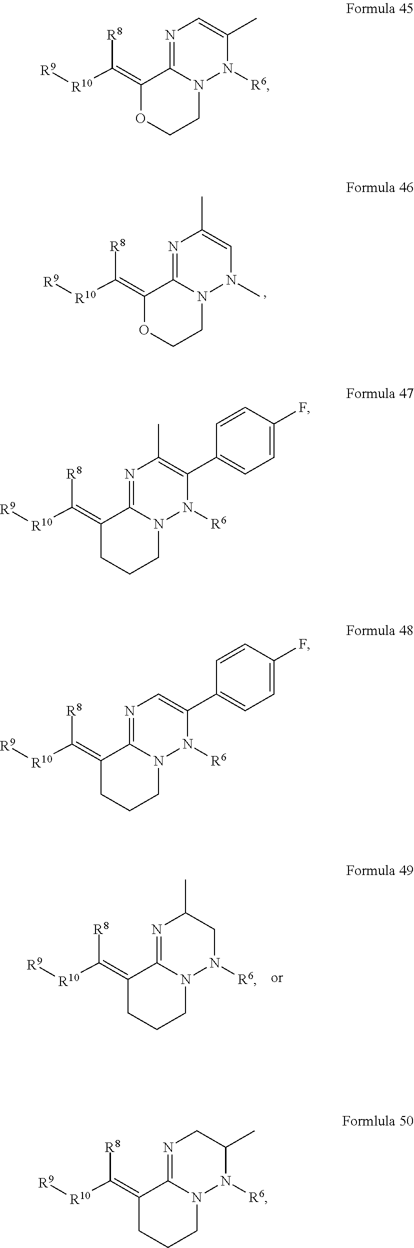
or pharmaceutically acceptable salts, of said compound, wherein
R⁶ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- can be unsubstituted or optionally substituted with 1-5 independently selected R²¹ substituents;
R⁸ is selected from the group consisting of H, alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl-, with each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- being unsubstituted or optionally substituted with 1-3 independently selected R²¹ substituents;
R⁹ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each R⁹ group is optionally substituted with 1-3 independently selected R²¹ substituents;
R¹⁰ is selected from the group consisting of a bond, alkyl-, alkenyl-, alkynyl-, aryl-, arylalkyl-, alkylaryl-, cycloalkyl-, cycloalkylalkyl-, heteroaryl-, heteroarylalkyl-, heterocyclyl-, heterocyclylalkyl- and the moieties: 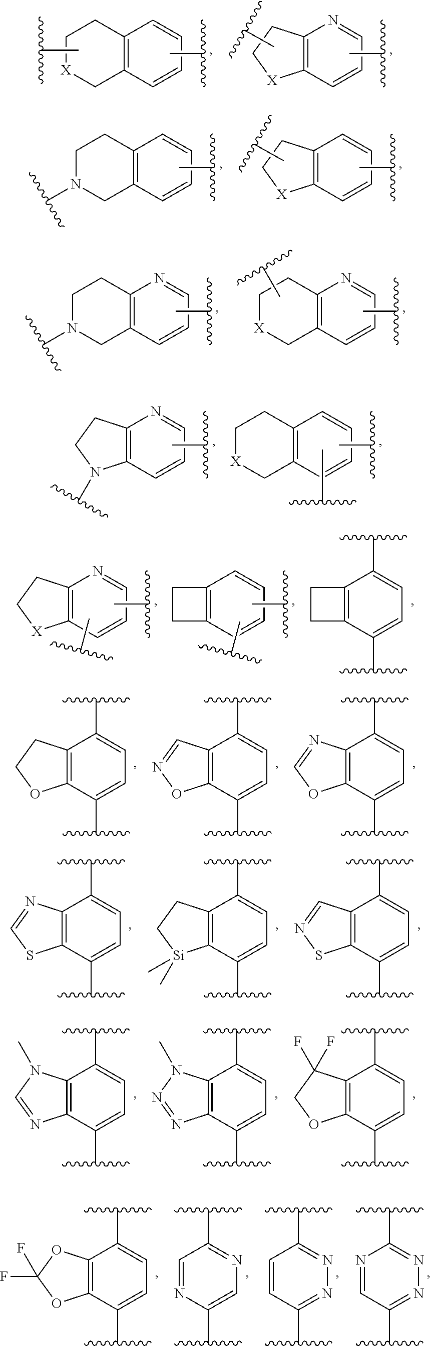 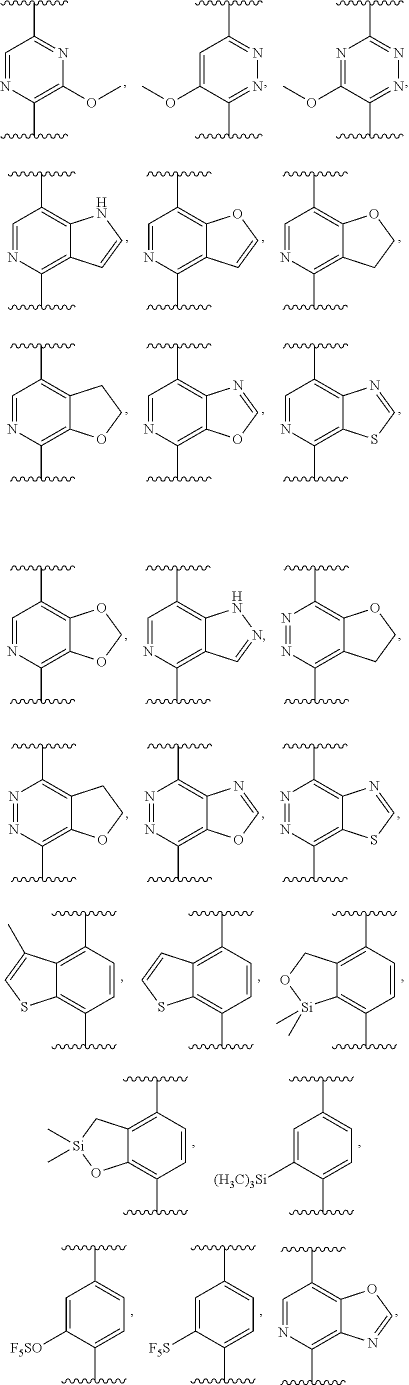 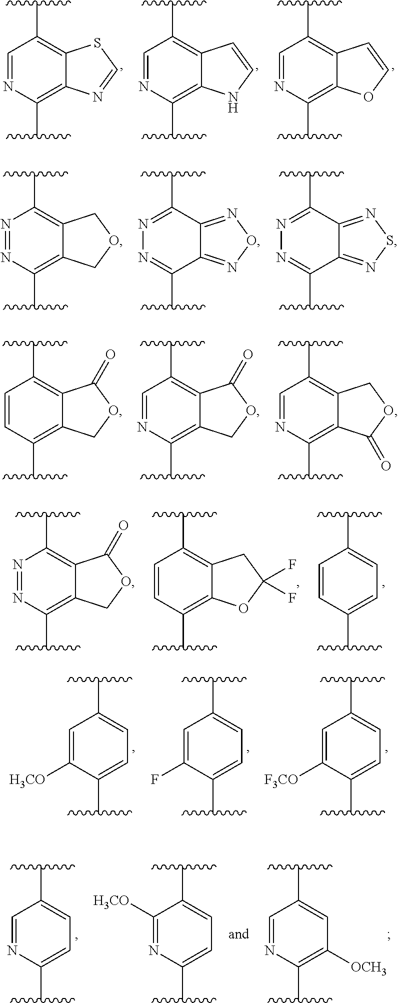
wherein X is O, N(R¹⁴) or S and wherein each R¹⁰ group (except for the bond) is optionally substituted with 1-3 independently selected R²¹ substituents;
each R²¹ group is independently selected from the group consisting of alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, halo, —ON, —OR¹⁵, —C(O)R¹⁵, —C(O)OR¹⁵, —C(O)N(R¹⁵)(R¹⁶), —SF₅, —OSF₅, —Si(R^{15A})₃, —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶)—N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁶)S(O)N(R¹⁶)(R¹⁷), —N(R¹⁶)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁸, ═NOR¹⁶, —N₃, —NO₂ and —S(O)₂R^{15A}; and wherein each of the R²¹ alkyl, cycloalkenyl, cycloalkyl, cycloalkylalkyl-, heterocyclyl, heterocyclylalkyl-, aryl, arylalkyl-, heteroaryl, heteroarylalkyl-, alkenyl and alkynyl groups is optionally substituted with 1 to 5 independently selected R²² groups;
each R²² is independently selected from the group consisting of: alkyl, cycloalkyl, cycloalkenyl, heterocyclyl, aryl, heteroaryl, halo, —CF₃, —CN, —OR¹⁵, —C(O)R¹⁶, —C(O)OR¹⁸, -alkyl-C(O)OR¹⁶, C(O)N(R¹⁵)(R¹⁶), —SF₅, —OSF₅, —Si(R^{15A})₃, —SR¹⁵, —S(O)N(R¹⁵)(R¹⁶), —S(O)₂N(R¹⁵)(R¹⁶), —C(═NOR¹⁵)R¹⁶, —P(O)(OR¹⁶)(OR¹⁶), —N(R¹⁵)(R¹⁶), -alkyl-N(R¹⁵)(R¹⁶), —N(R¹⁵)C(O)R¹⁶, —CH₂—N(R¹⁶)C(O)R¹⁶, —N(R¹⁵)S(O)R¹⁶, —N(R¹⁶)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R^{15A}; and
R¹⁴ is selected from the group consisting of H, alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, —CN, —C(O)R¹⁶, —C(O)OR¹⁶, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶), wherein each of said alkyl, alkenyl, alkynyl, cycloalkyl, cycloalkylalkyl-, cycloalkenyl, heterocyclyl, heterocyclylalkyl-, aryl, arylalkyl-, heteroaryl, and heteroarylalkyl- is optionally substituted with 1-5 independently selected R²¹ substitutents;
R¹⁵, R¹⁶ and R¹⁷ can be the same or different and are each independently selected from the group consisting of H, alkyl, alkenyl, alkynyl, cycloalkyl, cycloalkylalkyl-, heterocyclyl, heterocyclylalkyl-, aryl, arylalkyl-, heteroaryl, heteroarylalkyl-, arylcycloalkyl-, arylheterocyclyl-, (R¹⁸)ᵣ-alkyl, (R¹⁸)ᵣ-cycloalkyl, (R¹⁸)ᵣ-cycloalkylalkyl-, (R¹⁸)ᵣ-heterocyclyl, (R¹⁸)ᵣ-heterocyclylalkyl-, (R¹⁸)ᵣ-aryl, (R¹⁸)ᵣ-arylalkyl-, (R¹⁸)ᵣ-heteroaryl and (R¹⁸)ᵣ-heteroarylalkyl-; wherein r is 1-5;
each R¹⁸ is independently selected from the group consisting of alkyl, alkenyl, alkynyl, aryl, arylalkyl-, arylalkenyl-, arylalkynyl-, —NO₂, halo, heteroaryl, HO-alkyoxyalkyl-, CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkyla I kyl, —O-heterocyclylalkyl, —NH₂, —NHR²⁰, —N (alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N (alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);
or, alternately, two R¹⁸ moieties on adjacent carbons can be linked together to form: 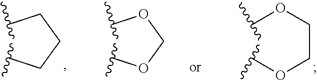
R¹⁹ is selected from the group consisting of: alkyl, cycloalkyl, aryl, arylalkyl- and heteroarylalkyl-;
R²⁰ is selected from the group consisting of: alkyl, cycloalkyl, aryl, halo substituted aryl, arylalkyl-, heteroaryl and heteroarylalkyl-.

31. A compound according to claim 30, wherein:
(1)
R⁶ is H, alkyl or aryl, which aryl can be unsubstituted or substituted with 1 to 3 independently selected R²¹ moieties;
R⁸ is H, alkyl or aryl;
R⁹ is selected from the group consisting of heteroaryl and heteroaryl substituted with 1-3 independently selected R²¹ groups; and
R¹⁰ is aryl, which can be unsubstituted or substituted with 1 to 3 independently selected R²¹ moieties, heteroaryl and heteroaryl substituted with 1-3 independently selected R²¹ groups, or a fused aryl ring selected from 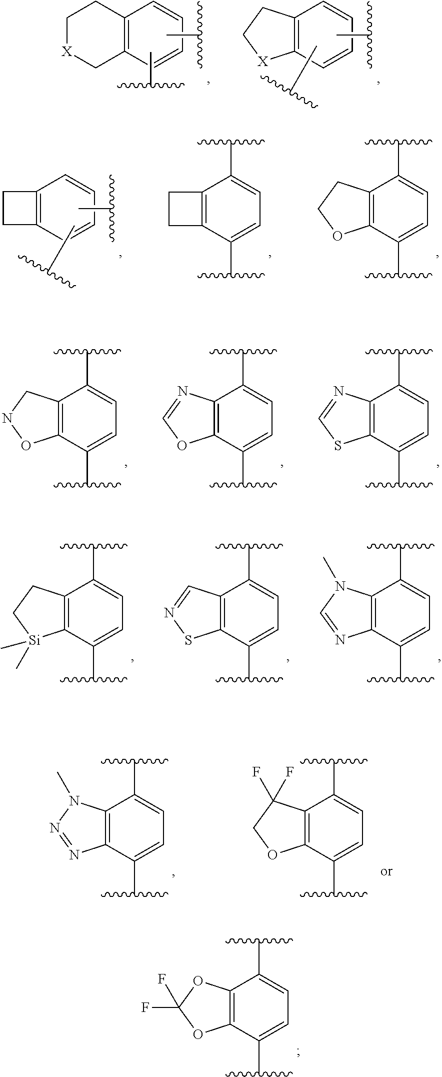
or
(2)
R⁶ is H, methyl or phenyl, which phenyl can be unsubstituted or substituted with 1 to 3 independently selected R²¹ moieties;
R⁸ is H or alkyl; and
R⁹—R¹⁰ — is selected from: 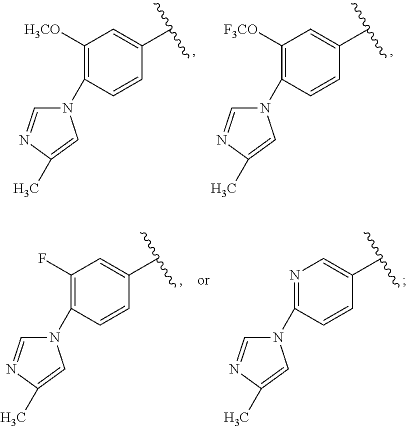
or
(3)
R⁶ is phenyl, which can be unsubstituted or substituted with 1 to 3 R²¹ moieties which can be the same or different and are independently selected from halo (preferably fluoro), SF₅, OSF₅, and Si(Me)₃;
R⁸ is H or alkyl; and
R⁹—R¹⁰— is

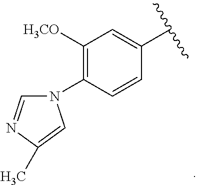

32. A compound according to claim 21 selected from the formulas 51-53: 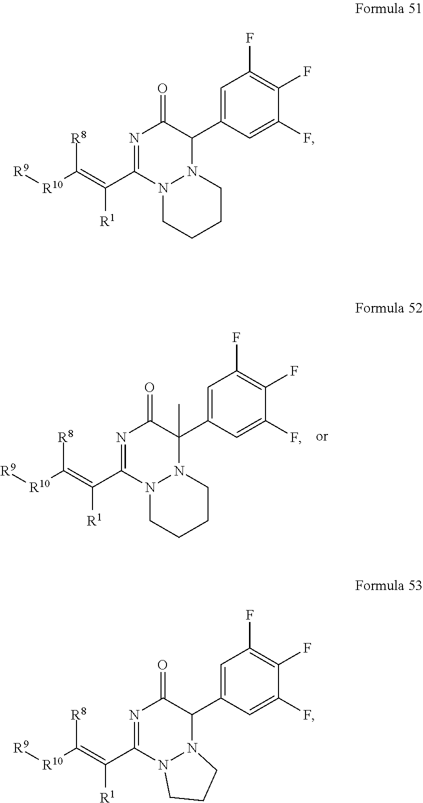
or pharmaceutically acceptable salts, solvates, esters or prod rugs of said compound, wherein
R¹ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- can be unsubstituted or optionally substituted with 1-5 independently selected R²¹ substituents;
R⁸ is selected from the group consisting of H, alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl-, with each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclylalkyl- being unsubstituted or optionally substituted with 1-3 independently selected R²¹ substituents;
R⁹ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclylalkyl-, wherein each R⁹ group is optionally substituted with 1-3 independently selected R²¹ substituents;
R¹⁰ is selected from the group consisting of a bond, alkyl-, alkenyl-, alkynyl-, aryl-, arylalkyl-, alkylaryl-, cycloalkyl-, cycloalkylalkyl-, heteroaryl-, heteroarylalkyl-, heterocyclyl-, heterocyclylalkyl- and the moieties: 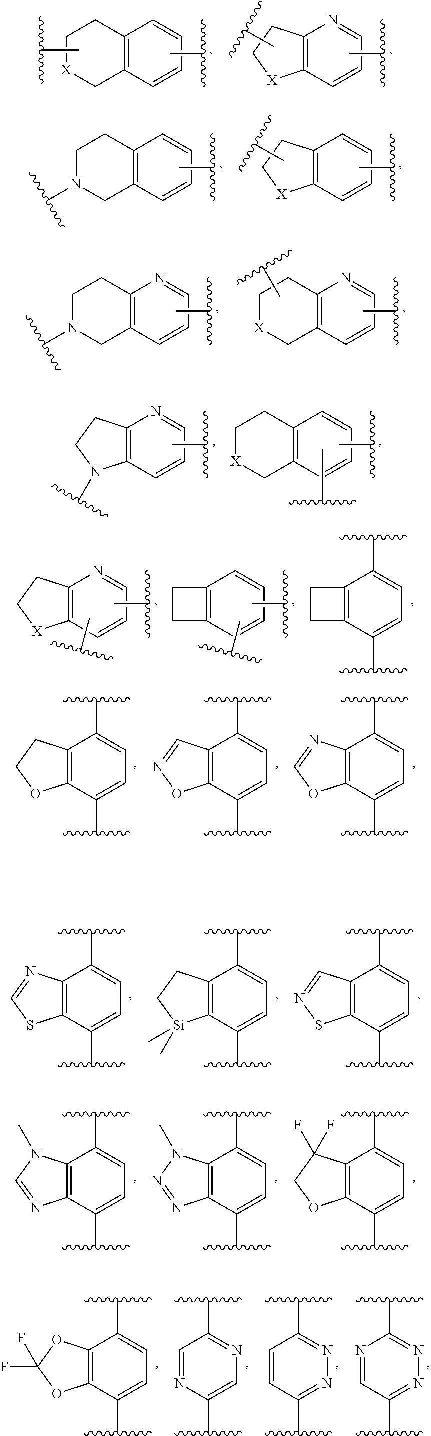 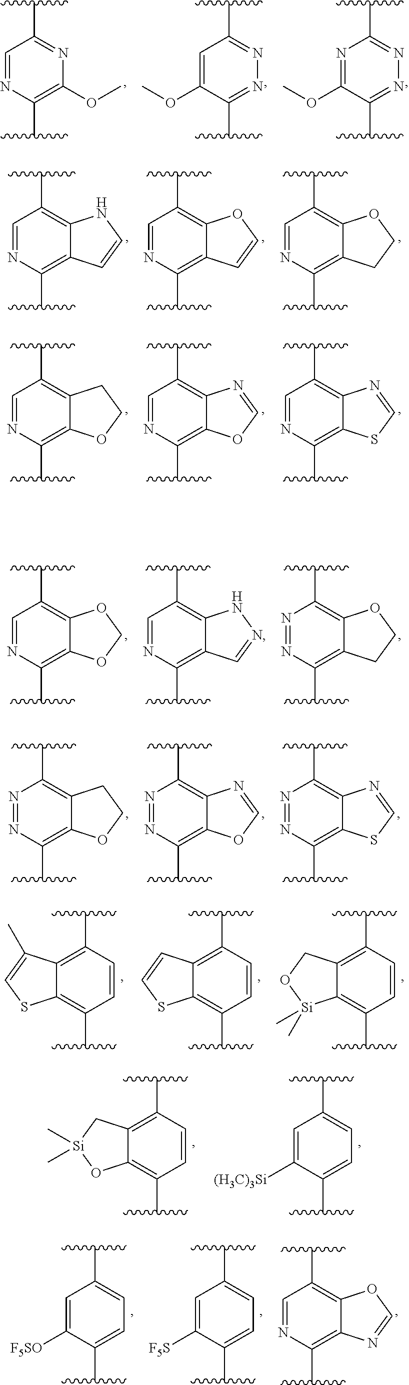 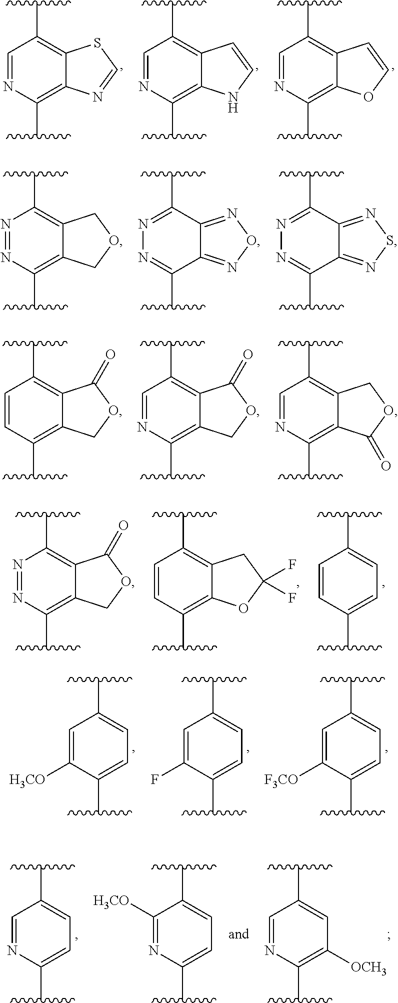
wherein X is O, N(R¹⁴) or S and wherein each R¹⁰ group (except for the bond) is optionally substituted with 1-3 independently selected R²¹ substituents;
each R²¹ group is independently selected from the group consisting of alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, halo, —CN, —OR¹⁵, —C(O)R¹⁵, —C(O)OR¹⁵, —C(O)N(R¹⁵)(R¹⁶), —SF₅, —OSF₅, —Si(R^{15A})₃, —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CF₁₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R^{15A}; and wherein each of the R²¹ alkyl, cycloalkenyl, cycloalkyl, cycloalkylalkyl-, heterocyclyl, heterocyclylalkyl-, aryl, arylalkyl-, heteroaryl, heteroarylalkyl-, alkenyl and alkynyl groups is optionally substituted with 1 to 5 independently selected R²² groups;
each R²² is independently selected from the group consisting of: alkyl, cycloalkyl, cycloalkenyl, heterocyclyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SF₅, —OSF₅, —Si(R^{15A})₃, —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁵)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and S(O)₂R^{15A}; and
R¹⁴ is selected from the group consisting of H, alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶), wherein each of said alkyl, alkenyl, alkynyl, cycloalkyl, cycloalkylalkyl-, cycloalkenyl, heterocyclyl, heterocyclylalkyl-, aryl, arylalkyl-, heteroaryl, and heteroarylalkyl- is optionally substituted with 1-5 independently selected R²¹ substitutents;
R¹⁵, R¹⁶ and R¹⁷ can be the same or different and are each independently selected from the group consisting of H, alkyl, alkenyl, alkynyl, cycloalkyl, cycloalkylalkyl-, heterocyclyl, heterocyclylalkyl-, aryl, arylalkyl-, heteroaryl, heteroarylalkyl-, arylcycloalkyl-, arylheterocyclyl-, (R¹⁸)ᵣ-alkyl, (R¹⁸)ᵣ-cycloalkyl, (R¹⁸)ᵣ-cycloalkylalkyl-, (R¹⁸)ᵣ-heterocyclyl, (R¹⁸)ᵣ-heterocyclylalkyl-, (R¹⁸)ᵣ-aryl, (R¹⁵), -arylalkyl-, (R¹⁸)ᵣ-heteroaryl and (R¹⁸)ᵣ heteroarylalkyl-; wherein r is 1-5;
each R¹⁸ is independently selected from the group consisting of alkyl, alkenyl, alkynyl, aryl, arylalkyl-, arylalkenyl-, arylalkynyl-, —NO₂, halo, heteroaryl, HO-alkyoxyalkyl-, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N (alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);
or, alternately, two R¹⁸ moieties on adjacent carbons can be linked together to form: 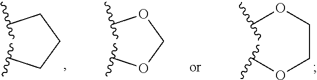
R¹⁹ is selected from the group consisting of: alkyl, cycloalkyl, aryl, arylalkyl- and heteroarylalkyl-;
R²⁰ is selected from the group consisting of: alkyl, cycloalkyl, aryl, halo substituted aryl, arylalkyl-, heteroaryl and heteroarylalkyl-.

33. A compound according to claim 32, wherein:
(1)
R¹ is H, alkyl or aryl, which aryl can be unsubstituted or substituted with 1 to 3 independently selected R²¹ moieties;
R⁸ is H, alkyl or aryl;
R⁹ is selected from the group consisting of heteroaryl and heteroaryl substituted with 1-3 independently selected R²¹ groups; and
R¹⁰ is aryl, which can be unsubstituted or substituted with 1 to 3 independently selected R²¹ moieties, heteroaryl and heteroaryl substituted with 1-3 independently selected R²¹ groups, or a fused aryl ring selected from 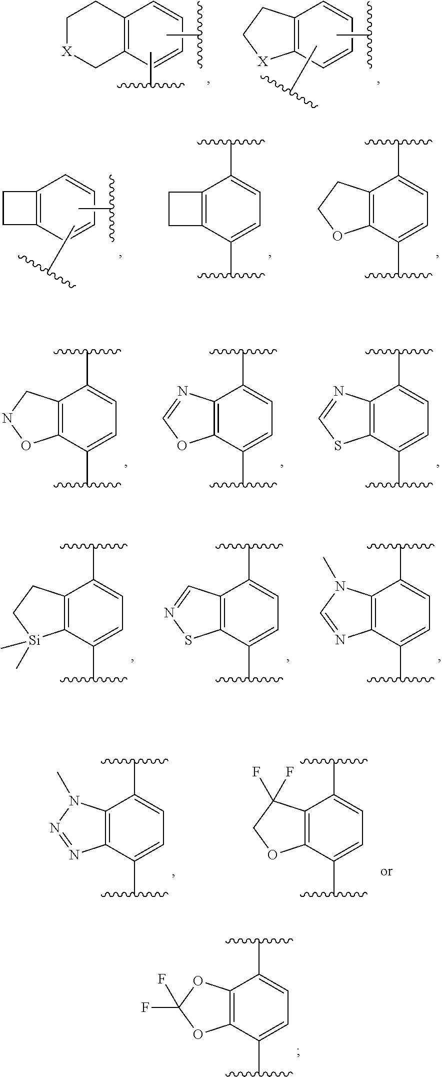
or
(2)
R¹ is H, methyl or phenyl, which phenyl can be unsubstituted or substituted with 1 to 3 independently selected R²¹ moieties;
R⁸ is H or alkyl; and
R⁹—R¹⁰— is selected from: 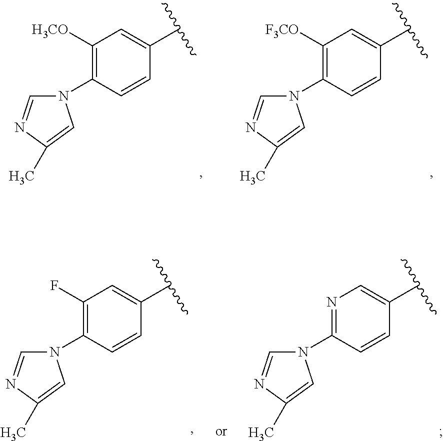
or
(3)
R¹ is phenyl, which can be unsubstituted or substituted with 1 to 3 R²¹ moieties which can be the same or different and are independently selected from halo, SF_{S}, OSF₅, and Si(Me)₃;
R⁸ is H or alkyl; and
R⁹—R¹⁰— is

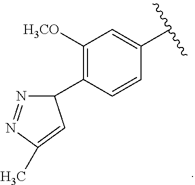

34. A compound according to claim 21 of the formula: 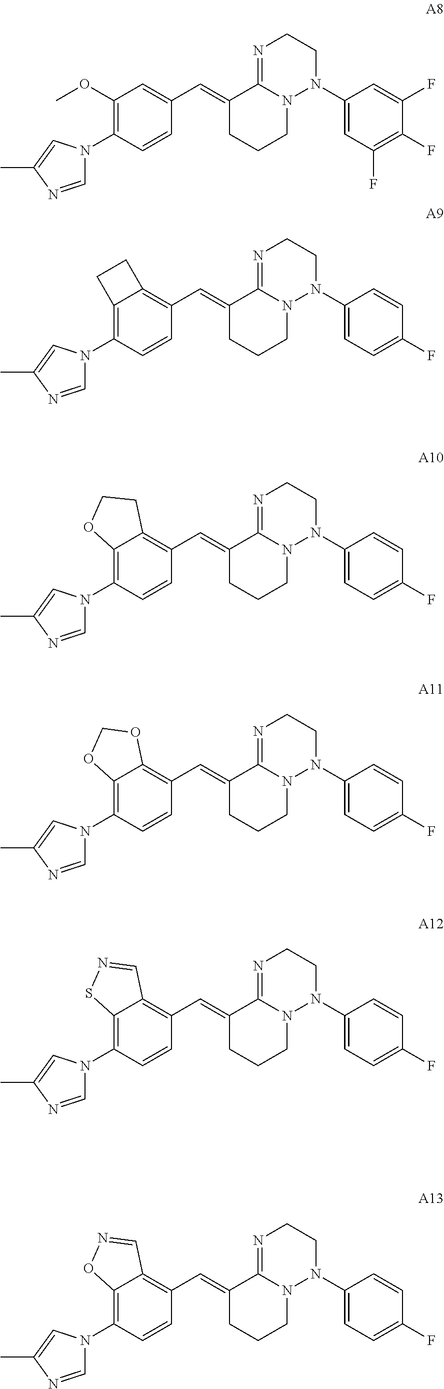 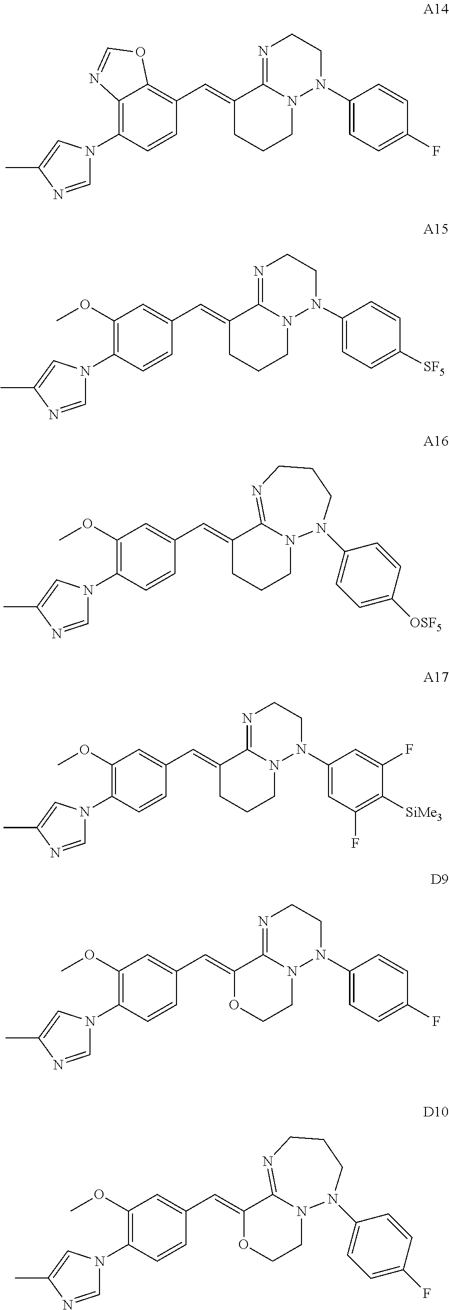 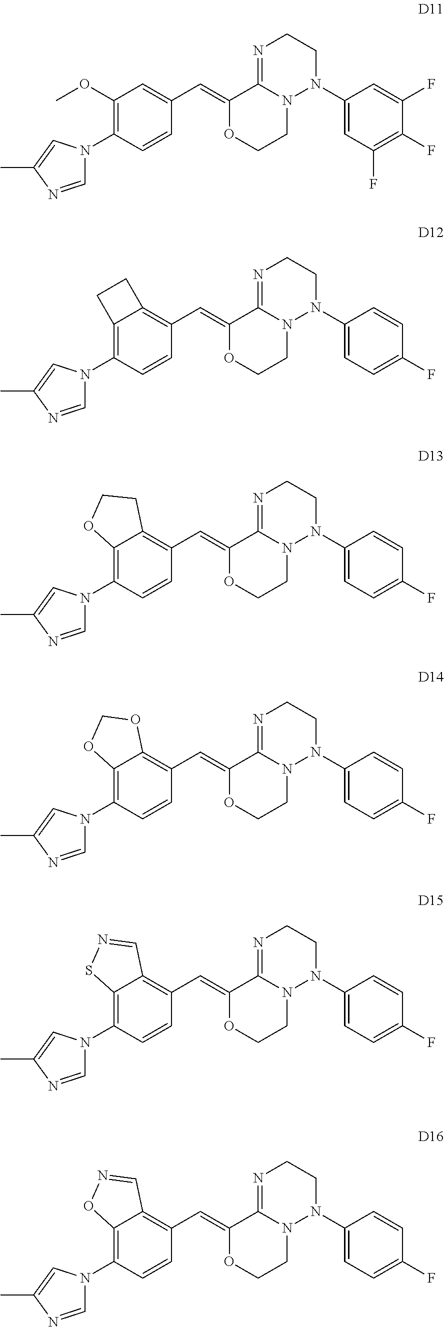 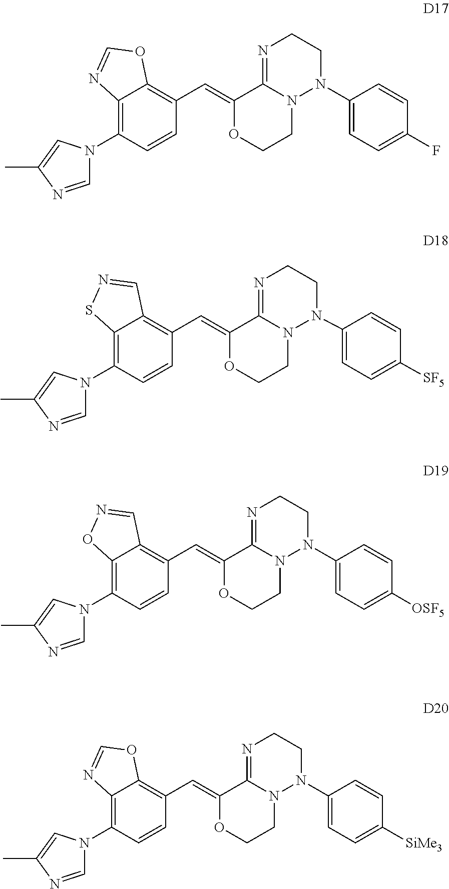 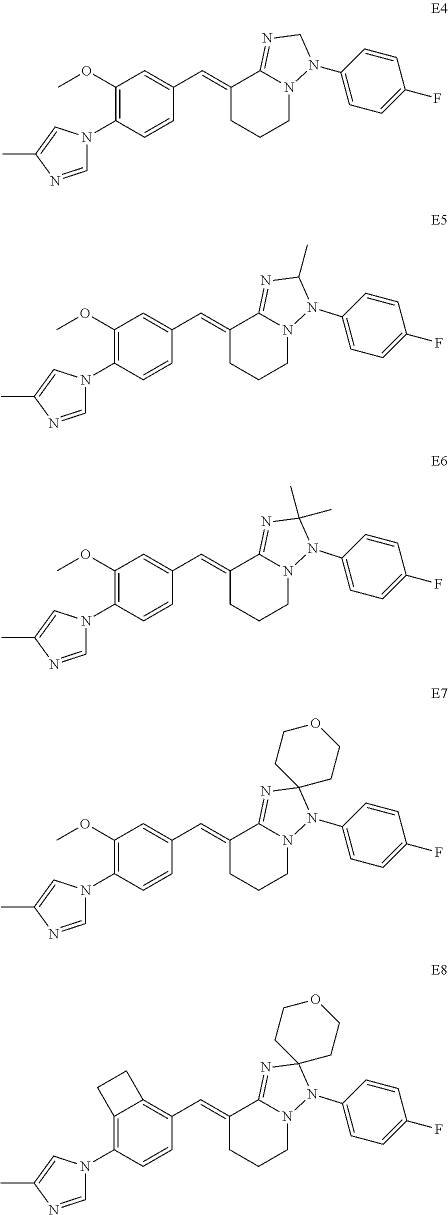 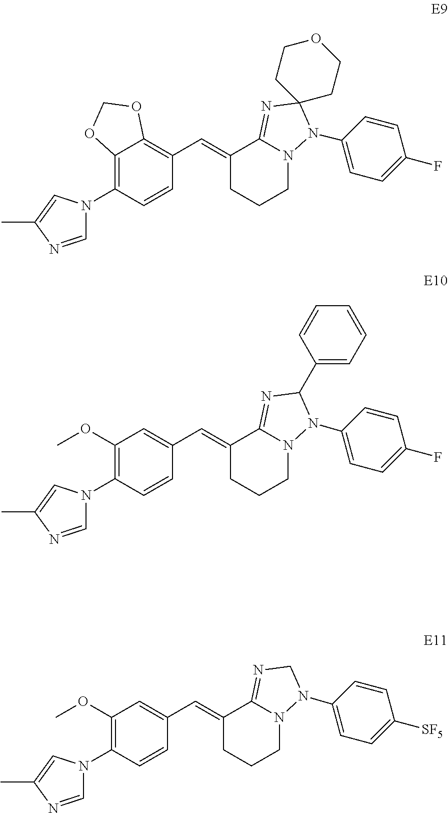 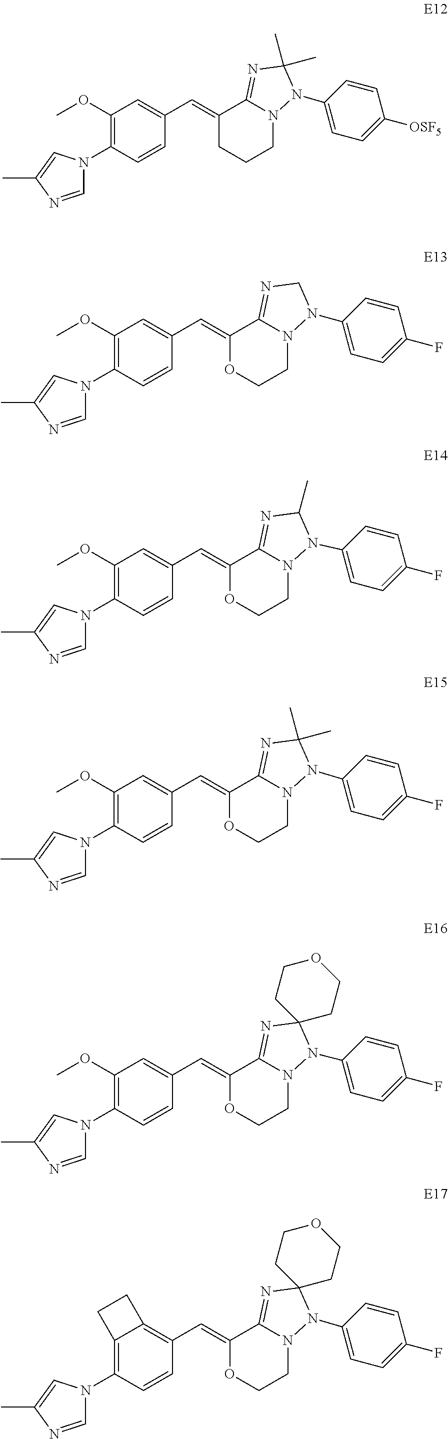 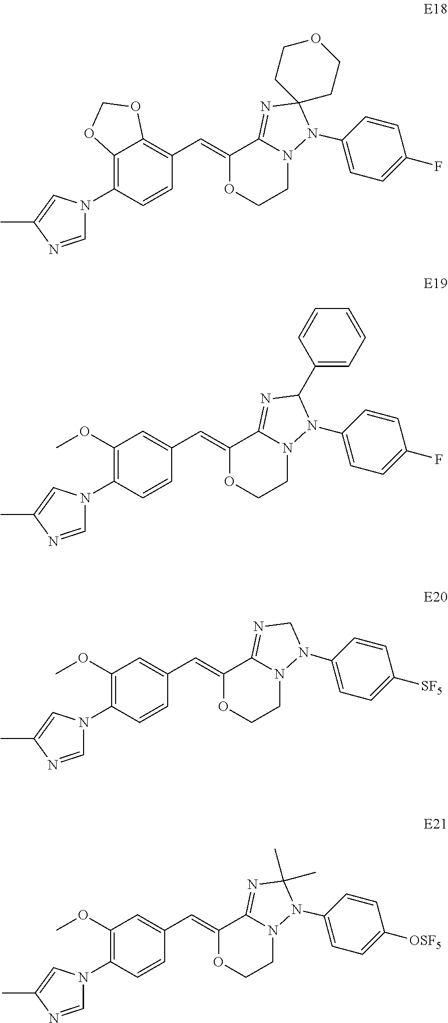 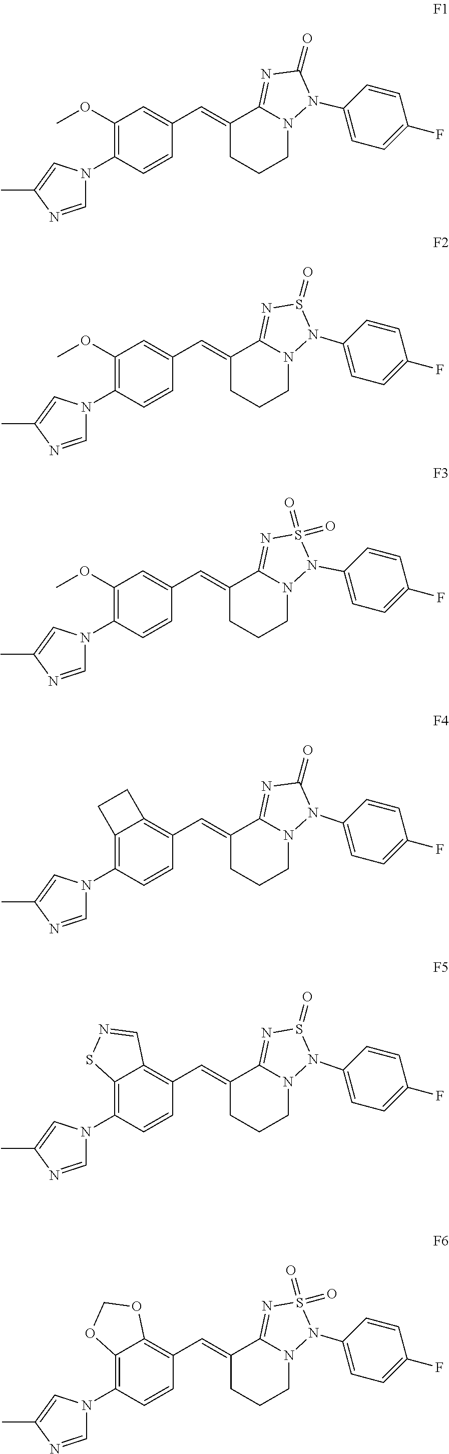 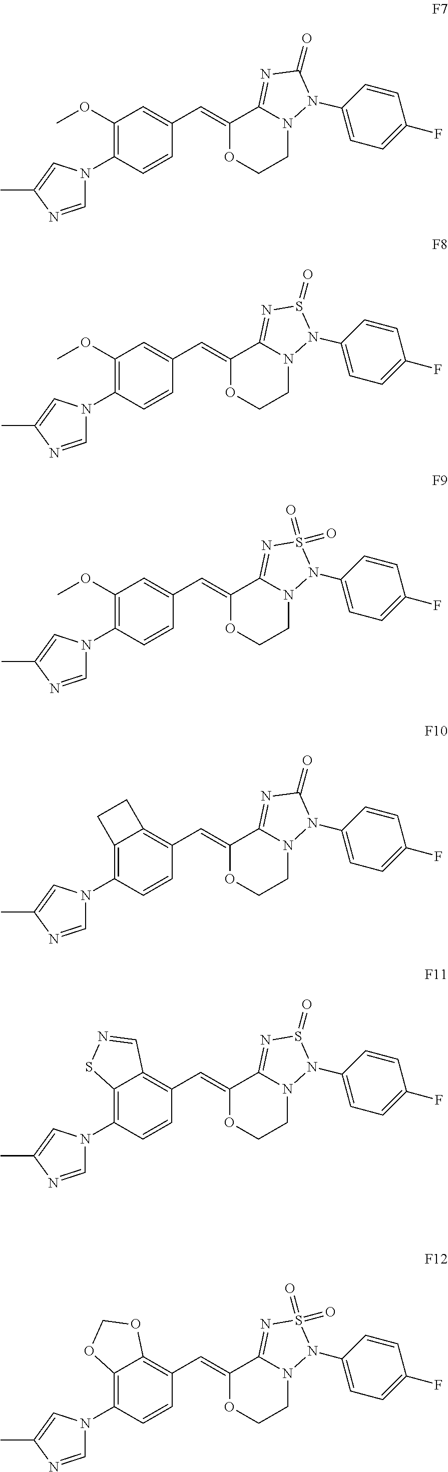 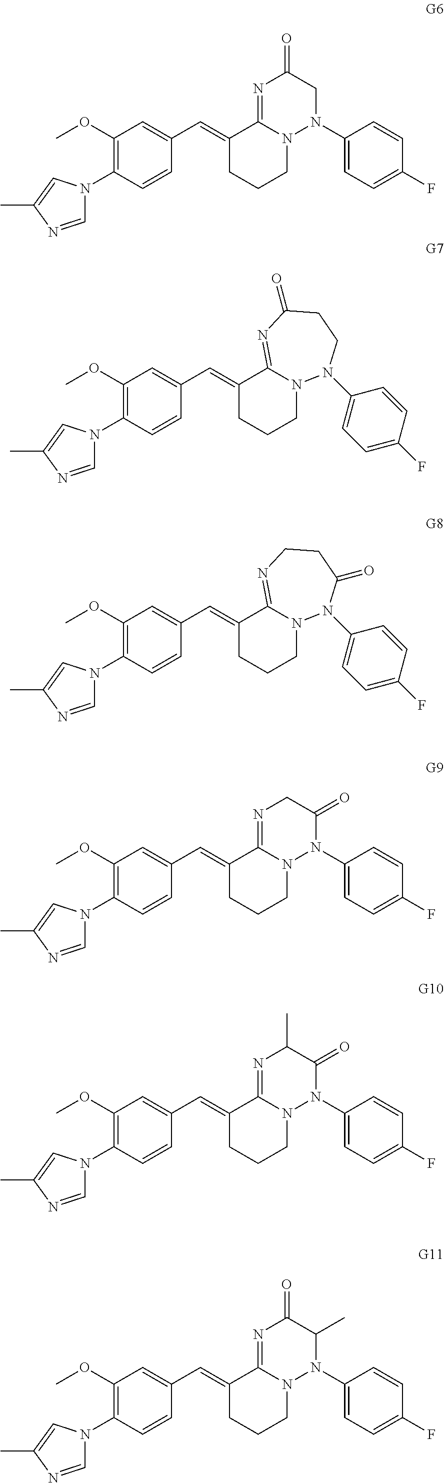 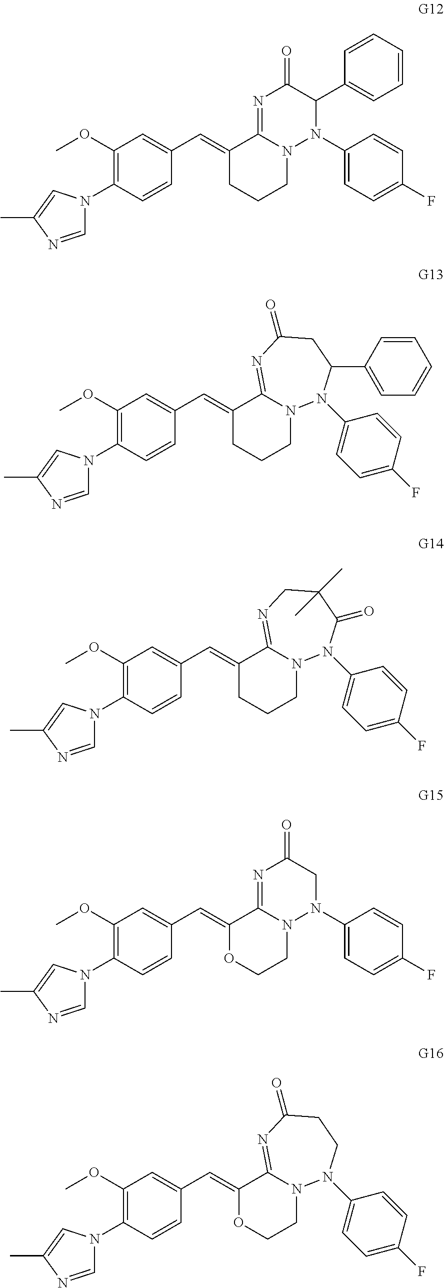 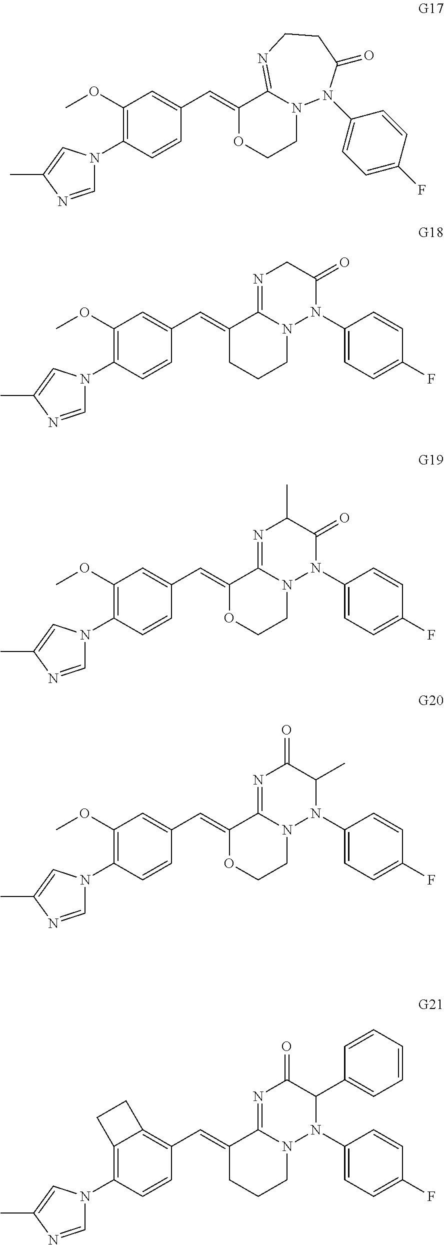 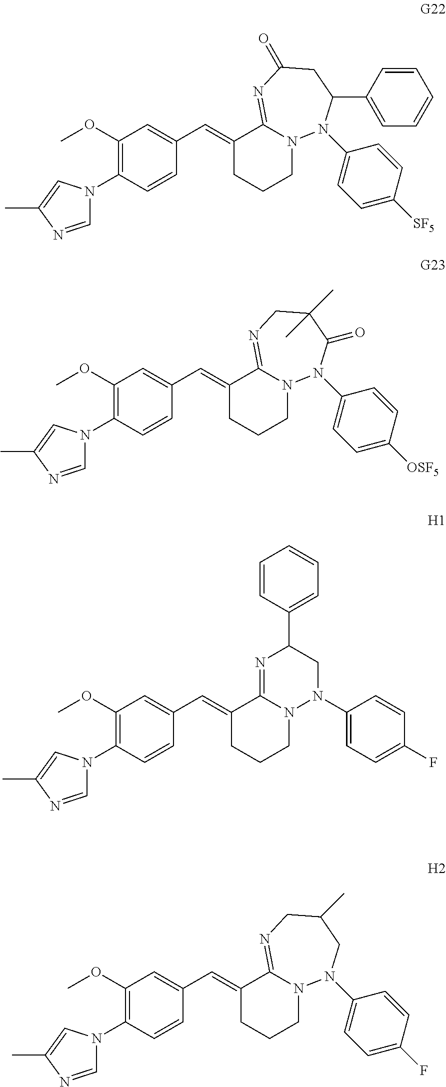 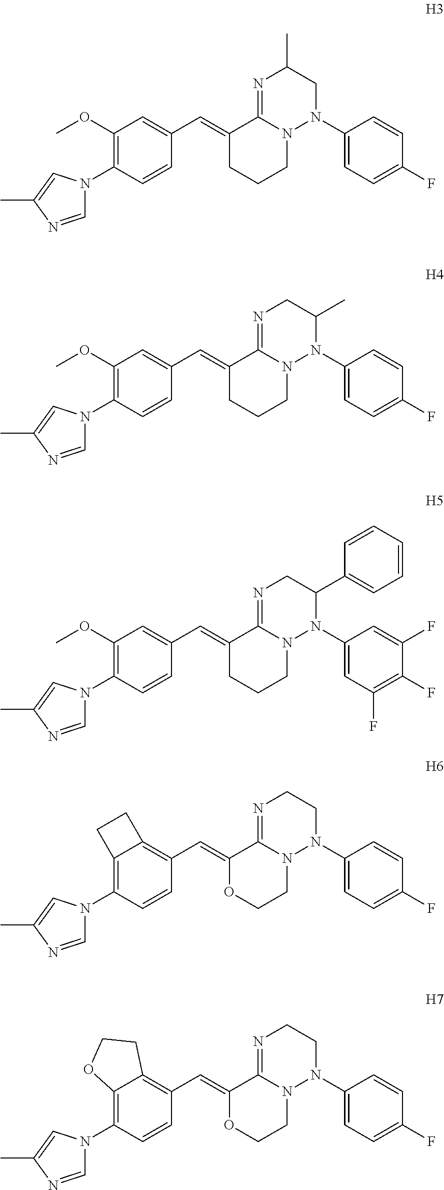 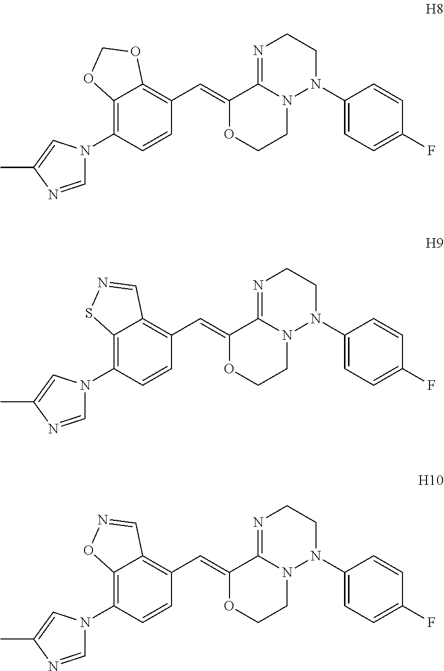 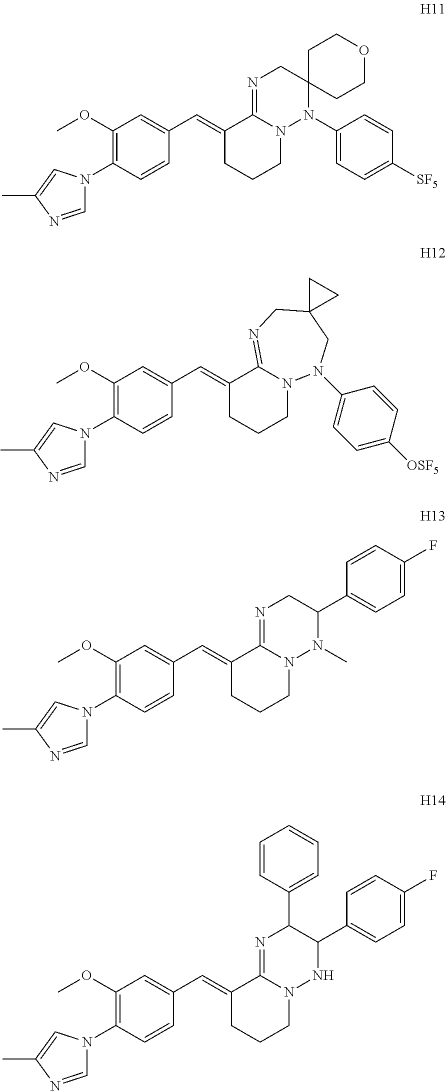 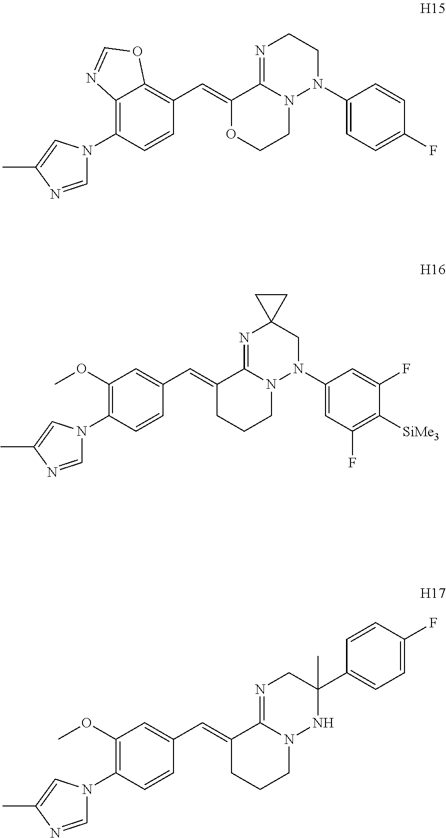 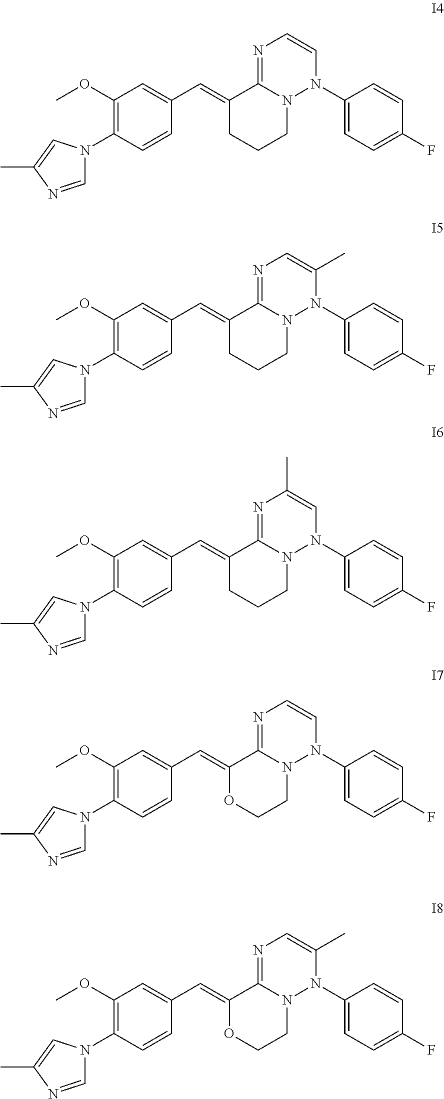 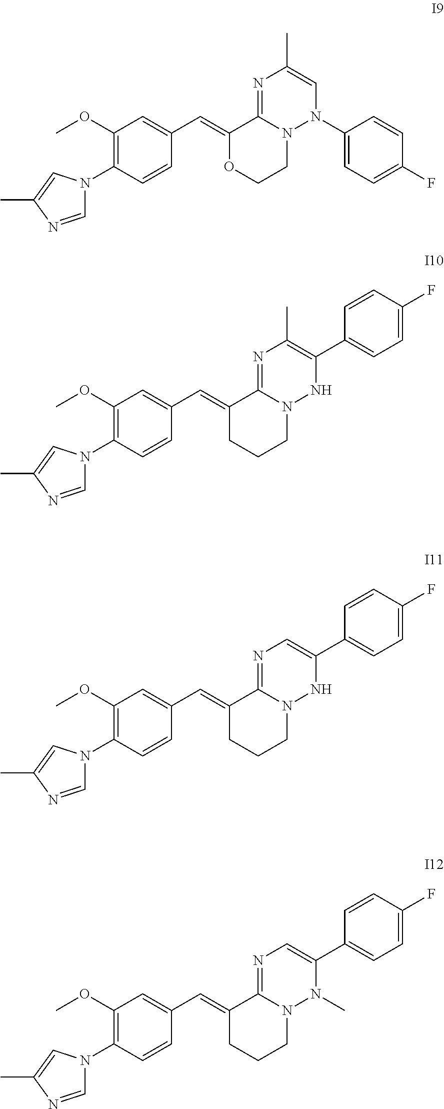 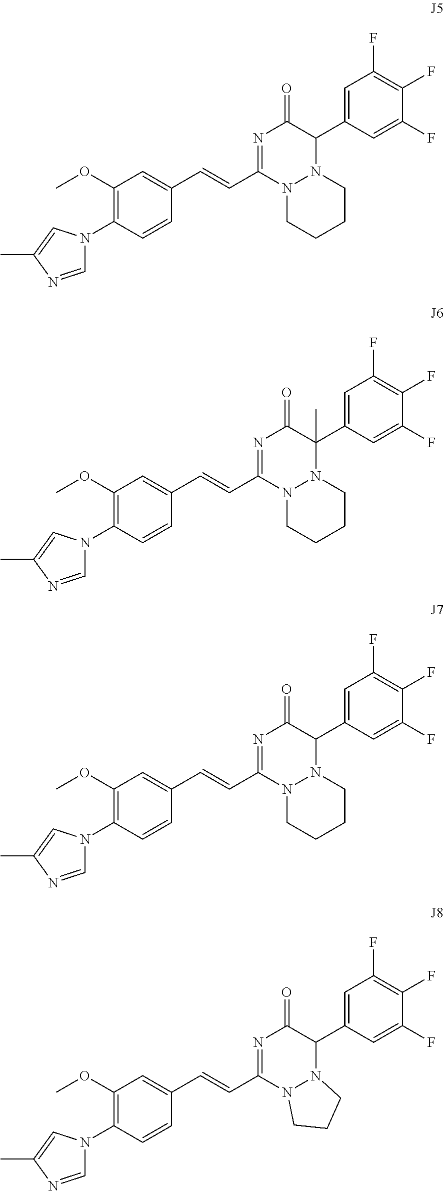 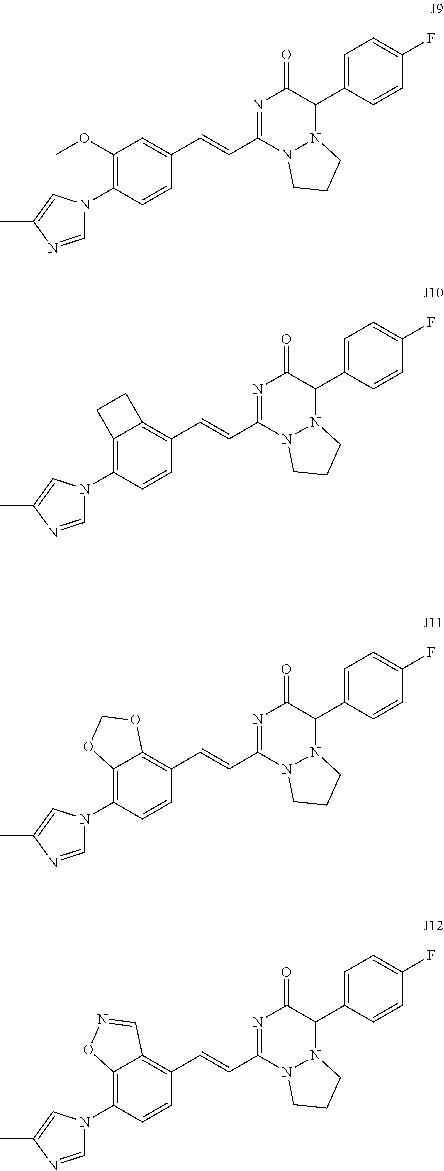 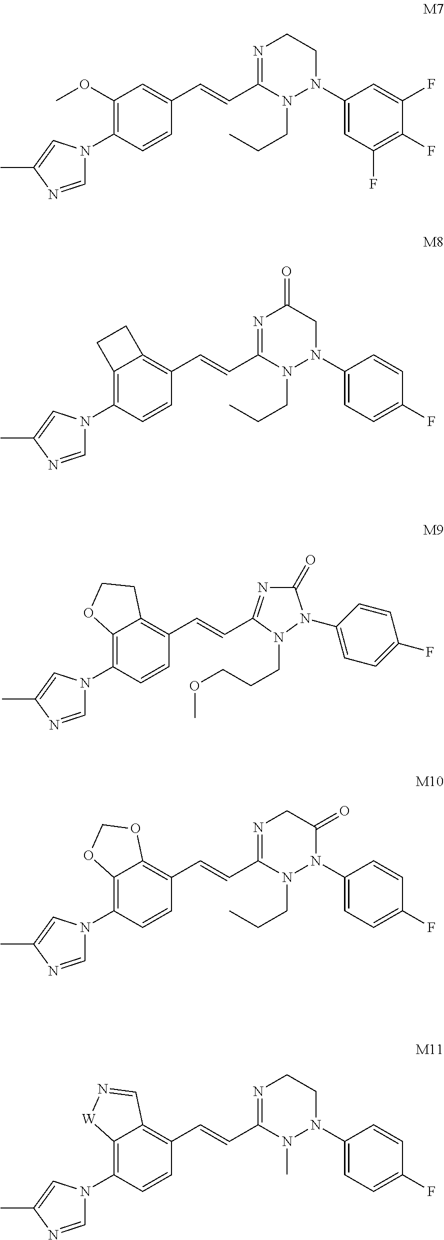 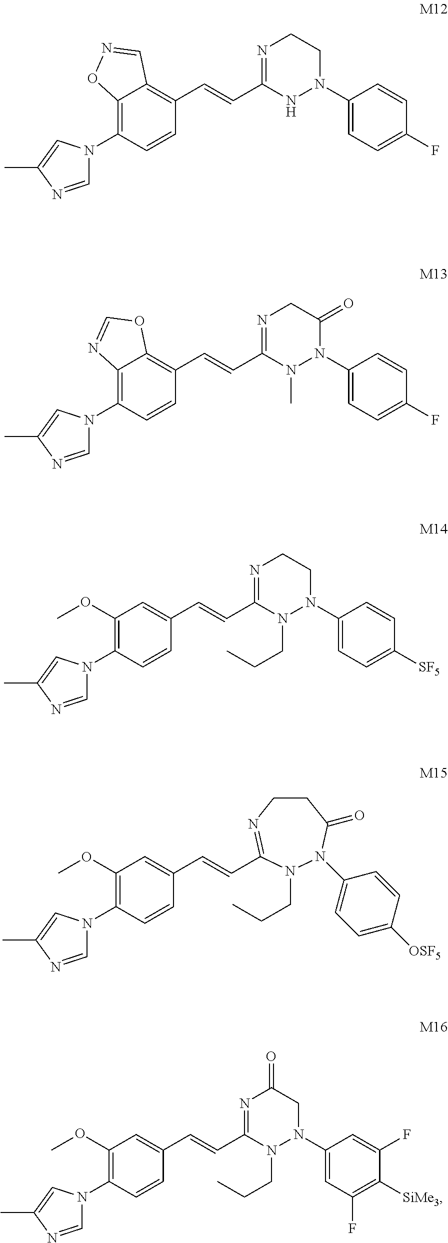
or a pharmaceutically acceptable salt thereof.

35. A pharmaceutical composition comprising a therapeutically effective amount of a compound of claim 21 or a pharmaceutically acceptable salt thereof, and a pharmaceutically acceptable carrier.

36. A method of modulating gamma-secretase comprising administering an effective amount of one or more compounds of claim 21 or a pharmaceutically acceptable salt thereof to a patient in need of such treatment.

37. A method of inhibiting the deposition of amyloid protein in, on or around neurological tissue, comprising administering an effective amount of one or more compounds of claim 21 or a pharmaceutically acceptable salt thereof to a patient in need of treatment.

38. A method of treating Alzheimer's disease, comprising administering an effective amount of one or more compounds of claim 21 or a pharmaceutically acceptable salt thereof to a patient in need of treatment.
